(19) United States
(12) Patent Application Publication
(10) Pub. No.: US 2024/0084354 A1
(43) Pub. Date: Mar. 14, 2024

(54) MEANS AND METHODS FOR REGULATING INTRACELLULAR TRAFFICKING OF SECRETORY OR CELL MEMBRANE-ANCHORED PROTEINS OF INTEREST

(71) Applicants: HONING BIOSCIENCES, Paris (FR); INSTITUT CURIE, Paris (FR); CENTRE NATIONAL DE LA RECHERCHE SCIENTIFIQUE, Paris (FR)

(72) Inventors: Franck PEREZ, Paris (FR); Zelia GOUVEIA, Paris (FR)

(73) Assignees: HONING BIOSCIENCES, Paris (FR); INSTITUT CURIE, Paris (FR); CENTRE NATIONAL DE LA RECHERCHE SCIENTIFIQUE, Paris (FR)

(21) Appl. No.: 18/252,941

(22) PCT Filed: Nov. 15, 2021
(86) PCT No.: PCT/EP2021/081735

§ 371 Date: May 15, 2023

Related U.S. Application Data
(60) Provisional application No. 63/113,452, filed on Nov. 13, 2020.

(30) Foreign Application Priority Data
Dec. 7, 2020 (EP) 20306514.9

(51) Int. Cl. C12P 21/00; C07K 14/36; C07K 14/54; C07K 14/55; C12N 15/85

(52) U.S. Cl. CPC C12P 21/005; C07K 14/36; C07K 14/5434; C07K 14/55; C12N 15/85; C07K 2319/04; C07K 2319/20; C12N 2740/15043; C12N 2830/002

(57) ABSTRACT

The present invention relates to a polynucleotide comprising a gene encoding a hook protein and a gene encoding a protein of interest, said protein of interest being either a secretory protein or a cell membrane-anchored protein, wherein:
said gene encoding the hook protein is under the control of a first transcription-activating signal,
said gene encoding the protein of interest is under the control of a second transcription-activating signal, said second transcription-activating signal allowing a lower rate or frequency of transcription initiation than the first transcription-activating signal,
said hook protein is fused to a cellular compartment-retention peptide, and
said protein of interest is fused to a hook protein-binding domain

It also related to vectors comprising the polynucleotide, cells comprising the polynucleotide or the vector and compositions comprising the same. It further relates to methods and uses for modulating the secretion or cell membrane-anchorage of a protein of interest, or for preventing and/or treating a disease in a subject in need thereof.

### FIELD OF INVENTION

The present invention relates to a polynucleotide comprising a gene encoding a hook protein and a gene encoding a protein of interest, said protein of interest being either a secretory protein or a cell membrane-anchored protein, wherein:
said gene encoding the hook protein is under the control of a first transcription-activating signal,
said gene encoding the protein of interest is under the control of a second transcription-activating signal, said second transcription-activating signal allowing a lower rate or frequency of transcription initiation than the first transcription-activating signal,
said hook protein is fused to a cellular compartment-retention peptide, and
said protein of interest is fused to a hook protein-binding domain.

It also related to vectors comprising the polynucleotide, cells comprising the polynucleotide or the vector and compositions comprising the same. It further relates to methods and uses for modulating the secretion or cell membrane-anchorage of a protein of interest, or for preventing and/or treating a disease in a subject in need thereof.

### BACKGROUND OF INVENTION

There are many routes for proper transport, modification and addressing of proteins in the secretory pathway of cells. A comprehensive view of the mechanisms and dynamics of cargo sorting in these multiple secretory pathways requires assays that allow the dissection of the routes specific cargos follow.

To address secretory traffic issues, several methods have been studies, especially methods that rely on the fusion of the protein of interest with conditional aggregation domains, resulting in the aggregation of the fusion protein in the endoplasmic reticulum (ER). One of such systems, named RUSH (Retention Using Selective Hooks), relies on the selective retention and release of cargo molecules from a donor compartment. RUSH is a two-state assay based on the reversible interaction of a hook protein fused to core streptavidin and stably anchored in the donor compartment with a reporter protein of interest fused to a streptavidin-binding peptide (SBP). Biotin addition causes a synchronous release of the reporter from the hook. The independent expression of the hook and the protein of interest was ensured using a CMV promoter and an IVS-IRES (synthetic intron-internal ribosome entry site) signal (see WO2010142785, also illustrated in FIG. 26A).

Nonetheless, despite a proper expression of this RUSH system in various cell lines, the Inventors have encountered difficulties implementing this system some cells, for instance, in primary cells. This impedes any use of the RUSH system in vivo. There remains thus a need for a system allowing to control the secretion or cell membrane-anchoring of proteins of interest, in particular of therapeutic interest such as, e.g., cytokines, in vivo, in the course of therapy for example, thereby preventing any side effects that could be associated with a systemic administration of said proteins of therapeutic interest in a subject.

The present invention provides an innovative technical solution which overcomes the previously mentioned limitations, as illustrated in FIG. 26B.

### SUMMARY

The present invention relates to a polynucleotide comprising a gene encoding a hook protein and a gene encoding a protein of interest, said protein of interest being either a secretory protein or a cell membrane-anchored protein, wherein:
said gene encoding the hook protein is under the control of a first transcription-activating signal,
said gene encoding the protein of interest is under the control of a second transcription-activating signal, said second transcription-activating signal allowing a lower rate or frequency of transcription initiation than the first transcription-activating signal,
said hook protein is fused to a cellular compartment-retention peptide, and
said protein of interest is fused to a hook protein-binding domain.

According to the invention, the hook protein is one of a pair of proteins comprising the hook protein and the hook protein-binding domain, wherein the hook protein has specific binding affinity for the hook protein-binding domain.

In one embodiment, the hook protein is a biotin-binding protein.

In one embodiment, the first transcription-activating signal is a selected from the group comprising CMV, SFFV, CAG, EF1, EF1A, GAL1, GAL10, GPD, ADH and GAP.

In one embodiment, the second transcription-activating signal is a selected from the group comprising vav, PGK, SV40, thymidine kinase promoter (TK), MSCV and UbC promoter.

In one embodiment, the first transcription-activating signal is a SFFV promoter, and the second transcription-activating signal is selected from the group comprising PGK, SV40, and UbC promoter.

In one embodiment, the cellular compartment-retention peptide is (i) a peptide capable of targeting and/or promoting localization of the hook protein in a cellular compartment or at the cell membrane, (ii) a peptide or peptidic domain capable of interacting with a cellular compartment-resident protein, and/or (ii) a peptide or peptidic domain derived from a transmembrane domain of a protein anchored in the membrane of the cellular compartment or cell membrane.

In one embodiment, the cellular compartment-retention peptide is a peptide or peptidic domain derived from a transmembrane domain of a protein anchored in the membrane of the cellular compartment or cell membrane or of a cellular compartment-resident protein.

In one embodiment, the cellular compartment-retention peptide is selected from the group comprising or consisting of endoplasmic reticulum-retention peptides, Golgi-retention peptides, mitochondrion-retention peptides, nucleus-retention peptides, vesicle-retention peptides and plasma membrane-retention peptides.

In one embodiment, the cellular compartment-retention peptide is an endoplasmic reticulum-retention peptide.

In one embodiment, the endoplasmic reticulum-retention peptide comprises an amino acid sequence selected from the amino acid sequences set forth in Table 1 (SEQ ID NOs: 10 to 38 or a RR, RXR, DXE, DIE, or SKK peptidic motif, wherein X is any amino acid residue), or the endoplasmic reticulum-retention peptide comprises an amino acid sequence of the isoform p33 of the invariant chain, of ribophorin I, of ribophorin II, of a SEC61 subunit, of cytochrome b5 or of a fragment thereof.

In one embodiment, the endoplasmic reticulum-retention peptide comprises a KDEL (SEQ ID NO: 10), K(X)KXX (SEQ ID NO: 17), RR, RXR, or RXXR (SEQ ID NO: 19) peptidic motif, wherein X is any amino acid residue.

In one embodiment, the hook protein is a biotin-binding protein

In one embodiment, the hook protein is a natural or synthetic biotin-binding protein belonging to the avidin-like superfamily.

In one embodiment, the hook protein is a biotin-binding protein selected from the group comprising avidin, streptavidin, tamavidin, bradavidin, rhizavidin, and derivatives thereof.

In one embodiment, the hook protein is a biotin-binding protein selected from the group comprising avidin, streptavidin, tamavidin, bradavidin, rhizavidin, neutravidin, extravidin, captavidin, and traptavidin.

In one embodiment, the hook protein is streptavidin.

In one embodiment, the hook protein-binding domain is a biotin-binding protein-binding protein or peptide or a derivative thereof.

In one embodiment, the hook protein-binding domain comprises an amino acid sequence selected from the amino acid sequences set forth in Table 9 (SEQ ID NOs: 605 to 634, DVE, VEA and EAW).

In one embodiment, the protein of interest is a cytokine.

In one embodiment, the protein of interest is a cytokine selected from the group comprising or consisting of interleukin-12 (IL-12) and interleukin-2 (IL-2).

The present invention also relates to a vector comprising the polynucleotide according to the present invention.

The present invention also relates to a system of at least two polynucleotides, comprising:
a) a first polynucleotide comprising a gene encoding a hook protein, and
b) a second polynucleotide comprising a gene encoding a protein of interest, said protein of interest being either a secretory protein or a cell membrane-anchored protein,
wherein:
   said gene encoding the hook protein is under the control of a first transcription-activating signal,
   said gene encoding the protein of interest is under the control of a second transcription-activating signal, said second transcription-activating signal allowing a lower rate or frequency of transcription initiation than the first transcription-activating signal,
   said hook protein is fused to a cellular compartment-retention peptide, and
   said protein of interest is fused to a hook protein-binding domain;
wherein the hook protein is one of a pair of proteins comprising the hook protein and the hook protein-binding domain, wherein the hook protein has specific binding affinity for the hook protein-binding domain;
preferably wherein the hook protein is a biotin-binding protein.

The present invention also relates to a cell comprising the polynucleotide according to the present invention, the vector according to the present invention, or the system of at least two polynucleotides according to the present invention.

The present invention also relates to a composition comprising the polynucleotide according to the present invention, the vector according to the present invention, the system of at least two polynucleotides according to the present invention, or the cell according to the present invention.

The present invention also relates to a method of modulating the secretion or cell membrane-anchorage of a protein of interest, comprising the steps of:
(a) transducing a cell with the polynucleotide according to the present invention, the vector according to the present invention, or the system of at least two polynucleotides according to the present invention,
(b) having the transduced cell of step (a) express a hook protein fused to a cellular compartment-retention peptide and the protein of interest fused to a hook protein-binding domain, thereby trapping said protein of interest, upon its expression, in said cell to a cellular compartment of the cell, and
(c) contacting said cell with a competing molecule, wherein said competing molecule binds to the hook protein,
thereby releasing said protein of interest from the cellular compartment of the cell and allowing its secretion or cell membrane-anchorage.

The present invention also relates to the polynucleotide according to the present invention, the vector according to the present invention, the system of at least two polynucleotides according to the present invention, the cell according to the present invention, or the composition according to the present invention, for use as a drug.

The present invention also relates to the polynucleotide according to the present invention, the vector according to the present invention, the system of at least two polynucleotides according to the present invention, the cell according to the present invention, or the composition according to the present invention, for use in a method of preventing and/or treating a disease in a subject in need thereof, wherein:
(a) in a first step, the polynucleotide according to the present invention, the vector according to the present invention, the system of at least two polynucleotides according to the present invention, the cell according to the present invention, or the composition according to the present invention is to be administered to the subject, thereby having a cell of the subject expressing a hook protein fused to a cellular compartment-retention peptide and a protein of interest fused to a hook protein-binding domain; and
(b) in a second step, a competing molecule is to be administered to the subject.

In one embodiment, the competing molecule is biotin or a derivative thereof.

In one embodiment, a biotin derivative has a structure of Formula (I):

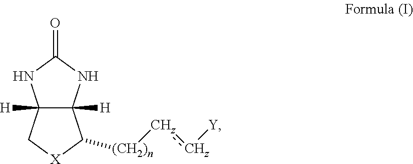

wherein:
X is selected from H₂, O, S, Se, SO, and SO₂,
Y is selected from CONH(CH₂)₄CH(NH₂)COOH, COOH, and OH,
n is 1, 2 or 3, and
z is 1 or 2.

In one embodiment, the biotin derivative is selected from the group consisting of biocytin, dethiobiotin, selenobiotin, biotin sulfoxide, oxybiotin, biotinol, norbiotin, homobiotin, α-dehydrobiotin, and biotin sulfone.

### DETAILED DESCRIPTION

The present invention relates to a polynucleotide comprising a gene encoding a hook protein and a gene encoding a protein of interest, said protein of interest being either a secretory protein or a cell membrane-anchored protein, wherein:
said gene encoding the hook protein is under the control of a first transcription-activating signal,
said gene encoding the protein of interest is under the control of a second transcription-activating signal, said second transcription-activating signal allowing a lower rate or frequency of transcription initiation than the first transcription-activating signal,
said hook protein is fused to a cellular compartment-retention peptide, and
said protein of interest is fused to a hook protein-binding domain.

The present invention also relates to a system of at least two polynucleotides, comprising
a) a first polynucleotide comprising a gene encoding a hook protein, and
b) a second polynucleotide comprising a gene encoding a protein of interest, said protein of interest being either a secretory protein or a cell membrane-anchored protein,
wherein:
   said gene encoding the hook protein is under the control of a first transcription-activating signal,
   said gene encoding the protein of interest is under the control of a second transcription-activating signal, said second transcription-activating signal allowing a lower rate or frequency of transcription initiation than the first transcription-activating signal,
   said hook protein is fused to a cellular compartment-retention peptide, and
   said protein of interest is fused to a hook protein-binding domain.

In the following, when referring to “the polynucleotide”, the term is also intended to encompass the system of at least two polynucleotides.

As used herein, the term “hook protein” refers to a protein capable of retaining a protein of interest containing a corresponding hook protein-binding domain in a cellular compartment by a specific interaction with said hook protein-binding domain fused to the protein of interest. In other words, the hook protein is one of a pair of proteins comprising the hook protein and a hook protein-binding domain, wherein the hook protein has specific binding affinity for the hook protein-binding domain. Hook proteins have been described in the system referred to as RUSH (Retention Using Selective Hooks) in Boncompain et al. (2012. Nat Methods. 9(5):493-8), WO2010142785 and WO201612623.

In one embodiment, the hook protein is a biotin-binding protein or a derivative thereof, wherein said derivative (such as, e.g., a fragment thereof, a variant thereof, an ester thereof, etc.) retains its ability to bind to biotin.

In one embodiment, the hook protein is a biotin-binding protein belonging to the avidin-like superfamily, as defined, e.g., on the InterPro database (Apweiler et al., 2001. Nucleic Acids Res. 29(1):37-40; Blum et al., 2021. Nucleic Acids Res. 49(D1):D344-D354).

In one embodiment, the hook protein is a natural or synthetic biotin-binding protein belonging to the avidin-like superfamily.

In one embodiment, the biotin-binding protein is selected from the group comprising or consisting of avidin, streptavidin, tamavidin, bradavidin, rhizavidin, and derivatives thereof, including any further developed or found equivalent molecule having an appropriate biotin-binding configuration. Non-limiting examples of avidin derivatives include NeutrAvidin™, Extravidin®, CaptAvidin™. Non-limiting examples of streptavidin derivatives include traptavidin (described in Chivers et al., 2010. Nat Methods. 7(5):391-3).

In one embodiment, the biotin-binding protein may be monomeric or oligomeric, either naturally or upon engineering. It is however to be understood that any protein that specifically binds to biotin can be used as a hook protein in the present invention.

As used herein, the term “avidin” refers to a homotetrameric protein produced in the oviducts of birds, reptiles and amphibians, and deposited in the whites of their eggs. An exemplary amino acid sequence of a monomer of avidin comprises or consists of SEQ ID NO: 1, corresponding to version 3 of UniProtKB accession number P02701.

Gallus gallus
SEQ ID NO: 1
MVHATSPLLLLLLLSLALVAPGLSARKCSLTGKWTNDLGSNMTIGAVNSR
GEFTGTYITAVTATSNEIKESPLHGTQNTINKRTQPTFGFTVNWKFSEST
TVFTGQCFIDRNGKEVLKTMWLLRSSVNDIGDDWKATRVGINIFTRLRTQ
KE

As used herein, the term “streptavidin” refers to a homotetrameric protein produced by the bacterium Streptomyces avidinii. An exemplary amino acid sequence of a monomer of streptavidin comprises or consists of SEQ ID NO: 2, corresponding to version 1 of UniProtKB accession number P22629.

Streptomyces avidinii
SEQ ID NO: 2
MRKIVVAAIAVSLTTVSITASASADPSKDSKAQVSAAEAGITGTWYNQLG
STFIVTAGADGALTGTYESAVGNAESRYVLTGRYDSAPATDGSGTALGWT
VAWKNNYRNAHSATTWSGQYVGGAEARINTQWLLTSGTTEANAWKSTLVG
HDTFTKVKPSAASIDAAKKAGVNNGNPLDAVQQ

Low-affinity and high-affinity streptavidin mutants are also encompassed herein. These mutants are well known in the art. Monomeric streptavidin mutants are also encompassed herein. These mutants are well known in the art.

As used herein, the term “tamavidin” refers to two homotetrameric or homodimeric proteins produced by the Tamogitake mushroom Pleurotus cornucopiae, named tamavidin 1 and tamavidin 2. An exemplary amino acid sequence of a monomer of tamavidin 1 comprises or consists of SEQ ID NO: 3, corresponding to version 1 of UniProtKB accession number B9A0T6. An exemplary amino acid sequence of a monomer of tamavidin 2 comprises or consists of SEQ ID NO: 4, corresponding to version 1 of UniProtKB accession number B9A0T7.

Pleurotus cornucopiae
SEQ ID NO: 3
MKDVQSLLTGTWYNELGSTMNLTANKDGSLTGTYHSNVGEVPPTYHLSGR
YNLQPPSGQGVTLGWAVSFENTSANVHSVSTWSGQYFSEPAEVILTQWLL
SRSSEREDLWQSTHVGHDEFSKTKPTKEKIAQAQLLRRGLKFE
Pleurotus cornucopiae
SEQ ID NO: 4
MSDVQSSLTGTWYNELNSKMELTANKDGTLTGKYLSKVGDVYVPYPLSGR
YNLQPPAGQGVALGWAVSWENSKIHSATTWSGQFFSESSPVILTQWLLSS
STARGDVWESTLVGNDSFTKTAPTEQQIAHAQLHCRAPRLK

As used herein, the term “bradavidin” refers to a homotetrameric protein produced by the nitrogen-fixing bacterium Bradyrhizobium diazoefficiens. An exemplary amino acid sequence of a monomer of bradavidin comprises or consists of SEQ ID NO: 5, corresponding to version 1 of UniProtKB accession number Q89IH6.

Bradyrhizobium diazoefficiens
SEQ ID NO: 5
MRHFNGMLLAMIASTSLIGPLPAYAQSVNWTWTNQYGSTLAITSFNSNTG
AITGTYTNNAANSCDEGKPQGVTGWLAYGNTGTAISFSVNFLGCGSTTVW
TGQLNNATGFQGLWYLSLAEAVAWNGISAGADTFTFSSGDKALLTKSGVD
LKAGSEKLSNTKK

As used herein, the term “rhizavidin” refers to a homodimeric protein produced by the nitrogen-fixing bacterium Rhizobium etli. An exemplary amino acid sequence of a monomer of rhizavidin comprises or consists of SEQ ID NO: 6, corresponding to version 2 of UniProtKB accession number Q8KKW2.

Rhizobium etli
SEQ ID NO: 6
MIRTNAVAALVFAVATSALAFDASNFKDFSSIASASSSWQNQSGSTMII
QVDSFGNVSGQYVNRAQGTGCQNSPYPLTGRVNGTFIAFSVGWNNSTEN
CNSATGWTGYAQVNGNNTEIVTSWNLAYEGGSGPAIEQGQDTFQYVPTT
ENKSLLKD

In one embodiment, the biotin-binding protein is streptavidin or a derivative thereof.

As used herein, the terms “NeutrAvidin™” and “Extravidin®” both refer to a chemically deglycosylated form of avidin.

As used herein, the term “CaptAvidin™” refers to a modified form of avidin comprising a nitrated tyrosine in its biotin-binding site.

In one embodiment, the hook protein is a FKBP-binding protein or a derivative thereof, wherein said derivative (such as, e.g., a fragment thereof, a variant thereof, an ester thereof, etc.) retains its ability to bind to FKBP and, preferably, to rapamycin or derivatives thereof.

In one embodiment, the FKBP-binding protein is selected from the group comprising or consisting of mTOR and derivatives thereof.

As used herein, the term “mTOR”, also referred to as “mammalian target of rapamycin”, “FK506-binding protein 12-rapamycin-associated protein”, “FRAP” refers to a phosphatidylinositol 3-kinase-related kinase. An exemplary amino acid sequence of mTOR comprises or consists of SEQ ID NO: 7, corresponding to version 1 of UniProtKB accession number P42345.

Homo sapiens
SEQ ID NO: 7
MLGTGPAAATTAATTSSNVSVLQQFASGLKSRNEETRAKAAKELQHYVT
MELREMSQEESTRFYDQLNHHIFELVSSSDANERKGGILAIASLIGVEG
GNATRIGRFANYLRNLLPSNDPVVMEMASKAIGRLAMAGDTFTAEYVEF
EVKRALEWLGADRNEGRRHAAVLVLRELAISVPTFFFQQVQPFFDNIFV
AVWDPKQAIREGAVAALRACLILTTQREPKEMQKPQWYRHTFEEAEKGF
DETLAKEKGMNRDDRIHGALLILNELVRISSMEGERLREEMEEITQQQL
VHDKYCKDLMGFGTKPRHITPFTSFQAVQPQQSNALVGLLGYSSHQGLM
GFGTSPSPAKSTLVESRCCRDLMEEKFDQVCQWVLKCRNSKNSLIQMTI
LNLLPRLAAFRPSAFTDTQYLQDTMNHVLSCVKKEKERTAAFQALGLLS
VAVRSEFKVYLPRVLDIIRAALPPKDFAHKRQKAMQVDATVFTCISMLA
RAMGPGIQQDIKELLEPMLAVGLSPALTAVLYDLSRQIPQLKKDIQDGL
LKMLSLVLMHKPLRHPGMPKGLAHQLASPGLTTLPEASDVGSITLALRT
LGSFEFEGHSLTQFVRHCADHFLNSEHKEIRMEAARTCSRLLTPSIHLI
SGHAHVVSQTAVQVVADVLSKLLVVGITDPDPDIRYCVLASLDERFDAH
LAQAENLQALFVALNDQVFEIRELAICTVGRLSSMNPAFVMPFLRKMLI
QILTELEHSGIGRIKEQSARMLGHLVSNAPRLIRPYMEPILKALILKLK
DPDPDPNPGVINNVLATIGELAQVSGLEMRKWVDELFIIIMDMLQDSSL
LAKRQVALWTLGQLVASTGYVVEPYRKYPTLLEVLLNFLKTEQNQGTRR
EAIRVLGLLGALDPYKHKVNIGMIDQSRDASAVSLSESKSSQDSSDYST
SEMLVNMGNLPLDEFYPAVSMVALMRIFRDQSLSHHHTMVVQAITFIFK
SLGLKCVQFLPQVMPTFLNVIRVCDGAIREFLFQQLGMLVSFVKSHIRP
YMDEIVTLMREFWVMNTSIQSTIILLIEQIVVALGGEFKLYLPQLIPHM
LRVFMHDNSPGRIVSIKLLAAIQLFGANLDDYLHLLLPPIVKLFDAPEA
PLPSRKAALETVDRLTESLDFTDYASRIIHPIVRTLDQSPELRSTAMDT
LSSLVFQLGKKYQIFIPMVNKVLVRHRINHQRYDVLICRIVKGYTLADE
EEDPLIYQHRMLRSGQGDALASGPVETGPMKKLHVSTINLQKAWGAARR
VSKDDWLEWLRRLSLELLKDSSSPSLRSCWALAQAYNPMARDLFNAAFV
SCWSELNEDQQDELIRSIELALTSQDIAEVTQTLLNLAEFMEHSDKGPL
PLRDDNGIVLLGERAAKCRAYAKALHYKELEFQKGPTPAILESLISINN
KLQQPEAAAGVLEYAMKHFGELEIQATWYEKLHEWEDALVAYDKKMDTN
KDDPELMLGRMRCLEALGEWGQLHQQCCEKWTLVNDETQAKMARMAAAA
AWGLGQWDSMEEYTCMIPRDTHDGAFYRAVLALHQDLFSLAQQCIDKAR
DLLDAELTAMAGESYSRAYGAMVSCHMLSELEEVIQYKLVPERREIIRQ
IWWERLQGCQRIVEDWQKILMVRSLVVSPHEDMRTWLKYASLCGKSGRL
ALAHKTLVLLLGVDPSRQLDHPLPTVHPQVTYAYMKNMWKSARKIDAFQ
HMQHFVQTMQQQAQHAIATEDQQHKQELHKLMARCFLKLGEWQLNLQGI
NESTIPKVLQYYSAATEHDRSWYKAWHAWAVMNFEAVLHYKHQNQARDE
KKKLRHASGANITNATTAATTAATATTTASTEGSNSESEAESTENSPTP
SPLQKKVTEDLSKTLLMYTVPAVQGFFRSISLSRGNNLQDTLRVLTLWF
DYGHWPDVNEALVEGVKAIQIDTWLQVIPQLIARIDTPRPLVGRLIHQL
LTDIGRYHPQALIYPLTVASKSTTTARHNAANKILKNMCEHSNTLVQQA
MMVSEELIRVAILWHEMWHEGLEEASRLYFGERNVKGMFEVLEPLHAMM
ERGPQTLKETSFNQAYGRDLMEAQEWCRKYMKSGNVKDLTQAWDLYYHV
FRRISKQLPQLTSLELQYVSPKLLMCRDLELAVPGTYDPNQPIIRIQSI
APSLQVITSKQRPRKLTLMGSNGHEFVFLLKGHEDLRQDERVMQLFGLV
NTLLANDPTSLRKNLSIQRYAVIPLSTNSGLIGWVPHCDTLHALIRDYR
EKKKILLNIEHRIMLRMAPDYDHLTLMQKVEVFEHAVNNTAGDDLAKLL
WLKSPSSEVWFDRRTNYTRSLAVMSMVGYILGLGDRHPSNLMLDRLSGK
ILHIDFGDCFEVAMTREKFPEKIPFRLTRMLTNAMEVTGLDGNYRITCH
TVMEVLREHKDSVMAVLEAFVYDPLLNWRLMDTNTKGNKRSRTRTDSYS
AGQSVEILDGVELGEPAHKKTGTTVPESIHSFIGDGLVKPEALNKKAIQ
IINRVRDKLTGRDFSHDDTLDVPTQVELLIKQATSHENLCQCYIGWCPF
W

Non-limiting examples of mTOR derivatives include the non-specific serine/threonine protein kinase, an exemplary amino acid sequence of which comprises or consists of SEQ ID NO: 8, corresponding to version 1 of UniProtKB accession number B1AKP8.

Homo sapiens
SEQ ID NO: 8
MNFEAVLHYKHQNQARDEKKKLRHASGANITNATTAATTAATATTTAST
EGSNSESEAESTENSPTPSPLQKKVTEDLSKTLLMYTVPAVQGFFRSIS
LSRGNNLQDTLRVLTLWFDYGHWPDVNEALVEGVKAIQIDTWLQVIPQL
IARIDTPRPLVGRLIHQLLTDIGRYHPQALIYPLTVASKSTTTARHNAA
NKILKNMCEHSNTLVQQAMMVSEELIRVAILWHEMWHEGLEEASRLYFG
ERNVKGMFEVLEPLHAMMERGPQTLKETSFNQAYGRDLMEAQEWCRKYM
KSGNVKDLTQAWDLYYHVFRRISKQLPQLTSLELQYVSPKLLMCRDLEL
AVPGTYDPNQPIIRIQSIAPSLQVITSKQRPRKLTLMGSNGHEFVFLLK
GHEDLRQDERVMQLFGLVNTLLANDPTSLRKNLSIQRYAVIPLSTNSGL
IGWVPHCDTLHALIRDYREKKKILLNIEHRIMLRMAPDYDHLTLMQKVE
VFEHAVNNTAGDDLAKLLWLKSPSSEVWFDRRTNYTRSLAVMSMVGYIL
GLGDRHPSNLMLDRLSGKILHIDFGDCFEVAMTREKFPEKIPFRLTRML
TNAMEVTGLDGNYRITCHTVMEVLREHKDSVMAVLEAFVYDPLLNWRLM
DTNTKGNKRSRTRTDSYSAGQSVEILDGVELGEPAHKKTGTTVPESIHS
FIGDGLVKPEALNKKAIQIINRVRDKLTGRDFSHDDTLDVPTQVELLIK
QATSHENLCQCYIGWCPFW

mTOR with SEQ ID NO: 7 and its derivative with SEQ ID NO: 8 comprise in particular a FKBP-binding domain, also capable of interacting with rapamycin or derivatives thereof. This FKBP-binding domain corresponds to amino acid residues 2012 to 2144 of SEQ ID NO: 7 or amino acid residues 217 to 349 of SEQ ID NO: 8, set forth in SEQ ID NO: 9. In one embodiment, the FKBP-binding protein or the derivative thereof comprises or consists of this FKBP-binding domain.

SEQ ID NO: 9
VSEELIRVAILWHEMWHEGLEEASRLYFGERNVKGMFEVLEPLHAMMER
GPQTLKETSFNQAYGRDLMEAQEWCRKYMKSGNVKDLTQAWDLYYHVFR
RISKQLPQLTSLELQYVSPKLLMCRDLELAVPGTY

The term “derivative”, when referring to a protein, includes homologs, fragments, mutants and combinations thereof.

As used herein, the term “homolog”, when referring to a protein, refers to a distinct protein from another family or species which is determined by functional, structural or genomic analyses to correspond to the original protein. Most often, homologs will have functional, structural, or genomic similarities. Techniques are known by which homologs of a protein can readily be cloned using genetic probes and PCR. The identity of cloned sequences as homologous can be confirmed using functional assays and/or by genomic mapping of the genes.

As used herein, the term “fragment”, when referring to a protein, refers to a portion of the protein retaining the same or substantially the same biological function, activity and/or local structure, with respect to the specific biological function, activity and/or local structure identified for the full-length protein. A skilled person will understand that the term encompasses peptides of any origin which have a sequence corresponding to the portion of the protein.

As used herein, the term “mutant”, when referring to a protein, refers to a protein in which one or more amino acids have been altered. Such alterations include addition and/or substitution and/or deletion and/or insertion of one or several amino acid residues at the N-terminal extremity, and/or the C-terminal extremity, and/or within the amino acid sequence of the protein. Preferably, a “mutant” has an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity or more to the amino acid sequence of the original protein or derivative thereof. Sequence identity can be global sequence identity or local sequence identity; preferably sequence identity is global sequence identity.

As used herein, the term “sequence identity” refers to the number of identical or similar amino acids in a comparison between a test and a reference protein or derivative thereof.

Sequence identity can be determined by sequence alignment of protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps, as follows:

$$sequence identity = \frac{number of identical positions}{lengths of the total aligned sequence} \times 100$$

As used herein, a “global alignment” is an alignment that aligns two sequences from beginning to end, aligning each letter in each sequence only once. An alignment is produced, regardless of whether or not there is similarity or identity between the sequences. For example, 60% sequence identity based on global alignment means that in an alignment of the full sequence of two compared sequences, each of 100 nucleotides in length, 60% of the residues are the same. It is understood that global alignment can also be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman & Wunsch, 1970. J Mol Biol. 48(3):443-53). Exemplary programs and software for performing global alignment are publicly available and include the Global Sequence Alignment Tool available at the National Center for Biotechnology Information (NCBI) website (http://ncbi.nlm.nih.gov), and the program available at http://deepc2.psi.iastate.edu/aat/align/align.html. A “global alignment” determines a “global sequence identity”.

As used herein, a “local alignment” is an alignment that aligns two sequence, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4):482-9). For example, 60% sequence identity based on “local alignment” means that in an alignment of the full sequence of two compared sequences of any length, a region of similarity or identity of 100 nucleotides in length has 60% of the residues that are the same in the region of similarity or identity. A “local alignment” determines a “local sequence identity”.

For purposes herein, sequence identity can be determined by standard alignment algorithm programs used with default gap penalties established by each supplier. Default parameters for the GAP program can include:
(1) a unary comparison matrix (containing a value of 1 for identities and 0 for non-identities) and the weighted comparison matrix of Gribskov & Burgess (1986. Nucleic Acids Res. 14(16):6745-63), as described by Schwartz & Dayhoff (1979. Matrices for detecting distant relationships. In Dayhoff (Ed.), Atlas of protein sequences. 5:353-358. Washington, DC: National Biomedical Research Foundation);
(2) a penalty of 3.0 for each gap and an additional 0.10 penalty for each symbol in each gap; and
(3) no penalty for end gaps.

Whether any two proteins have amino acid sequences that are at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more “identical”, or other similar variations reciting a percent identity, can be determined using known computer algorithms based on local or global alignment (see, e.g., wikipedia.org/wiki/Sequence_alignment_software, providing links to dozens of known and publicly available alignment databases and programs).

Generally, for purposes herein, sequence identity is determined using computer algorithms based on global alignment, such as the Needleman-Wunsch Global Sequence Alignment tool available from NCBI/BLAST (http://blast.ncbi.nlm.nih.gov/Blast.cgi?Web&Page_BlastHome); LAlign (William Pearson implementing the Huang and Miller algorithm [Huang & Miller, 1991. Adv Appl Math. 12(3):337-57); and program from Xiaoqui Huang available at http://deepc2.psi.iastate.edu/aat/align/align.html.

Typically, the full-length sequence of each of the compared proteins is aligned across the full-length of each sequence in a global alignment. Local alignment also can be used when the sequences being compared are substantially the same length.

Therefore, as used herein, the term “identity” represents a comparison or alignment between a test and a reference protein or derivative thereof.

In one exemplary embodiment, “at least 60% of sequence identity” refers to percent identities from 60 to 100% relative to the reference protein or derivative thereof. Identity at a level of 60% or more is indicative of the fact that, assuming for exemplification purposes a test and reference protein or derivative thereof length of 100 amino acids are compared, no more than 40% (i.e., 40 out of 100) of amino acids in the test protein differ from those of the reference protein or derivative thereof. Such differences can be represented as point mutations randomly distributed over the entire length of an amino acid sequence or they can be clustered in one or more locations of varying length up to the maximum allowable, e.g., 40/100 amino acid difference (approximately 60% identity). Differences can also be due to deletions or truncations of amino acid residues. Differences are defined as amino acid substitutions, insertions or deletions. Depending on the length of the compared sequences, at the level of homologies or identities above about 85-90%, the result can be independent of the program and gap parameters set; such high levels of identity can be assessed readily, often without relying on software.

According to the invention, the hook protein is fused to a cellular compartment-retention peptide.

As used herein, the term “cellular compartment-retention peptide” refers to any protein, protein domain or peptide which is resident of a cellular compartment. The term “resident” is intended to mean that said protein, protein domain or peptide is in majority located in the given cellular compartment. Typically, at least 70%, preferably at least 75%, 80%, 85%, 90%, 95%, 96%, 97%, 98%, 99% or more of said protein, protein domain or peptide is located in said cellular compartment at steady-state in a host cell.

In one embodiment, the cellular compartment-retention peptide is a peptide capable of targeting and/or promoting localization of the hook protein in a cellular compartment or at the cell membrane. In one embodiment, the cellular compartment-retention peptide is a peptide or peptidic domain capable of interacting with a cellular compartment-resident protein. In one embodiment, the cellular compartment-retention peptide is a peptide or peptidic domain derived from a transmembrane domain of a protein anchored in the membrane of the cellular compartment or cell membrane. In one embodiment, the cellular compartment-retention peptide is a peptide or peptidic domain of a protein derived from a cellular compartment-resident protein.

Examples of cellular compartments include, but are not limited to, the endoplasmic reticulum (ER), the ER-Golgi intermediate compartment (ERGIC), the Golgi apparatus, the mitochondrion, the nucleus, vesicles and cell membrane. In particular, vesicles include those involved in protein degradation mechanisms, such as, e.g., peroxisomes and lysosomes; transport vesicles, involved in material transport between cellular compartments; secretory vesicles, involved in material excretion from the cell; and extracellular vesicles, such as, e.g., exosomes, ectosomes and microvesicles.

In one embodiment, the cellular compartment-retention peptide is selected from the group comprising or consisting of endoplasmic reticulum-retention peptides, Golgi-retention peptides, mitochondrion-retention peptides, nucleus-retention peptides, vesicle-retention peptides and plasma membrane-retention peptides.

In one embodiment, the endoplasmic reticulum-retention peptide comprises or consists of any amino acid sequence set forth in Table 1.

TABLE 1
ENDOPLASMIC RETICULUM-RETENTION PEPTIDES

| SEQ | |
|---|---|
| ID NO: | Amino acid sequence |
| 10 | KDEL |
| 11 | KTEL |
| 12 | HNEL |
| 13 | KEDL |
| 14 | HDEL |
| 15 | KEEL |
| 16 | QEDL |
| 17 | K(X)KXX, wherein X is any amino acid |
| | residue and parenthesis indicate |
| | presence or absence. |
| 18 | HKXK, wherein X is any amino acid residue |
| — | RR, wherein X is any amino acid residue |
| — | RXR, wherein X is any amino acid residue |
| 19 | RXXR, wherein X is any amino acid residue |
| 20 | EFFDAXE, wherein X is any amino acid |
| | residue |
| — | DXE, wherein X is any amino acid residue |
| — | DIE |
| — | SKK |
| 21 | MAGARRGWRLAPVRRGVCGPRARPLMRPLWLLFAVSFFGW |
| | TGALDG |
| 22 | MGSLWSKISQLFVDA |
| 23 | MRVKEKYQHLWRWGWRWGTMLLGMLMIC |
| 24 | MRLSWFRVLTVLSICLSAVAT |
| 25 | MDPVVVLGLCLSCLLLLSLWKQSYGGGKL |
| 26 | HALSYWKPFLVNMCVATVLTAGAYLCYRFLFNSNT |
| 27 | SPRGSRPSWGPTDPRRRSRNLGKVIDTLTCGFADLMGYIPL |
| | VGA |
| 28 | MALWMRFLPLLALLVLWEPKPAQA |
| 29 | MEAMWLLCVALAVLAWG |
| 30 | IPHDLCHNGEKSKKPSKIKSLFKKKSK |
| 31 | RPSNNNTTTTTTTDATQPHHHHHHHRHRDAGVKNVINNSKT |
| | TESSSSSSAAKEKPKHKKGLLHKLKKKL |
| 32 | VVQAITFIFKSLGLKCVQFLPQVMPTFLNVIRVCDGAIRE |
| 33 | TFCSTALLITALALVCTLLYL |
| 34 | GVMLGSIFCALITMLGHI |
| 35 | FLAAFVVMLCITIVTIVVYIYLV |
| 36 | WGVLAGIAYFSMVGNWAKVLVVLLLFAGV |
| 37 | TAPASINTPN |
| 38 | WWQLTLGAICALPLAGLLACCA |

In one embodiment, the endoplasmic reticulum-retention peptide comprises or consists of a KDEL (SEQ ID NO: 10), K(X)KXX (SEQ ID NO: 17), RR, RXR, or RXXR (SEQ ID NO: 19) peptidic motif, wherein X is any amino acid residue.

In one embodiment, the endoplasmic reticulum-retention peptide comprises or consists of the isoform p33 of the invariant chain (Ii), or of a fragment thereof comprising its endoplasmic reticulum signal peptide.

As used herein, the term “invariant chain” or “Ii”, also referred to as “HLA class II histocompatibility antigen gamma chain” or “CD74”, is a protein that resides in the luminal side of endoplasmic reticulum membrane. An exemplary amino acid sequence of the p33 isoform of Ii comprises or consists of SEQ ID NO: 39, corresponding to version 3 of UniProtKB accession number P04233-1.

Homo sapiens
SEQ ID NO: 39
MHRRRSRSCREDQKPVMDDQRDLISNNEQLPMLGRRPGAPESKCSRGAL
YTGFSILVTLLLAGQATTAYFLYQQQGRLDKLTVTSQNLQLENLRMKLP
KPPKPVSKMRMATPLLMQALPMGALPQGPMQNATKYGNMTEDHVMHLLQ
NADPLKVYPPLKGSFPENLRHLKNTMETIDWKVFESWMHHWLLFEMSRH
SLEQKPTDAPPKVLTKCQEEVSHIPAVHPGSFRPKCDENGNYLPLQCYG
SIGYCWCVFPNGTEVPNTRSRGHHNCSESLELEDPSSGLGVTKQDLGPV
PM

In one embodiment, the endoplasmic reticulum-retention peptide comprises or consists of ribophorin I or II, or of a fragment thereof comprising their endoplasmic reticulum signal peptide.

As used herein, the term “ribophorin I”, also referred to as “Dolichyl-diphosphooligosaccharide-protein glycosyltransferase subunit 1”, is an endoplasmic reticulum-specific protein that mediates nascent protein translocation across the endoplasmic reticulum. An exemplary amino acid sequence of ribophorin I comprises or consists of SEQ ID NO: 40, corresponding to version 1 of UniProtKB accession number P04843. The endoplasmic reticulum signal peptide of ribophorin I comprises or consists of amino acid residues 1 to 23 of SEQ ID NO: 40, set forth in SEQ ID NO: 41.

Homo sapiens
SEQ ID NO: 40
MEAPAAGLFLLLLLGTWAPAPGSASSEAPPLINEDVKRTVDLSSHLAKV
TAEVVLAHLGGGSTSRATSFLLALEPELEARLAHLGVQVKGEDEEENNL
EVRETKIKGKSGRFFTVKLPVALDPGAKISVIVETVYTHVLHPYPTQIT
QSEKQFVVFEGNHYFYSPYPTKTQTMRVKLASRNVESYTKLGNPTRSED
LLDYGPFRDVPAYSQDTFKVHYENNSPFLTITSMTRVIEVSHWGNIAVE
ENVDLKHTGAVLKGPFSRYDYQRQPDSGISSIRSFKTILPAAAQDVYYR
DEIGNVSTSHLLILDDSVEMEIRPRFPLFGGWKTHYIVGYNLPSYEYLY
NLGDQYALKMRFVDHVFDEQVIDSLTVKIILPEGAKNIEIDSPYEISRA
PDELHYTYLDTFGRPVIVAYKKNLVEQHIQDIVVHYTFNKVLMLQEPLL
VVAAFYILFFTVIIYVRLDFSITKDPAAEARMKVACITEQVLTLVNKRI
GLYRHFDETVNRYKQSRDISTLNSGKKSLETEHKALTSEIALLQSRLKT
EGSDLCDRVSEMQKLDAQVKELVLKSAVEAERLVAGKLKKDTYIENEKL
ISGKRQELVTKIDHILDAL
SEQ ID NO: 41
MEAPAAGLFLLLLLGTWAPAPGS

As used herein, the term “ribophorin II”, also referred to as “Dolichyl-diphosphooligosaccharide-protein glycosyltransferase subunit 2”, is an endoplasmic reticulum-specific protein that mediates nascent protein translocation across the endoplasmic reticulum. An exemplary amino acid sequence of ribophorin II comprises or consists of SEQ ID NO: 42, corresponding to version 3 of UniProtKB accession number P04844. The endoplasmic reticulum signal peptide of ribophorin I comprises or consists of amino acid residues 1 to 22 of SEQ ID NO: 42, set forth in SEQ ID NO: 43.

Homo sapiens
SEQ ID NO: 42
MAPPGSSTVFLLALTIIASTWALTPTHYLTKHDVERLKASLDRPFTNLE
SAFYSIVGLSSLGAQVPDAKKACTYIRSNLDPSNVDSLFYAAQASQALS
GCEISISNETKDLLLAAVSEDSSVTQIYHAVAALSGFGLPLASQEALSA
LTARLSKEETVLATVQALQTASHLSQQADLRSIVEEIEDLVARLDELGG
VYLQFEEGLETTALFVAATYKLMDHVGTEPSIKEDQVIQLMNAIFSKKN
FESLSEAFSVASAAAVLSHNRYHVPVVVVPEGSASDTHEQAILRLQVTN
VLSQPLTQATVKLEHAKSVASRATVLQKTSFTPVGDVFELNFMNVKFSS
GYYDFLVEVEGDNRYIANTVELRVKISTEVGITNVDLSTVDKDQSIAPK
TTRVTYPAKAKGTFIADSHQNFALFFQLVDVNTGAELTPHQTFVRLHNQ
KTGQEVVFVAEPDNKNVYKFELDTSERKIEFDSASGTYTLYLIIGDATL
KNPILWNVADVVIKFPEEEAPSTVLSQNLFTPKQEIQHLFREPEKRPPT
VVSNTFTALILSPLLLLFALWIRIGANVSNFTFAPSTIIFHLGHAAMLG
LMYVYWTQLNMFQTLKYLAILGSVTFLAGNRMLAQQAVKRTAH
SEQ ID NO: 43
MAPPGSSTVFLLALTIIASTWA

In one embodiment, the endoplasmic reticulum-retention peptide comprises or consists of a SEC61 subunit, or of a fragment thereof comprising its endoplasmic reticulum signal peptide.

As used herein, the term “SEC61” refers to an endoplasmic reticulum-gated pore translocon complex, comprising three subunits: Sec61α, Sec61β and Sec61γ. An exemplary amino acid sequence of Sec61α comprises or consists of SEQ ID NO: 44, corresponding to version 2 of UniProtKB accession number P61619. An exemplary amino acid sequence of Sec61β comprises or consists of SEQ ID NO: 45, corresponding to version 2 of UniProtKB accession number P60468. An exemplary amino acid sequence of Sec61γ comprises or consists of SEQ ID NO: 46, corresponding to version 1 of UniProtKB accession number P60059.

Homo sapiens
SEQ ID NO: 44
MAIKFLEVIKPFCVILPEIQKPERKIQFKEKVLWTAITLFIFLVCCQIP
LFGIMSSDSADPFYWMRVILASNRGTLMELGISPIVTSGLIMQLLAGAK
IIEVGDTPKDRALFNGAQKLFGMIITIGQSIVYVMTGMYGDPSEMGAGI
CLLITIQLFVAGLIVLLLDELLQKGYGLGSGISLFIATNICETIVWKAF
SPTTVNTGRGMEFEGAIIALFHLLATRTDKVRALREAFYRQNLPNLMNL
IATIFVFAVVIYFQGFRVDLPIKSARYRGQYNTYPIKLFYTSNIPIILQ
SALVSNLYVISQMLSARFSGNLLVSLLGTWSDTSSGGPARAYPVGGLCY
YLSPPESFGSVLEDPVHAVVYIVFMLGSCAFFSKTWIEVSGSSAKDVAK
QLKEQQMVMRGHRETSMVHELNRYIPTAAAFGGLCIGALSVLADFLGAI
GSGTGILLAVTIIYQYFEIFVKEQSEVGSMGALLF
Homo sapiens
SEQ ID NO: 45
MPGPTPSGTNVGSSGRSPSKAVAARAAGSTVRQRKNASCGTRSAGRTTS
AGTGGMWRFYTEDSPGLKVGPVPVLVMSLLFIASVFMLHIWGKYTRS
Homo sapiens
SEQ ID NO: 46
MDQVMQFVEPSRQFVKDSIRLVKRCTKPDRKEFQKIAMATAIGFAIMGF
IGFFVKLIHIPINNIIVGG

In one embodiment, the endoplasmic reticulum-retention peptide comprises or consists of cytochrome b5, or of a fragment thereof comprising its endoplasmic reticulum transmembrane domain.

As used herein, the term “cytochrome b5” refers to a tail-anchored protein of the endoplasmic reticulum. An exemplary amino acid sequence of cytochrome b5 comprises or consists of SEQ ID NO: 47, corresponding to version 2 of UniProtKB accession number P00167. The endoplasmic reticulum transmembrane domain of cytochrome b5 comprises or consists of amino acid residues 109 to 131 of SEQ I) NO: 47, set forth in SEQ ID NO: 48.

Homo sapiens
SEQ ID NO: 47
MAEQSDEAVKYYTLEEIQKHNHSKSTWLILHHKVYDLTKFLEEHPGGEE
VLREQAGGDATENFEDVGHSTDAREMSKTFIIGELHPDDRPKLNKPPET
LITTIDSSSSWWTNWVIPAISAVAVALMYRLYMAED
SEQ ID NO: 48
WWTNWVIPAISAVAVALMYRLYM

In one embodiment, the Golgi-retention peptide comprises or consists of any amino acid sequence set forth in Table 2.

TABLE 2
GOLGI-RETENTION PEPTIDES

| SEQ | |
|---|---|
| ID NO: | Amino acid sequence |
| — | KXD, wherein X is any amino acid residue |
| — | KXE, wherein X is any amino acid residue |
| 49 | YXXXXLL, wherein X is any amino acid |
| | residue |
| 50 | LXXKN, wherein X is any amino acid residue |
| 51 | YKGL |
| 52 | YAGL |
| 53 | YKNL |
| 54 | YEGL |
| 55 | LLWKN |
| 56 | QVSAPA |
| 57 | CPSDSEEDEG |
| 58 | DDFEDSESTDTEEE |
| 59 | MAKTAMAYKEKMKELSMLSLICSCFYPEPRNINI |
| 60 | MASPGSGFWSFGSEDGSGDSENPGTAR |
| 61 | RLLDSSNATWQRDQPDTHRLSRLDAHVMSM |
| 62 | WEKIMXXFF, wherein X is any amino acid |
| | residue |
| 63 | MKFREPLLGGSAAMPGASLQRACRLLVAVCALHLGVTLVYY |
| | LA |

In one embodiment, the Golgi-retention peptide comprises or consists of a KXD or KXE peptidic motif, wherein X is any amino acid residue.

In one embodiment, the Golgi-retention peptide comprises or consists of golgin-84, or of a fragment thereof comprising its Golgi transmembrane domain.

As used herein, the term “golgin-84”, also referred to as “golgin subfamily A member 5”, refers to a protein found in the Golgi cisternae and in the tubulo-vesicular structures of the cis-Golgi network. An exemplary amino acid sequence of golgin-84 comprises or consists of SEQ ID NO: 64, corresponding to version 3 of UniProtKB accession number Q8TBA6. The Golgi transmembrane domain of golgin-84 comprises or consists of amino acid residues 699 to 719 of SEQ ID NO: 64, set forth in SEQ ID NO: 65.

Homo sapiens
SEQ ID NO: 64
MSWFVDLAGKAEDLLNRVDQGAATALSRKDNASNIYSKNTDYTELHQQ
NTDLIYQTGPKSTYISSAADNIRNQKATILAGTANVKVGSRTPVEASH
PVENASVPRPSSHFVRRKKSEPDDELLFDFLNSSQKEPTGRVEIRKEK
GKTPVFQSSQTSSVSSVNPSVTTIKTIEENSFGSQTHEAASNSDSSHE
GQEESSKENVSSNAACPDHTPTPNDDGKSHELSNLRLENQLLRNEVQS
LNQEMASLLORSKETQEELNKARARVEKWNADHSKSDRMTRGLRAQVD
DLTEAVAAKDSQLAVLKVRLQEADQLLSTRTEALEALQSEKSRIMQDQ
SEGNSLQNQALQTFQERLHEADATLKREQESYKQMQSEFAARLNKVEM
ERQNLAEAITLAERKYSDEKKRVDELQQQVKLYKLNLESSKQELIDYK
QKATRILQSKEKLINSLKEGSGFEGLDSSTASSMELEELRHEKEMQRE
EIQKLMGQIHQLRSELQDMEAQQVNEAESAREQLQDLHDQIAGQKASK
QELETELERLKQEFHYIEEDLYRTKNTLQSRIKDRDEEIQKLRNQLTN
KTLSNSSQSELENRLHQLTETLIQKQTMLESLSTEKNSLVFQLERLEQ
QMNSASGSSSNGSSINMSGIDNGEGTRLRNVPVLFNDTETNLAGMYGK
VRKAASSIDQFSIRLGIFLRRYPIARVFVIIYMALLHLWVMIVLLTYT
PEMHHDQPYGK
SEQ ID NO: 65
VFVIIYMALLHLWVMIVLLTY

In one embodiment, the endoplasmic reticulum-retention peptide comprises or consists of giantin, or of a fragment thereof comprising its Golgi transmembrane domain.

As used herein, the term “giantin”, also referred to as “Golgin subfamily B member 1”, refers to a protein forming intercisternal cross-bridges of the Golgi. An exemplary amino acid sequence of giantin comprises or consists of SEQ ID NO: 6, corresponding to version 2 of UniProtKB accession number Q14789. The Golgi transmembrane domain of giantin comprises or consists of amino acid residues 3236 to 3256 of SEQ ID NO: 6, set forth in SEQ ID NO: 67.

Homo sapiens
SEQ ID NO: 66
MLSRLSGLANVVLHELSGDDDTDQNMRAPLDPELHQESDMEFNNTTQE
DVQERLAYAEQLVVELKDIIRQKDVQLQQKDEALQEERKAADNKIKKL
KLHAKAKLTSLNKYIEEMKAQGGTVLPTEPQSEEQLSKHDKSSTEEEM
EIEKIKHKLQEKEELISTLQAQLTQAQAEQPAQSSTEMEEFVMMKQQL
QEKEEFISTLQAQLSQTQAEQAAQQVVREKDARFETQVRLHEDELLQL
VTQADVETEMQQKLRVLQRKLEEHEESLVGRAQVVDLLQQELTAAEQR
NQILSQQLQQMEAEHNTLRNTVETEREESKILLEKMELEVAERKLSFH
NLQEEMHHLLEQFEQAGQAQAELESRYSALEQKHKAEMEEKTSHILSL
QKTGQELQSACDALKDQNSKLLQDKNEQAVQSAQTIQQLEDQLQQKSK
EISQFLNRLPLQQHETASQTSFPDVYNEGTQAVTEENIASLQKRVVEL
ENEKGALLLSSIELEELKAENEKLSSQITLLEAQNRTGEADREVSEIS
IVDIANKRSSSAEESGQDVLENTFSQKHKELSVLLLEMKEAQEEIAFL
KLQLQGKRAEEADHEVLDQKEMKQMEGEGIAPIKMKVFLEDTGQDFPL
MPNEESSLPAVEKEQASTEHQSRTSEEISLNDAGVELKSTKQDGDKSL
SAVPDIGQCHQDELERLKSQILELELNFHKAQEIYEKNLDEKAKEISN
LNQLIEEFKKNADNNSSAFTALSEERDQLLSQVKELSMVTELRAQVKQ
LEMNLAEAERQRRLDYESQTAHDNLLTEQIHSLSIEAKSKDVKIEVLQ
NELDDVQLQFSEQSTLIRSLQSQLQNKESEVLEGAERVRHISSKVEEL
SQALSQKELEITKMDQLLLEKKRDVETLQQTIEEKDQQVTEISFSMTE
KMVQLNEEKFSLGVEIKTLKEQLNLLSRAEEAKKEQVEEDNEVSSGLK
QNYDEMSPAGQISKEELQHEFDLLKKENEQRKRKLQAALINRKELLQR
VSRLEEELANLKDESKKEIPLSETERGEVEEDKENKEYSEKCVTSKCQ
EIEIYLKQTISEKEVELQHIRKDLEEKLAAEEQFQALVKQMNQTLQDK
TNQIDLLQAEISENQAIIQKLITSNTDASDGDSVALVKETVVISPPCT
GSSEHWKPELEEKILALEKEKEQLQKKLQEALTSRKAILKKAQEKERH
LREELKQQKDDYNRLQEQFDEQSKENENIGDQLRQLQIQVRESIDGKL
PSTDQQESCSSTPGLEEPLFKATEQHHTQPVLESNLCPDWPSHSEDAS
ALQGGTSVAQIKAQLKEIEAEKVELELKVSSTTSELTKKSEEVFQLQE
QINKQGLEIESLKTVSHEAEVHAESLQQKLESSQLQIAGLEHLRELQP
KLDELQKLISKKEEDVSYLSGQLSEKEAALTKIQTEIIEQEDLIKALH
TQLEMQAKEHDERIKQLQVELCEMKQKPEEIGEESRAKQQIQRKLQAA
LISRKEALKENKSLQEELSLARGTIERLTKSLADVESQVSAQNKEKDT
VLGRLALLQEERDKLITEMDRSLLENQSLSSSCESLKLALEGLTEDKE
KLVKEIESLKSSKIAESTEWQEKHKELQKEYEILLQSYENVSNEAERI
QHVVEAVRQEKQELYGKLRSTEANKKETEKQLQEAEQEMEEMKEKMRK
FAKSKQQKILELEEENDRLRAEVHPAGDTAKECMETLLSSNASMKEEL
ERVKMEYETLSKKFQSLMSEKDSLSEEVQDLKHQIEGNVSKQANLEAT
EKHDNQTNVTEEGTQSIPGETEEQDSLSMSTRPTCSESVPSAKSANPA
VSKDFSSHDEINNYLQQIDQLKERIAGLEEEKQKNKEFSQTLENEKNT
LLSQISTKDGELKMLQEEVTKMNLLNQQIQEELSRVTKLKETAEEEKD
DLEERLMNQLAELNGSIGNYCQDVTDAQIKNELLESEMKNLKKCVSEL
EEEKQQLVKEKTKVESEIRKEYLEKIQGAQKEPGNKSHAKELQELLKE
KQQEVKQLQKDCIRYQEKISALERTVKALEFVQTESQKDLEITKENLA
QAVEHRKKAQAELASFKVLLDDTQSEAARVLADNLKLKKELQSNKESV
KSQMKQKDEDLERRLEQAEEKHLKEKKNMQEKLDALRREKVHLEETIG
EIQVTLNKKDKEVQQLQENLDSTVTQLAAFTKSMSSLQDDRDRVIDEA
KKWERKFSDAIQSKEEEIRLKEDNCSVLKDQLRQMSIHMEELKINISR
LEHDKQIWESKAQTEVQLQQKVCDTLQGENKELLSQLEETRHLYHSSQ
NELAKLESELKSLKDQLTDLSNSLEKCKEQKGNLEGIIRQQEADIQNS
KFSYEQLETDLQASRELTSRLHEEINMKEQKIISLLSGKEEAIQVAIA
ELRQQHDKEIKELENLLSQEEEENIVLEEENKKAVDKTNQLMETLKTI
KKENIQQKAQLDSFVKSMSSLQNDRDRIVGDYQQLEERHLSIILEKDQ
LIQEAAAENNKLKEEIRGLRSHMDDLNSENAKLDAELIQYREDLNQVI
TIKDSQQKQLLEVQLQQNKELENKYAKLEEKLKESEEANEDLRRSFNA
LQEEKQDLSKEIESLKVSISQLTRQVTALQEEGTLGLYHAQLKVKEEE
VHRLSALFSSSQKRIAELEEELVCVQKEAAKKVGEIEDKLKKELKHLH
HDAGIMRNETETAEERVAELARDLVEMEQKLLMVTKENKGLTAQIQSF
GRSMSSLQNSRDHANEELDELKRKYDASLKELAQLKEQGLLNRERDAL
LSETAFSMNSTEENSLSHLEKLNQQLLSKDEQLLHLSSQLEDSYNQVQ
SFSKAMASLQNERDHLWNELEKFRKSEEGKQRSAAQPSTSPAEVQSLK
KAMSSLQNDRDRLLKELKNLQQQYLQINQEITELHPLKAQLQEYQDKT
KAFQIMQEELRQENLSWQHELHQLRMEKSSWEIHERRMKEQYLMAISD
KDQQLSHLQNLIRELRSSSSQTQPLKVQYQRQASPETSASPDGSQNLV
YETELLRTQLNDSLKEIHQKELRIQQLNSNFSQLLEEKNTLSIQLCDT
SQSLRENQQHYGDLLNHCAVLEKQVQELQAGPLNIDVAPGAPQEKNGV
HRKSDPEELREPQQSFSEAQQQLCNTRQEVNELRKLLEEERDQRVAAE
NALSVAEEQIRRLEHSEWDSSRTPIIGSCGTQEQALLIDLTSNSCRRT
RSGVGWKRVLRSLCHSRTRVPLLAAIYFLMIHVLLILCFTGHL
SEQ ID NO: 67
VPLLAAIYFLMIHVLLILCFT

In one embodiment, the endoplasmic reticulum-retention peptide comprises or consists of TGN46, or of a fragment thereof comprising its Golgi transmembrane domain.

As used herein, the term “TGN46”, also referred to as “Trans-Golgi network integral membrane protein 2”, refers to a protein found in the trans-Golgi network. An exemplary amino acid sequence of TGN46 comprises or consists of SEQ ID NO: 68, corresponding to version 4 of UniProtKB accession number 043493. The Golgi transmembrane domain of TGN46 comprises or consists of amino acid residues 382 to 402 of SEQ ID NO: 68, set forth in SEQ ID NO: 69.

Homo sapiens
SEQ ID NO: 68
MRFVVALVLLNVAAAGAVPLLATESVKQEEAGVRPSAGNVSTHPSLSQ
RPGGSTKSHPEPQTPKDSPSKSSAEAQTPEDTPNKSGAEAKTQKDSSN
KSGAEAKTQKGSTSKSGSEAQTTKDSTSKSHPELQTPKDSTGKSGAEA
QTPEDSPNRSGAEAKTQKDSPSKSGSEAQTTKDVPNKSGADGQTPKDG
SSKSGAEDQTPKDVPNKSGAEKQTPKDGSNKSGAEEQGPIDGPSKSGA
EEQTSKDSPNKVVPEQPSRKDHSKPISNPSDNKELPKADTNQLADKGK
LSPHAFKTESGEETDLISPPQEEVKSSEPTEDVEPKEAEDDDTGPEEG
SPPKEEKEKMSGSASSENREGTLSDSTGSEKDDLYPNGSGNGSAESSH
FFAYLVTAAILVAVLYIAHHNKRKIIAFVLEGKRSKVTRRPKASDYQR
LDQKS
SEQ ID NO: 69
SSHFFAYLVTAAILVAVLYIA

In one embodiment, the mitochondrion-retention peptide comprises or consists of any amino acid sequence set forth in Table 3.

TABLE 3
MITOCHONDRION-RETENTION PEPTIDES

| SEQ | |
|---|---|
| ID NO: | Amino acid sequence |
| 70 | MDLLPPKPKYNPLRNESLSSLEEGASGSTPPE |
| 71 | MSFFNISRRFYSQIVKKSVKIKR |
| 72 | MFAAVARRTATGTLPGAVTLFGRKSFEGYYRDYPLHPRNFGV |
| | MASLF |
| 73 | LVPRYRGLFHHISKLDGGVRFL |
| 74 | MIFPVARYALRWLRRPEDRAFSRAAMEMALRGVR |
| 75 | MAGRLLGKALAAVSLSLALASVTIRSSRCRGIQAFRNSFSSS |
| | WFHL |
| 76 | PNNSGFIYQQHGGQQKRARFDHQ |
| 77 | MKSFITRNKTAI |
| 78 | MFRMLAKASVTLGSRAASWVRNM |
| 79 | MFSNLSKRWAQRTLSKSFYSTATGAASKSGKLTQKLVTAGV |
| | AAAGITASTLLYADSLTAEA |
| 80 | MLSSSFERN |
| 81 | MAFWGWRAAAALRLWGRVVERVEAGGGVGPFQACGCRLV |
| | LGGRDDVSAGLRGSHGARGEPLDP |
| 82 | MRRMPRCGIRLPLLRPS |
| 83 | MVDRGLGRHLWRLTRRGPPAAADAVAPRPLMGFYEAATQN |
| | QADCQLWALLRRGLTTASTLR |
| 84 | MAFLRSMWGVLSALGRSGAELCTGCGSRLRSPFSFVYLPRWF |
| 85 | HSLLPALCDSKIRSKALGSDHCPITLYLAL |
| 86 | AIRILVCEGSGLLAAANDILAARAQRPAARGSTSGGESRLRG |
| | ERARP |
| 87 | RGFWANLMQILRDITGVTAICSFLYTKLGIAPFGDPVTMF |
| 88 | MRTVFSQIPRFKQVNQYIRM |
| 89 | MAAACRSVKGL |
| 90 | MISASRAAAARLVGTAASRSPAAARPQDGWNGLSHEAFRFVS |
| | RRDY |
| 91 | RHFWADSKSS |
| 92 | MTVPYLNSNRNVASYLQSNS |
| 93 | MYRYLAKALLPSRAGPAALGSAANHSAALLGRGRGQPAAAS |
| | QPGLALAARRHY |
| 94 | MFIRNFVNIIGSQKSITKTIARNYFSDNSKLIIPRHG |
| 95 | MLRFTNCSCKTFVKSSYKLNIRRMNSSFRTETDAFGEIHVPA |
| | DKYWGAQTQRSFQNFKIGGARERMPLPLVHAFGVLKKSAAIV |
| | NESLGGLD |
| 96 | RHSTRTQHLSVETSY |
| 97 | VVRVTRTWVPKGL |
| 98 | MLATRALSLIGKRAISTSVCLRAHGSVVKS |
| 99 | VRGHPKSRGNFPP |
| 100 | MLARGLPLRSALVKACPPILSTVGEGWGHHRVGTGEGAG |
| 101 | FSRSFSTPLPLSTKTLISLSPPHRTFAVRA |
| 102 | TRTWVPKGLKSPP |
| 103 | VTHGYNVQRYNQLRKNFGFYRAYSSLRGCKSVFRRI |
| 104 | MEFIYIYMNFFFFFSVGRKFKKFNKVRVVR |
| 105 | MVGRNSAIAAGVCGALFIGYCIYFDRKRRSDPNFK |
| 106 | LLRLIGLTTIFSATALGFLAHKRGLLVRV |
| 107 | MALLRGVFIVAAKRTP |
| 108 | MSTPSIVIASARTA |
| 109 | KTLLSLALVGACITLGAYLGHK |
| 110 | MKSFITRNKTAILATVAATGTAIGAYYYY |
| 111 | MLSLRQSIRFFK |
| 112 | MGRTVVVLGGGISGLAASYHLSRAPCPP |
| 113 | MLKYKPLLKISKNCEAAILRASKTRLNTIRAYGSTVPKSKSF |
| | EQDSRKRTQSWTALRVGAILAATSSVAYLNWHNGQIDN |
| 114 | CLRRSWNSVTYFQNIGRISL |
| 115 | MVLPRLYTATSRAAFKAAKQSAPLLST |
| 116 | MQRSIFARF |
| 117 | MLSNLRILLNKAALRKAHTSMVRNFRYGKPVQ |
| 118 | MATSASSHLSKAIKHMYMKLPQG |
| 119 | VAVHNFLNEGAWNEIVEWERRFGKGLM |
| 120 | PKDMTPKAALLQVLGRINSKYATEPPFDRHDWYV |
| 121 | ESRKGQERFNRWFLTGMTVAGVVLLGSLFSRK |
| 122 | MANLMMRLPISLRSFSVSASSS |
| 123 | MWRCGGRQGLGVLRR |
| 124 | MAVFRSGLLVLTTPLASLAPRLASILTSAA |
| 125 | RALAHGVRVL |
| 126 | MRSYEFHLARMSGATLCVVTGYRLLTSKWLADRIEDYRQRVI |
| 127 | RPTGFWENLKRILRDITGITALCGFLYNKLGMAPFG |
| 128 | TELLVGIATVSGCGAVVYCISK |
| 129 | RYSLKVLLSYHGWMFAEHGKMSRST |
| 130 | MSVLTPLLLRGLTGSARRLPVPRAKIHSL |

In one embodiment, the nucleus-retention peptide comprises or consists of any amino acid sequence set forth in Table 4.

TABLE 4
NUCLEUS-RETENTION PEPTIDES

| SEQ ID NO: | Amino acid sequence |
|---|---|
| 131 | TAKRS |
| 132 | KKKGSKTS |
| 133 | GKISKHWTGI |
| 134 | SYNDFGNYNNQSSNFGPMKGGNFGGRSSGPYGGGGQYFAKPRNQGGYGG |
| 135 | SNFGPMKGGNFGGRSSGPY |
| 136 | PPTKKIR |
| 137 | KNQKQGKGKTCKKGKK |
| 138 | KRKRGRPGRPPSTNKKPRK |
| 139 | RKRR |
| 140 | KRGKK |
| 141 | RKKRR |
| 142 | KKSRKEKK |
| 143 | RSGGNHERRNGRGGRGGYNRRNNGYHPY |
| 144 | SKKRSLED |
| 145 | PPRSKKRI |
| 146 | KSAKISKPLH |
| 147 | KNKEISMP |
| 148 | KKMIEEKKAVLKRKMK |
| 149 | NFNHIHKRIRRVADKYLSG |
| 150 | CNKRKYSLN |
| 151 | GKRKK |
| — | EED |
| 152 | KKKRER |
| 153 | EEKRKR |
| 154 | GDRRAAPARP |
| 155 | MISEALRKAIGKR |
| 156 | MSERKRREKL |
| 157 | GGGXXXKNRRXXXXXXRGGRN, wherein X is any amino acid residue |
| 158 | GNLDIQRPKRKRKNSRVTFSEDDEI |
| 159 | INPEDVDPSVGRFRNMVQTAVVPVKKKRVEG |
| 160 | GSGQIQLWQFLLELLSDSANASCITWEGTNGEFKMTDPDEVARRWGERKSKPNMNYDKLSRALRYYYDKN |
| | IMTKVHGKRYAYKF |
| 161 | HRKYEAPRHXXXXXXPRKR, wherein X is any amino acid residue |
| 162 | HSAELALNVITKKRKGYEDD |
| 163 | DYVSKKSKHEEEEWTDDDLVESL |
| 164 | KGRKR |
| 165 | KGVKRKADTTTP |
| 166 | KILKKRHI |
| 167 | KKIR |
| 168 | KKYR |
| 169 | KKKKRK |
| 170 | KKQALKEKELGNDAYKKK |
| 171 | KKSTALSRELGKIMRRR |
| 172 | KKKTVINDLLHYKKEK |
| 173 | KKXXXXXXXXXKKRK, wherein X is any amino acid residue |
| 174 | KNLEAIVSVIAERNKKK |
| 175 | KRKKXKKKSKK, wherein X is any amino acid residue |
| 176 | KRKXXXXXXXXXXKELQKQITK, wherein X is any amino acid residue |
| 177 | KRQQLKENNKLRR |
| 178 | KRVAKRKLIEQNRERRRK |
| 179 | KRXXXXXXXXXKTKK, wherein X is any amino acid residue |
| 180 | KSKKQK |
| 181 | KSRKRKL |
| 182 | KTLETVPLERKKREK |
| 183 | KVLKKRRL |
| 184 | KXXKXXXXK, wherein X is any amino acid residue |
| 185 | LXXXXXXXXXXXXXXXXXXXXXXXXXXXXXXRXXXXXXXXXXXXXXXXXXXXXXXXXXXXXXXXXXXXXX |
| | XXXXXXXXXXXXXXXXXXR, wherein X is any amino acid residue |
| 186 | NKIPIKDLLNPQ |
| 187 | MEQRGQNAPAASGARKRHGPGPREARGARPGPRVPKTLVLVVAAVLLLVSAESA |
| 188 | MGTLYARHVNAGSTGPIKSTIRIYFKPKHVKAWIPRPPRLCQYEKAKNVNFQPSGVTTTRQSI |
| 189 | CNGCRNMLLYPRGATNVCCALC |
| 190 | CANCRTTLMYPYGAPSVKCAVC |
| 191 | CGGCRTLLMYTRGATSVRCSCC |
| 192 | MVQLRPRASR |
| 193 | KRFKRRWMVRKMKTKK |
| 194 | RRDKTLLKEKRKRREELFIEQKKRK |
| 195 | NYKKPKL |
| 196 | PDGKRKRK |
| 197 | PEEVKKRKKAV |
| 198 | PKKKSRKSS |
| 199 | PKRRTARKQLPKR |
| 200 | PLKRKKL |
| 201 | PPXXR, wherein X is any amino acid residue |
| 202 | PRPR |
| 203 | REKKKFLKRR |
| 204 | REKKRQFEMKRKLH |
| 205 | RESGKKRKRKRLKPT |
| 206 | RKCSQTQCPRKVIK |
| 207 | RKRKLPCDTPGQGLTCSGEKRRR |
| 208 | RKRPVRRR |
| 209 | RKXXXXXXXXXXRKXXXXXXXXXXKK, wherein X is any amino acid residue |
| 210 | RLKKLKCSKXXXXXXXXXXXXKTKR, wherein X is any amino acid residue |
| 211 | RLRRDAGGRGGVYEHLGGAPRRRK |
| 212 | GKGRPRRGFKTRRTQKSS |
| 213 | RRERXXXXRPRKIPR, wherein X is any amino acid residue |
| 214 | RRGLKR |
| 215 | RRKRQR |
| 216 | RRKRR |
| 217 | RRRGL |
| 218 | RRRR |
| 219 | RSRARSASLGTTTQGWDPPPLRRPSRARRR |
| 220 | RGGRKRPLRPPLVSLARTPLCRRR |
| 221 | RVTIRTVRVRRPPKGKHRK |
| 222 | STPXKRLI, wherein X is any amino acid residue |
| 223 | SGVVLPSTGVVKSSGGGGGGDSDHSDLEASVVKEAESSRVVDPEKRPRKRGRKPANG |
| 224 | DVKIIGREAMIRVQSSKNNHPAARVMGALKDLDLEL |
| 225 | GREEPLNHVEAERQRREKLNQRFYALRAVVPNVSKMDKASLLGDAISYINELKAK |
| 226 | SHAAGSKQHESIPGKAKKPKVKKKEKGKKEKGKKKEAPH |
| 227 | SKLCLGWLWGM |
| 228 | TKKQKT |
| 229 | VRILESWFAKNIENPYLDT |
| 230 | VRVNVKREYDHMNGSRESPVDCSVSKCNKLVGGGEANPMNYNSYMDEKNGPPPPNMTTNERRVIV |
| 231 | YHYGLLTCESCKGFFKRTVQNQKRYTCIENQNCQIDKTQRKRCPYCRFKKCI |
| 232 | DVGMKLEAVRADRMRGGRNKFGPMYKRDRALKQQKK |
| 233 | YNDFGNYNNQSSNFGPMKGGNFGGRSSGPY |
| 234 | LRLTLLELVRRLNGNG |
| 235 | RRRKVEAGRTKLAHFRQRKTKGDSSHSEKKTAKRK |
| 236 | RRRHIVRKRTLRR |
| 237 | KRRQQKIRKYTMRR |
| 238 | RRWKEELDEELQRKR |
| 239 | KKLKKKK |
| 240 | KKYIDGQKKKCGEERRRV |
| 241 | RKRKL |
| 242 | KRRR |
| 243 | KRSAEGGNPPKPLKKLR |
| 244 | PKKLRPP |
| 245 | RRXXXXXXXXXXXXXXXXXXXXXXXXXXRKGFYKRKQCKPSRGRKRG, |
| | wherein X is any amino acid residue |
| 246 | EGKVTKRKHDNEGSGSKRPKV |
| 247 | RXXXXXXXXXXXHRGFYRKRQCRSSQGQRRG, wherein X is any amino acid residue |
| 248 | PEKKKEKQEKK |
| 249 | GRKKRRQRRRA |
| 250 | MRKTKLAPT |
| 251 | SHSSMAKRAKKESQDFI |
| 252 | AVEGKPLSRAAQLRERRKDLHVSGKPSPRYALKKRALEAKKNK |
| 253 | SGYGKVSRRGGHQNSYKPY |
| 254 | RDRGYDKADREEGKERRHHRREELAPY |
| 255 | GEGERPAQNEKRKEKNIKRGGNRFEPYANPTKR |
| 256 | FKSSQEEVRSYSDPPLKFMSVQRPGPYSPS |
| 257 | RKXXLXVYXXVIXKRXXXKR, wherein X is any amino acid residue |
| 258 | KKKRKR |
| 259 | KRGSRGGKKGRK |
| 260 | KKGR |
| 261 | KHSQSRR |
| 262 | KNRRAKCRQQR |
| 263 | KPNKRK |
| 264 | KRRAGDLLEDSPKRPK |
| 265 | KRRK |
| 266 | RARKRLK |
| 267 | KRRRER |
| 268 | KARKRLK |
| 269 | MEEAVTMAPAAVSSAVVGDPMEYNAILRRKLEEDLE |
| 270 | MPTEERVRKKESNRESARRSRYRKAAHLKEL |
| 271 | KRKGDEVDGVDEVAKKKSKK |
| 272 | GKKNQKHKLKM |
| 273 | KRKGTTRGMGAKSRKFINMYGFDPTDFSYI |
| 274 | KKQRYMEIQETLKK |
| — | KRK |
| 275 | FKRKHKKDISQNKRAVRR |
| 276 | KRKXXXXXXXXKKXK, wherein X is any amino acid residue |
| 277 | NVRQLLSGEQGADILMVQEAGSLPSSAVRTSRVIQHGGTPIEEYTWNLGTRSRPNMVYIYYSRLDVGANR |
| | VNLAIVS |
| 278 | KKRR |
| 279 | RKRKR |
| 280 | KRPR |
| 281 | RKQVSSHIQVLARRKLR |
| 282 | KRCFKKGAR |
| 283 | KCYRFRVKKNHRHR |
| 284 | RPRK |
| 285 | KQRHLDKKQR |
| 286 | KCLKFYSK |
| 287 | KRKKRK |
| 288 | KRKXXXXXXXXXXXXKR, wherein X is any amino acid residue |
| 289 | LKCLRYRFKKH |
| 290 | KRPRSPSS |
| 291 | KRKVLGSSQNSSGSEASETPVKRRK |
| 292 | PDRKRLRK |
| 293 | PRGSGPRRAAST |
| 294 | RRKTTPSYSGQYRTARR |
| 295 | ARRPSCSPEQHGGKVARLQPPPTKAQPA |
| 296 | RGGRRGRRRGRGRGG |
| 297 | PAAKRVKLD |
| 298 | RQRRNELKRS |
| 299 | MHGDTPTLHEYMLDLQPETTDLYCYEQLSDSSEEEDE |
| 300 | MRHKRSAKRTKR |
| 301 | RKRRKR |
| 302 | IASRRGLVRYSRIGQRGSMH |
| 303 | MNKIPIKDLLNPQ |
| 304 | VRILESWFAKNIENPY |
| 305 | GRRKAAKRKWAAQ |
| 306 | KKRKRR |
| 307 | RYKMKRAR |
| 308 | RGRKSSGFPKSVKSR |
| 309 | LRDGRAKLARD |
| 310 | KCHKDLPR |
| 311 | ARKRKPSP |
| 312 | REPADLEMNLTVPVRR |
| 313 | RRTQISSDHFPVGVSRR |
| 314 | RSKR |
| 315 | RRPALRSPPLGTRKRK |
| 316 | DIPPRPKRARVNLR |
| 317 | PIKKK |
| 318 | GSIIRKWN |
| 319 | KRKXXXXXXXXXKRRKXXXKRR, wherein X is any amino acid residue |
| 320 | FDYTPNSKRQRCGM |
| 321 | KKKKKEEEGEGKKK |
| 322 | KRPRP |
| 323 | RGRRRRQR |
| 324 | RKRRR |
| 325 | PPRIYPQLPSAPT |
| 326 | PRPRKIPR |
| 327 | HSHKKKKIRTSPTFRRPKTLRLRRQPKYPRKSAPRRNKLDHY |
| 328 | PKKNRLRR |
| 329 | KRKRRP |
| 330 | RRRXXXXXXXXXXXKRRK, wherein X is any amino acid residue |
| 331 | SKRVAKRKL |
| 332 | RRERNKMAAAKCRNRRR |
| 333 | KRMRNRIAASKCRKRKL |
| 334 | PLLKKIKQ |
| 335 | PAKRARRG |
| 336 | KVNSRKRRKEVPGPNGATEED |
| 337 | LKDVRKRKLGPGH |
| 338 | YKRPCKRSFIRFI |
| 339 | KKEKKKSKK |
| 340 | GKKRSKA |
| 341 | RAIKRRPGLDFDDDGEGNSKFLR |
| 342 | RKRKKMPASQRSKRRKT |
| 343 | KRPACTLKPECVQQLLVCSQEAKK |
| 344 | PRGRRQPIPKARQP |
| 345 | PRRGPR |
| 346 | KKSKKGRQEALERLKKA |
| 347 | RKEWLTNFMEDRRQRKL |
| 348 | KKQTTLAFKPIKKGKKR |
| 349 | RKCLQAGMNLEARKTKK |
| 350 | GKKKYKLKH |
| 351 | KKKYKLK |
| 352 | KSKKKAQ |
| 353 | RQARRNRRRRWR |
| 354 | KRAAEDDEDDDVDTKKQK |
| 355 | RRKGKEK |
| 356 | RRSMKRK |
| 357 | RKRAFHGDDPFGEGPPDKK |
| 358 | MAPSAKATAAKKAVVKGTNGKKALKVRTSATFRLPKTLKLAR |
| 359 | KTRKHRG |
| 360 | KHRKHPG |
| 361 | KKKKRKREK |
| 362 | NQSSNFGPMKGGNFGGRSSGPYGGGGQYFAKPRNQGGY |
| 363 | YNNQSSNFGPMKGGN |
| 364 | MNKIPIKDLLNPG |
| 365 | PQSRKKLR |
| 366 | KRQRXXXXXXXXXXXXKKSKK, wherein X is any amino acid residue |
| 367 | VNEAFETLKRC |
| 368 | CKRKTTNADRRKA |
| 369 | QRKRQK |
| 370 | HRIEEKRKRTYETFKSI |
| 371 | PPQKKIKS |
| 372 | SXGTKRSZXXM, wherein X is any amino acid residue |
| 373 | TKRSXXXM, wherein X is any amino acid residue |
| 374 | PPRKKRTVV |
| 375 | KRKKEMANKSAPEAKKKK |
| 376 | KRXXXXXKKKL, wherein X is any amino acid residue |
| 377 | KRPAATKKAGQAKKKK |
| 378 | PQPKKKP |
| 379 | KRXPQPKKKP, wherein X is any amino acid residue |
| 380 | RIRKKLR |
| 381 | LKRKLQR |
| 382 | RRMKWKK |
| 383 | KVTKNKS |
| 384 | KRRGKPGP |
| 385 | PYLNKRKGKP |
| 386 | PKKARED |
| 387 | VSRKRPR |
| 388 | APKRKSGVSKC |
| 389 | EEDGPQKKKRRL |
| 390 | TEKKQGKSILYD |
| 391 | REKKEKEQKEK |
| 392 | LEKKVKKKFDW |
| 393 | YLTQETNKVETYKEQPLKTPGKKKKGKP |
| 394 | RVHPYQR |
| 395 | SDREDGNYCPPVKRERTS |
| 396 | KKKRERLD |
| 397 | MPKTRRRPRRSQRKRPPT |
| 398 | PRRRK |
| 399 | KRPMNAFMVWAQAARRK |
| 400 | RPRRK |
| 401 | KRPMNAFIVWSRDQRRK |
| 402 | APTKRKGS |
| 403 | PNKKKRK |
| 404 | PKKKRKV |
| 405 | SDKKVRSRLIE |
| 406 | KKKRRSREK |
| 407 | RKRIREDRKATTAQKVQQMKQRLNENERKRKR |
| 408 | KDCVINKHHRNRCQYCRLQR |
| 409 | GRKRKKRT |
| 410 | PKRPRDRHDGELGGRKRARG |
| 411 | EYLSRKGKLEL |
| 412 | KAKRQR |
| 413 | IKYFKKFPKD |
| 414 | RKRKK |
| 415 | MASQGTKRSYEQM |
| 416 | KRGINDRNFWRGENGRKTR |
| 417 | RRMLGAPLRQRRVR |
| 418 | VRTTKGKRKRIDV |
| 419 | RSSRGKRRRIE |
| 420 | KRKXXXXXXXXXKIR, wherein X is any amino acid residue |
| 421 | KRKRXXXXXXXXXKK, wherein X is any amino acid residue |
| 422 | RLRKXXXRKFKK, wherein X is any amino acid residue |
| 423 | RKDRRGGRMLKHKRQRDDGEGRGEVGSAGDMRAANLWPSPLMIKRSKK |
| 424 | KRXXXXXXKRSR, wherein X is any amino acid residue |
| 425 | RQKRXXXXXXXXXKRRSSPSKDGR, wherein X is any amino acid residue |
| 426 | KRXXXXXXXXXXXKKTR, wherein X is any amino acid residue |
| 427 | KRKXXXKXKKAKV, wherein X is any amino acid residue |
| 428 | RKKRKXXXXXXKKSK, wherein X is any amino acid residue |
| 429 | KKXXXXXXXXXXRKRGRPRK, wherein X is any amino acid residue |
| 430 | KKXXXXRLRKK, wherein X is any amino acid residue |
| 431 | KKRARXXXXXXXXRQRKK, wherein X is any amino acid residue |
| 432 | KLRXXXXXXXRREIQKR, wherein X is any amino acid residue |
| 433 | RAIKTKXXXXXXXKLKSK, wherein X is any amino acid residue |
| 434 | RKXXXXXXXXXXRKLKK, wherein X is any amino acid residue |
| 435 | GKRQVLIRP |
| 436 | GIIKPISKH |
| 437 | KKRK |
| 438 | KKLKK |
| 439 | DNPGKKRKSTDDNEGPSPKRRVIT |
| 440 | CSVKRKRDDD |
| 441 | SKPFSCGRSGKGHKRKTPFGNTERKTPVKKL |
| 442 | KRELELLKHNPKRRKIT |
| 443 | RRRK |
| 444 | YIFEVKKPEDEVLICIQQRPKRSTRREG |
| 445 | MADAFEVHYIADWADISSEYPKLHILTTSGRVLSVLAELS |
| 446 | MMPSSVSWGILLAGLCCLVPVSLA |
| 447 | MAGPATQSPMKLMALQLLLWHSALWTVQEA |
| 448 | MQRVNMIMAESPSLITICLLGYLLSAECTVFLDHENANKILNRPKR |
| 449 | MKGSLLLLLVSNLLLCQSVAP |
| 450 | MKWVTFISLLFLFSSAYSRGVFRR |
| 451 | MWWRLWWLLLLLLLLPMWA |
| 452 | MLFNLRILLNNAAFRNGHNFMVRNFRCGQPLQ |
| 453 | MSVLTPLLLRGLTGSARRLPVPRAKIHSL |
| 454 | MSVLTPLLLRGLTGSARRLPVPRAKIHSL |
| 455 | MVTKITLSPQNFRIQKQETTLLKEKSTEKNSLAKSILAVKNHFIELRSKLSERFISHKNTMLSFVDTRTL |
| | LLLAVTSCLATCQ |
| 456 | MGSSQAPRMGSVGGHGLMALLMAGLILPGILA |
| 457 | MAGIFYFLFSFLFGICD |
| 458 | MENRLLRVFLVWAALTMDGASA |
| 459 | MARQGCFGSYQVISLFTFAIGVNLCLG |
| 460 | MSRLPVLLLLQLLVRPGLQ |
| 461 | MKQQKRLYARLLTLLFALIFLLPHSSASA |
| 462 | MATPLPPPSPRHLRLLRLLLSG |
| 463 | MKAPGRLVLIILCSVVFS |
| 464 | MLQLWKLLCGVLT |
| 465 | MLYLQGWSMPAVA |
| 466 | MDNVQPKIKHRPFCFSVKGHVKMLRLDIINSLVTTVFMLIVSVLALIP |
| 467 | MPCLDQQLTVHALPCPAQPSSLAFCQVGFLTA |
| 468 | MKTLFNPAPAIADLDPQFYTLSDVFCCNESEAEILTGLTVGSAADA |
| 469 | MKPLLVVFVFLFLWDPVLA |
| 470 | MSCSLKFTLIVIFFTCTLSSS |
| 471 | MVLTKPLQRNGSMMSFENVKEKSREGGPHAHTPEEELCFVVTHTPQVQTTLNLFFHIFKVLTQPLSLLWG |
| 472 | MATPPFRLIRKMFSFKVSRWMGLACFRSLAAS |
| 473 | MSFFQLLMKRKELIPLVVFMTVAAGGASS |
| 474 | MVSALRGAPLIRVHSSPVSSPSVSGPAALVSCLSSQSSALS |
| 475 | MMGSPVSHLLAGFCVWVVLG |
| 476 | MASMAAVLTWALALLSAFSATQA |
| 477 | MVLMWTSGDAFKTAYFLLKGAPLQFSVCGLLQVLVDLAILGQATA |
| 478 | MDFVAGAIGGVCGVAVGYPLDTVKVRIQTEPLYTGIWHCVRDTYHRERVWGFYRGLSLPVCTVSLVSS |
| 479 | MEKPLFPLVPLHWFGFGYTALVVSGGIVGYVKTGSVPSLAAGLLFGSLA |
| 480 | MGLLLPLALCILVLC |
| 481 | MGIQTSPVLLASLGVGLVTLLGLAVG |
| 482 | MSDLLLLGLIGGLTLLLLLTLLAFA |
| 483 | METVVIVAIGVLATIFLASFAALVLVCRQ |
| 484 | MAGSVECTWGWGHCAPSPLLLWTLLLFAAPFGLLG |
| 485 | MMPSRTNLATGIPSSKVKYSRLSSTDDGYIDLQFKKTPPKIPYKAIALATVLFLIGA |
| 486 | MALPQMCDGSHLASTLRYCMTVSGTVVLVAGTLCFA |
| 487 | MKKWFVAAGIGAGLLMLSSAA |
| 488 | MKQSTIALALLPLLFTPVTKA |
| 489 | MKKTAIAIAVALAGFATVAQA |
| 490 | MKKLMLAIFFSVLSFPSFSQS |
| 491 | MKKNIAFLLASMFVFSIATNAYA |
| 492 | MFAKRFKTSLLPLFAGFLLLFHLVLAGPAAAS |
| 493 | MRFPSIFTAVLFAASSALA |
| 494 | MRFPSIFTTVLFAASSALA |
| 495 | MRFPSIFTSVLFAASSALA |
| 496 | MRFPSIFTHVLFAASSALA |
| 497 | MRFPSIFTIVLFAASSALA |
| 498 | MFPSIFTFVLFAASSALA |
| 499 | MRFPSIFTEVLFAASSALA |
| 500 | MRFPSIFTGVLFAASSALA |
| 501 | MRSSPLLRSAVVAALPVLALA |
| 502 | MGAAAVRWHLCVLLALGTRGRL |
| 503 | MRSSLVLFFVSAWTALA |
| 504 | MLRGPGPGRLLLLAVLCLGTSVRCTETGKSKR |
| 505 | MLRGPGPGLLLLAVQCLGTAVPSTGA |
| 506 | MRRGALTGLLLVLCLSVVLRAAPSATSKKRR |

In one embodiment, the vesicle-retention peptide is a lysosome-retention peptide.

In one embodiment, the lysosome-retention peptide comprises or consists of any amino acid sequence set forth in Table 5.

TABLE 5
LYSOSOME-RETENTION PEPTIDES

| SEQ | |
|---|---|
| ID NO: | Amino acid sequence |
| 507 | DSWGILFSHP |
| 508 | DEXXXLLI, wherein X is any amino acid |
| | residue |
| 509 | EXXXLL, wherein X is any amino acid |
| | residue |
| 510 | EEEXXXXXXXLI, wherein X is any amino |
| | acid residue |
| 511 | YTPL |
| 512 | NQPLLT |
| 513 | RRPGQRRPPPPPPPPPPPPPPPPPLPPLPLPPL |
| 514 | RGQGSMDEGTADERAPLIRTYXXVIFLWCM, wherein |
| | X is any amino acid residue |
| 515 | EKITLL |
| 516 | SSLKILSKG |
| 517 | PNQALLRILKETEFKKIKVLGSG |
| 518 | YLLNT |
| 519 | YGSI |
| 520 | GYDQL |
| 521 | YFPQA |
| 522 | LLDLLDE |
| 523 | LKQETEVELYNEFPEPMKLDKN |
| 524 | RRRAVVKEPLLPPEDDTRDN |
| 525 | PGYRHV |
| 526 | GYXXI, wherein X is any amino acid |
| | residue |
| 527 | GTEKCVWGPSYWCQNME |
| 528 | KXXXQ, wherein X is any amino acid |
| | residue |
| 529 | EQEPLL |
| 530 | YPAF |
| 531 | YEMA |
| 532 | GYXXVILFWCM, wherein X is any amino acid |
| | residue |
| 533 | SLLKGRQGIY |
| 534 | ETERLL |
| 535 | EEHSLL |
| 536 | DDSDEDLL |
| 537 | EQERLL |
| 538 | CINCCKVL |

In one embodiment, the vesicle-retention peptide is a peroxisome-retention peptide.

In one embodiment, the peroxisome-retention peptide comprises or consists of any amino acid sequence set forth in Table 6.

TABLE 6
PEROXISOME-RETENTION PEPTIDES

| SEQ | |
|---|---|
| ID NO: | Amino acid sequence |
| — | GRV |
| — | SKV |
| — | SLM |
| — | SKL |
| — | SKI |
| — | HRM |
| — | SRL |
| — | CKL |
| — | NKL |
| — | ARF |
| — | AKL |
| — | SQL |
| — | QKL |
| — | ANL |
| — | AKI |
| — | SSL |
| 539 | KXXXXXXILXLFILKLFI, wherein X is any amino |
| | acid residue |
| 540 | KVFFSRLIQILKIMVPRTFC |
| 541 | MTVPYLNSNRNVASYLQSNS |
| 542 | SVXXXXXQL, wherein X is any amino acid |
| | residue |
| 543 | MNGDQNSDVYAQEKQDFVQH |
| 544 | MHRLQVVLGHLAGRPESSSALQAAPC |
| 545 | EALREALQHCPKNKL |
| 546 | LSSNSKF |
| 547 | GFATKFYTEEGNLDWVYNNTPVFFI |
| 548 | MERLRQIASQATAASA |
| 549 | KANL |
| 550 | RPSI |
| 551 | APKI |

In one embodiment, the vesicle-retention peptide is a secretory vesicle-retention peptide.

In one embodiment, the secretory vesicle-retention peptide comprises or consists of any amino acid sequence set forth in Table 7.

TABLE 7
SECRETORY VESICLE-RETENTION PEPTIDES

| SEQ | |
|---|---|
| ID NO: | Amino acid sequence |
| 552 | MKELTMLSLSFFFVSALLAAAANPLLSAA |
| 553 | MRISKPHLRSISIQCYLCLLLNSHFLTEAGIHVFILGCFSAGL |
| | PKTEA |
| 554 | MNKIYSLKYSHITGGLIAVSELSGRVSSRATGKKKHKRILALC |
| | FLGLLQSSYSFA |
| 555 | MARQGCLGSFQVISLFTFAISVNICLG |
| 556 | MQRARPTLWAAALTLLVLLRGPPVARA |
| 557 | MKSVYFVAGLFIMLAQGSWQ |
| 558 | MNIKKFAKQATVLTFTTALLAGGATQAFA |
| 559 | MQQDGTQQDRIKQSPAPLNGMSRRGFLGGAGTLALATASGLLL |
| | PGTAHA |
| 560 | MGSYALPRSGVRRSIRVLLLALVVGVLGTATALIAPPGAHA |
| 561 | MNKVFKVIWCKTSQTWIAVSELSKAFSLSTTTDIPKKTKIFIA |
| | AAPLLFLSENTNA |
| 562 | SDPRLRQFLQKSK |
| 563 | CQDLTTESNLLAC |
| 564 | LPTDTTTFKRIFLKRM |

In one embodiment, the plasma membrane-retention peptide comprises or consists of any amino acid sequence set forth in Table 8.

TABLE 8
PLASMA MEMBRANE-RETENTION PEPTIDES

| SEQ | |
|---|---|
| ID NO: | Amino acid sequence |
| 565 | MRVKEKYQHLWRWGWKWGTMLLGILMICSATE |
| 566 | CAAL |
| 567 | KKKKKK |
| 568 | RRRRR |
| 569 | MRLLLALLGVLLSVPGPPVLS |
| 570 | CSIMNLMCGSTC |
| 571 | GHKSEEKREKMKRTLLKDWKTRLSYFLQNSSTPGKPKTGKKS |
| | KQQ |
| 572 | RSTLKLTTLQCQYSTVMD |
| 573 | RQGLHNMEDVYELIENSH |
| 574 | MDCRKMARFSYSVIWIMAISKVFELGLVAG |
| 575 | MPAWGALFLLWATAEA |
| 576 | RDYR |
| 577 | KMALRVALNNKQSGQITVKTSSSDHLSLAIAGLVPIALSIYQ |
| | KFKPGVSPSYSIY |
| 578 | MGSKIVQVFLMLALFATSALA |
| 579 | MNSKAMQALIFLGFLATSCLA |
| 580 | MGAAASIQTTVN |
| — | SVM |
| 581 | FALLGTHGASG |
| 582 | RRRTFLK |
| 583 | MGGKWSKSSV |
| 584 | DDPERE |
| 585 | EEANTGENNSLLHPMS |
| 586 | SRRGLV |
| 587 | SRRRFL |
| 588 | SRRQFI |
| 589 | QRRDFL |
| 590 | MNKIYSIKYSAATGGLIAVSELAKKVICKTNRKISAALLSLA |
| | VISYTNIIYA |
| 591 | MNPNQKIITIGSICMVIGIVSLMLQIGNIISIWVSHSIQT |
| 592 | LRCLACSCFRTPVWPR |
| 593 | MGCGCSSHPE |
| 594 | MPFVNKQFN |
| 595 | DEQNAKNAAQDRNSNKSSKGFFSKLGCC |
| 596 | MLCCMRRTKQ |
| 597 | VTNGSTYILVPLSH |
| 598 | AETENFV |
| 599 | RARHRRNVDRVSIGSYRT |
| 600 | YEDQ |
| 601 | LLVTSLLLCELPHPAFLIP |

According to the invention, the protein of interest is a secretory protein or a cell membrane-anchored protein.

By “secretory protein”, it is meant a protein which resides, even transiently, in the secretory apparatus of a eukaryotic cell, such as, without limitation, the endoplasmic reticulum (ER), the ER-Golgi intermediate compartment (ERGIC), the Golgi apparatus, and any vesicles involved in transport between them, as well as vesicles involved in protein degradation mechanisms via the proteasome and lysosome. A secretory protein can ultimately be secreted outside of the cell, or can remain in the secretory apparatus.

By “cell membrane-anchored protein”, also called “intrinsic protein”, it is meant a protein having one or several regions or domains that are embedded in the phospholipid bilayer of a cell. A cell membrane-anchored protein may span the entire phospholipid bilayer, and extend, to some extent, on each side of the phospholipid bilayer; or they may be only partially inserted in the phospholipid bilayer, and extend on one side only of the phospholipid bilayer, either extracellular or intracellular.

In one embodiment, the protein of interest is selected from the group comprising or consisting of cytokines, cytokine receptors, growth factors, cell receptors, major histocompatibility complexes (MHC) or proteins thereof, T-cell receptors (TCR) and proteins thereof, hormones, hormone receptors, antibodies or antigen-binding fragments thereof, chimeric antigen receptors (CARs), neurotransmitters, proteases, adhesion proteins, extracellular matrix proteins, and derivatives thereof.

Examples of cytokines include, but are not limited to, interleukins (such as, e.g., IL-1α, IL-1β, IL-1ra, IL-2, IL-3, IL-4, IL-5, IL-6, IL-7, IL-9, IL-10, IL-11, IL-12, IL-13, IL-14, IL-15, IL-16, IL-17A, IL-17B, IL-17C, IL-17D, IL-17E, IL-18, IL-19, IL-20, IL-21, IL-22, IL-23, IL-24, IL-26, IL-27, IL-28, IL-29, IL-30, IL-31, IL-32, IL-33, IL-34, IL-35, IL-36α, IL-36ρ, IL-36γ, IL-36ra, IL-37, IL-38, IFNα, IFNβ, IFNγ, IFNκ, IFNω, GM-CSF, oncostatin M, leukemia inhibitory factor, ciliary neurotrophic factor, cardiotrophin-1), chemokines (such as, e.g., chemokine C-C motif ligand (CCL) 1, CCL2/MCP1, CCL3/MIP1α, CCL4/MIP1β, CCL5/RANTES, CCL6, CCL7, CCL8/MCP2, CCL9, CCL11, CCL12, CCL13, CCL14, CCL15, CCL16, CCL17, CCL18/PARC/DCCK1/AMAC1/MIP4, CCL19, CCL20, CCL21, CCL22, CCL23, CCL24, CCL25, CCL26, CCL27, CCL28, chemokine C-X-C motif ligand (CXCL) 1, CXCL2, CXCL3, CXCL4, CXCL5, CXCL6, CXCL7, CXCL8/IL-8, CXCL9, CXCL10, CXCL11, CXCL12, CXCL13, CXCL14, CXCL15, CXCL16, CXCL17, fractalkine, chemokine C motif ligand (XCL) 1 and XCL2) and tumor necrosis factors (such as, e.g., tumor necrosis factor (TNF) a, lymphotoxin, OX40L, CD40LG, Fas ligand, CD70, CD153, 4-1BB ligand, TNF-related apoptosis-inducing ligand (TRAIL), receptor activator of nuclear factor κ-B ligand (RANKL), a proliferation-inducing ligand (APRIL), B-cell activating factor (BAFF) and ectodysplasin A (EDA)).

Examples of cytokine receptors include, but are not limited to, interleukin receptors (such as, e.g., IL-1R, IL-1R1, IL-1R2, IL-2R, IL-2RA, IL-2RB, IL-3R, IL-3RA, IL4R, IL-5R, IL-5RA, IL-6RA, IL-7R, IL-7RA, IL-9R, IL-10R, IL-10RA, IL-10RB, IL-11R, IL-11RA, IL-12R, IL-12RB1, IL-12RB2, IL-13R, IL-13RA1, IL-13RA2, IL-15R, IL-17R, IL-17RA, IL-17RB, IL-17RC, IL-17RD, IL-17RE, IL-18R, IL-18R1, IL-20R, IL-20RA, IL-20RB, IL-21R, IL-22R, IL-22RA1, IL22RA2, IL-23R, IL-27, IL-27RA, IL-28R, IFNα/βR, IFNγR, CNTFR, GM-CSFR, LIFR and OSMR), chemokine receptors (such as, e.g., CCR1, CCR11, CCR2, CCRL2, CCR3, CCR4, CCR5, CCR6, CCR7, CCR8, CCR9, CCR10, CXCR1, CXCR2, CXCR3, CXCR4, CXCR5, CXCR6, CXCR7, CX3CR1, XCR1, CCBP2 and CMKLR1) and tumor necrosis factor receptors (such as, e.g., TNFR1, TNFR2, LTBR, CD134, CD40, FasR, DcR3, CD27, CD30, CD137, DR3, DR4, DR5, DR6, DcR1, DcR2, RANK, osteoprotegerin, TWEAKR, TACI, BAFFR, HVEM, NGFR, BCMA, GITR, TAJ/TROY and EDA2R).

Examples of growth factors include, but are not limited to, fibroblast growth factor (FGF) 1, FGF2, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF23, transforming growth factor (TGF) α, epidermal growth factor (EGF), heparin-binding EGF-like growth factor (HB-EGF), transforming growth factor (TGF) β, insulin-like growth factor (IGF) 1, IGF2, Platelet-derived growth factor (PDGF) subunit A (PDGFA), PDGF subunit B (PDGFB), PDGF subunit C (PDGFC), PDGF subunit D (PDGFD), vascular endothelial growth factor (VEGF)-A, VEGF-B, VEGF-C, VEGF-D, placental growth factor (PGF), nerve growth factor (NGF) and hepatocyte growth factor (HGF).

Examples of cell receptors include, but are not limited to, cell surface receptors (such as, e.g., prostaglandin receptors, protease-activated receptors, neurotransmitter receptors, purinergic receptors, biogenic amine receptors, olfactory receptors, secretin receptors, metabotropic glutamate receptors, pheromone receptors, cAMP receptors, frizzled, smoothened, purinergic receptors, serine/threonine-specific protein kinases, receptor tyrosine kinase, guanylate cyclase, asialoglycoprotein receptors, tumor necrosis factor receptor, immunoglobulin superfamily, N-acetylglucosamine receptors, neuropilins, transferrin receptors, ectodysplasin A receptor (EDAR), lipoprotein receptor-related protein, and progestin and adipoQ receptor (PAQR)) and transmembrane receptors (such as, e.g., antibody receptors—including FcεRI, FcγRI, FcγRII, FcγRIII, neonatal Fc receptor, FcαRI, Fcα/μR and polymeric immunoglobulin receptor—, antigen receptors—including BCR, CD21, CD19, CD81, CD22, CD79, MHC, TCR, CD8, CD4, CD3, CD3γ, CD3δ, CD3ε and CD3ζ—, cytokine receptors, killer-cell IG-like receptors —including KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3 and KIR3DS1—, and leukocyte IG-like receptors—including LILRA1, LILRA2, LILRA3, LILRA4, LILRA5, LILRA6, LILRB1, LILRB2, LILRB3, LILRB4, LILRB5, LILRA6 and LILRA5).

Examples of MHC and proteins thereof include, but are not limited to, MHC class I, MHC class II, MHC class I α1 protein, MHC class I α2 protein, MHC class I α3 protein, β2-microglobulin, MHC class II α protein and MHC class II β protein.

Examples of TCR and proteins thereof include, but are not limited to, TCRα, TCRβ, TCRγ, TCRδ, CD3γ, CD3δ, CD3ε, CD3ζ, CD4 and CD8.

Examples of hormones include, but are not limited to, GnRH, TRH, dopamine, CRH, GHRH, somatostatin, MCH, oxytocin, vasopressin, FSH, LH, TSH, prolactin, POMC, CLIP, ACTH, MSH, endorphins, lipotropin, GH, aldosterone, cortisol, cortisone, DHEA, DHEA-S, androstenedione, epinephrine, norepinephrine, thyroid hormone T3, thyroid hormone T4, calcitonin, PTH, testosterone, AMH, inhibin, estradiol, progesterone, activin, relaxin, GnSAF, hCG, HPL, estrogen, glucagon, insulin, amylin, pancreatic polypeptide, melatonin, N,N-dimethyltryptamine, 5-methoxy-N,N-dimethyltryptamine, thymosin α1, beta thymosins, thymopoietin, thymulin, gastrin, ghrelin, CCK, GIP, GLP-1, secretin, motilin, VIP, enteroglucagon, peptide YY, IGF-1, IGF-2, leptin, adiponectin, resistin, osteocalcin, renin, EPO, calcitriol, prostaglandin, ANP and BNP.

Examples of hormone receptors include, but are not limited to, corticotropin-releasing hormone receptors (CRHR), follicle-stimulating hormone receptor (FSHR), gonadotropin-releasing hormone receptor (GnRHR), thyrotropin-releasing hormone receptor (TRHR), somatostatin, vasopressin receptor 1A (V1AR), vasopressin V1b receptor (V1BR), vasopressin receptor 2 (V2R), oxytocin receptor (OXTR), luteinizing hormone/choriogonadotropin receptor (LHCGR), thyrotropin receptor (TSHR), atrial natriuretic peptide receptor, calcitonin receptor (CT), cholecystokinin A receptor, cholecystokinin B receptor and vasoactive intestinal peptide receptor (VIPR).

Examples of antibodies or antigen-binding fragments thereof include, but are not limited to, monoclonal antibodies, polyclonal antibodies, bispecific antibodies, multispecific antibodies, antibody fragments, and antibody mimetics, such as, e.g., scFv, di-scFv, tri-scFv, single domain antibodies, nanobodies, bispecific T-cell engagers (BiTEs), Fab, F(ab′)2, Fab′, chemically linked Fab, X-Link Fab, tandem-scFv/BiTE, diabodies, tandem diabodies, diabody-Fc fusions, tandem diabody-Fc fusion, tandem diabody-CH3 fusion, tetra scFv-Fc fusion, dual variable domain immunoglobulin, knob-hole, strand exchange engineered domain, CrossMab, quadroma-derived bispecific antibody, single domain based antibody, affibodies, affilins, affimers, affitins, alphabodies, anticalins, avimer, DARPins, Kunitz domain peptides, monobodies and nanoCLAMPs.

Examples of CARs include, but are not limited to, first generation CARs (comprising an extracellular scFv, a hinge region, a transmembrane domain, and one or more intracellular signaling domains among which CD3), second generation CARs (comprising, in addition to the preceding, a co-stimulatory domain such as CD28 or 4-1BB), third generation CARs (comprising, in addition to the preceding, multiple co-stimulatory domains such as CD28/4-1BB or CD28-OX40) and fourth generation CARs (comprising, in addition to the preceding, factors enhancing T-cell expansion, persistence and anti-tumoral activity such as IL-2, IL-5, IL-12 or co-stimulatory ligands).

Examples of neurotransmitters include, but are not limited to, agmatine, aspartic acid, glutamic acid, glutathione, glycine, GSNO, GSSG, kynurenic acid, NAA, NAAG, proline, serine, GABA, GABOB, GHB, α-alanine, β-alanine, hypotaurine, sarcosine, taurine, T-HCA, 6-OHM, dopamine, epinephrine, normelatonin, norepinephrine, serotonin, histamine, dynorphin A, dynorphin B, big dynorphin, leumorphin, α-neoendorphin, β-neoendorphin, endomorphin-1, endomorphin-2, α-endorphin, β-endorphin, γ-endorphin, met-enkephalin, leu-enkephalin, adrenorphin, amidorphin, hemorphin, nociception, opiorphin, spinorphin, valorphin, bradykinins, tachykinins, neuromedin B, neuromedin N, neuromedin S, neuromedin U, orexin A, orexin B, angiotensin, bombesin, calcitonin gene-related peptide, carnosine, cocaine- and amphetamine-regulated transcript, delta sleep-inducing peptide, FMRFamide, galanin, galanin-like peptide, gastrin-releasing peptide, ghrelin, neuropeptide AF, neuropeptide FF, neuropeptide SF, neuropeptide VF, neuropeptide S, neuropeptide Y, neurophysins, neurotensin, pancreatic polypeptide, pituitary adenylate cyclase-activating peptide, RVD-Hpα and VGF.

Examples of proteases include, but are not limited to, alanyl aminopeptidase, aminopeptidase B, aspartyl aminopeptidase, leucyl/cystinyl aminopeptidase, leucyl aminopeptidase, glutamyl aminopeptidase, methionine aminopeptidase 1, methionine aminopeptidase 2, cathepsin C, dipeptidyl peptidase-4, tripeptidyl peptidase I, tripeptidyl peptidase II, angiotensin-converting enzyme, cathepsin A, DD-transpeptidase, carboxypeptidase A, carboxypeptidase A2, carboxypeptidase B, cathepsin A, carboxypeptidase E, glutamate carboxypeptidase II, metalloexopeptidase, serine protease, cysteine protease, aspartic acid protease, metalloendopeptidase, threonine endopeptidase, proteasome endopeptidase complex, HslU-HslV peptidase, amyloid precursor protein alpha secretase, amyloid precursor protein beta-secretase 1, amyloid precursor protein beta-secretase 2, amyloid precursor protein gamma secretase and staphylokinase.

Examples of adhesion proteins include, but are not limited to, neural cell adhesion molecule (NCAM), intercellular adhesion molecule—(ICAM) 1, ICAM-2, ICAM-3, ICAM-4, ICAM-5, vascular cell adhesion molecule 1 (VCAM-1), platelet endothelial cell adhesion molecule (PECAM-1), L1 cell adhesion molecule (LiCAM), nectin, integrins (such as, e.g., lymphocyte function-associated antigen 1 (LFA-1), integrin alphaXbeta2, macrophage-1 antigen, Integrin α4β1 (VLA-4), and glycoprotein IIb/IIIa), cadherins (such as, e.g., cadherin 1, cadherin 2, cadherin 3, cadherin 4, cadherin 5, cadherin 6, cadherin 8, cadherin 9, cadherin 10, cadherin 11, cadherin 12, cadherin 15, cadherin 16, cadherin 17, desmoglein 1, desmoglein 2, desmoglein 3, desmoglein 4, desmocollin 1, desmocollin 2, desmocollin 3, protocadherin 1, protocadherin 15, and T-cadherin), selectins (such as, e.g., E-selectin, L-selectin, and P-selectin), CD22, CD24, CD44, CD146 and CD164.

Examples of extracellular matrix proteins include, but are not limited to, collagen (such as, e.g., alpha-1 type I collagen, alpha-2 type I collagen, alpha-1 type II collagen, alpha-1 type III collagen, alpha-1 type IV collagen, alpha-2 type IV collagen, alpha-3 type IV collagen, alpha-4 type IV collagen, alpha-5 type IV collagen, alpha-6 type IV collagen, alpha-1 type V collagen, alpha-2 type V collagen, alpha-3 type V collagen, alpha-1 type VI collagen, alpha-2 type VI collagen, alpha-3 type VI collagen, alpha-5 type VI collagen, alpha-1 type VII collagen, alpha-1 type VIII collagen, alpha-2 type VIII collagen, alpha-1 type IX collagen, alpha-2 type IX collagen, alpha-1 type X collagen, alpha-1 type XI collagen, alpha-2 type XI collagen, alpha-1 type XII collagen, alpha-1 type XIII collagen, alpha-1 type XIV collagen, alpha-1 type XV collagen, alpha-1 type XVI collagen, alpha-1 type XVII collagen, alpha-1 type XVIII collagen, endostatin, alpha-1 type XIX collagen, alpha-1 type XX collagen, alpha-1 type XXI collagen, alpha-1 type XXII collagen, alpha-1 type XXIII collagen, alpha-1 type XXIV collagen, alpha-1 type XXV collagen, alpha-1 type XXVI collagen, alpha-1 type XXVII collagen, alpha-1 type XXVIII collagen, prolyl hydroxylase, lysyl hydroxylase, cartilage associated protein, leprecan, ADAMTS2, procollagen peptidase and lysyl oxidase), laminin (such as, e.g., laminin α-1, laminin α-2, laminin α-3, laminin α-4, laminin α-5, laminin β-1, laminin β-2, laminin β-3, laminin β-4, laminin γ-1, laminin γ-2, and laminin γ-3), ALCAM, elastin, tropoelastin, vitronectin, FRAS1, FREM2, decorin, FAM20C, extracellular matrix protein 1 (ECM1), matrix gla protein, alpha-tectorin, beta-tectonin, keratin, cytokeratin, gelatin, reticulin and cartilage oligomeric matrix protein.

In one embodiment, the protein of interest is a cytokine.

In one embodiment, the protein of interest is a cytokine selected from the group comprising or consisting of interleukin-12 (IL-12) and interleukin-2 (IL-2).

In one embodiment, the protein of interest may be wild-type or mutated.

By “wild-type”, it is meant any protein of interest which is encoded by a “wild-type gene”—a nucleic acid sequence which encodes a protein capable of having normal physiological function in vivo, although its sequence may differ from the known, published sequence, as long as the changes result in amino acid substitutions having little or no effect on the biological activity—, and which is capable of having normal physiological function in vivo. By contrast, a protein of interest is said to be “mutated” when the gene encoding such protein of interest has been modified, by insertion, deletion or substitution of one or several nucleotide residues, or even of large regions. A mutated protein of interest can have, e.g., an increased or decreased physiological function compared to the wild-type protein of interest, and/or can be truncated; and/or can be conjugated to a chemical moiety or another peptide or protein.

In particular, for naturally secreted or cell membrane anchored proteins of interest, their signal peptide can be replaced by the signal peptide from another secreted or cell membrane anchored protein. By way of example, such signal peptide can be the signal peptide of tissue plasminogen activator with SEQ ID NO: 602, or the signal peptide of CCL5 with SEQ ID NO: 603.

According to the invention, the protein of interest is fused to a hook protein-binding domain.

In one embodiment, the hook protein-binding domain is a biotin-binding protein-binding protein or peptide, or a derivative thereof, wherein said derivative retains its ability to bind to a biotin-binding protein or a derivative thereof (such as, e.g., avidin, streptavidin, tamavidin, bradavidin, rhizavidin, and derivatives thereof) as described above.

Hence, the hook protein-binding domain is a domain from, e.g., an avidin-binding protein, streptavidin-binding protein (SBP), tamavidin-binding protein, bradavidin-binding protein, rhizavidin-binding protein, etc.

In one embodiment, the biotin-binding protein-binding protein or peptide comprises or consists of any amino acid sequence set forth in Table 9.

TABLE 9
BIOTIN-BINDING PROTEIN-BINDING
PROTEINS OR PEPTIDES

| SEQ | |
|---|---|
| ID NO: | Amino acid sequence |
| 605 | DEKTTGWRGGHVVEGLAGELEQLRARLEHHPQGQREP |
| 606 | AWRHPQFGG |
| 607 | AWRHPQFGGAWRHPQFGG |
| 608 | AWRHPQFGGAWRHPQFGGAWRHPQFGG |
| 609 | WSHPQFEK |
| 610 | WSHPQFEKWSHPQFEK |
| 611 | WSHPQFEKWSHPQFEKWSHPQFEK |
| 612 | WSGPQFEK |
| 613 | WSGPQFEKWSGPQFEK |
| 614 | WSGPQFEKWSGPQFEKWSGPQFEK |
| 615 | WHRWPWLVSG |
| 616 | WHRWPWLVSGWHRWPWLVSG |
| 617 | WHRWPWLVSGWHRWPWLVSGWHRWPWLVSG |
| 618 | WHWWYWALDR |
| 619 | WHWWYWALDRWHWWYWALDR |
| 620 | WHWWYWALDRWHWWYWALDRWHWWYWALDR |
| 621 | WX₁HPQFX₂X₃, wherein X₁ represents any |
| | amino acid residue, and X₂ and X₃ both |
| | represent G, or where X₂ represents E and |
| | X₃ represents R or K |
| — | DVE |
| — | VEA |
| — | EAW |
| 622 | VEAW |
| 623 | DVEA |
| 624 | DVEAW |
| 625 | DVXAW, wherein X represents E, D or G |
| 626 | DVXAWL, wherein X represents E, D or G |
| 627 | DVEAWL |
| 628 | DVX₁AWLX₂, wherein X₁ represents E, D or |
| | G; and X₂ represents G or D |
| 629 | DVX₁AWLX₂X₃, wherein X₁ represents E, D or |
| | G; X₂ represents G or D; and X₃ represents |
| | A or E |
| 630 | DVX₁AWLX₂X₃R, wherein X₁ represents E, D or |
| | G; X₂ represents G or D; and X₃ represents |
| | A or E |
| 631 | DVEAWLGAR |
| 632 | DVX₁AWLX₂X₃RVPLVET, wherein X₁ represents |
| | E, D or G; X₂ represents G or D; and X₃ |
| | represents A or E |
| 633 | DRATPY |
| 634 | SEKLSNTK |

One skilled in the art can readily identify suitable biotin-binding protein-binding proteins or peptides, using, e.g., the SABFinder tool (He et al., 2016. Biomed Res Int. 2016: 9175143).

In one embodiment, the hook protein-binding domain is a FKBP protein or a derivative thereof, wherein said derivative retains its ability to bind to a FKBP-binding protein, as described above; and, preferably, to rapamycin or derivatives thereof.

As used herein, the term “FKBP”, also called “FK506 binding protein”, refers to a family of prolyl esterase proteins. Examples of FKBP proteins include, but are not limited to, FKBP1A, FKBP1B, FKBP2, FKBP3, FKBP5, FKBP6, FKBP7, FKBP8, FKBP9, FKBP9L, FKBP10, FKBP11, FKBP14, FKBP15, and FKBP52.

In one embodiment, the hook protein-binding domain is FKBP1A or a derivative thereof.

As used herein, the term “FKBP1A”, also called “FKBP12”, refers to a protein for which an exemplary amino acid sequence comprises or consists of SEQ ID NO: 635, corresponding to version 2 of UniProtKB accession number P62942.

Homo sapiens
SEQ ID NO: 635
MGVQVETISPGDGRTFPKRGQTCVVHYTGMLEDGKKFDSSRDRNKPFK
FMLGKQEVIRGWEEGVAQMSVGQRAKLTISPDYAYGATGHPGIIPPHA
TLVFDVELLKLE

One skilled in the art will readily understand that the hook protein and hook protein-binding domain should be chosen selected to operate in pairs, i.e., where the hook protein is a biotin-binding protein or a derivative thereof, the hook protein-binding domain is a biotin-binding protein-binding protein or peptide or a derivative thereof; where the hook protein is a FKBP-binding protein or a derivative thereof, the hook protein-binding domain is a FKBP protein or a derivative thereof.

Additionally, the present disclosure also encompasses pairs of hook proteins and hook protein-binding domains which are complementary to those described above. For example, it is conceivable that the hook protein be a biotin-binding protein-binding protein or peptide or a derivative thereof, and the hook protein-binding domain be a biotin-binding protein or a derivative thereof; or that the hook protein be a FKBP protein or a derivative thereof and the hook protein-binding domain be a FKBP-binding protein or a derivative thereof.

In one embodiment, a third partner may be necessary to achieve chemically induced dimerization of the hook protein and the hook protein-binding domain.

In one embodiment, a third partner is necessary, e.g., where the pair of hook protein and hook protein-binding domain comprises a FKBP-binding protein or a derivative thereof and a FKBP protein or a derivative thereof.

In the latter case, the third partner may be any ligand able to mediate the interaction between the FKBP-binding protein or a derivative thereof and the FKBP protein or a derivative thereof.

In one embodiment, the ligand able to mediate the interaction between the FKBP-binding protein or a derivative thereof and the FKBP protein or a derivative thereof is selected from the group comprising or consisting of FK506 (also termed “tacrolimus”), FK1012 (i.e., a dimer of tacrolimus wherein two tacrolimus units are linked at their vinyl groups), FKCsA (i.e., a FK506-cyclosporin A fusion,), rapamycin, and analogs thereof. In particular, analogs of rapamycin (also termed “rapalogs”) include, but are not limited to, C16-(S)-7-methylindolerapamycin (also termed “AP21967” or “C16-AiRap”), C16-(S)-3-methylindolerapamycin (also termed “C16-iRap”), C20-methallylrapamycin (also termed “C20-Marap”), and C16-(S)-butylsulfonamidorapamycin (also termed “BS-Rap”).

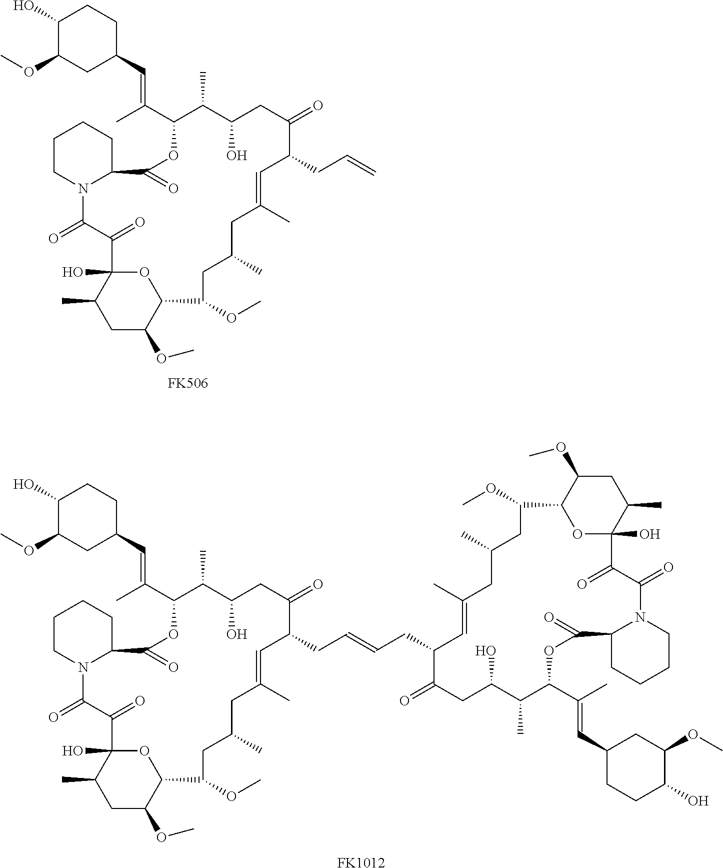

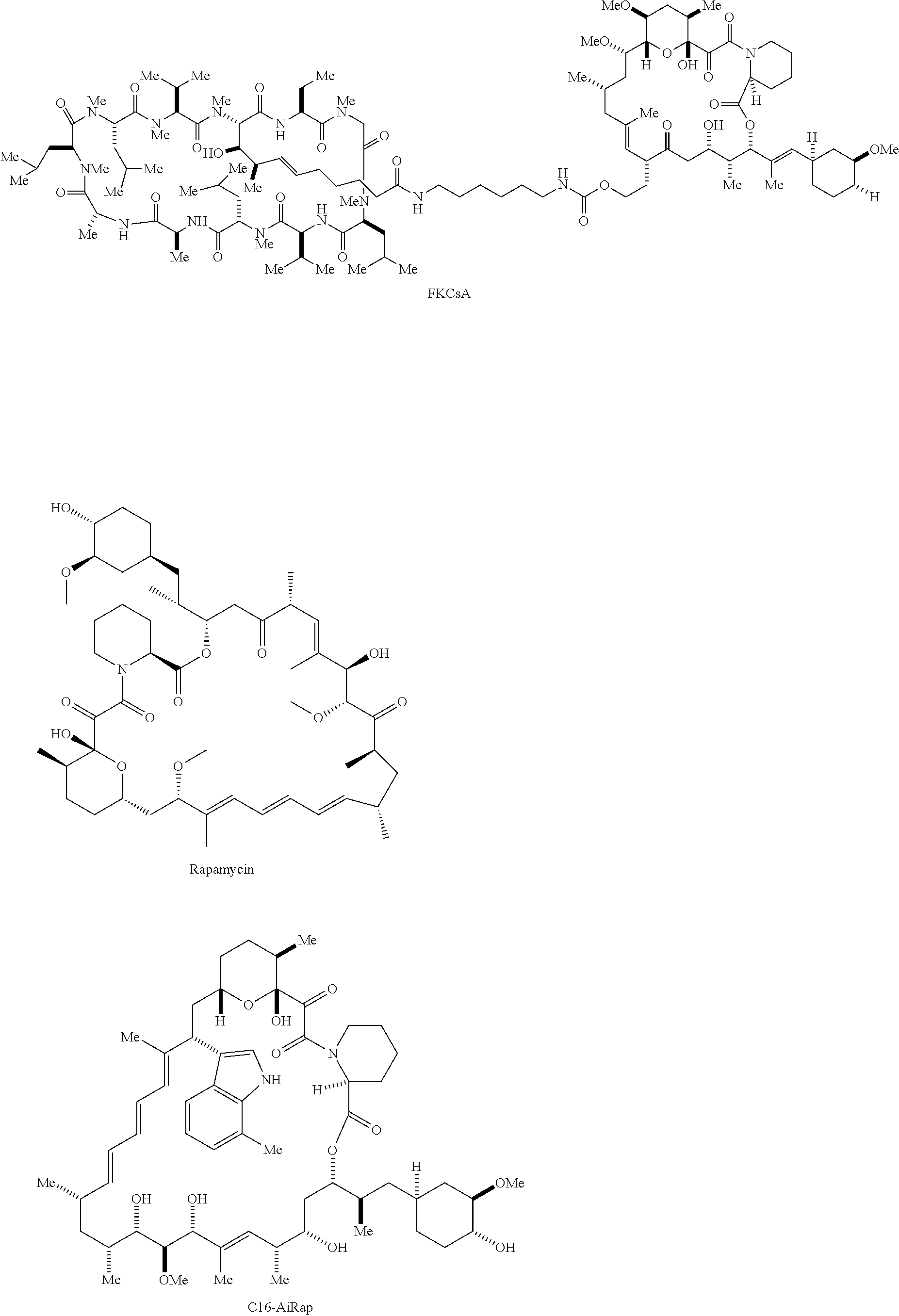

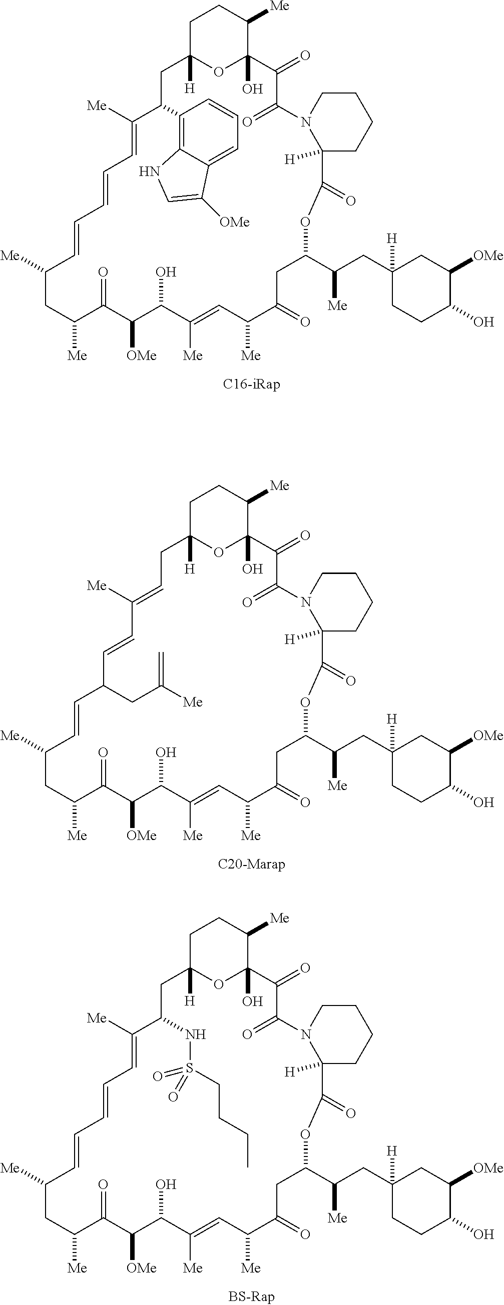

According to the invention, the gene encoding the hook protein is under the control of a first transcription-activating signal, and the gene encoding the protein of interest is under the control of a second transcription-activating signal, said second transcription-activating signal allowing an equal or lower rate or frequency of transcription initiation than the first transcription-activating signal, preferably a lower rate or frequency of transcription initiation than the first transcription-activating signal.

In one embodiment, the first transcription-activating signal allows a 1.25-fold, 1.5-fold, 2-fold, 3-fold, 4-fold, 5-fold, 10-fold or more, higher expression of the hook protein compared to the protein of interest.

In one embodiment, the second transcription-activating signal allows a 1.25-fold, 1.5-fold, 2-fold, 3-fold, 4-fold, 5-fold, 10-fold or more, lower expression of the protein of interest compared to the hook protein.

In one embodiment, the first transcription-activating signal allows a 5%, 10%, 15%, 20%, 25%, 30%, 35%, 40%, 45%, 50%, 55%, 60%, 65%, 70%, 75%, 80%, 85%, 90%, 95%, 100% or more higher rate or frequency of transcription initiation than the second transcription-activating signal.

In one embodiment, the second transcription-activating signal allows a 5%, 10%, 15%, 20%, 25%, 30%, 35%, 40%, 45%, 50%, 55%, 60%, 65%, 70%, 75%, 80%, 85%, 90%, 95%, 100% or more lower rate or frequency of transcription initiation than the first transcription-activating signal.

Examples of transcription-activating signals include, but are not limited to, promoters, enhancers and element response domains.

As used herein, the term “promoter” refers to a DNA sequence to which proteins bind to initiate transcription of a single stranded RNA from the DNA downstream of it. A promoter is typically located upstream (i.e., towards the 5′-region of the sense strand) near the transcription start site of the gene. Promoters can typically be about 100-1000 base pairs long. Promoters can be either natural or synthetic. Promoters can additionally be constitutive or inducible. Promoters can additionally be bidirectional promoters. The term “bidirectional promoter” refers to a typically short (<1 kbp) DNA sequence to which proteins initiating transcription bind to direct transcription, in both forward and reverse orientations, of two adjacent genes said to be in a “head-to-head arrangement”. A bidirectional promoter may thus be referred to as a “pair of sense and antisense transcription-activating signals”.

As used herein, the term “enhancer” refers to a cis-acting DNA sequence to which transcriptional activators bind to increase the likelihood that transcription of a particular gene will occur. Enhancers can typically be about 50-1500 base pairs long.

As used herein, the term “response element domain” refers to a short DNA sequence to which transcription factors bind to regulate, i.e., activate or inhibit, transcription of a gene. Examples of response elements include, but are not limited to, cAMP response element (CRE), B recognition element, AhR-, dioxin- or xenobiotic-responsive element, hypoxia-responsive elements, estrogen response elements (EREs), androgen response elements (AREs), serum response element (SRE), retinoic acid response elements (RAREs), peroxisome proliferator hormone response elements (PPREs), metal-responsive element (MRE), DNA damage response element (DRE), IFN-stimulated response elements (ISREs), ROR-response element, glucocorticoid response element (GRE), calcium-response element CaRE1, antioxidant response element (ARE), p53 response element, thyroid hormone response element, growth hormone response element (GHRE), sterol response element, polycomb response elements (PREs), vitamin D response element (VDRE), and Rev response element (RRE).

In one embodiment, the first and/or the second transcription-activating signals may independently from each other be a prokaryotic or eukaryotic transcription-activating signal; preferably the first and the second transcription-activating signals are eukaryotic transcription-activating signals.

One skilled in the art is familiar with transcription-activating signals, and can readily chose a first and a second transcription-activating signal, said second transcription-activating signal allowing an equal or lower rate or frequency of transcription initiation than the first transcription-activating signal, preferably a lower rate or frequency of transcription initiation than the first transcription-activating signal.

In one embodiment, the first and/or the second transcription-activating signal is a promoter. In one embodiment, the first and/or the second promoter may independently from each other be a natural or a synthetic promoter.

In one embodiment, the first transcription-activating signal is a promoter allowing a high rate of expression of the gene encoding the hook protein. In one embodiment, the first transcription-activating signal is a promoter allowing a high rate or frequency of transcription initiation. Such promoters may be referred to as “strong promoters” in the art.

Examples of promoters allowing a high rate of expression of the gene encoding the hook protein and/or allowing a high rate or frequency of transcription initiation include, but are not limited to, CMV, SFFV, CAG, EF1, EF1A, GAL1, GAL10, GPD, ADH and GAP.

In one embodiment, the first transcription-activating signal is a spleen focus forming virus (SFFV) promoter. An exemplary sequence of SFFV promoter comprises or consists of the nucleic acid sequence with SEQ ID NO: 639.

SEQ ID NO: 639
gtaacgccattttgcaaggcatggaaaaataccaaaccaagaatagaga
agttcagatcaaggggggtacatgaaaatagctaacgttgggccaaaca
ggatatctgcggtgagcagtttcggccccggcccggggccaagaacaga
tggtcaccgcagtttcggccccggcccgaggccaagaacagatggtccc
cagatatggcccaaccctcagcagtttcttaagacccatcagatgtttc
caggctcccccaaggacctgaaatgaccctgcgccttatttgaattaac
caatcagcctgcttctcgcttctgttcgcgcgcttctgcttcccgagct
ctataaaagagctcacaacccctcactcggcgcgccagtcctccgacag
actgagtcgcccggg

In one embodiment, the second transcription-activating signal is a promoter allowing a medium or low rate of expression of the gene encoding the protein of interest. In one embodiment, the second transcription-activating signal is a promoter allowing a low or medium rate or frequency of transcription initiation. Such promoters may be referred to as “weaker promoters” in the art.

Examples of promoters allowing a low rate of expression of the gene encoding the protein of interest and/or allowing a low or medium rate or frequency of transcription initiation include, but are not limited to, vav, PGK, SV40, thymidine kinase promoter (TK), MSCV and UbC promoter.

In one embodiment, the second transcription-activating signal is selected from the group comprising or consisting of a phosphoglycerate kinase (PGK) promoter, a ubiquitin C (UbC) promoter and a simian virus 40 (SV40) promoter.

In one embodiment, the second transcription-activating signal is a phosphoglycerate kinase (PGK) promoter. An exemplary sequence of PGK promoter comprises or consists of the nucleic acid sequence with SEQ ID NO: 640.

SEQ ID NO: 640
ttgcgccttttccaaggcagccctgggtttgcgcagggacgcggctgct
ctgggcgtggttccgggaaacgcagcggcgccgaccctgggtctcgcac
attcttcacgtccgttcgcagcgtcacccggatcttcgccgctaccctt
gtgggccccccggcgacgcttcctgctccgcccctaagtcgggaaggtt
ccttgcggttcgcggcgtgccggacgtgacaaacggaagccgcacgtct
cactagtaccctcgcagacggacagcgccagggagcaatggcagcgcgc
cgaccgcgatgggctgtggccaatagcggctgctcagcagggcgcgccg
agagcagcggccgggaaggggcggtgcgggaggcggggtgtggggcggt
agtgtgggccctgttcctgcccgcgcggtgttccgcattctgcaagcct
ccggagcgcacgtcggcagtcggctccctcgttgaccgaatcaccgacc
tctctccccag

In one embodiment, the second transcription-activating signal is a ubiquitin C (UbC) promoter. An exemplary sequence of UbC promoter comprises or consists of the nucleic acid sequence with SEQ I) NO: 641.

SEQ ID NO: 641
ggtgcagcggcctccgcgccgggttttggcgcctcccgcgggcgccccc
ctcctcacggcgagcgctgccacgtcagacgaagggcgcagcgagcgtc
ctgatccttccgcccggacgctcaggacagcggcccgctgctcataaga
ctcggccttagaaccccagtatcagcagaaggacattttaggacgggac
ttgggtgactctagggcactggttttctttccagagagcggaacaggcg
aggaaaagtagtcccttctcggcgattctgcggagggatctccgtgggg
cggtgaacgccgatgattatataaggacgcgccgggtgtggcacagcta
gttccgtcgcagccgggatttgggtcgcggttcttgtttgtggatcgct
gtgatcgtcacttggtgagtagcgggctgctgggctggccggggctttc
gtggccgccgggccgctcggtgggacggaagcgtgtggagagaccgcca
agggctgtagtctgggtccgcgagcaaggttgccctgaactgggggttg
gggggagcgcagcaaaatggcggctgttcccgagtcttgaatggaagac
gcttgtgaggcgggctgtgaggtcgttgaaacaaggtggggggcatggt
gggcggcaagaacccaaggtcttgaggccttcgctaatgcgggaaagct
cttattcgggtgagatgggctggggcaccatctggggaccctgacgtga
agtttgtcactgactggagaactcggtttgtcgtctgttgcgggggcgg
cagttatggcggtgccgttgggcagtgcacccgtacctttgggagcgcg
cgccctcgtcgtgtcgtgacgtcacccgttctgttggcttataatgcag
ggtggggccacctgccggtaggtgtgcggtaggcttttctccgtcgcag
gacgcagggttcgggcctagggtaggctctcctgaatcgacaggcgccg
gacctctggtgaggggagggataagtgaggcgtcagtttctttggtcgg
ttttatgtacctatcttcttaagtagctgaagctccggttttgaactat
gcgctcggggttggcgagtgtgttttgtgaagttttttaggcacctttt
gaaatgtaatcatttgggtcaatatgtaattttcagtgttagactagta
aa

In one embodiment, the second transcription-activating signal is a simian virus 40 (SV40) promoter. An exemplary sequence of SV40 promoter comprises or consists of the nucleic acid sequence with SEQ ID NO: 642.

SEQ ID NO: 642
ctgtggaatgtgtgtcagttagggtgtggaaagtccccaggctccccag
caggcagaagtatgcaaagcatgcatctcaattagtcagcaaccaggtg
tggaaagtccccaggctccccagcaggcagaagtatgcaaagcatgcat
ctcaattagtcagcaaccatagtcccgcccctaactccgcccatcccgc
ccctaactccgcccagttccgcccattctccgccccatggctgactaat
tttttttatttatgcagaggccgaggccgcctctgcctctgagctattc
cagaagtagtgaggaggcttttttggaggcctaggcttttgcaaaaagc
t

Examples of inducible promoters include, but are not limited to, DAN1, HXT7, AOX1, FLD1, TRE or UAS.

In one embodiment, the first and second transcription-activating signals are a pair of sense and antisense transcription-activating signals of a bidirectional promoter.

In one embodiment, genes transcription is under the control of at least one bidirectional promoter, one gene transcription being in antisense and another gene transcription being in sense. Said bidirectional promoter may be eukaryote, prokaryote or viral. Said viral bidirectional promoter may by selected in the group of viral vectors, comprising, or consisting of, but without being limited to, lentivirus, gammaretrovirus, nodavirus or encephalomyocarditis virus.

In one embodiment, the polynucleotide does not comprise an internal ribosome entry site (IRES) sequence between the gene encoding the hook protein and the gene encoding the protein of interest.

As used herein, the term “IRES”, or “internal ribosome entry site”, refers to a nucleotide sequence that promotes the initiation of translation in a cap-independent manner.

Examples of IRES include, but are not limited to, viral IRES from Picornaviruses such as, e.g., polio virus (PV), encephalomyocarditis virus (EMCV), foot-and-mouth disease virus (FMDV); IRES from Flaviviruses such as, e.g., hepatitis C virus (HCV); IRES from Pestiviruses such as, e.g., classical swine fever virus (CSFV); IRES from retroviruses such as, e.g., murine leukemia virus (MLV); IRES from Lentiviruses such as, e.g., simian immunodeficiency virus (SIV); cellular mRNA IRES such as, e.g., those from translation initiation factors (eIF4G, DAPS, and the like), from transcription factors (c-Myc, NF-κB-repressing factor (NRF), and the like), from growth factors (vascular endothelial growth factor (VEGF), fibroblast growth factor (FGF-2), platelet-derived growth factor B (PDGF B), and the like), from homeotic genes (antennapedia, and the like), from survival proteins (X-linked inhibitor of apoptosis (XIAP), Apaf-1, and the like) or from chaperones (immunoglobulin heavy-chain binding protein BiP, and the like).

In one embodiment, the polynucleotide does not comprise an encephalomyocarditis virus (ECMV) IRES sequence between the gene encoding the hook protein and the gene encoding the protein of interest.

In one embodiment, the ECMV IRES sequence comprises or consists of SEQ ID NO: 636.

Encephalomyocarditis virus
SEQ ID NO: 636
gagggcccggaaacctggccctgtcttcttgacgagcattcctagggg
tctttcccctctcgccaaaggaatgcaaggtctgttgaatgtcgtgaa
ggaagcagttcctctggaagcttcttgaagacaaacaacgtctgtagc
gaccctttgcaggcagcggaaccccccacctggcgacaggtgcctctg
cggccaaaagccacgtgtataagatacacctgcaaaggcggcacaacc
ccagtgccacgttgtgagttggatagttgtggaaagagtcaaatggct
ctcctcaagcgtattcaacaaggggctgaaggatgcccagaaggtacc
ccattgtatgggatctgatctggggcctcggtgcacatgctttacatg
tgtttagtcgaggttaaaaaacgtctaggccccccgaaccacggggac
gtggttttcctttgaaaaacacgatgataa

In one embodiment, the polynucleotide does not comprise an intervening sequence (IVS) between the gene encoding the hook protein and the gene encoding the protein of interest.

As used herein, the term “IVS”, or “intervening sequence”, also termed “RNA intervening sequence”, refers to an intronic sequence known in the art to stabilize mRNA.

In one embodiment, the IVS comprises or consists of SEQ ID NO: 637.

SEQ ID NO: 637
gaattaattcgctgtctgcgagggccagctgttggggtgagtactccct
ctcaaaagcgggcatgacttctgcgctaagattgtcagtttccaaaaac
gaggaggatttgatattcacctggcccgcggtgatgcctttgagggtgg
ccgcgtccatctggtcagaaaagacaatctttttgttgtcaagcttgag
gtgtggcaggcttgagatctggccatacacttgagtgacaatgacatcc
actttgcctttctctccacaggtgtccactcccaggtccaactgcaggt
cg

In one embodiment, the polynucleotide further comprises at least one suicide gene.

As used herein, the term “suicide gene” refers to a gene capable of inducing cell apoptosis upon expression of said gene. In the frame of the present invention, suicide genes may be utilized to eliminate cells comprising the polynucleotide of the invention.

In one embodiment, the suicide gene may be an inducible suicide gene.

In one embodiment, the suicide gene is a gene encoding the caspase 9 protein or a variant thereof.

In one embodiment, the suicide gene is a gene encoding a metabolic enzyme, such as, e.g., a herpes simplex virus thymidine kinase (HSV-TK) or cytosine deaminase (CD).

In one embodiment, the suicide gene is a gene encoding a cytochrome P450 4B1 (CYP₄B1) mutant.

The present invention also relates to a vector, comprising the polynucleotide described above.

The present invention also relates to a system of at least two vectors comprising
a) a first vector comprising the first polynucleotide of the system of at least two polynucleotides described above, and
b) a second vector comprising the second polynucleotide of the system of at least two polynucleotides described above.

In the case of a system of at least two vectors, said at least two vectors are typically, but not exclusively, of the same type. In the following, when referring to “the vector”, the term is also intended to encompass the system of at least two vectors.

As used herein, the term “vector” refers to a nucleic acid molecule into which a polynucleotide can be inserted for transport between different genetic environments and/or for expression in a host cell. If the vector carries regulatory elements for transcription of the polynucleotide inserted in the vector (which, in the sense of the present invention, is the case with a polynucleotide itself comprising transcription-activating signals), the vector may be referred to as an “expression vector”.

In one embodiment, the vector allows expression of the polynucleotide in a host cell and/or transfer of the polynucleotide to a host cell. In one embodiment, the vector is suitable for long-term expression of the polynucleotide in a host cell and/or stable transfer of the polynucleotide to a host cell, such as, e.g., by one or several of replication of the polynucleotide, expression of the polynucleotide, maintenance of the polynucleotide in extrachromosomal form, or integration of the polynucleotide into the genome of the host cell. Certain vectors are capable of autonomous replication in a host cell into which they are introduced (such as, e.g., bacterial vectors comprising a bacterial origin of replication, or episomal mammalian vectors). Other vectors (such as, e.g., non-episomal mammalian vectors) can be integrated into the genome of a host cell upon introduction into the host cell, and thereby are replicated along with the host genome.

In one embodiment, the vector is a plasmid. A “plasmid” refers to a circular double stranded DNA loop into which the polynucleotide can be subcloned.

In one embodiment, the vector is a viral or pseudoviral vector. A “viral vector” refers to a recombinantly produced virus or viral particle that comprises a polynucleotide to be delivered into a host cell, either in vivo, ex vivo or in vitro.

Examples of viral vectors include, but are not limited to, retroviral vectors, adenoviral vectors, adeno-associated viral vectors, herpes viral vectors, alphaviral vectors, and poxviral vectors.

In one embodiment, the viral vector is a retroviral vector. Retroviral vectors are vectors derived from a retrovirus, the latter including the genus of lentivirus, the genus of alpharetrovirus (such as, e.g., avian leukosis virus), the genus of betaretrovirus (such as, e.g., mouse mammary tumor virus), the genus of gammaretrovirus (such as, e.g., murine leukemia virus and feline leukemia virus), the genus of deltaretrovirus (such as, e.g., bovine leukemia virus and human T-lymphotropic virus), and the genus of epsilonretrovirus (such as, e.g., Walleye dermal sarcoma virus).

In one embodiment, the viral vector is a lentiviral vector. Examples of lentiviruses include, but are not limited to, human immunodeficiency viruses (HIV-1 or HIV-2), simian immunodeficiency virus (S1V), feline immunodeficiency virus (FIV), equine infections anemia (EIA), caprine arthritis encephalitis virus (CAEV), bovine immunodeficiency virus (BIV), and visna virus.

In one embodiment, the viral vector is an adenoviral vector. Adenoviral vectors are vectors derived from an adenovirus.

In one embodiment, the viral vector is an adeno-associated viral vector. Adeno-associated viral vectors are vectors derived from an adeno-associated virus (AAV). Examples of AAV include, but are not limited to, AAV1, AAV2, AAV3, AAV4, AAV5, AAV6, AAV7, AAV8, AAV9, AAV10, AAV11, AAV12, AAV13 and pseudotypes thereof (i.e., a mix of a capsid and genome from two different AAV serotypes).

In one embodiment, the viral vector is a herpes viral vector. Herpes viral vectors are vectors derived from a herpes virus. Examples of herpesvirus include, but are not limited to, herpes simplex virus 1 (HSV-1), herpes simplex virus 2 (HSV-2), varicella zoster virus, Epstein-Barr virus and cytomegalovirus.

In one embodiment, the viral vector is an alphaviral vector. Alphaviral vectors are vectors derived from an alphavirus. Examples of alphaviruses include, but are not limited to, Ross river virus, Sindbis virus (SIN), Semliki forest virus (SFV), and Venezuelan equine encephalitis virus (VEE).

In one embodiment, the viral vector is a poxviral vectors. Poxviral vectors are vectors derived from a poxvirus. Examples of poxviruses include, but are not limited to, vaccinia virus.

In one embodiment, the viral vector is an oncolytic viral vector. Oncolytic viral vectors are vectors derived from an oncolytic virus. These oncolytic viruses can selectively replicate in cancer cells, and subsequently spread within a tumor without affecting normal tissue. Alternatively, oncolytic viruses can infect and kill target cells without causing damage to normal tissues. Oncolytic viruses can also effectively induce immune responses to themselves as well as to an infected tumor cell.

Typically, oncolytic viruses fall into two classes: (1) viruses that naturally replicate preferentially in cancer cells and are non-pathogenic in humans (such as, without limitation, autonomous parvoviruses, myxoma virus, Newcastle disease virus (NDV), reovirus, and Seneca valley virus); and (2) viruses that are genetically manipulated for use as vaccine vectors (such as, without limitation, measles virus, poliovirus, and vaccinia virus). Additionally, oncolytic viruses may include those genetically engineered with mutations and/or deletions in genes required for replication in normal but not in cancer cells (such as, without limitation, adenovirus, herpes simplex virus, and vesicular stomatitis virus).

The present invention also relates to a cell comprising the polynucleotide (or the system of at least two polynucleotides) described above, or the vector (or the system of at least two vectors) described above.

In one embodiment where a third partner is necessary to achieve chemically induced dimerization of the hook protein and the hook protein-binding domain, as described above, the cell further comprises such third partner. Examples of suitable third partners have been described above.

In one embodiment, the cell is a eukaryotic cell. In one embodiment, the cell is an animal cell, preferably a mammal cell. In one embodiment, the cell is a human cell.

In one embodiment, the cell is a primary cell. In one embodiment, the cell is a cultured cell.

As used herein, the term “primary cell” refers to a cell that is or has been directly obtained from a subject (such as, e.g., a human) in the absence of culture. Typically, though not necessarily, a primary cell is capable of undergoing ten or fewer passages in vitro before senescence and/or cessation of proliferation. In contrast, a “cultured cell” is a cell that has been maintained and/or propagated in vitro for ten or more passages. Cultured cells include cell lines and primary cultured cells. As used herein, the term “cell line” refers to cells that are cultured in vitro, including primary cell lines, finite cell lines, continuous cell lines, and transformed cell lines, but does not require, that the cells be capable of an infinite number of passages in culture. Cell lines may be generated spontaneously or by transformation.

In one embodiment, the cell is obtained from a cell line. Examples of cell lines include, but are not limited to, 1301, 293, 293T, 380, 3T3, 5637, 8305C, 92.1, A-172, A-204, A-498, A-704, A 2058, A2780, A549, A8/D1, AC, ACHN, ACN, AF-2 cl.9B5, μAKR/11-D, AKR/12B-1, AKR/12B-2, AKR/12B-3, AKR/13B, AKR/14C, ALL-PO, AMALA, B104, B104-1-1, B16-F10, B1647, B3/AN, B9, B95.8, BAE-1, BALB/3T3 cl. A31, Bat lung cells, BOB, BRL-3A, BPH-1, BT-474, BT-549, BV-173, BV-2, BxPC-3, C2-Rev7, C33A, C6, CA46, Caco-2, Caki-2, CALOGERO, Calu-1, Calu-6, CAS-1, CaSki, CEINGE CL3, Cf2Th, CFPAC-1, CHO, CHO-K1, CHO-SSR1, CHO-SSR2, CM-S/un, CM-S/Tum, COLO 205, COLO 320DMF, COLO 699N, COLO 741, COLO 800, COLO 853, COLO 858, COR-L23, COS-1, COS-7, CTLL-2, DAUDI, DBTRG.05MG, DH-82, DLD-1, DMS-79, DOHH2, DU-145, E.Derm, F9, FAO, FTC-133, FTC-238, G-361, GDM-1, GF-D8, GH3, GH4-C1, GI-ME-N, GPE 86, GS-9L, H9, H-EMC-SS, HCT-15, HCT-116, HCT-8, HECV, HEK 293, HEK 293T, HEK 293A, HEL 92.1.7, HeLa, HeLa 382, HeLa 422, HeLa 432, HeLa S3, Hep G2, Hepa 1-clc7, HFFF2, HGC-27, HL-60, HOS, Hs578T, Hs913T, HT 1197, HT-1080, HT-29, HTC, HuP-T3, HuP-T4, HUVEC, IM-9, IMR-32, IMR-5, IST-EBV-TW1.1, IST-EBV-TW1.2, IST-EBV-TW2.1, IST-EBV-TW2.2, IST-EBV-TW3.1, IST-EBV-TW3.2, IST-EBV-TW4.A, IST-EBV-TW4.B, IST-EBV-TW5.A, IST-EBV-TW5.B, IST-EBV-TW6.A, IST-EBV-TW6.B, IST-SL1, IST-SL2, IST-MEL1, IST-MEL2, IST-MEL3, IST-MELA 16, IST-MES1, IST-MES2, J774A.1, JM2, JTC-27, K-562, KARPAS-422, KYSE-30, L1210, L6C5, L6H2, L929, LB4, LB-B7, LB-F9, LNCap.FGC, LoVo, LS 180, LTK-, M07e, Mab 62B1-PC, Mab 8A-PC, Mab 8F6-PC, McA-RH7777, MCF7, MCF7-382, MCF7-422, MCF7-432, MCF7-488X1, MCF7-490X1, MCF7-492X1, MDA-MB-231, MDA-MB-415, MDA-MB-436, MDA-MB-453, MDA-MB-468, ME-180, MeCo 05, MEG-01, MEGR 07, MEL 290, MeMo 05, MEMOR 06, MES-SA, MEWO, MG-1361, MG-63, MH1C1, MiCl1 (S⁺L⁻), MOLT-4, MONO-MAC-6, MPP 89, MRC-5, MSTO-211H, MTP-GFP, MylLu, NCI-H1650, NCI-H1975, NCI-H292, NCI-H727, Neuro-2a, NIH-3T3, NT2-D1, NULLI-SCC1, OCI-AML2, P19, P388.D1(cl3124), P3X63Ag8, P3X63Ag8U.1, PA-1, PA317, PC-12, PC-3, PF-382, PF97387, PG-4 (S+L), PSN1, RAJI, RAW 264.7, RBL-1, Rj2.2.5, R082-W-1, ROV-S, RPMI 7932, RRAm1, RT-1, S⁺L⁻CAT2, Saos-2, SC-1, SH-SY5Y, SHM-D33, SiHa, SIRC, SK-BR-3, SK-HEP-1, SK-LU-1, SK-MEL-5, SK-MEL-24, SK-MEL-28, SK-MES-1, SK-N-AS, SK-N-BE(2), SK-N-BE(2)-C, SK-N-F1, SK-N-SH, SR-4987, SUP-T1, SW1353, SW48, SW480, SW620, SW837, T47D, T84, TE671 Subline 2, TF-1, THP-1, THP-1h, THP-1l, TT, U-937, U-2 OS, U251 MG, U87/DK, U87 MG, U87/WT, U-937, UPMM 3, V-79, VA-ES-BJ, Vero, WEHI-164, WEHI-3B, WEHI-3B/cpx, WiDr, WOP, XC, Y79 or ZR-75-1.

In one embodiment, the cell is a stem cell. Examples of stem cells include, but are not limited to, hematopoietic stem cells, mesenchymal stem cells, neural stem cells, epithelial stem cells, skin stem cells, embryonic stem cells, induced pluripotent stem cells (iPSCs), pancreatic progenitor cells, hepatocyte precursor cells, and chondrogenic stem cells.

In one embodiment, the cell is an immune cell. Examples of immune cells include, but are not limited to, natural killer (NK) cells, natural killer T (NKT) cells, CD8⁺ T cells, CD4⁺ T cells, helper T cells, Tₕ1 cells, Tₕ2 cells, Tₕ17 cells, Tₕ21 cells, Tₕ23 cells, memory T (Tmem) cell, regulatory T (Treg) cells, γδ-T cells, mucosal-associated invariant T (MAIT) cells, macrophages, monocytes, plasmacytoid dendritic cells, conventional dendritic cells, eosinophils, basophils, plasma cells, neutrophils, cytotoxic induced T cells (CTLs), tumour infiltrating T cells, innate lymphoid cells, B cells, mast cells, pro-T cells and cytokine-induced killer cells.

In one embodiment, the cell is a fat cell or an adipocyte.

In on embodiment, the cell is a hepatocyte.

In one embodiment the cell is a neural cell.

In one embodiment, the cell is tumor cell.

The present invention also relates to a composition comprising the polynucleotide (or the system of at least two polynucleotides) described above, the vector (or the system of at least two vectors) described above, or the cell described above.

In one embodiment where a third partner is necessary to achieve chemically induced dimerization of the hook protein and the hook protein-binding domain, as described above, the composition further comprises such third partner. Examples of suitable third partners have been described above.

In one embodiment, the composition is a pharmaceutical composition, and further comprises at least one pharmaceutically acceptable excipient.

The term “pharmaceutically acceptable excipient” refers to a solid, semi-solid or liquid component of a pharmaceutical composition or a vaccine composition that is not an active ingredient, and that does not produce an adverse, allergic or other untoward reaction when administered to an animal, preferably to a human. The most of these pharmaceutically acceptable excipients are described in detail in, e.g., Allen (Ed.), 2017. Ansel's pharmaceutical dosage forms and drug delivery systems (11^{th} ed.). Philadelphia, PA: Wolters Kluwer; Remington, Allen & Adeboye (Eds.), 2013. Remington: The science and practice of pharmacy (22^{nd} ed.). London: Pharmaceutical Press; and Sheskey, Cook & Cable (Eds.), 2017. Handbook of pharmaceutical excipients (8^{th} ed.). London: Pharmaceutical Press; each of which is herein incorporated by reference in its entirety.

Pharmaceutically acceptable excipients include, but are not limited to, water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol®, vegetable oils, and the like. One may additionally include suitable preservatives, stabilizers, antioxidants, antimicrobials, and buffering agents, such as, e.g., BHA, BHT, citric acid, ascorbic acid, tetracycline, and the like.

Other examples of pharmaceutically acceptable excipients that may be used in the composition of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In addition, some pharmaceutically acceptable excipients may include, surfactants (e.g., hydroxypropylcellulose); suitable carriers, such as, e.g., solvents and dispersion media containing, e.g., water, ethanol, polyol (e.g., glycerol, propylene glycol, and liquid polyethylene glycol, and the like), suitable mixtures thereof, and vegetable oils, such as, e.g., peanut oil and sesame oil; isotonic agents, such as, e.g., sugars or sodium chloride; coating agents, such as, e.g., lecithin; agents delaying absorption, such as, e.g., aluminum monostearate and gelatin; preservatives, such as, e.g., benzalkonium chloride, benzethonium chloride, chlorobutanol, thimerosal and the like; buffers, such as, e.g., boric acid, sodium and potassium bicarbonate, sodium and potassium borates, sodium and potassium carbonate, sodium acetate, sodium biphosphate and the like; tonicity agents, such as, e.g., dextran 40, dextran 70, dextrose, glycerin, potassium chloride, propylene glycol, sodium chloride; antioxidants and stabilizers, such as, e.g., sodium bisulfite, sodium metabisulfite, sodium thiosulfite, thiourea and the like; nonionic wetting or clarifying agents, such as, e.g., polysorbate 80, polysorbate 20, poloxamer 282 and tyloxapol; viscosity modifying agents, such as, e.g., dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose; and the like.

In one embodiment, the composition is a medicament.

The present invention also relates a method of modulating the secretion or the cell membrane-anchorage of a protein of interest, comprising the steps of:
(a) transducing a cell with the polynucleotide (or the system of at least two polynucleotides) described above or the vector (or the system of at least two vectors) described above,
(b)
   (i) having the transduced cell of step (a) express a hook protein fused to a cellular compartment-retention peptide and the protein of interest fused to a hook protein-binding domain, and
   (ii) optionally, where a third partner is necessary to achieve chemically induced dimerization of the hook protein and the hook protein-binding domain, as described above, contacting the transduced cell with a third partner, in conditions suitable for the third partner to interact with the hook protein and the hook protein-binding domain expressed therein,
   thereby trapping said protein of interest, upon its expression, in said cell to a cellular compartment of the cell, and
(c)
   (i) contacting said cell with a competing molecule, wherein said competing molecule binds to the hook protein, and/or
   (ii) where the transduced cell is contacted with a third partner at step (b)(ii), interrupting the contact of the transduced cell with the third partner,
   thereby releasing said protein of interest from the cellular compartment of the cell and allowing its secretion or cell membrane-anchorage.

The present invention also relates the polynucleotide (or the system of at least two polynucleotides) described above or the vector (or the system of at least two vectors) described above, for use in method of modulating the secretion or the cell membrane-anchorage of a protein of interest, comprising the steps of:
(a) transducing a cell with the polynucleotide (or the system of at least two polynucleotides) described above or the vector (or the system of at least two vectors) described above,
(b)
   (i) having the transduced cell of step (a) express a hook protein fused to a cellular compartment-retention peptide and the protein of interest fused to a hook protein-binding domain, and
   (ii) optionally, where a third partner is necessary to achieve chemically induced dimerization of the hook protein and the hook protein-binding domain, as described above, contacting the transduced cell with a third partner, in conditions suitable for the third partner to interact with the hook protein and the hook protein-binding domain expressed therein,
   thereby trapping said protein of interest, upon its expression, in said cell to a cellular compartment of the cell, and
(c)
   (i) contacting said cell with a competing molecule, wherein said competing molecule binds to the hook protein, and/or
   (ii) where the transduced cell is contacted with a third partner at step (b)(ii), interrupting the contact of the transduced cell with the third partner, thereby releasing said protein of interest from the cellular compartment of the cell and allowing its secretion or cell membrane-anchorage.

By “secretion”, it is meant a process whereby the protein of interest is transported from the inside of the cell to the outside of the cell, preferably via a process that does not involve concomitant cell death. Additionally, the term “secretion” encompassed those steps of the process whereby the protein of interest, prior to being transported outside of the cell, trips through the secretory apparatus of the cell.

By “cell membrane-anchorage”, it is meant a process whereby the protein of interest is embedded in the phospholipid bilayer of a cell. “Cell membrane-anchorage” can either result in the protein of interest spanning the entire phospholipid bilayer, and extending, to some extent, on each side of the phospholipid bilayer; or being only partially inserted in the phospholipid bilayer, and extending on one side only of the phospholipid bilayer, either extracellular or intracellular.

Examples of cellular compartments include, but are not limited to, the endoplasmic reticulum (ER), the ER-Golgi intermediate compartment (ERGIC), the Golgi apparatus, the mitochondrion, the nucleus, vesicles and cell membrane. In particular, vesicles include those involved in protein degradation mechanisms, such as, e.g., peroxisomes and lysosomes; transport vesicles, involved in material transport between cellular compartments; secretory vesicles, involved in material excretion from the cell; and extracellular vesicles, such as, e.g., exosomes, ectosomes and microvesicles.

Examples of suitable third partners have been described above.

In one embodiment, the competing molecule comprises or consists of a hook protein-binding domain.

In one embodiment, the competing molecule competes with the hook protein-binding domain fused to the protein of interest for binding to the hook protein.

In one embodiment, the competing molecule and the hook protein-binding domain fused to the protein of interest are identical.

In one embodiment, the competing molecule and the hook protein-binding domain fused to the protein of interest are different.

In one embodiment, the competing molecule binds to the hook protein with a better binding affinity than does the hook protein-binding domain fused to the protein of interest.

In one embodiment, the cell is contacted with the competing molecule at a concentration ranging from about 1 μM to about 500 μM, preferably from about 1 μM to about 100 μM, preferably from about 10 μM to about 100 μM. In one embodiment, the cell is contacted with the competing molecule at a concentration of about 1 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 150 μM, 200 μM, 250 μM, 300 μM, 350 μM, 400 μM, 450 μM, or 500 μM.

In one embodiment, the competing molecule is biotin or a derivative thereof.

As used herein, the term “biotin”, also called “vitamin H”, “vitamin B7”, “vitamin B8” or “2,3,3a,4,6,6a-hexahydro-2-oxo-1H-thieno[3,4-d]imidazole-4-pentanoic acid”, refers to a water-soluble B vitamin with the following formula:

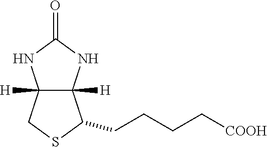 [SMILES: O=C(O)CCCC[C@@H]1SC[C@@H]2NC(=O)N[C@@H]21]

In one embodiment, derivatives of biotin include compounds of Formula (I):

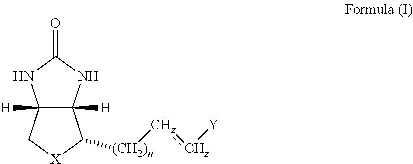

wherein:
X is selected from H₂, O, S, Se, SO, and SO₂,
Y is selected from CONH(CH₂)₄CH(NH₂)COOH, COOH, and OH,
n is 1, 2 or 3, and
z is 1 or 2.

In particular, derivatives of biotin include, but are not limited to, biocytin, dethiobiotin, selenobiotin, biotin sulfoxide, oxybiotin, biotinol, norbiotin, homobiotin, α-dehydrobiotin, and biotin sulfone, wherein “X”, “n”, “z” and “Y” in Formula (I) are defined as follows:

| Name | X | n | z | Y |
|---|---|---|---|---|
| Biocytin | S | 2 | 2 | CONH(CH₂)₄CH(NH₂)COOH |
| Dethiobiotin | H₂ | 2 | 2 | COOH |
| Selenobiotin | Se | 2 | 2 | COOH |
| Biotin sulfoxide | SO₂ | 2 | 2 | COOH |
| Oxybiotin | O | 2 | 2 | COOH |
| Biotinol | S | 2 | 2 | OH |
| Norbiotin | S | 1 | 2 | COOH |
| Homobiotin | S | 3 | 2 | COOH |
| α-dehydrobiotin | S | 2 | 1 | COOH |
| Biotin sulfone | SO | 2 | 2 | COOH |

In one embodiment, the competing molecule is a ligand able to mediate the interaction between the FKBP-binding protein or a derivative thereof and the FKBP protein or a derivative thereof.

In one embodiment, the ligand able to mediate the interaction between the FKBP-binding protein or a derivative thereof and the FKBP protein or a derivative thereof is selected from the group comprising or consisting of FK506, FK1012, FKCsA, rapamycin, and analogs thereof, such as, e.g., C16-AiRap, C16-iRap, C20-Marap and BS-Rap, described above.

As an example, the third partner can be rapamycin, and FK506 can be used as a competing molecule, therefore be added, either with or without interrupting the contact of the transduced cell with rapamycin at step (c).

The present invention further relates to a method of preventing and/or treating a disease in a subject in need thereof, said method comprising:
(a) in a first step, administering to said subject:
   (i) the polynucleotide (or the system of at least two polynucleotides) described above, the vector (or the system of at least two vectors) described above, the cell described above, or the composition described above, thereby having a cell of the subject expressing a hook protein fused to a cellular compartment-retention peptide and a protein of interest fused to a hook protein-binding domain, and
   (ii) optionally, where a third partner is necessary to achieve chemically induced dimerization of the hook protein and the hook protein-binding domain, as described above, with a third partner; and
(b) in a second step:
   (i) administering to said subject a competing molecule, and/or
   (ii) optionally, where the subject was administered with a third partner at step (a)(ii), interrupting administration of the third partner,
thereby preventing or treating the disease in said subject.

The present invention also relates to the polynucleotide (or the system of at least two polynucleotides) described above, the vector (or the system of at least two vectors) described above, the cell described above, or the composition described above, for use in a method of preventing and/or treating a disease in a subject in need thereof, wherein
(a) in a first step:
   (i) the polynucleotide (or the system of at least two polynucleotides) described above, the vector (or the system of at least two vectors) described above, the cell described above, or the composition described above, is to be administered to the subject, thereby having a cell of the subject expressing a hook protein fused to a cellular compartment-retention peptide and a protein of interest fused to a hook protein-binding domain,
   (ii) optionally, where a third partner is necessary to achieve chemically induced dimerization of a hook protein and a hook protein-binding domain, as described above, the subject is further to be administered with the third partner, and
(b) in a second step:
   (i) a competing molecule is to be administered to the subject, and/or
   (ii) optionally, where the subject was administered with a third partner at step (a)(ii), administration of said third partner to the subject is interrupted.

The term “preventing” and its derivatives refers to prophylactic measures, wherein the aim is to inhibit or delay the occurrence of the targeted disease(s) or the onset of clinical symptoms associated with the targeted disease(s). Those in need of prevention include those not affect with the targeted disease(s).

The term “treating” and its derivatives refers to therapeutic measures, wherein the aim is to abrogate, slow down, lessen and/or reverse the progression of the targeted disease(s) or of the clinical symptoms associated with the targeted disease(s). Those in need of treatment include those already with the targeted disease(s) as well those suspected to have the targeted disease(s).

Examples of suitable third partners have been described above.

In one embodiment, the competing molecule comprises or consists of a hook protein-binding domain.

In one embodiment, the competing molecule competes with the hook protein-binding domain fused to the protein of interest for binding to the hook protein.

In one embodiment, the competing molecule and the hook protein-binding domain fused to the protein of interest are identical.

In one embodiment, the competing molecule and the hook protein-binding domain fused to the protein of interest are different.

In one embodiment, the competing molecule binds to the hook protein with a better binding affinity than does the hook protein-binding domain fused to the protein of interest.

In one embodiment, the competing molecule is to be administered to said subject at a concentration ranging from about 1 μM to about 500 μM, preferably from about 1 μM to about 100 μM, preferably from about 10 μM to about 100 μM. In one embodiment, the competing molecule is to be administered to said subject at a concentration of about 1 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 150 μM, 200 μM, 250 μM, 300 μM, 350 μM, 400 μM, 450 μM, or 500 μM.

In one embodiment, the competing molecule is to be administered to said subject about 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, 4 weeks, or more after administration of the polynucleotide (or the system of at least two polynucleotides) described above, the vector (or the system of at least two vectors) described above, the cell described above, or the composition described above.

In one embodiment, administration of the competing molecule can be acute, or chronic.

In one embodiment, the competing molecule is to be administered to said subject every 4 hours, 6 hours, 12 hours, 1 day, 2 days, 3 days, 4 days, 5 days, 6 days, 1 week, 2 weeks, 3 weeks, 4 weeks, or more.

Examples of competing molecules have been described above. In one embodiment, the competing molecule is biotin or a derivative thereof. Biotin and its derivatives have been described above. In one embodiment, the competing molecule is a ligand able to mediate the interaction between the FKBP-binding protein or a derivative thereof and the FKBP protein or a derivative thereof. Ligands able to mediate the interaction between the FKBP-binding protein or a derivative thereof and the FKBP protein or a derivative thereof have been described above.

In one embodiment, the disease is selected from the group comprising or consisting of cancers, autoimmune diseases, inflammatory diseases, metabolic and endocrine diseases, neurodegenerative diseases, infectious diseases, and genetic disorders.

Examples of cancers include, but are not limited to, adenofibroma, adenoma, agnogenic myeloid metaplasia, AIDS-related malignancies, ameloblastoma, anal cancer, angiofollicular mediastinal lymph node hyperplasia, angiokeratoma, angiolymphoid hyperplasia with eosinophilia, angiomatosis, anhidrotic ectodermal dysplasia, anterofacial dysplasia, apocrine metaplasia, apudoma, asphyxiating thoracic dysplasia, astrocytoma (including, e.g., cerebellar astrocytoma and cerebral astrocytoma), atriodigital dysplasia, atypical melanocytic hyperplasia, atypical metaplasia, autoparenchymatous metaplasia, basal cell hyperplasia, benign giant lymph node hyperplasia, bile duct cancer (including, e.g., extrahepatic bile duct cancer), bladder cancer, bone cancer, brain tumour (including, e.g., brain stem glioma, cerebellar astrocytoma glioma, malignant glioma, supratentorial primitive neuroectodermal tumours, visual pathway and hypothalamic glioma, ependymoma, medulloblastoma, gestational trophoblastic tumour glioma, and paraganglioma), branchionia, female breast cancer, male breast cancer, bronchial adenomas/carcinoids, bronchopulmonary dysplasia, cancer growths of epithelial cells, pre-cancerous growths of epithelial cells, metastatic growths of epithelial cells, carcinoid heart disease, carcinoid tumour (including, e.g., gastrointestinal carcinoid tumour), carcinoma (including, e.g., carcinoma of unknown primary origin, adrenocortical carcinoma, islet cells carcinoma, adeno carcinoma, adeoncortical carcinoma, basal cell carcinoma, basosquamous carcinoma, bronchiolar carcinoma, Brown-Pearce carcinoma, cystadenocarcinoma, ductal carcinoma, hepatocarcinoma, Krebs carcinoma, papillary carcinoma, oat cell carcinoma, small cell lung carcinoma, non-small cell lung carcinoma, squamous cell carcinoma, transitional cell carcinoma, Walker carcinoma, Merkel cell carcinoma, and skin carcinoma), cementoma, cementum hyperplasia, cerebral dysplasia, cervical cancer, cervical dysplasia, cholangioma, cholesteatoma, chondroblastoma, chondroectodermal dysplasia, chordoma, choristoma, chrondroma, cleidocranial dysplasia, colon cancer, colorectal cancer, local metastasized colorectal cancer, congenital adrenal hyperplasia, congenital ectodermal dysplasia, congenital sebaceous hyperplasia, connective tissue metaplasia, craniocarpotarsal dysplasia, craniodiaphysial dysplasia, craniometaphysial dysplasia, craniopharyngioma, cylindroma, cystadenoma, cystic hyperplasia (including, e.g., cystic hyperplasia of the breast), cystosarconia phyllodes, dentin dysplasia, denture hyperplasia, diaphysial dysplasia, ductal hyperplasia, dysgenninoma, dysplasia epiphysialis hemimelia, dysplasia epiphysialis multiplex, dysplasia epiphysialis punctate, ectodermal dysplasia, Ehrlich tumour, enamel dysplasia, encephaloophthalmic dysplasia, endometrial cancer (including, e.g., ependymoma and endometrial hyperplasia), ependymoma, epithelial cancer, epithelial dysplasia, epithelial metaplasia, esophageal cancer, Ewing's family of tumours (including, e.g., Ewing's sarcoma), extrahepatic bile duct cancer, eye cancer (including, e.g., intraocular melanoma and retinoblastoma), faciodigitogenital dysplasia, familial fibrous dysplasia of jaws, familial white folded dysplasia, fibroma, fibromuscular dysplasia, fibromuscular hyperplasia, fibrous dysplasia of bone, florid osseous dysplasia, focal epithelial hyperplasia, gall bladder cancer, ganglioneuroma, gastric cancer (including, e.g., stomach cancer), gastrointestinal carcinoid tumour, gastrointestinal tract cancer, gastrointestinal tumours, Gaucher's disease, germ cell tumours (including, e.g., extracranial germ cell tumours, extragonadal germ cell tumours, and ovarian germ cell tumours), giant cell tumour, gingival hyperplasia, glioblastoma, glomangioma, granulosa cell tumour, gynandroblastoma, hamartoma, head and neck cancer, hemangioendothelioma, hemangioma, hemangiopericytoma, hepatocellular cancer, hepatoma, hereditary renal-retinal dysplasia, hidrotic ectodermal dysplasia, histiocytonia, histiocytosis, hypergammaglobulinemia, hypohidrotic ectodermal dysplasia, hypopharyngeal cancer, inflammatory fibrous hyperplasia, inflammatory papillary hyperplasia, intestinal cancers, intestinal metaplasia, intestinal polyps, intraocular melanoma, intravascular papillary endothelial hyperplasia, kidney cancer, laryngeal cancer, leiomyoma, leukemia (including, e.g., acute lymphoblastic leukemia, acute lymphocytic leukemia, acute myeloid leukemia, acute myelogenous leukemia, acute hairy cell leukemia, acute B-cell leukemia, acute T-cell leukemia, acute HTLV leukemia, chronic lymphoblastic leukemia, chronic lymphocytic leukemia, chronic myeloid leukemia, chronic myelogenous leukemia, chronic hairy cell leukemia, chronic B-cell leukemia, chronic T-cell leukemia, and chronic HTLV leukemia), Leydig cell tumour, lip and oral cavity cancer, lipoma, liver cancer, lung cancer (including, e.g., small cell lung cancer and non-small cell lung cancer), lymphangiomyoma, lymphaugioma, lymphoma (including, e.g., AIDS-related lymphoma, central nervous system lymphoma, primary central nervous system lymphoma, Hodgkin's lymphoma, non-Hodgkin's lymphoma, Hodgkin's lymphoma during pregnancy, non-Hodgkin's lymphoma during pregnancy, mast cell lymphoma, B-cell lymphoma, adenolymphoma, Burkitt's lymphoma, cutaneous T-cell lymphoma, large cell lymphoma, and small cell lymphoma), lymphopenic thymic dysplasia, lymphoproliferative disorders, macroglobulinemia (including, e.g., Waldenstrom's macroglobulinemia), malignant carcinoid syndrome, malignant mesothelioma, malignant thymoma, mammary dysplasia, mandibulofacial dysplasia, medulloblastoma, meningioma, mesenchymoma, mesonephroma, mesothelioma (including, e.g., malignant mesothelioma), metaphysial dysplasia, metaplastic anemia, metaplastic ossification, metaplastic polyps, metastatic squamous neck cancer (including, e.g., metastatic squamous neck cancer with occult primary), Mondini dysplasia, monostotic fibrous dysplasia, mucoepithelial dysplasia, multiple endocrine neoplasia syndrome, multiple epiphysial dysplasia, multiple myeloma/plasma cell neoplasm, mycosis fungoides, myelodysplastic syndrome, myeloid metaplasia, myeloproliferative disorders, chronic myeloproliferative disorders, myoblastoma, myoma, myxoma, nasal cavity and paranasal sinus cancer, nasopharyngeal cancer, prostatic neoplasm, colon neoplasm, abdomen neoplasm, bone neoplasm, breast neoplasm, digestive system neoplasm, liver neoplasm, pancreas neoplasm, peritoneum neoplasm, endocrine glands neoplasm (including, e.g., adrenal neoplasm, parathyroid neoplasm, pituitary neoplasm, testicles neoplasm, ovary neoplasm, thymus neoplasm, and thyroid neoplasm), eye neoplasm, head and neck neoplasm, nervous system neoplasm (including, e.g., central nervous system neoplasm and peripheral nervous system neoplasm), lymphatic system neoplasm, pelvic neoplasm, skin neoplasm, soft tissue neoplasm, spleen neoplasm, thoracic neoplasm, urogenital tract neoplasm, neurilemmoma, neuroblastoma, neuroepithelioma, neurofibroma, neurofibromatosis, neuroma, nodular hyperplasia of prostate, nodular regenerative hyperplasia, oculoauriculovertebral dysplasia, oculodentodigital dysplasia, oculovertebral dysplasia, odontogenic dysplasia, odontoma, opthalmomandibulomelic dysplasia, oropharyngeal cancer, osteoma, ovarian cancer (including, e.g., ovarian epithelial cancer and ovarian low malignant potential tumour), pancreatic cancer (including, e.g., islet cell pancreatic cancer and exocrine pancreatic cancer), papilloma, paraganglioma, nonchromaffin paraganglioma, paranasal sinus and nasal cavity cancer, paraproteinemias, parathyroid cancer, periapical cemental dysplasia, pheochromocytoma (including, e.g., penile cancer), pineal and supratentorial primitive neuroectodermal tumours, pinealoma, pituitary tumour, plasma cell neoplasm/multiple myeloma, plasmacytoma, pleuropulmonary blastoma, polyostotic fibrous dysplasia, polyps, pregnancy cancer, pre-neoplastic disorders (including, e.g., benign dysproliferative disorders such as benign tumours, fibrocystic conditions, tissue hypertrophy, intestinal polyps, colon polyps, esophageal dysplasia, leukoplakia, keratoses, Bowen's disease, Farmer's skin, solar cheilitis, and solar keratosis), primary hepatocellular cancer, primary liver cancer, primary myeloid metaplasia, prostate cancer, pseudoachondroplastic spondyloepiphysial dysplasia, pseudoepitheliomatous hyperplasia, purpura, rectal cancer, renal cancer (including, e.g., kidney cancer, renal pelvis, ureter cancer, transitional cell cancer of the renal pelvis and ureter), reticuloendotheliosis, retinal dysplasia, retinoblastoma, salivary gland cancer, sarcomas (including, e.g., uterine sarcoma, soft tissue sarcoma, carcinosarcoma, chondrosarcoma, fibrosarcoma, hemangiosarcoma, Kaposi's sarcoma, leiomyosarcoma, liposarcoma, lymphangiosarcoma, myosarcoma, myxosarcoma, rhabdosarcoma, sarcoidosis sarcoma, osteosarcoma, Ewing sarcoma, malignant fibrous histiocytoma of bone, and clear cell sarcoma of tendon sheaths), sclerosing angioma, secondary myeloid metaplasia, senile sebaceous hyperplasia, septooptic dysplasia, Sertoli cell tumour, Sezary syndrome, skin cancer (including, e.g., melanoma skin cancer and non-melanoma skin cancer), small intestine cancer, spondyloepiphysial dysplasia, squamous metaplasia (including, e.g., squamous metaplasia of amnion), stomach cancer, supratentorial primitive neuroectodermal and pineal tumours, supratentorial primitive neuroectodermal tumours, symptomatic myeloid metaplasia, teratoma, testicular cancer, theca cell tumour, thymoma (including, e.g., malignant thymoma), thyroid cancer, trophoblastic tumours (including, e.g., gestational trophoblastic tumours), ureter cancer, urethral cancer, uterine cancer, vaginal cancer, ventriculoradial dysplasia, verrucous hyperplasia, vulvar cancer, Waldenstrom's macroglobulinemia, and Wilms' tumour.

Examples of autoimmune diseases include, but are not limited to, alopecia areata, ankylosing spondylitis, arthritis, antiphospholipid syndrome, autoimmune Addison's disease, autoimmune hemolytic anemia, autoimmune inner ear disease (also known as Menibre's disease), autoimmune lymphoproliferative syndrome, autoimmune thrombocytopenic purpura, autoimmune hemolytic anemia, autoimmune hepatitis, Bechet's disease, Crohn's disease, diabetes mellitus type 1, glomerulonephritis, Graves' disease, Guillain-Barre syndrome, inflammatory bowel disease, lupus nephritis, multiple sclerosis, myasthenia gravis, pemphigus, pernicous anemia, polyarteritis nodosa, polymyositis, primary biliary cirrhosis, psoriasis, Raynaud's phenomenon, rheumatic fever, rheumatoid arthritis, scleroderma, Sjögren's syndrome, systemic lupus erythematosus, ulcerative colitis, vitiligo, and Wegener's granulomatosis.

Examples of inflammatory disease include, but are not limited to, abdominal aortic aneurysm (AAA), acne, acute disseminated encephalomyelitis, acute leukocyte-mediated lung injury, Addison's disease, adult respiratory distress syndrome, AIDS dementia, allergic asthma, allergic conjunctivitis, allergic rhinitis, allergic sinusitis, alopecia areata, Alzheimer's disease, anaphylaxis, angioedema, ankylosing spondylitis, antiphospholipid antibody syndrome, asthma, atopic dermatitis, autoimmune hemolytic anemia, autoimmune hepatitis, autoimmune inner ear disease, Behcet's syndrome, blepharitis, bronchitis, bullous pemphigoid, Chagas' disease, chronic inflammatory diseases, chronic obstructive pulmonary disease, coagulative necrosis, coeliac disease, collagenous colitis, conjunctivitis, contact dermatitis, coronary heart disease, cutaneous necrotizing venulitis, cystic fibrosis, dermatitis, dermatomyositis, diabetes mellitus type 1, diabetes mellitus type 2, distal proctitis, diversion colitis, dry eye, eczema, encephalitis, endometriosis, endotoxin shock, epilepsy, erythema multiforme, erythema nodosum, fibrinoid necrosis, fibromyalgia, giant-cell arteritis (Horton's disease), goodpasture's syndrome, gouty arthritis, graft-versus-host disease (such as, e.g., acute graft-versus-host disease, chronic graft-versus-host disease, and the like), Graves' disease, Guillain-Barre syndrome, Hashimoto's disease, hay fever, hyperacute transplant rejection, hyperlipidemia, idiopathic thrombocytopenic purpura, indeterminate colitis, infective colitis, inflammatory bowel disease (IBD) (such as, e.g., Crohn's disease, ulcerative colitis, colitis, and the like), inflammatory liver disorder, insect bite skin inflammation, interstitial cystitis, iritis, ischaemic colitis, lichen planus, liquefactive necrosis, lupus erythematosus, lymphocytic colitis, meningitis, metabolic syndrome, multiple sclerosis, myasthenia gravis, myocarditis, narcolepsy, nephritis, obesity, pancreatitis, Parkinson's disease, pemphigus vulgaris, periodontal gingivitis, periodontitis, pernicious anaemia, polymyalgia rheumatica, polymyositis, postmenopausal-induced metabolic syndrome, primary biliary cirrhosis, psoriasis, retinitis, rheumatoid arthritis, rheumatoid spondylitis, rhinoconjunctivitis, scleroderma, shingles, Sjogren's syndrome, smooth muscle proliferation disorders, solar dermatitis, steatosis, systemic lupus erythematosus (SLE), tuberculosis, urticaria, uveitis, vasculitis, vitiligo, and Wegener's granulomatosis.

Examples of metabolic diseases include, but are not limited to, diabetes (e.g., type 1 diabetes, type 2 diabetes, gestational diabetes), hyperglycemia, insulin resistance, impaired glucose tolerance, hyperinsulinism, diabetic complication, dyslipidemia, hypercholesterolemia, hypertriglyceridemia, HDL hypocholesterolemia, LDL hypercholesterolemia and/or HLD non-cholesterolemia, VLDL hyperproteinemia, dyslipoproteinemia, apolipoprotein A-1 hypoproteinemia, metabolic syndrome, syndrome X, obesity, non-alcoholic fatty liver disease (NAFLD), non-alcoholic steatohepatitis, and adrenal leukodystrophy.

Examples of neurodegenerative diseases include, but are not limited to, Parkinson's disease and related disorders (including, e.g., Parkinson's disease, Parkinson-dementia, autosomal recessive PARK2 and PARK6-linked Parkinsonism, atypical parkinsonian syndromes, including, progressive supranuclear palsy, corticobasal degeneration syndrome, Lewy bodies dementia, multiple system atrophy, Guadeloupean Parkinsonism and Lytigo-bodig disease), motor neuron diseases (including, e.g., amyotrophic lateral sclerosis, frontotemporal dementia, progressive bulbar palsy, pseudobulbar palsy, primary lateral sclerosis, progressive muscular atrophy, spinal muscular atrophy and post-polio syndrome), neuro-inflammatory diseases, Alzheimer's disease and related disorders (including, e.g., early stage of an Alzheimer's disorder, mild stage of an Alzheimer's disorder, moderate stage of an Alzheimer's disorder, mild to moderate stage of an Alzheimer's disorder, advanced stage of an Alzheimer's disorder, mild cognitive impairment, vascular dementia, mixed dementia, Pick's disease, argyrophilic grain disease, posterior cortical atrophy, and Wernicke-Korsakoff syndrome), prion diseases, lysosomal storage diseases, leukodystrophies, Huntington's disease, multiple sclerosis, Down syndrome, spinal and bulbar muscular atrophy, HIV-associated neurocognitive disorder, Tourette syndrome, autosomal dominant spinocerebellar ataxia, Friedreich's ataxia, dentatorubral pallidoluysian atrophy, myotonic dystrophy, schizophrenia, age associated memory impairment, autism and autism spectrum disorders, attention-deficit hyperactivity disorder, chronic pain, alcohol-induced dementia, progressive non-fluent aphasia, semantic dementia, spastic paraplegia, fibromyalgia, post-Lyme disease, neuropathies, withdrawal symptoms, Alpers' disease, cerebro-oculo-facio-skeletal syndrome, Wilson's disease, Cockayne syndrome, Leigh's disease, neurodegeneration with brain iron accumulation, dyskinesia, dystonia (including, e.g., status dystonicus, spasmodic torticollis, Meige's syndrome, and blepharospasm), athetosis, chorea, choreoathetosis, opsoclonus myoclonus syndrome, myoclonus, myoclonic epilepsy, akathisia, tremor (including, e.g., essential tremor, and intention tremor), restless legs syndrome, stiff-person syndrome, alpha-methylacyl-CoA racemase deficiency, Andermann syndrome, Arts syndrome, Marinesco-Sjögren syndrome, mitochondrial membrane protein-associated neurodegeneration, pantothenate kinase-associated neurodegeneration, polycystic lipomembranous osteodysplasia with sclerosing leukoencephalopathy, riboflavin transporter deficiency neuronopathy, and ataxia telangiectasia.

Examples of infectious diseases include, but are not limited to, Acinetobacter infections, actinomycosis, African sleeping sickness (also named African trypanosomiasis), AIDS (acquired immunodeficiency syndrome), amoebiasis, anaplasmosis, angiostrongyliasis, anisakiasis, anthrax, Arcanobacterium haemolyticum infection, Argentine hemorrhagic fever, ascariasis, aspergillosis, Astrovirus infection, babesiosis, Bacillus cereus infection, bacterial meningitis, bacterial pneumonia, bacterial vaginosis, Bacteroides infection, balantidiasis, bartonellosis, Baylisascaris infection, BK virus infection, Black Piedra, blastocystosis, blastomycosis, Bolivian hemorrhagic fever, botulism, Brazilian hemorrhagic fever, brucellosis, bubonic plague, Burkholderia infection, Buruli ulcer, Calicivirus infection, campylobacteriosis, candidiasis, capillariasis, Carrion's disease, cat-scratch disease, cellulitis, Chagas disease (also named American trypanosomiasis), chancroid, chickenpox, chikungunya, chlamydia, Chlamydophila pneumoniae infection, cholera, chromoblastomycosis, chytridiomycosis, clonorchiasis, Clostridium difficile colitis, coccidioidomycosis, Colorado tick fever, common cold (also named acute viral rhinopharyngitis or acute coryza), coronavirus disease 2019 (also named COVID-19), Creutzfeldt-Jakob disease, Crimean-Congo hemorrhagic fever, cryptococcosis, cryptosporidiosis, cutaneous larva migrans, cyclosporiasis, cysticercosis, cytomegalovirus infection, dengue fever, desmodesmus infection, dientamoebiasis, diphtheria, diphyllobothriasis, dracunculiasis, Ebola hemorrhagic fever, echinococcosis, ehrlichiosis, enterobiasis, enterococcus infection, enterovirus infection, epidemic typhus, erythema infectiosum, exanthem subitem, fasciolasis, fasciolopsiasis, fatal familial insomnia, filariasis, Clostridium perfringens poisoning, free-living amebic infection, Fusobacterium infection, gas gangrene, geotrichosis, Gerstmann-Sträussler-Scheinker syndrome, giardiasis, glanders, gnathostomiasis, gonorrhea, granuloma inguinale, group A streptococcal infection, group B streptococcal infection, Haemophilus influenzae infection, hand foot and mouth disease, hantavirus pulmonary syndrome, Heartland virus disease, Helicobacter pylori infection, hemolytic-uremic syndrome, hemorrhagic fever with renal syndrome, hendra virus infection, hepatitis A, hepatitis B, hepatitis C, hepatitis D, hepatitis E, herpes simplex, histoplasmosis, hookworm infection, human bocavirus infection, human ewingii ehrlichiosis, human granulocytic anaplasmosis, human metapneumovirus infection, human monocytic ehrlichiosis, human papillomavirus infection, human parainfluenza virus infection, hymenolepiasis, infant botulism, infectious mononucleosis, influenza (also named flu), isosporiasis, Kawasaki disease, keratitis, Kingella kingae infection, kuru, lassa fever, legionellosis, leishmaniasis, leprosy, leptospirosis, listeriosis, lyme disease, lymphatic filariasis, lymphocytic choriomeningitis, malaria, Marburg hemorrhagic fever, measles, middle East respiratory syndrome (also named MERS), melioidosis (also named Whitmore's disease), meningitis, meningococcal disease, metagonimiasis, microsporidiosis, molluscum contagiosum, monkeypox, mumps, murine typhus, mycoplasma pneumonia, Mycoplasma genitalium infection, mycetoma, myiasis, neonatal conjunctivitis, nipah virus infection, nocardiosis, onchocerciasis (also named river blindness), opisthorchiasis, paracoccidioidomycosis (also named South American blastomycosis), paragonimiasis, pasteurellosis, pediculosis capitis, pediculosis corporis, pediculosis pubis, pelvic inflammatory disease, pertussis (also named whooping cough), plague, pneumococcal infection, pneumocystis pneumonia, pneumonia, poliomyelitis, Prevotella infection, primary amoebic meningoencephalitis, progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley fever, Rocky Mountain spotted fever, rotavirus infection, rubella, salmonellosis, scabies, scarlet fever, schistosomiasis, sepsis, severe acute respiratory syndrome (also named SARS), shigellosis, shingles, smallpox (also named variola), sporotrichosis, staphylococcal food poisoning, staphylococcal infection, strongyloidiasis, subacute sclerosing panencephalitis, syphilis, taeniasis, tetanus, tinea barbae, tinea blanca, tinea capitis, tinea corporis, tinea cruris, tinea manum, tinea nigra, tinea pedis, tinea unguium, tinea versicolor, toxocariasis, toxoplasmosis, trachoma, trichinosis, trichomoniasis, trichuriasis, tuberculosis, tularemia, typhoid fever, typhus fever, Ureaplasma urealyticum infection, Valley fever, Venezuelan equine encephalitis, Venezuelan hemorrhagic fever, Vibrio vulnificus infection, Vibrio parahaemolyticus enteritis, viral pneumonia, West Nile fever, Yersinia pseudotuberculosis infection, yersiniosis, yellow fever, zeaspora, zika fever, and zygomycosis.

Examples of genetic disorders include, but are not limited to, hemophilia, sickle-cell anemia, Down syndrome, Tay-Sachs disease, cystic fibrosis, cerebral palsy, Marfan syndrome, muscular dystrophies (including, e.g., Duchenne's muscular dystrophy, Becker's muscular dystrophy, facioscapulohumeral muscular dystrophy, and myotonic muscular dystrophy), ataxia-telangiectasia, Hurler syndrome, Usher syndrome, factor VII deficiency, familial atrial fibrillation, Hailey-Hailey disease, McArdle disease, mucopolysaccharidosis, nephropathic cystinosis, polycystic kidney disease, Rett syndrome, spinal muscular atrophy (SMA), X-linked nephrogenic diabetes insipidus (XNDI), X-linked retinitis pigmentosa, and color blindness.

In one embodiment, the subject in need thereof is an animal. Examples of animals include, but are not limited to, mammals, birds, reptiles, amphibians, fishes, insects and mollusks.

In one embodiment, the subject in need thereof is a non-human animal, including, but not limited to, a farm animal—or an animal of agricultural value (such as, e.g., cattle, cows, bison, pigs, swine, sheep, goats, horses, donkeys, alpacas, llamas, deer, elks, moose, ostriches, emus, ducks, geese, chickens, partridges, quails, pheasants, minks, salmons, codfishes, catfishes, herrings, trout, basses, perches, flounders, sharks, tuna fishes, cancers, lobsters, crayfishes, snails, clams, oysters, and the like), a companion animal (such as, e.g., dog, cats, rabbits, rodents, fishes, snakes and the like), and a non-human primate (such as, e.g., great apes including chimpanzees, gorillas, and orangutans; lesser apes, including gibbons; Old World monkeys; New World monkeys; and prosimians, including tarsiers, lemurs, and lorises).

In one embodiment, the subject in need thereof is a human.

In one embodiment, the subject in need thereof is an adult (e.g., a subject above the age of 18 in human years or a subject after reproductive capacity has been attained). In one embodiment, the subject in need thereof is a child (e.g., a subject below the age of 18 in human years or a subject before reproductive capacity has been attained).

In one embodiment, the subject in need thereof is a male. In one embodiment, the subject in need thereof is a female.

In one embodiment, the subject in need thereof is/was diagnosed with a disease, preferably selected from the group comprising or consisting of cancers, autoimmune diseases, inflammatory diseases, metabolic and endocrine diseases, neurodegenerative diseases, infectious diseases, and genetic disorders. In one embodiment, the subject in need thereof is at risk of developing a disease, preferably selected from the group comprising or consisting of cancers, autoimmune diseases, inflammatory diseases, metabolic and endocrine diseases, neurodegenerative diseases, infectious diseases, and genetic disorders.

The present invention also relates to a kit or a kit-of-parts, comprising:
(a) the polynucleotide (or the system of at least two polynucleotides) described above or the vector (or the system of at least two vectors) described above; (b) the competing molecule, as described above;
(c) optionally, a third partner, in particular where such third partner is necessary to achieve chemically induced dimerization of a hook protein and a hook protein-binding domain, as described above.

Examples of suitable competing molecule have been described above.

Examples of suitable third partners have been described above.

### BRIEF DESCRIPTION OF THE DRAWINGS

FIGS. 1A-E are a set of immunofluorescence photographs of transduced HeLa cells.

FIG. 1A is an immunofluorescence photograph of HeLa cells transduced with IL-2-SBP-eGFP.

FIG. 1B is an immunofluorescence photograph of HeLa cells transduced with CCL5-SBP-eGFP.

FIG. 1C is an immunofluorescence photograph of HeLa cells transduced with CXCL10-SBP-eGFP.

FIG. 1D is an immunofluorescence photograph of HeLa cells transduced with CCL19-SBP-eGFP.

FIG. 1E is an immunofluorescence photograph of HeLa cells transduced with IFNg-SBP-eGFP.

FIGS. 2A-E are a set of western blot photographs of transduced HeLa cells.

FIG. 2A is a western blot photograph of HeLa cells transduced with IL-2-SBP-eGFP.

FIG. 2B is a western blot photograph of HeLa cells transduced with CCL5-SBP-eGFP.

FIG. 2C is a western blot photograph of HeLa cells transduced with CXCL10-SBP-eGFP.

FIG. 2D is a western blot photograph of HeLa cells transduced with CCL19-SBP-eGFP.

FIG. 2E is a western blot photograph of HeLa cells transduced with IFNg-SBP-eGFP.

FIGS. 3A-D are a set of immunofluorescence photographs of transduced HeLa cells.

FIG. 3A is an immunofluorescence photograph of HeLa cells transduced with TNF-SBP-eGFP.

FIG. 3B is an immunofluorescence photograph of HeLa cells transduced with IL-7-SBP-eGFP.

FIG. 3C is an immunofluorescence photograph of HeLa cells transduced with IL-15-SBP-eGFP.

FIG. 3D is an immunofluorescence photograph of HeLa cells transduced with tPa6-IL-15-SBP-eGFP.

FIGS. 4A-E are a set of immunofluorescence photographs of transduced HeLa cells.

FIG. 4A is an immunofluorescence photograph of HeLa cells transduced with CXCL9-SBP-CH.

FIG. 4B is an immunofluorescence photograph of HeLa cells transduced with IL-12b-p2a-IL-12a-SBP-CH.

FIG. 4C is an immunofluorescence photograph of HeLa cells transduced with IL-21-SBP-eGFP.

FIG. 4D is an immunofluorescence photograph of HeLa cells transduced with GM-CSF-SBP-eGFP.

FIG. 4E is an immunofluorescence photograph of HeLa cells transduced with IL-8-SBP-eGFP.

FIGS. 5A-B are a set of immunofluorescence photographs of transduced HeLa cells.

FIG. 5A is an immunofluorescence photograph of HeLa cells transduced with SPCCL5-IL-2-SBP-eGFP.

FIG. 5B is an immunofluorescence photograph of HeLa cells transduced with CCL5-SBP-eGFP_{hibit}.

FIGS. 6A-B are a set of western blot photographs of transduced HeLa cells.

FIG. 6A is a western blot photograph of GFP and Vinculin revelation in cell medium of SPCCL5-IL-2-SBP-eGFP, IL-2-SBP-eGFP and CCL5-SBP-eGFP transduced HeLa cells.

FIG. 6B is a western blot photograph of GFP and Vinculin revelation in cell lysate of SPCCL5-IL-2-SBP-eGFP, IL-2-SBP-eGFP and CCL5-SBP-eGFP transduced HeLa cells.

FIGS. 7A-D are a set of immunofluorescence photographs of transduced HeLa cells.

FIG. 7A is an immunofluorescence photograph of HeLa cells transduced with IL-4-SBP-eGFP.

FIG. 7B is an immunofluorescence photograph of HeLa cells transduced with IFNa2-SBP-eGFP.

FIG. 7C is an immunofluorescence photograph of HeLa cells transduced with CCL21-SBP-eGFP.

FIG. 7D is an immunofluorescence photograph of HeLa cells transduced with SPCCL5-IL-36-SBP-eGFP.

FIGS. 8A-B are a set of western blot photographs of transduced HeLa cells.

FIG. 8A is a western blot photograph of GFP and Vinculin revelation in cell medium of IL-4-SBP-eGFP, IFNa2-SBP-eGFP, CCL21-SBP-eGFP and SPCCL5-IL-36-SBP-eGFP transduced HeLa cells.

FIG. 8B is a western blot photograph of GFP and Vinculin revelation in cell lysate of IL-4-SBP-eGFP, IFNa2-SBP-eGFP, CCL21-SBP-eGFP and SPCCL5-IL-36-SBP-eGFP transduced HeLa cells.

FIGS. 9A-D are a set of graphs representing GFP fluorescence in transduced HeLa cells.

FIG. 9A is a graph representing GFP fluorescence in HeLa cells transduced with IL-4-SBP-eGFP.

FIG. 9B is a graph representing GFP fluorescence in HeLa cells transduced with IFNa2-SBP-eGFP.

FIG. 9C is a graph representing GFP fluorescence in HeLa cells transduced with CCL21-SBP-eGFP.

FIG. 9D is a graph representing GFP fluorescence in HeLa cells transduced with SPCCL5-IL-36-SBP-eGFP.

FIGS. 10A-C are a set of graphs representing cytokine activity from transduced cell line.

FIG. 10A is a graph representing cytokine activity in a reporter cell line from HeLa transduced with SPCCL5-IL-2-SBP-eGFP, IL-2-SBP-eGFP or CCL5-SBP-eGFP.

FIG. 10B is a graph representing IL-2-induced proliferation of reporter cell line from Hela transduced with SPCCL5-IL-2-SBP-eGFP, IL-2-SBP-eGFP or CCL5-SBP-eGFP.

FIG. 10C is a graph representing NFκB-response of reporter cell line from HeLa transduced with SPCCL5-IL-2-SBP-eGFP, IL-2-SBP-eGFP or CCL5-SBP-eGFP.

FIGS. 11A-B are a set of western blot photographs of transduced HeLa cell lines used to induce the activity of the reporter cell.

FIG. 11A is a western blot photograph of GFP and lamin revelation in cell medium of SPCCL5-IL-2-SBP-eGFP, IL-2-SBP-eGFP and CCL5-SBP-eGFP transduced HeLa cell lines used to induce the activity of the reporter cell.

FIG. 11B is a western blot photograph of GFP and lamin revelation in cell lysate of SPCCL5-IL-2-SBP-eGFP, IL-2-SBP-eGFP and CCL5-SBP-eGFP transduced HeLa cell lines used to induce the activity of the reporter cell.

FIG. 12A-B are a set of a graph and a western blot photograph of Rh30-luciferase transduced cells.

FIG. 12A is a graph representing IFNγ activity from a reporter cell line by Rh30-luciferase cells transduced with IFNg-SBP-eGFP or SPCCL5-IL-2-SBP-eGFP.

FIG. 12B is a western blot photograph of GFP and vinculin revelation in Rh30 cells transduced with IFNg-SBP-eGFP used to induce the activity of the reporter cell.

FIGS. 13A-C are a set of immunofluorescence photographs of transduced Jurkat cells.

FIG. 13A is an immunofluorescence photograph of Jurkat cells transduced with IL-2-SBP-eGFP.

FIG. 13B is an immunofluorescence photograph of Jurkat cells transduced with CCL5-SBP-eGFP.

FIG. 13C is an immunofluorescence photograph of Jurkat cells transduced with CXCL10-SBP-eGFP.

FIGS. 14A-C are a set of graphs representing GFP fluorescence in transduced Jurkat cells.

FIG. 14A is a graph representing GFP fluorescence in Jurkat cells transduced with IL-2-SBP-eGFP.

FIG. 14B is a graph representing GFP fluorescence in Jurkat cells transduced with CCL5-SBP-eGFP.

FIG. 14C is a graph representing GFP fluorescence in Jurkat cells transduced with CXCL10-SBP-eGFP.

FIGS. 15A-C are a set of western blot photograph s of GFP staining in transduced Jurkat cells.

FIG. 15A is a western blot photograph of GFP staining in Jurkat cells transduced with IL-2-SBP-eGFP.

FIG. 15B is a western blot photograph of GFP staining in Jurkat cells transduced with CCL5-SBP-eGFP.

FIG. 15C is a western blot photograph of GFP staining in Jurkat cells transduced with CXCL10-SBP-eGFP.

FIGS. 16A-D are a set of graphs representing GFP fluorescence in transduced Jurkat cells.

FIG. 16A is a graph representing GFP fluorescence in Jurkat cells transduced with CCL5-SBP-eGFP_{hibit}.

FIG. 16B is a graph representing GFP fluorescence in Jurkat cells transduced with CCL19-SBP-eGFP.

FIG. 16C is a graph representing GFP fluorescence in Jurkat cells transduced with IFNg-SBP-eGFP.

FIG. 16D is a graph representing GFP fluorescence in Jurkat cells transduced with TNF-SBP-eGFP.

FIGS. 17A-C are a set of graphs representing GFP fluorescence in transduced Jurkat cells.

FIG. 17A is a graph representing GFP fluorescence in Jurkat cells transduced with IL-7-SBP-eGFP.

FIG. 17B is a graph representing GFP fluorescence in Jurkat cells transduced with IL-15-SBP-eGFP.

FIG. 17C is a graph representing GFP fluorescence in Jurkat cells transduced with tPa6-IL-15-SBP-eGFP.

FIGS. 18A-C are a set of graphs representing GFP fluorescence in transduced primary CD8⁺ T cells.

FIG. 18A is a graph representing GFP fluorescence in primary CD8⁺ T cells transduced with IL-2-SBP-eGFP.

FIG. 18B is a graph representing GFP fluorescence in primary CD8⁺ T cells transduced with CCL5-SBP-eGFP.

FIG. 18C is a graph representing GFP fluorescence in primary CD8⁺ T cells transduced with CXCL10-SBP-eGFP.

FIGS. 19A-C are a set of graphs representing GFP fluorescence in transduced primary T cells.

FIG. 19A is a graph representing GFP fluorescence in primary T cells transduced with CCL19-SBP-eGFP.

FIG. 19B is a graph representing GFP fluorescence in primary T cells transduced with IFNg-SBP-eGFP.

FIG. 19C is a graph representing GFP fluorescence in primary T cells transduced with TNF-SBP-eGFP.

FIGS. 20A-D are a set of graphs representing GFP fluorescence in transduced primary T cells.

FIG. 20A is a graph representing GFP fluorescence in primary T cells transduced with IL-7-SBP-eGFP.

FIG. 20B is a graph representing GFP fluorescence in primary T cells transduced with IL-15-SBP-eGFP.

FIG. 20C is a graph representing GFP fluorescence in primary T cells transduced with tPa6-IL-15-SBP-eGFP.

FIG. 20D is a graph representing GFP fluorescence in primary T cells transduced with CCL5-SBP-eGFP_{Hibit}.

FIG. 21 is a set of photographs extracted from a movie of real-time cell imaging of CCL5-SBP-eGFP transduced primary macrophages.

FIG. 22 is a graph representing the percentage of HEK293FT cells transduced with a hook protein under the control of strong promoter sFFv and a cytokine under the control of a weaker promoter PGK (pPGK-IL-2 GFP or pPGK-CCL5 GFP), downstream of IVS-IRES (ivsIRES-IL-2 GFP or ivsIRES-CCL5 GFP) or stronger promoter sFFv x(prsFFv-IL-2 GFP or prsFFV-CCL5 GFP) vector.

FIGS. 23 A-F are a set of graphs representing GFP fluorescence in Jurkat cell.

FIG. 23A is a graph representing GFP fluorescence in Jurkat cells transduced with Hook under the control of strong promoter sFFv and the cytokine under the control of a weaker promoter PGK said cytokine being IL-2-GFP.

FIG. 23B is a graph representing GFP fluorescence in Jurkat cells transduced with Hook under the control of strong promoter sFFv and the cytokine under the control of a weaker promoter PGK said cytokine being CCL5 GFP.

FIG. 23C is a graph representing GFP fluorescence in Jurkat cells transduced with Hook under the control of strong promoter sFFv and the cytokine downstream of IVS-IRES said cytokine being IL-2-GFP.

FIG. 23D is a graph representing GFP fluorescence in Jurkat cells transduced with Hook under the control of strong promoter sFFv and the cytokine downstream of IVS-IRES said cytokine being CCL5 GFP.

FIG. 23E is a graph representing GFP fluorescence in Jurkat cells transduced with Hook under the control of strong promoter sFFv and the cytokine under the control of a strong promoter sFFv said cytokine being IL-2-GFP.

FIG. 23F is a graph representing GFP fluorescence in Jurkat cells transduced with Hook under the control of strong promoter sFFv and the cytokine under the control of a strong promoter sFFv said cytokine being CCL5 GFP.

FIGS. 24A-B are a set of graphs representing Rh30-induced cell death by IFNγ-activated T cells.

FIG. 24A is a graph representing Rh30-induced cell death by IFNγ-activated T cells from two donors assessed using a Bioluminescent assay.

FIG. 24B is a graph representing Rh30-induced cell death by IFNγ-activated T cells from one donor assessed using a Real-time cell death analysis.

FIG. 25 is a graph representing the release of the CCL5-SBP-NLuc in MCA205 mouse fibrosarcoma cell line implanted subcutaneously in immunodeficient NGS mice.

FIGS. 26A-B are a set of schemas presenting RUSH technologies.

FIG. 26A is a schema of RUSH technology described in WO2010142785.

FIG. 26B is a schema of RUSH technology as described in the present invention.

FIG. 27 is a graph representing the percentage of HEK293FT cells transduced with a hook protein under the control of a strong promoter sFFv or weaker promoter PGK, and a cytokine under the control of a weaker promoter PGK ([prsFFv-pPGK CCL5 GFP]; [pPGK-pPGK CCL5 GFP]), UbC [prsFFv-pUCB CCL5 GFP] or SV40 [prsFFv-pSV40 CCL5 GFP] or a strong promoter sFFv [prsFFv-prsFFv CCL5 GFP].

FIG. 28 is a graph representing the percentage of Jurkat cells transduced with a hook protein under the control of a strong promoter sFFv or weaker promoter PGK, and a cytokine under the control of a weaker promoter PGK ([prsFFv-pPGK CCL5 GFP]; [pPGK-pPGK CCL5 GFP]), UbC [prsFFv-pUCB CCL5 GFP] or SV40 [prsFFv-pSV40 CCL5 GFP] or a strong promoter sFFv [prsFFv-prsFFv CCL5 GFP].

FIGS. 29A-B are a set of western blot photographs of transduced HeLa cells.

FIG. 29A is a western blot photograph of GFP and vinculin revelation in cell medium of HeLa cells transduced with a hook protein under the control of a strong promoter sFFv and a cytokine (CCL5) fused to eGFP under the control of a strong promoter sFFv [prsFFv], a weaker promoter PGK [pPGK], UbC [pUBC] or SV40 [SV40], or downstream of an IVS-IRES [ivsIRES].

FIG. 29B is a western blot photograph of GFP and vinculin revelation in cell extract of HeLa cells transduced with a hook protein under the control of a strong promoter sFFv and a cytokine (CCL5) fused to eGFP under the control of a strong promoter sFFv [prsFFv], a weaker promoter PGK [pPGK], UbC [pUBC] or SV40 [SV40], or downstream of an IVS-IRES [ivsIRES].

FIGS. 30A-B is a are a set of western blot photographs of transduced Jurkat cells.

FIG. 30A is a western blot photograph of GFP revelation in cell medium of Jurkat cells transduced with a hook protein under the control of a strong promoter sFFv and a cytokine (CCL5) fused to eGFP under the control of a strong promoter sFFv [prsFFv], a weaker promoter PGK [pPGK], or downstream of an IVS-IRES [ivsIRES].

FIG. 30B is a western blot photograph of GFP revelation in cell extract of Jurkat cells transduced with a hook protein under the control of a strong promoter sFFv and a cytokine (CCL5) fused to eGFP under the control of a strong promoter sFFv [prsFFv], a weaker promoter PGK [pPGK], or downstream of an IVS-IRES [ivsIRES].

FIGS. 31A-E are a set of five graphs representing GFP fluorescence in Jurkat cells transduced with a hook protein under the control of a strong promoter sFFv or a weaker promoter PGK, and a cytokine (CCL5) fused to eGFP under the control of a strong promoter sFFv or a weaker promoter PGK, UBC or SV40. Cells were non-treated [NT]or treated with biotin at different time points (15 minutes [15 min], 60 minutes [60 min] or overnight [ON]).

FIG. 31A: Jurkat cells transduced with a hook protein under the control of a strong promoter sFFv and a cytokine (CCL5) fused to eGFP under the control of a weaker promoter PGK.

FIG. 31B: Jurkat cells transduced with a hook protein under the control of a strong promoter sFFv and a cytokine (CCL5) fused to eGFP under the control of a strong promoter sFFv.

FIG. 31C: Jurkat cells transduced with a hook protein under the control of a strong promoter sFFv and a cytokine (CCL5) fused to eGFP under the control of a weaker promoter UbC.

FIG. 31D: Jurkat cells transduced with a hook protein under the control of a strong promoter sFFv and a cytokine (CCL5) fused to eGFP under the control of a weaker promoter SV40.

FIG. 31E: Jurkat cells transduced with a hook protein under the control of a weaker promoter PGK and a cytokine (CCL5) fused to eGFP under the control of a weaker promoter PGK.

FIGS. 32A-B are a set of graphs representing the geometric mean of GFP fluorescence in transduced Jurkat cells.

FIG. 32A is a graph representing the geometric mean of GFP fluorescence in transduced Jurkat cells with a hook protein under the control of a strong promoter sFFv, and a cytokine (CCL5) fused to eGFP under the control of a weaker promoter PGK [prsFFv-pPGK CCL5] or SV40 [prsFFv-SV40 CCL5]. JURKAT WT are non-transduced cells. Cells were non-treated [NT] or treated with biotin at different time points (15 minutes [15 min], 60 minutes [60 min] or overnight [ON]).

FIG. 32B is a graph representing the geometric mean of GFP fluorescence in transduced Jurkat cells with a hook protein under the control of a strong promoter sFFv or weaker promoter PGK, and a cytokine (CCL5) fused to eGFP under the control of a strong promoter sFFv [prsFFv-prsFFv CCL5], or a weaker promoter UbC [prsFFv-pUbC CCL5] or PGK [pPGK-pPGK CCL5]. JURKAT WT are non-transduced cells. Cells were non-treated [NT] or treated with biotin at different time points (15 minutes [15 min], 60 minutes [60 min] or overnight [ON]).

### EXAMPLES

The present invention is further illustrated by the following examples.

### Example 1

The expression of several cytokines in a RUSH system in a model cell line, i.e., HeLa cells, was evaluated.

Material

Cells

HeLa cells were cultured in DMEM (Dulbecco's Modified Eagle medium) supplemented with 10% Fetal Bovine Serum (FBS), 1 mM sodium pyruvate and 100 μM penicillin and streptomycin.

Test Items

Several RUSH systems were tested, with a double promoter in a lentiviral vector comprising a streptavidin fused to a KDEL endoplasmic reticulum-retention signal peptide (SEQ ID NO: 10) under control of the sFFv strong promoter followed by a cytokine fused to a streptavidin binding peptide (SBP) with SEQ ID NO: 605 and to eGFP under the control of a weaker promoter PGK.

“IL-2-SBP-eGFP” refers to a RUSH system as afore-described, wherein said cytokine fused to SBP and to eGFP is Interleurkin-2 (IL-2).

“CCL5-SBP-eGFP” refers to a RUSH system as afore-described, wherein said cytokine fused to SBP and eGFP is C-C Chemokine Ligand 5 (CCL5).

“CXCL10-SBP-eGFP” refers to a RUSH system as afore-described, wherein said cytokine fused to SBP and eGFP is C-X-C Chemokine Ligand 5 (CXCL10).

“CCL19-SBP-eGFP” refers to a RUSH system as afore-described, wherein said cytokine fused to SBP and eGFP is C-C Chemokine Ligand 19 (CCL19).

“IFNg-SBP-eGFP” refers to a RUSH system as afore-described, wherein said cytokine fused to SBP and eGFP is Interferon gamma (IFNγ).

Methods

Test Items

RUSH systems constructions were obtained by insertion of a streptavidin tagged with KDEL (SEQ ID NO: 10) for luminal ER retention under the control of a sFFv promoter in lentiviral plasmid. Cytokines tagged with an SBP, generally in C-terminus followed by a fluorescent protein eGFP, were inserted after the PGK promoter downstream of streptavidin constructs afore-described. In all the cytokines, the signal peptide of origin was maintained, unless otherwise stated. The cytokine sequences were all generated by gene synthesis using gblock (Integrated DNA Technologies) or Twist bioscience technology.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

HeLa cells were plated on a cell culture plate and transduced with said lentiviral particle at a MOI between 1 and 5 for 2 days.

Transduced HeLa cells were plated on coverslip for immunofluorescence assay.

Transduced cells either received no treatment or were treated with 40 μM of biotin.

Cells for immunofluorescence assay were either treated or not with 40 μM or higher of biotin for 15 minutes, 75 minutes or 16 hours (steady-state).

Cells for western blotting were treated either treated or not with 40 μM biotin or higher for 60 minutes or over-night (0/N).

Immunofluorescence

Cells coated onto coverslips were washed once in 1×PBS buffer, fixed in 3% of paraformaldehyde (PFA) for 10-15 minutes at room temperature, then washed twice and alternatively incubated with 50 mM of NH₄Cl-1×PBS for 5 minutes at room temperature to quench free aldehydes. The cells were then permeabilized using a solution of PBS containing 0.5% Bovine Serum Albumin (BSA) and 0.05% saponin (Saponin, Sigma-Aldrich) for 15 minutes at room temperature. The coverslips were mounted in Mowiol supplemented with DAPI (4′,6-diamidino-2-phenylindole) for DNA staining.

Western Blotting

After biotin treatment, the supernatant was recovered and centrifuged for removal of the detached or dead cells at 300 g, 4° C., for 5-10 minutes and kept on ice. While the cells (defined hereafter as pellet) were incubated with protein loading buffer 1×(5 mM Tris-HCl, pH 7.0, 30 mM ethylenediaminetetra acid (EDTA), pH 8.0; 0.01% bromophenol blue, 5% glycerol) for 5 minutes at room temperature, scratched and transfer to a new tube followed by denaturation at 95-100° C. for 10-20 minutes. The supernatant (from adherent or suspension cells) were incubated with StrataClean Resin (Agilent) to collect and concentrate protein present in the supernatant, for at least 2 hours at 4° C. with orbital agitation. Then, the resin was separated from the supernatant by centrifugation (10000-12000 g, 4° C., 5-10 minutes), wash twice in cold 1×PBS (10000-12000 g, 4° C., 5-10 minutes), re-suspended in protein loading buffer 1× and denaturated at 95-100° C. for 10-20 minutes. The supernatant of the protein loading buffer was then recovered after centrifugation at 10000-12000 g, for 5 minutes at room temperature. Western blots were done under reducing conditions. Proteins were subjected to criterion TGX Stain Free, 4-20% gel electrophoresis (15 V, 60 minutes; Biorad), transferred using Protein Blotting Using the Trans-Blot® Turbo™ Transfer System (Biorad) according to the manufacturer's instructions. The membrane was washed two to three times in H₂O and once in PBS, 0.05% Tween-20 and blocked using 5% skim milk in 0.05% Tween-20 PBS for 1 hour at room temperature. Cytokines were detected using monoclonal anti-GFP (1/1000; Roche) in 5% skim milk in 0.1% Tween-20 PBS for 1 hour at room temperature or overnight (0/N) at 4° C., followed by the respective horseradish peroxidase (HRP) conjugated secondary polyclonal antibody (1/15000) in 5% skim milk in 0.1% Tween-20 PBS for 1 hour at room temperature. Peroxidase activity was revealed using SuperSignal™ West Pico PLUS Chemiluminescent Substrate (Pierce) in a photoradiograph (ChemiDoc MP Imaging System, Biorad).

Alternatively, the membranes were stained with a loading control, after HRP activity from the first stain was quenched using 15% of hydrogen peroxidase in 0.1% Tween-20 PBS for 30 minutes to 1 hour at room temperature. The loading control used was anti-vinculin (1/2000; Sigma) or anti-Lamin B1 (1/5000; Abcam) in 5% skim milk in 0.1% Tween-20 PBS for 1 hour at room temperature or overnight (0/N) at 4° C., followed by the respective horseradish peroxidase (HRP) conjugated secondary polyclonal antibody (1/15000) in 5% skim milk in 0.1% Tween-20 PBS for 1 hour at room temperature. The molecular weight (M) ladder used was PageRuler Plus Prestained Protein Ladder (Thermofisher).

Results

GFP Staining in HeLa Cells

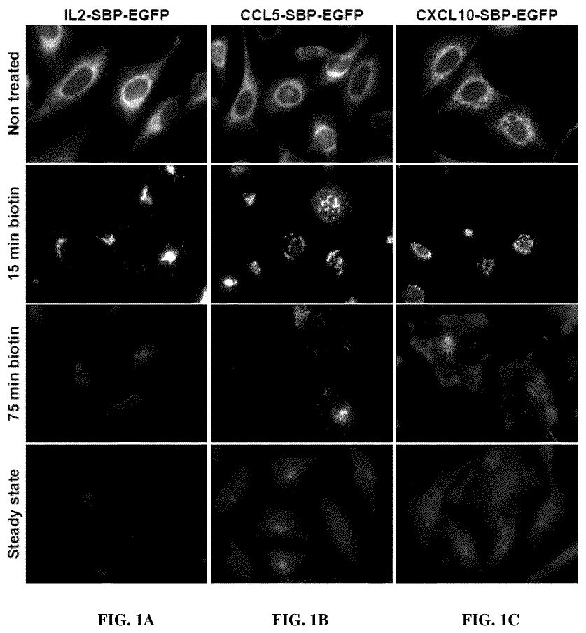

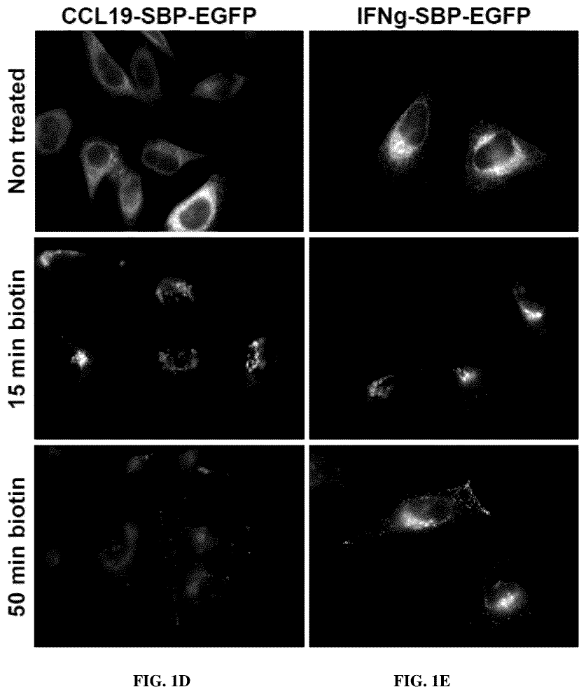

Immunofluorescence images of HeLa cells transduced with IL-2-SBP-eGFP, CCL5-SBP-eGFP, CXCL10-SBP-eGFP, CCL19-SBP-eGFP or IFNg-SBP-eGFP are respectively shown in FIGS. 1A, 1B, 1C, 1D and 1E.

In HeLa cells, the cytokines IL-2-SBP-eGFP, CCL5-SBP-eGFP, CXCL10-SBP-eGFP, CCL19-SBP-eGFP and IFNg-SBP-eGFP in the absence of biotin were retained in the endoplasmic reticulum (ER) (respectively FIGS. 1A, 1B, 1C, 1D and 1E) and upon biotin addition, 15 minutes later, they trafficked to the Golgi and then to the cell surface followed by their secretion to the medium at 50 or 75 minutes.

At steady state or in the presence of biotin for at least 16 hours, no or very low intracellular GFP was detected, suggesting complete secretion of the cytokine IL-2-SBP-eGFP, CCL5-SBP-eGFP and CXCL10-SBP-eGFP (respectively FIGS. 1A, 1B, and 1C). The cytokines CCL19-SBP-eGFP and IFNg-SBP-eGFP after 50 minutes with biotin, no or lower intracellular GFP was detected, suggesting secretion of cytokine to the medium FIGS. 1D and 1E).

GFP Revelation in HeLa Cells

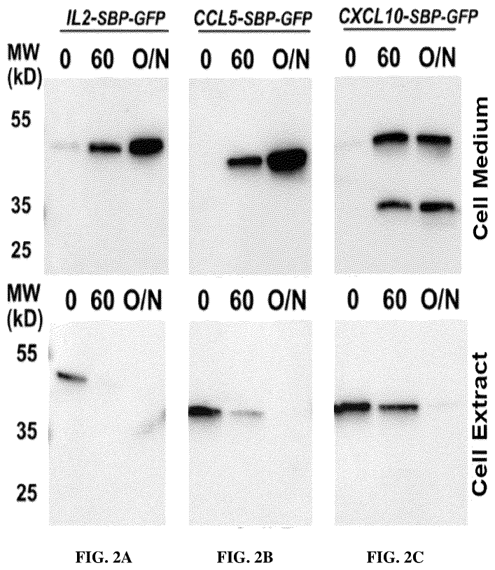

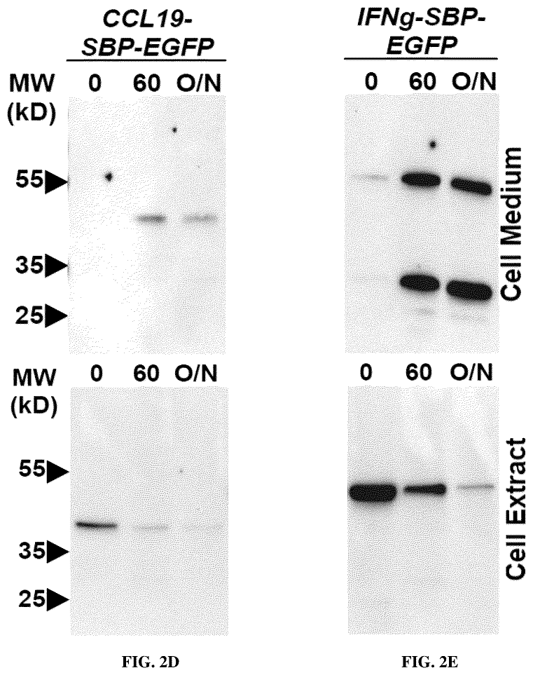

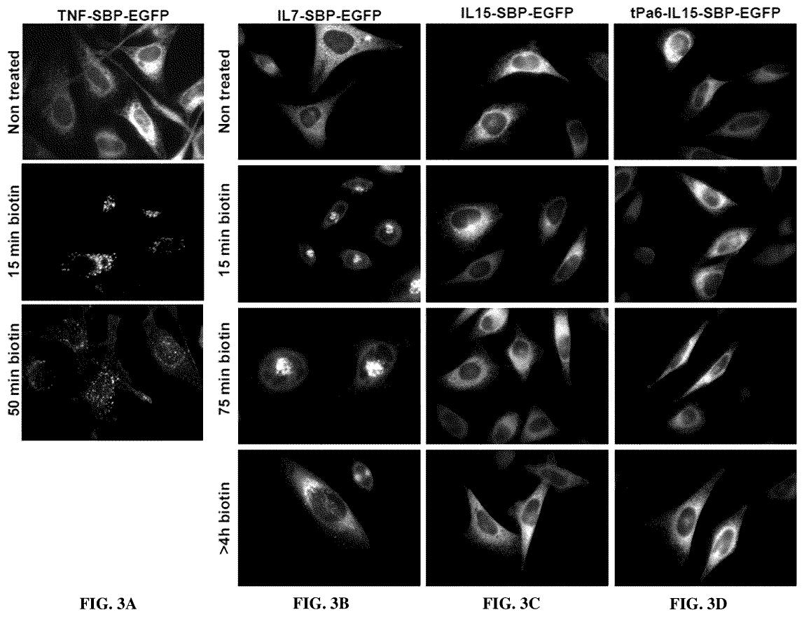

Western blot photographs of HeLa cells transduced with IL-2-SBP-eGFP, CCL5-SBP-eGFP, CXCL10-SBP-eGFP, CCL19-SBP-eGFP or IFNg-SBP-eGFP are respectively shown in FIGS. 2A, 2B, 2C, 2D and 2E.

IL-2 leaking was visible in the absence of biotin, as a weak band in the cell medium could be observed at 50 kDa (FIG. 2A), corresponding to the size of this cytokine, while no such phenomenon could be observed for CCL5 (FIG. 2B) or CXCL10 (FIG. 2C). This suggests that the leaking in RUSH is dependent of the different cytokine.

Upon addition of biotin for 60 minutes, a strong band in the cell medium was observed for all cytokines, as it is secreted and at overnight (0/N). These results are in accordance with what was observed for the cell extract, in which a strong band was observed in the absence of biotin, due to cytokine-cell retention and the addition of biotin led to a decrease in the band intensity or to its absence due to cytokine secretion in the medium (FIGS. 2A, 2B and 2C).

Similar results were obtained for the cytokine CCL19 and IFNγ (FIGS. 2D and 2E), with no leaking observed for CCL19, while IFNγ was slightly secreted in the absence of biotin, while upon biotin addition, a significant increase in their secretion was detected in culture medium.

### Example 2

The expression of other cytokines in a RUSH system in a model cell line, i.e., HeLa cells, was evaluated.

Material

Cells

HeLa cells cultured in DMEM (Dulbecco's modified Eagle medium) supplemented with 10% Fetal Bovine Serum, 1 mM sodium pyruvate and 100 sM of penicillin and streptomycin.

Test Items

Several RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“TNF-SBP-eGFP” refers to a RUSH system as afore-described, wherein said cytokine fused to SBP and to eGFP is Tumour Necrosis Factor (TNF).

“IL-7-SBP-eGFP” refers to a RUSH system as afore-described, wherein said cytokine fused to SBP and to eGFP is Interleukin-7 (IL-7).

“IL15-SBP-eGFP” refers to a RUSH system as afore-described, wherein said cytokine fused to SBP and to eGFP is Interleukin-15 (IL-15).

“tPa6-IL-15-SBP-eGFP” refers to a RUSH system as afore-described, wherein said cytokine fused to SBP and to eGFP is Interleukin-15 (IL-15) wherein “tPas6” Tissue Plasminogen Activation signal peptide (with SEQ ID NO: 602) replaces the native IL-15 peptide signal.

Methods

Test Items

RUSH systems constructions were obtained as previously presented in Example 1 methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

HeLa cells were plated on a cell culture plate and transduced with said lentiviral particle at a MOI between 1 and 5 for 2 days.

Transduced HeLa cells were plated on coverslip for immunofluorescence assay.

Transduced cells either received no treatment or were treated with 40 μM of biotin.

Cells for immunofluorescence assay were either treated or not with 40 μM biotin for 15 minutes, 50 minutes, 75 minutes or more than 4 hours (>4 h).

Immunofluorescence

Immunofluorescence assays were performed as previously described in Example 1, methods section.

Results

GFP Staining in HeLa Cells

Immunofluorescence images of HeLa cells transduced with TNF-SBP-eGFP, IL-7-SBP-eGFP, IL-15-SBP-eGFP or tPa6-IL-15-SBP-eGFP are respectively shown in FIGS. 3A, 3B, 3C and 3D.

The cytokines TNF-SBP-eGFP, IL-7-SBP-eGFP, IL-15-SBP-eGFP and tPa6-IL-15-SBP-eGFP in the absence of biotin was retained in the endoplasmic reticulum (ER) (respectively FIGS. 3A, 3B, 3C and 3D).

The cytokine TNF, upon biotin addition, trafficked from the ER to the Golgi (15 minutes) and to the cell surface at 50 minutes (FIG. 3A). TNF is a cytokine with a transmembrane domain that is cleaved by the TNFα converting enzyme (TACE) when reaching the cell surface; however, in the HeLa cells, this enzyme is not presence and thus, TNF remains at the cell surface (FIG. 3A).

IL-7 was also retained in the ER in the absence of biotin and trafficked to the Golgi after 15 minutes with biotin and, contrarily to the other cytokines, remained in the Golgi after 75 minutes with biotin; it was only after 4 hours that we observed some dots at the membrane and loss of intensity of GFP in the Golgi, suggesting partial secretion of IL-7 (FIG. 3B).

IL-15 is a cytokine that was described to have low expression, with an impaired traffic when its receptor is not present. As described in US20160102128, the tPa6 signal peptide was added to IL-15 in place of native peptide signals. HeLa expressed IL-15-SBP-eGFP properly; however, upon biotin addition, even at later time points, the protein remained in the ER (FIG. 3C). Similar results were obtained for the optimized sequence tPa6-IL-15, contrarily to what was suggested by US20160102128 (FIG. 3D).

### Example 3

The expression of other cytokines in a RUSH system in a model cell line, i.e., HeLa cells, was evaluated.

Material

Cells

HeLa cells cultured in DMEM (Dulbecco's modified Eagle medium) supplemented with 10% Fetal Bovine Serum, 1 mM sodium pyruvate and 100 sM of penicillin and streptomycin.

Test Items

Several RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP or CH (Cherry fluorescent protein) under the control of a weaker promoter PGK.

“CXCL9-SBP-CH” refers to a RUSH system as afore-described, wherein said cytokine fused to SBP and to CH is C-X-C Chemokine Ligand 9 (CXCL9).

“IL-12b-p2a-IL-12a-SBP-CH” refers to a RUSH system as afore-described, wherein said cytokine fused to SBP and to CH is Interleukin-12(IL-12) composed of two subunits, IL-12b and IL-12a, separated by a p2a self-cleavage peptide (IL-12b-p2a-IL-12a).

“IL-21-SBP-eGFP” refers to a RUSH system as afore-described, wherein said cytokine fused to SBP and to eGFP is Interleukin-21 (IL-21).

“GM-CSF-SBP-eGFP” refers to a RUSH system as afore-described, wherein said cytokine fused to SBP and to eGFP is granulocyte-macrophage colony stimulating factor (GM-CSF).

“IL-8-SBP-eGFP” refers to a RUSH system as afore-described, wherein said cytokine fused to SBP and to eGFP is Interleukin-8 (IL-8).

Methods

Test Items

RUSH systems constructions were obtained as previously presented in Example 1 methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

HeLa cells were plated on a cell culture plate and transduced with said lentiviral particle at a MOI between 1 and 5 for 2 days.

Transduced HeLa cells were plated on coverslip for immunofluorescence assay.

Transduced cells either received no treatment or were treated with 40 μM of biotin.

Cells for immunofluorescence assay were either treated or not with 40 μM biotin for 15 minutes, 60 minutes, 90 minutes or 3 hours.

Immunofluorescence

Immunofluorescence assays were performed as previously described in Example 1, methods section.

Results

GFP Staining in HeLa Cells

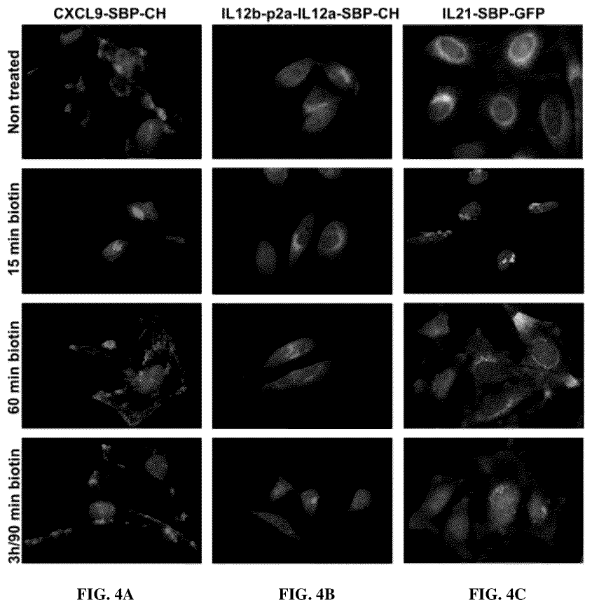

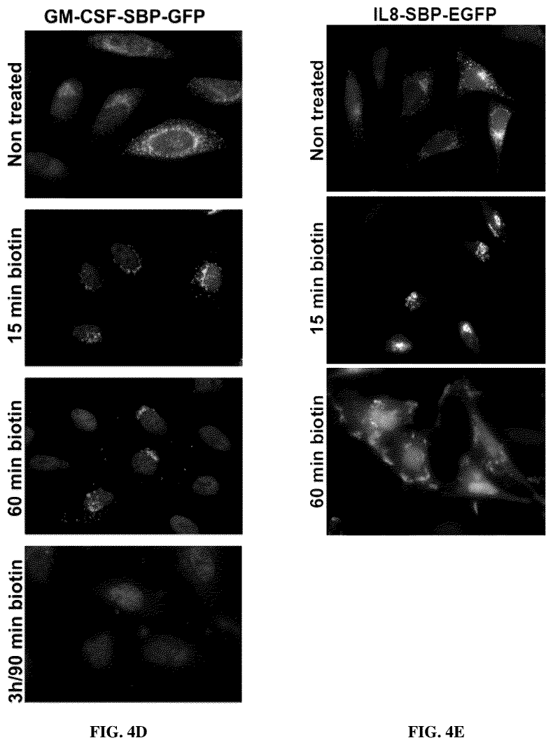

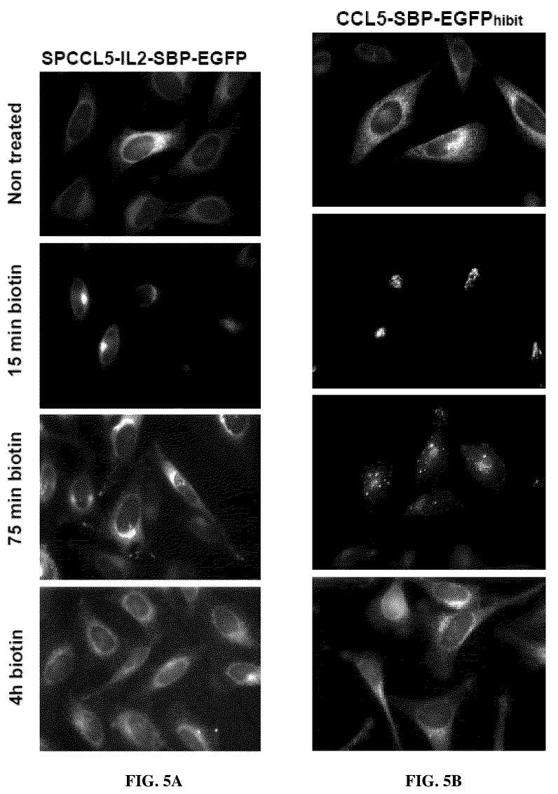

Immunofluorescence images of HeLa cells transduced with CXCL9-SBP-CH, IL-12b-p2a-IL-12a-SBP-CH, IL-21-SBP-eGFP, GM-CSF-SBP-eGFP or IL-8-SBP-eGFP are respectively shown in FIGS. 4A, 4B, 4C, 4D and 4E.

IL-12b-p2a-IL-12a-SBP-CH, IL-21-SBP-eGFP, GM-CSF-SBP-eGFP and IL-8-SBP-eGFP were well retained in the ER (respectively FIGS. 4B, 4C, 4D and 4E), with exception of CXCL9-SBP-CH (FIG. 4A).

CXCL9 in the absence of biotin was localized at the cell surface/focal adhesion, suggesting that a portion of this protein was not retained in the ER and trafficked to the cell surface/focal adhesion and presumably got attached to the plate surface. The presence of the cytokine at the cell surface/focal adhesion were also observed for CXCL10 (FIG. 1C) and IL-8 after more than 60 minutes in the presence of biotin (FIG. 4E). After 15 minutes with biotin, the cytokines CXCL9, IL-21 and GM-CSF trafficked to Golgi, and after 60 minutes, to the cell surface where they were then secreted (respectively, FIGS. 4A, 4C and 4D). For CXCL9 (FIG. 4A), after 60 minutes, an increase in the intensity of cherry at the focal adhesion was observed due to the arrival of the cytokine, similarly to IL-21 (FIG. 4C). However, after 90 minutes or 3 hours, CXCL9 remained at the focal adhesion/attaches to cell plate (FIG. 4A), while IL-21 (FIG. 4C) seemed to be secreted to the medium as the GFP intensity in the cell decreased as well as in the focal adhesion.

For IL-12 (FIG. 4B), a Golgi localization was observed after 15 minutes in the presence of biotin, but a portion of it remain in the ER and in the cytosol; after 60 minutes, only a small portion was secreted as suggested by the presence of some dots at the cell surface and by a slightly decrease in intracellular cherry intensity (FIG. 4C). Experiments to further evaluate the traffic of IL-12 using p2a or a linker between both subunits of IL-12 are undergoing.

The traffic of IL-8 (FIG. 4E) under the RUSH control was also studied, and its traffic was similar to that of CXCL10 (FIG. 1C).

### Example 4

The expression of several cytokines in a RUSH system in a model cell line, i.e., HeLa cells, was evaluated. New constructs to prevent cytokine leaking were evaluated.

Material

Cells

HeLa cells cultured in DMEM (Dulbecco's modified Eagle medium) supplemented with 10% Fetal Bovine Serum, 1 mM sodium pyruvate and 100 sM of penicillin and streptomycin.

Test Items

Several RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“SPCCL5-IL-2-SBP-eGFP” refers to a RUSH system as afore-described, wherein said cytokine fused to SBP and to eGFP is Interleurkin-2 (IL-2) using the signal peptide of CCL5 (SPCCL5) with SEQ ID NO: 603.

“CCL5-SBP-eGFPh_{hibit}” refers to a RUSH system as afore-described, wherein said cytokine fused to SBP and to eGFP containing HiBit tag (SEQ ID NO: 604) for its quantitative determination using bioluminescence is C-C Chemokine Ligand 5 (CCL).

Methods

Test Items

RUSH systems constructions were obtained as previously described in Example 1, methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

HeLa cells were plated on a cell culture plate and transduced with said lentiviral particle at a MOI between 1 and 5 for 2 days.

Transduced HeLa cells were plated on coverslip for immunofluorescence assay.

Transduced cells either received no treatment or were treated with 40 μM of biotin.

Cells for immunofluorescence assay were either treated or not with 40 μM biotin for 15 minutes, 75 minutes or 4 hours.

Cells for western blotting were treated either treated or not with 40 μM biotin for 60 minutes or overnight (O/N).

Immunofluorescence

Immunofluorescence assays were performed as previously described in Example 1, methods section.

Western Blotting

Western blot was performed as previously described in Example 1, methods section.

Results

GFP Staining in HeLa Cells

Immunofluorescence images of HeLa cells transduced with SPCCL5-IL-2-SBP-eGFP, or CCL5-SBP-eGFP_{hibit} are respectively shown in FIG. 5A and FIG. 5B.

As previously mentioned, some leaking was observed for the cytokine IL-2-SBP-eGFP (FIG. 1A), contrarily to CCL5-SBP-eGFP, thus IL-2 natural signal peptide (IL-2) was exchanged with the signal peptide of the non-leaking cytokine, CCL5 (SPCCL5-IL-2), aiming to improve IL-2 retention. Traffic of SPCCL5-IL-2-SBP-eGFP (FIG. 5A) was assessed and the retention of the cytokine in the ER was checked. After 15 minutes with biotin treatment, the cytokine was localized in the Golgi; however, at 75 minutes, a high percentage of the cytokine still remained in the Golgi, suggesting a delay in the traffic in comparison to IL-2 (FIG. 1A). At 4 hours, most of the cytokine had been secreted.

CCL5-SBP-eGFP_{hibit} (FIG. 5B) traffic was similar to the CCL5-SBP-eGFP (FIG. 1B), suggesting that the presence of the HiBit tag does not impact CCL5 traffic.

GFP Revelation in HeLa Cells

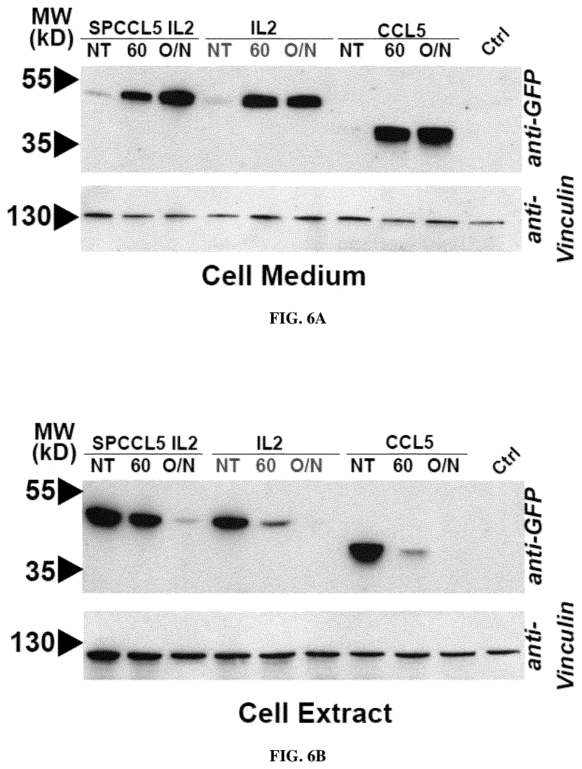

Western blotting showed that both SPCCL5-IL-2-SBP-eGFP and IL-2-SBP-eGFP were leaking, as a small band was observed at the NT (non-treated), contrarily to CCL5-SBP-eGFP; after 60 minutes of biotin, all cytokines were secreted (FIG. 6A).

For SPCCL5-IL-2-SBP-eGFP, the highest secretion was observed after O/N with biotin (FIG. 6A).

Yet, all cytokines were properly secreted under biotin treatment, diminishing cytokines' presence inside cells (FIG. 6B).

### Example 5

The expression of several cytokines in a RUSH system in a model cell line, i.e., HeLa cells, was evaluated. New constructs to prevent cytokine leaking were evaluated.

Material

Cells

HeLa cells cultured in DMEM (Dulbecco's modified Eagle medium) supplemented with 10% Fetal Bovine Serum, 1 mM sodium pyruvate and 100 sM of penicillin and streptomycin.

Test Items

Several RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“IL-4-SBP-eGFP” refers to a RUSH system as afore-described, wherein said cytokine fused to SBP and to eGFP is Interleukin-4 (IL-4).

“IFNa2-SBP-eGFP” refers to a RUSH system as afore-described, wherein said cytokine fused to SBP and to eGFP is Interferon alpha 2 (IFNa2).

“CCL21-SBP-eGFP” refers to a RUSH system as afore-described, wherein said cytokine fused to SBP and to eGFP is C-C Chemokine Ligand 21 (CCL).

“SPCCL5-IL-36-SBP-eGFP” refers to a RUSH system as afore-described, wherein said cytokine fused to SBP and to eGFP is Interleurkin-36 (IL-36) using the signal peptide of CCL5 (SPCCL5) with SEQ ID NO: 603. The cytokine IL-36 alpha has a pro-peptide in N-terminus and, in order to be functional, this pro-peptide needs to be cleaved. For its expression using RUSH system, the pro-peptide was removed and the signal peptide of CCL5 was inserted in the N-terminus of the functional cytokine.

Methods

Test Items

RUSH systems constructions were obtained as previously described in Example 1, methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

HeLa cells were plated on a cell culture plate and transduced with said lentiviral particle at a MOI between 1 and 5 for 2 days.

Transduced HeLa cells were plated on coverslip for immunofluorescence assay.

Transduced cells either received no treatment or were treated with 40 μM of biotin.

Cells for immunofluorescence assay were either treated or not with 40 μM biotin for 15 minutes, 70 minutes or more than 3 hours.

Cells for western blotting were treated either treated or not with 40 μM biotin for 60 minutes or overnight (0/N).

Immunofluorescence

Immunofluorescence assays were performed as previously described in Example 1, methods section.

Western Blotting

Western blot was performed as previously described in Example 1, methods section.

Flow Cytometry

After incubation with biotin, the cells were immediately transferred to ice, to inhibit or slow down the traffic of the cargo, followed by centrifugation (300 g, 4° C., 5 minutes), washed twice in cold 1×PBS (300 g, 4° C., 5 minutes) and incubated with live/dead fixable staining (20 min, on ice; Thermofisher). The cells were then washed twice in cold PBS (300 g, 4° C., 5 minutes) or FACS buffer (1×PBS, 1% BSA, 0.05% sodium azide, 1 mL EDTA 0.5 M, filtered and kept at 4° C.) and, when not analyzed immediately, the cells were fixed in 3% PFA-1×PBS (10 min, RT) and washed twice in 1×PBS.

Results

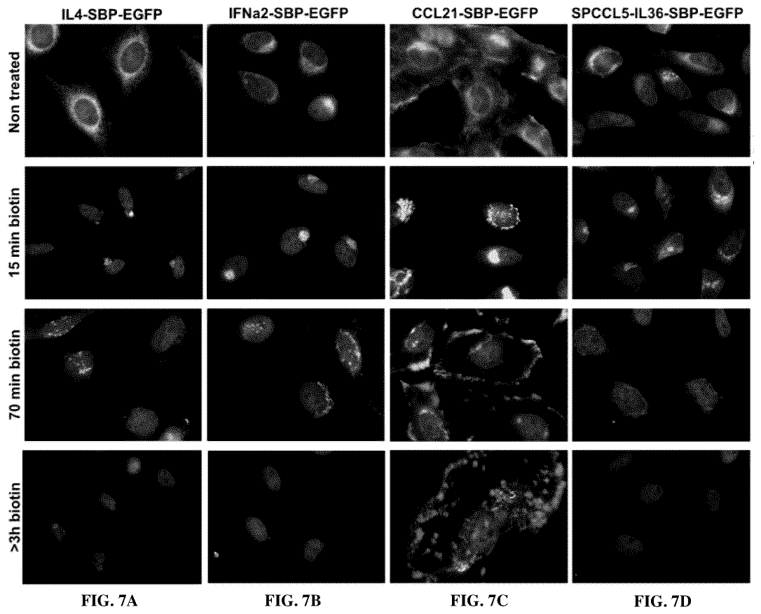

GFP Staining in HeLa Cells Immunofluorescence images of HeLa cells transduced with IL-4-SBP-eGFP, IFNa2-SBP-eGFP, CCL21-SBP-eGFP and SPCCL5-IL-36-SBP-eGFP are respectively shown in FIGS. 7A, 7B, 7C and 7D.

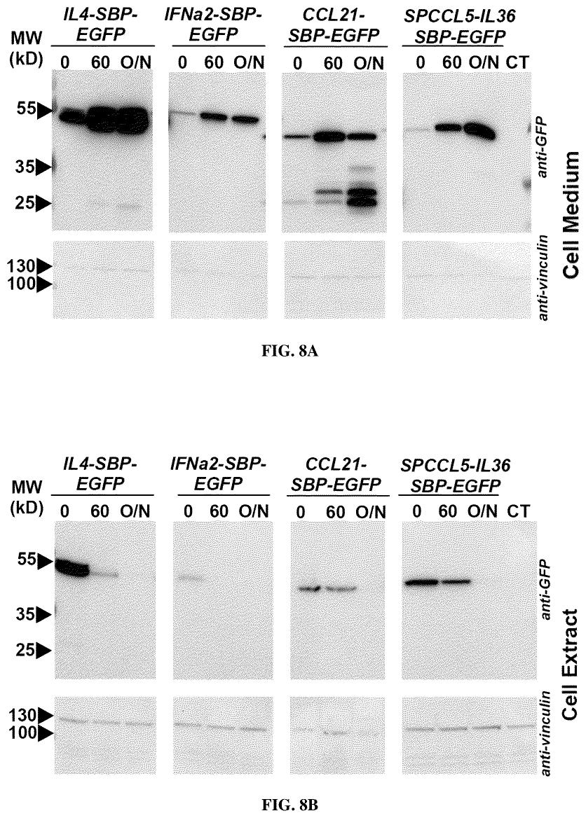

Western blot photographs of culture media and cell extract of HeLa cells transduced with IL-4-SBP-eGFP, IFNa2-SBP-eGFP, CCL21-SBP-eGFP and SPCCL5-IL-36-SBP-eGFP are respectively shown in FIGS. 8A and 8B.

In HeLa cells, the cytokines IL-4-SBP-eGFP, IFNa2-SBP-eGFP and IL-36a with the signal peptide from CCL5 (SPCCL5), SPCCL5-IL-36-SBP-eGFP, were retained in the endoplasmic reticulum (ER) in the absence of biotin (respectively, FIGS. 7A, 7B and 7D); upon biotin addition, 15 minutes later, they trafficked to the Golgi and then to the cell surface followed by their secretion to the medium at 70 minutes. At steady state, i.e., in the presence of biotin for at least 3 hours, no or very low intracellular GFP was detected, suggesting complete secretion of the cytokines IL-4-SBP-eGFP, IFNa2-SBP-eGFP and SPCCL5-IL-36-SBP-eGFP (respectively FIGS. 7A, 7B and 7D). These results were consistent with western blot GFP revelation of IL-4-SBP-eGFP, IFNa2-SBP-eGFP and SPCCL5-IL-36-SBP-eGFP (FIGS. 8A and 8B). The highest secretion was only observed after 60 minutes of biotin, despite some leakage in the absence of biotin (FIGS. 8A and 8B).

The cytokine CCL21-SBP-eGFP (FIG. 7C) was localized at the ER in the absence of biotin, but some leaking of the cytokine was also observed, that got attached into the coverslips. The majority of the cytokine was still retained at the ER, and reached the Golgi at 15 minutes, and was secreted at 70 minutes, attaching to the coverslips. After more than 3 hours with biotin, the cytokine was secreted and similarly to 70 minutes with biotin, it attached to the coverslip upon secretion, presumably at the focal adhesion. These observations were consistent with western blot GFP revelation of CCL21-SBP-eGFP (FIGS. 8A and 8B).

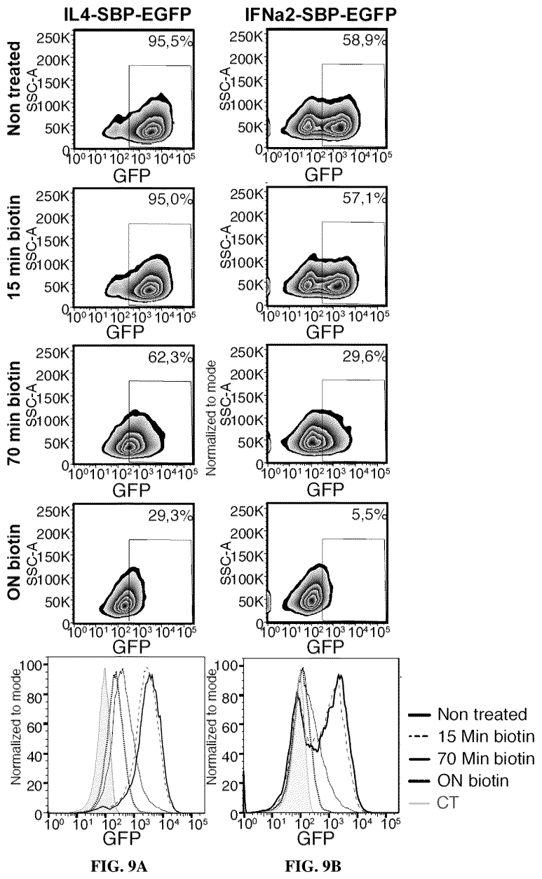

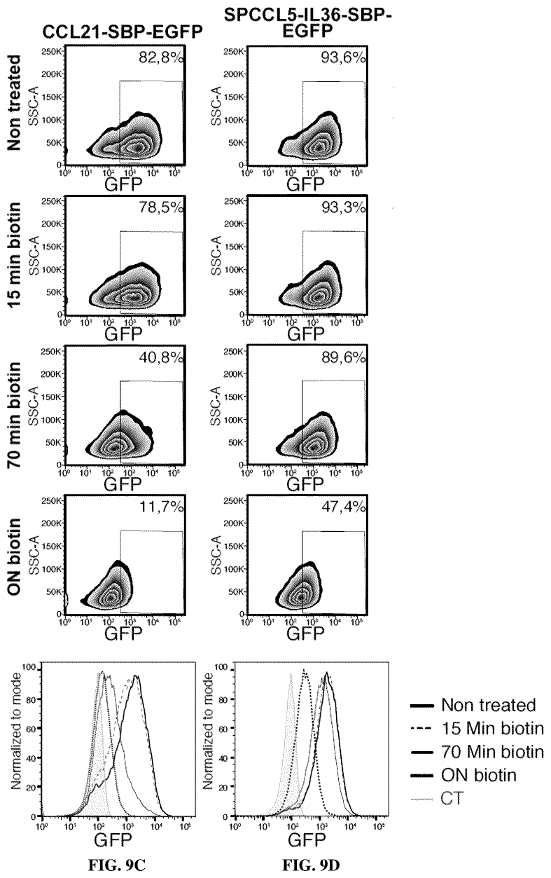

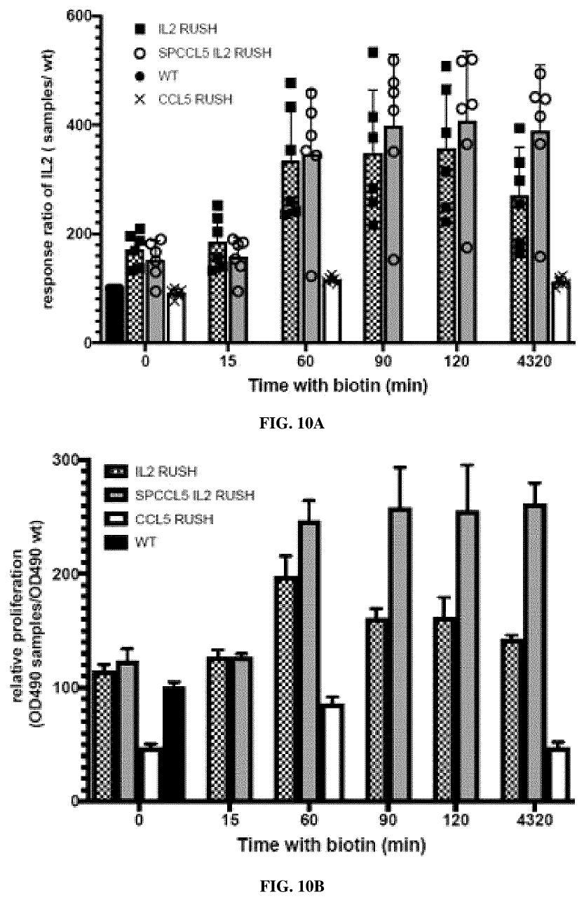

Flow cytometry staining graphs of HeLa cells transduced with IL-4-SBP-eGFP, IFNa2-SBP-eGFP, CCL21-SBP-eGFP and SPCCL5-IL-36-SBP-eGFP are respectively shown in FIGS. 9A, 9B, 9C and 9D.

A decrease in the intensity of GFP was observed as biotin was added to the cells for the cytokines IL-4, IFNa2, CCL21 and SPCCL5-IL-36 (FIGS. 9A, 9B, 9C and 9D), with the highest decreases reached O/N with biotin, suggesting that the majority was secreted into the cell medium.

### Example 6

The biological activity of IL-2 cytokine using its natural signal peptide (IL-2 RUSH) or the signal peptide of CCL5 cytokine with SEQ ID NO: 603 (SPCCL5 IL-2 RUSH) fused to SBP and eGFP in RUSH system was evaluated using a reporter cell line (HEK blue IL-2, Invitrogen) and CTLL2-NFκB (Mock et al., 2020. Sci Rep. 10(1):3234).

Material

Cells

HeLa cells cultured in DMEM (Dulbecco's modified Eagle medium) supplemented with 10% Fetal Bovine Serum, 1 mM sodium pyruvate and 100 μM of penicillin and streptomycin.

HEK-Blue reporter cells cultured in DMEM (Dulbecco's modified Eagle medium) supplemented with 10% Fetal Bovine Serum, 1 mM sodium pyruvate and 100 μM of normocin.

CTLL-2 NFκB cells cultivated with IL-2 (Miltenyi) in Roswell Park Memorial Institute (RPMI)-1640 medium (Gibco) supplemented with 10% FBS (Gibco), 1× antibiotic-antimycoticum (Gibco), 2 mM ultraglutamine (Lonza), 25 mM HEPES (Gibco) and 50 sM p-mercaptoethanol (Sigma Aldrich) at 37° C. and 5% of CO₂.

Test Items

Several RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“IL-2-SBP-eGFP” refers to a RUSH system as afore-described, wherein said cytokine fused to SBP and to eGFP is Interleurkin-2 (IL-2).

“CCL5-SBP-eGFP” refers to a RUSH system as afore-described, wherein said cytokine fused to SBP and to eGFP is C-C Chemokine Ligand 5 (CCL5).

“SPCCL5-IL-2-SBP-eGFP” refers to a RUSH system as afore-described, wherein said cytokine fused to SBP and to eGFP is Interleurkin-2 (IL-2) using the signal peptide of CCL5 (SPCCL5) with SEQ ID NO: 603.

Methods

Test Items

RUSH systems constructions were obtained as presented in Example 1, methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

HeLa cells were plated on cell culture plate and transduced with said lentiviral particle at a MOI between 1 and 5 for 2 days.

Transduced HeLa cells were plated on coverslip for immunofluorescence assay.

Transduced cells either received no treatment or were treated with 40 μM of biotin.

Cells were treated with 40 μM biotin for 0 minute, 15 minutes, 60 minutes, 90 minutes, 120 minutes or 4320 minutes (=72 hours).

After treatment of the cells, the cell medium (1500 μL) was collected and centrifuged (300 g, 4° C., 5-10 minutes) to remove dead cells.

Cytokine Activity

The cell medium of the RUSH-transduced cells was added into a cell culture 96-well plate (flat bottom) at a dilution 1/80 to 1/100 followed by the platting of HEK-Blue cells at approximately 50 000 cells/well. On the next day (after approximately 24 hours), 20 μL of the supernatant of these cells was transferred to a new plate and 180 μL of QUANTI-Blue substrate was added and incubated at 37° C. and 5% of C02 for 1-3 hours. The absorbance was then measured at 620 nm. The response ratio of the cytokines was determined by dividing the value of absorbance of the treated cells by non-treated cells. The values were them normalized to WT cells line.

The relative proliferation and NFκB response measured by luminescence in CTLL2 NFκB was determined as described by Mock et al. (2020. Sci Rep. 10(1):3234). Briefly, CTLL2 NFκB were starved for about 24 hours, i.e., incubated for 24 hours without IL-2 to reduce the background due to the presence of IL-2 in the medium. After starvation, the cells (approximately 50 000 cells/well) in a cell culture 96-well plate (flat bottom) were incubated with the supernatant of the transduced cells either non-treated or treated with biotin diluted at 1/80 to a final volume of 200 μL. The cells were incubated for 72 hours at 37° C. and 5% of C02.

To determine the activity of NFκB, 20 μL of the cell supernatant were transferred to a white opaque 96 well plate (PerkinElmer) and 80 μL of 2 mg/mL of coelenterazine (Carl Roth) in phosphate buffered saline (PBS) was added. Luminescence was measured immediately in the plate reader CLARIOstar®.

For CTLL-2 proliferation, CellTiter 96 Aqueous One Solution (Promega) was used according to the manufacturer's instructions. Briefly, 100 μL of the cells medium were removed and 20 μL of CellTiter 96 Aqueous One Solution (Promega) were added and incubated at least for 1 hours at 37° C. and 5% of CO₂ before reading the absorbance at 490 nm. The relative proliferation or luminescence was determined by dividing the absorbance of treated cells by non-treated cells.

In all the assays, IL-2 (Miltenyi) or IFNγ (STEMCELL) commercially available were used as positive controls.

Western Blotting

Western blot was performed as previously described in Example 1, methods section.

Results

Cytokine Relative Activity

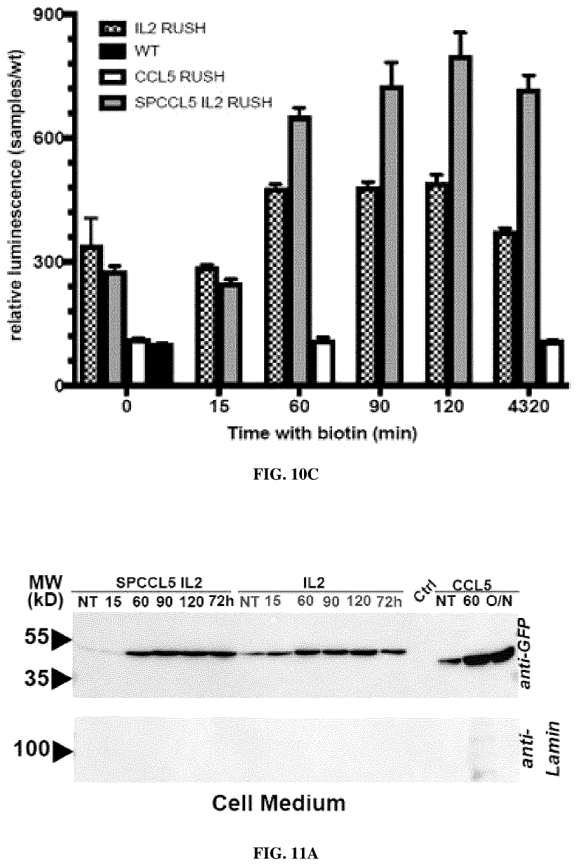

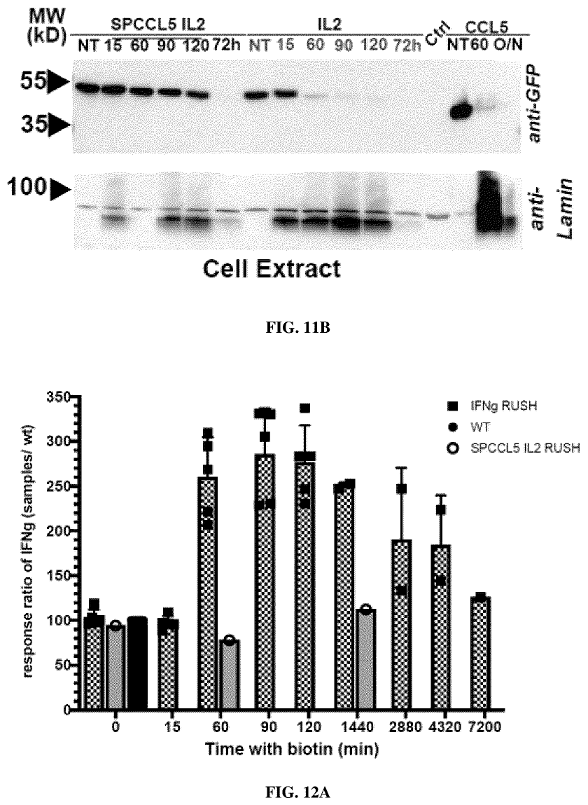

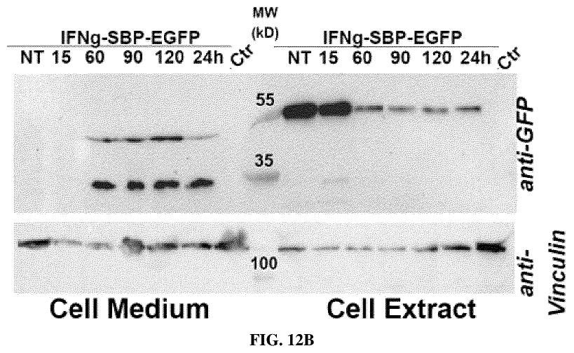

Graph representing cytokine activity in reporter cell line transduced with SPCCL5-IL-2-SBP-eGFP, IL-2-SBP-eGFP or CCL5-SBP-eGFP is presented in FIG. 10A.

Graph representing IL-2 induced proliferation of reporter cell line transduced with SPCCL5-IL-2-SBP-eGFP, IL-2-SBP-eGFP or CCL5-SBP-eGFP is presented in FIG. 10B.

Graph representing NFκB-response of reporter cell line transduced with SPCCL5-IL-2-SBP-eGFP, IL-2-SBP-eGFP or CCL5-SBP-eGFP is presented in FIG. 10C.

Of note, NFκB-response is induced by IL-2 stimulation.

Western blot photographs in reporter cell line transduced with SPCCL5-IL-2-SBP-eGFP, IL-2-SBP-eGFP or CCL5-SBP-eGFP are presented in FIGS. 11A and 11B.

In the absence of biotin, both SPCCL5-SBP-eGFP and IL-2-SBP-eGFP induced a small response by the reporter cell line, suggesting that a small proportion of the cytokine was not retained in the ER. Similar results were obtained by western blot (FIG. 11A), in which some leaking was observed in the absence of biotin. Moreover, in western blot, a better retention for SPCCL5-IL-2-SBP-eGFP was observed in comparison to IL-2-SBP-eGFP, which was not significant when using the reporter cell lines (FIG. 10A).

The addition of biotin led to cytokine secretion and consequently to the increase of response by the reporter cell line HEKBlue IL-2(FIG. 10A). Similar results were obtained when using CTLL-2 NFκB reporter cell line (FIG. 10B).

In absence of biotin, CTLL-2-NFκB cell proliferated for both SPCCL5 IL-2 and IL-2 due to lower concentration of IL-2 in the medium by RUSH leakage (FIG. 10A). When biotin was added for more than 60 minutes, the amount of IL-2 in the cell medium increased and consequently, a significant increase in cell proliferation was observed. The proliferation of CTLL-2-NFκB cell slightly decreased after 4320 minutes for IL-2, contrarily to SPCCL5 IL-2 that seemed to better sustain cell activation.

The expression of secreted nanoluc by CTLL-2 NFκB cells was also evaluated, as upon IL-2 stimulation, these cells induce NFκB activation for expression of secreted nanoluc (FIG. 10C). Similar results as for cell proliferation were obtained (FIG. 10). These results were in accordance to the western blot (FIG. 11).

### Example 7

The biological activity of IFN gamma (IFNγ) cytokine fused to SBP and eGFP in a RUSH system was evaluated using a reporter cell line (HEK blue IFNγ, Invitrogen).

Material

Cells

Rh30-luciferase cells cultured in DMEM (Dulbecco's modified Eagle medium) supplemented with 10% Fetal Bovine Serum, 1 mM sodium pyruvate and 100 μM of penicillin and streptomycin.

HEK-Blue reporter cells cultured in DMEM (Dulbecco's modified Eagle medium) supplemented with 10% Fetal Bovine Serum, 1 mM sodium pyruvate and 100 sM of normocin.

Test Items

RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“IFNg-SBP-eGFP” refers to a RUSH system as afore-described, wherein said cytokine fused to SBP and to eGFP is Interferon gamma (IFNγ).

“SPCCL5-IL-2-SBP-eGFP” refers to a RUSH system as afore-described, wherein said cytokine fused to SBP and to eGFP is Interleurkin-2 (IL-2) using the signal peptide of CCL5 (SPCCL5) with SEQ ID NO: 603.

Methods

Test Items

RUSH systems constructions were obtained as described in Example 1, methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

Rh30 cells were plated into culture plates and transduced with said lentiviral particle at a MOI between 1 and 5 for 2 days.

Transduced Rh30 cells were plated into culture plates for western blotting.

Transduced cells either received no treatment or were treated with 40 μM of biotin for 15 minutes, 60 minutes, 90 minutes, 120 minutes, 1440 minutes (=24 hours), 2880 minutes (=48 hours), 4320 minutes (=72 hours) or 7200 minutes (=120 hours).

After treatment of the cells, the cell medium (1500 pL) was collected and centrifuged (300 g, 4° C., 5-10 minutes) to remove dead cells.

Cytokine Activity

The cell medium of the RUSH-transduced cells was added into a cell culture 96-well plate (flat bottom) at a dilution 1/80 to 1/100 followed by the platting of HEK-Blue cells at approximately 50 000 cells/well. On the next day (after approximately 24 hours), 20 μL of the supernatant of these cells were transferred to a new plate and 180 μL of QUANTI-Blue substrate were added and incubated at 37° C. and 5% of C02 for 1-3 hours. The absorbance was then measured at 620 nm. The response ratio of the cytokines was determined by dividing the value of absorbance of the treated cells by non-treated cells. The values were then normalized to WT cells line.

In all the assays, IFNγ (STEMCELL) commercially available was used as positive control.

Western Blotting

Western blot was performed as previously described in Example 1, methods section.

Results

IFNγ Relative Activity

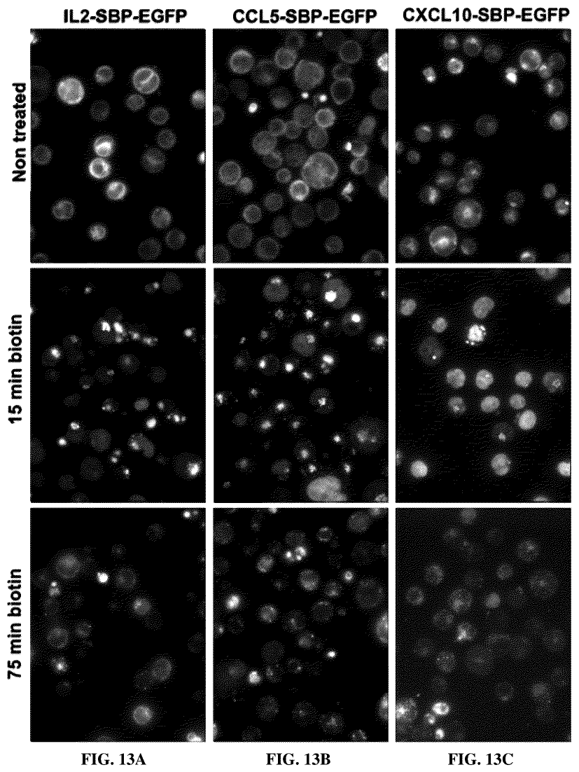

Rh30 transduced with IFNγ RUSH, in absence of biotin, efficiently retained the cytokine and thus no response was induced by the reporter cell line (FIG. 12A). Similarly, in the western blot, no band was observed in the cell medium in the absence of biotin (FIG. 12B).

Upon biotin addition, IFNγ was secreted in the medium (FIG. 12B), inducing a response by the reporter cell line that was maintained until 1440 minutes (FIG. 12A). At later time points with biotin, the response induced by IFNγ decreased, most probably due to cytokine degradation (FIG. 12A).

### Example 8

The expression of cytokines in a RUSH system in a model T cell line, i.e., Jurkat cells, was evaluated.

Material

Cells

Jurkat cells cultured in Roswell Park Memorial Institute (RPMI)-1640 supplemented with 10% FBS or RPMI medium supplemented with 14 μg/mL of avidin to chelate the existing biotin present in the medium.

Test Items

Several RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“IL-2-SBP-eGFP” refers to a RUSH system as afore-described, wherein said cytokine fused to SBP and to eGFP is Interleurkin-2 (IL-2).

“CCL5-SBP-eGFP” refers to a RUSH system as afore-described, wherein said cytokine fused to SBP and to eGFP is C-C Chemokine Ligand 5 (CCL5).

“CXCL10-SBP-eGFP” refers to a RUSH system as afore-described, wherein said cytokine fused to SBP and to eGFP is C-X-C Chemokine Ligand 5 (CXCL10).

“CCL19-SBP-eGFP” refers to a RUSH system as afore-described, wherein said cytokine fused to SBP and to eGFP is C-C Chemokine Ligand 19 (CCL19).

“IFNg-SBP-eGFP” refers to a RUSH system as afore-described, wherein said cytokine fused to SBP and to eGFP is Interferon gamma (IFNγ).

“TNF-SBP-eGFP” refers to a RUSH system as afore-described, wherein said cytokine fused to SBP and to eGFP is Tumour Necrosis Factor (TNF).

“IL-7-SBP-eGFP” refers to a RUSH system as afore-described, wherein said cytokine fused to SBP and to eGFP is Interleukin-7 (IL-7).

“IL-15-SBP-eGFP” refers to a RUSH system as afore-described, wherein said cytokine fused to SBP and to eGFP is Interleukin-e15 (IL-15).

“tPa6-IL-15-SBP-eGFP” refers to a RUSH system as afore-described, wherein said cytokine fused to SBP and to eGFP is Interleukin-15 (IL-15) wherein “tPas6” Tissue Plasminogen Activation signal peptide (with SEQ ID NO: 602) replaces the native IL-15 peptide signal.

“CCL5-SBP-eGFP_{hibit}” refers to a RUSH system as afore-described, wherein said cytokine fused to SBP and to eGFP containing HiBit tag (SEQ ID NO: 604) for its quantitative determination using bioluminescence is C-C Chemokine Ligand 5 (CCL).

Methods

Test Items

RUSH systems constructions were obtained as previously described in Example 1, methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

Jurkat cells were plated into culture plates and transduced with said lentiviral particle at a MOI between 1 and 5 for 3 days.

Jurkat cells were plated into culture plates for western blotting or flow cytometry or plated onto coverslips for immunofluorescence.

Transduced cells either received no treatment or were treated with 40 μM of biotin for 15 minutes, 60-75 minutes or overnight (0/N).

Immunofluorescence

Immunofluorescence assays were performed as previously described in Example 1, methods section.

Western Blotting

Western blot was performed as previously described in Example 1, methods section.

Flow Cytometry

The RPMI medium was supplemented with 14 μg/mL of avidin to chelate the existing biotin present in the medium.

After incubation with biotin, the cells were immediately transfer on ice, to inhibit or slow down the traffic of the cargo, followed by centrifugation (300 g, 4° C., 5 minutes), washed twice in cold 1×PBS (300 g, 4° C., 5 minutes) and incubated with live/dead fixable staining (20 minutes, on ice; Thermofisher). The cells were then washed twice in cold PBS (300 g, 4° C., 5 minutes) or FACS buffer (1×PBS, 1% BSA, 0.05% sodium azide, 1 mL EDTA 0.5 M, filtered and kept at 4° C.) and, when not analyzed immediately, the cells were fixed in 3% PFA-1×PBS (10 minutes, RT) and washed twice in 1×PBS.

Results

GFP Staining in Jurkat Cells

Immunofluorescence images of Jurkat cells transduced with IL-2-SBP-eGFP, CCL5-SBP-eGFP or CXCL10-SBP-eGFP are respectively shown in FIGS. 13A, 13B and 13C.

Jurkat cells transduced with IL-2-SBP-eGFP (FIG. 13A), CCL5-SBP-eGFP (FIG. 13B) and CXCL10-SBP-eGFP (FIG. 13C), non-treated with biotin, showed a higher intracellular GFP expression; then, upon biotin addition, at 15 minutes, GFP-positive Golgi-like structures were observed. At 75 minutes with biotin, the intensity of GFP significantly decreased and dots appeared at the cell surface, suggesting that the cytokine was secreted in the medium (FIGS. 13A, 13B and 13C).

GFP Revelation in Jurkat Cells

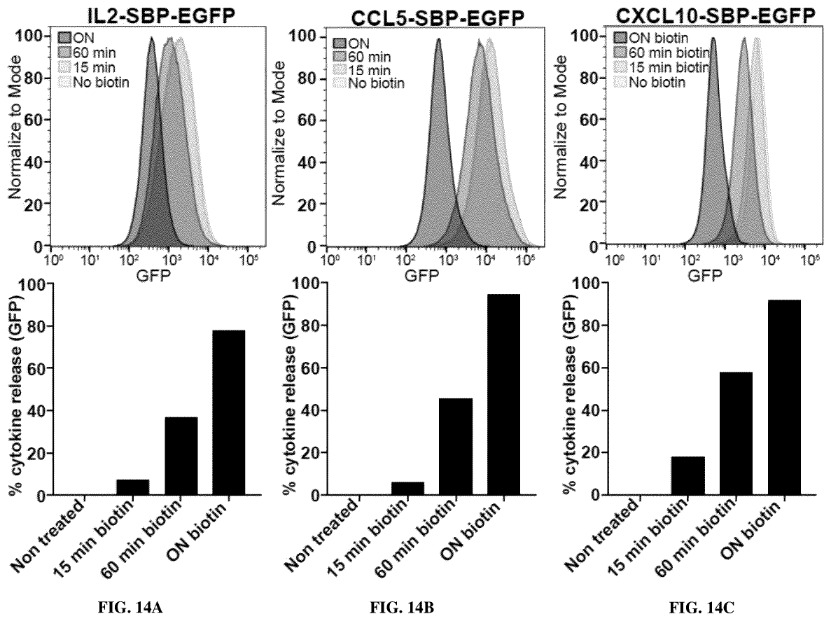

Flow cytometry staining graphs of Jurkat cells transduced with IL-2-SBP-eGFP, CCL5-SBP-eGFP or CXCL10-SBP-eGFP are respectively shown in FIGS. 14A, 14B and 14C.

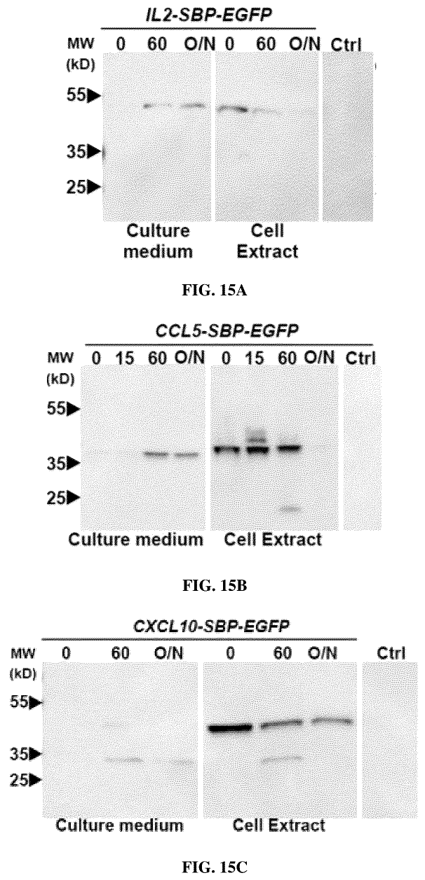

Western blot photographs of Jurkat cells transduced with IL-2-SBP-eGFP, CCL5-SBP-eGFP or CXCL10-SBP-eGFP are respectively shown in FIGS. 15A, 15B and 15C.

Flow cytometry was used to evaluate GFP expression that should be proportional to the amount of intracellular cytokine. Without biotin treatment, IL-2-SBP-eGFP- (FIG. 14A), CCL5-SBP-eGFP- (FIG. 14B) and CXCL10-SBP-eGFP- (FIG. 14C) transduced cells mostly expressed GFP. Upon addition of biotin, the intracellular GFP decreased significantly, mainly at later time points (60 minutes and O/N biotin), suggesting that a high proportion of the cytokine was secreted (FIGS. 14A, 14B and 14C). These results were represented in histogram (FIGS. 14A, 14B and 14C) with the GFP expression normalized to non-treated cells (highest GFP expression).

These results were confirmed by western blot (FIGS. 15A, 15B and 15C). Moreover, by western blot in the absence of biotin (time 0), no cytokine was detected in the medium; only at 60 minutes, IL-2-SBP-eGFP, CCL5-SBP-eGFP and CXCL10-SBP-eGFP (respectively FIGS. 15A, 15B and 15C) were detected in the medium; evidently, these cytokines were detected in the cell extract at time 0, since they were retained in the cell (FIGS. 15A, 15B and 15C).

### Example 9

The expression of cytokines in a RUSH system in a model T cell line, i.e., Jurkat cells, was evaluated.

Material

Cells

Jurkat cells cultured in Roswell Park Memorial Institute (RPMI)-1640 supplemented with 10% FBS or RPMI medium supplemented with 14 pg/mL of avidin to chelate the existing biotin present in the medium.

Test Items

Several RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“CCL19-SBP-eGFP” refers to a RUSH system as afore-described, wherein said cytokine fused to SBP and to eGFP is C-C Chemokine Ligand 19 (CCL19).

“IFNg-SBP-eGFP” refers to a RUSH system as afore-described, wherein said cytokine fused to SBP and to eGFP is Interferon gamma (IFNγ).

“TNF-SBP-eGFP” refers to a RUSH system as afore-described, wherein said cytokine fused to SBP and to eGFP is Tumour Necrosis Factor (TNF).

“IL-7-SBP-eGFP” refers to a RUSH system as afore-described, wherein said cytokine fused to SBP and to eGFP is Interleukin-7 (IL-7).

“IL-15-SBP-eGFP” refers to a RUSH system as afore-described, wherein said cytokine fused to SBP and to eGFP is Interleukin-15 (IL-15).

“tPa6-IL-15-SBP-eGFP” refers to a RUSH system as afore-described, wherein said cytokine fused to SBP and to eGFP is Interleukin-15 (IL-15) wherein “tPas6” Tissue Plasminogen Activation signal peptide (with SEQ ID NO: 602) replaces the native IL-15 peptide signal.

“CCL5-SBP-eGFP_{hibit}” refers to a RUSH system as afore-described, wherein said cytokine fused to SBP and to eGFP containing HiBit tag (SEQ ID NO: 604) for its quantitative determination using bioluminescence is C-C Chemokine Ligand 5 (CCL).

Methods

Test Items

RUSH systems constructions were obtained by insertion of a streptavidin tagged with KDEL (SEQ ID NO: 10) for luminal ER retention under control of sFFv promoter in lentiviral plasmid. Cytokines tagged with a streptavidin binding peptide, generally in C-terminus followed by a fluorescent protein eGFP were inserted after the PGK promoter downstream sFFv streptavidin construct afore-described. The signal peptide of origin was maintained. The cytokine sequences were all generated by gene synthesis using gblock (Integrated DNA Technologies) or Twist bioscience technology.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

Jurkat cells were plated into culture plates and transduced with said lentiviral particle at a MOI between 1 and 5 for 3 days.

Jurkat cells were plated into culture plates for flow cytometry.

Transduced cells either received no treatment or were treated with 40 μM of biotin for 6 hours.

Flow Cytometry

The RPMI medium was supplemented with 14 pg/mL of avidin to chelate the existing biotin present in the medium.

After incubation with biotin, the cells were immediately transfer on ice, to inhibit or slow down the traffic of the cargo, followed by centrifugation (300 g, 4° C., 5 minutes), washed twice in cold 1×PBS (300 g, 4° C., 5 minutes) and incubated with live/dead fixable staining (20 minutes, on ice; Thermofisher). The cells were then washed twice in cold PBS (300 g, 4° C., 5 minutes) or FACS buffer (1×PBS, 1% BSA, 0.05% sodium azide, 1 mL EDTA 0.5 M, filtered and kept at 4° C.) and, when not analyzed immediately, the cells were fixed in 3% PFA-1×PBS (10 minutes, RT) and washed twice in 1×PBS.

Results

GFP Staining in Jurkat Cells

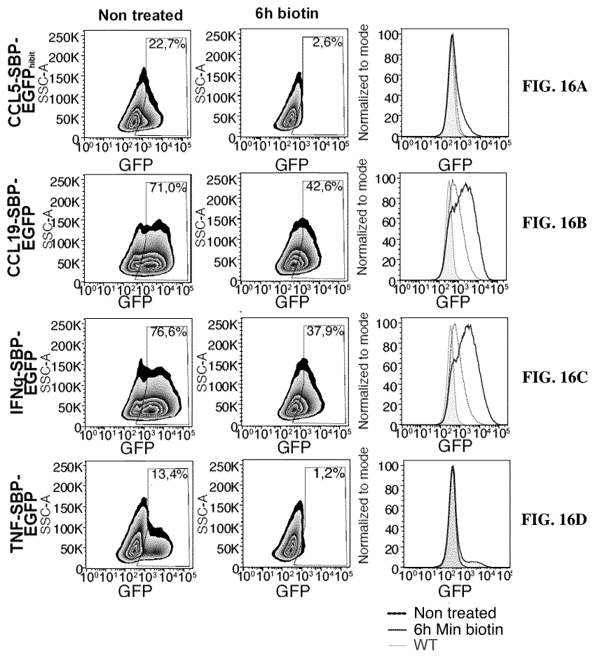

Flow cytometry staining graphs of Jurkat cells transduced with CCL5-SBP-eGFP^{hibit}, CCL19-SBP-eGFP, IFNg-SBP-eGFP or TNF-SBP-eGFP are respectively shown in FIGS. 16A, 16B, 16C and 16D.

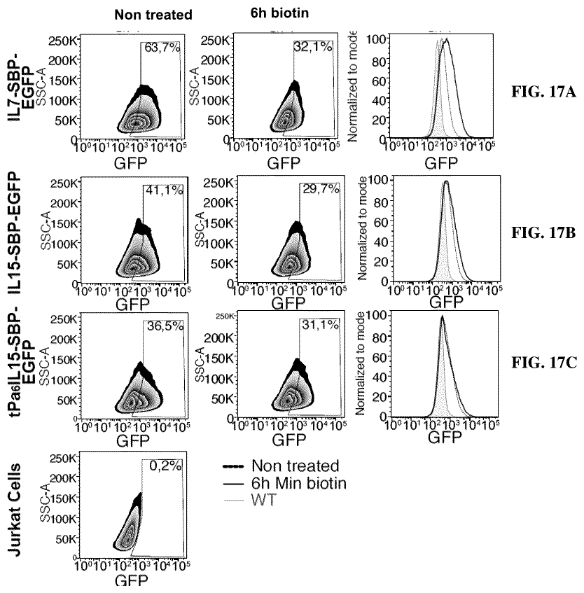

Flow cytometry staining graphs of Jurkat cells transduced with IL-7-SBP-eGFP, IL-15-SBP-eGFP or tPa6-IL-15-SBP-eGFP are respectively shown in FIGS. 17A, 17B and 17C.

Jurkat cells transduced with CCL5-SBP-eGFP_{hibit} (FIG. 16A), CCL19-SBP-eGFP (FIG. 16B), IFNg-SBP-eGFP (FIG. 16C), TNF-SBP-eGFP (FIG. 16D), IL-7-SBP-eGFP (FIG. 17A), IL-15-SBP-eGFP (FIG. 17B) or tPa6-IL-15-SBP-eGFP (FIG. 17C), non-treated with biotin, showed a proper intracellular GFP expression.

A decrease in the intensity of GFP was observed as biotin was added to the cells for the cytokines CCL19 (FIG. 16B), IFNγ (FIG. 16C) and TNF (FIG. 16D), within 6 hours, suggesting that they were secreted into the cell medium. IL-7 (FIG. 17A) and CCL5_{hibit} (FIG. 16A), also showed a decrease in GFP-expressing cells upon biotin addition due to cellular secretion. IL-15 (FIG. 17B) and tPa6-IL-15 (FIG. 17C) were well expressed in Jurkat cells and, when biotin was added, a small decrease in GFP-expressing cells (5-10%) could be observed, due to cytokine secretion.

### Example 10

The expression of cytokines in a RUSH system in primary CD8⁺ T cells was evaluated.

Material

Cells

T cells isolated from leukocyte reduction system chamber (LRSC) from blood of healthy donors and cultured in TexMacs buffer containing recombinant IL-7 (10 ng/mL, Miltenyi) and IL-15 (10 ng/mL, Miltenyi), and activated using T Cell TransAct™ human in TexMacs buffer containing recombinant IL-7 (10 ng/mL, Miltenyi) and IL-15 (10 ng/mL, Miltenyi). T cells, when frozen in CryoStor® CS10, were let resting for at least 16 hours in TexMacs buffer containing recombinant IL-7 (10 ng/mL, Miltenyi) and IL-15 (10 ng/mL, Miltenyi), and activated on the following day using T Cell TransAct™ human.

Test Items

Several RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“IL-2-SBP-eGFP” refers to a RUSH system as afore-described, wherein said cytokine fused to SBP and to eGFP is Interleurkin-2 (IL-2).

“CCL5-SBP-eGFP” refers to a RUSH system as afore-described, wherein said cytokine fused to SBP and to eGFP is C-C Chemokine Ligand 5 (CCL5).

“CXCL10-SBP-eGFP” refers to a RUSH system as afore-described, wherein said cytokine fused to SBP and to eGFP is C-X-C Chemokine Ligand 5 (CXCL10).

“CCL19-SBP-eGFP” refers to a RUSH system as afore-described, wherein said cytokine fused to SBP and to eGFP is C-C Chemokine Ligand 19 (CCL19).

“IFNg-SBP-eGFP” refers to a RUSH system as afore-described, wherein said cytokine fused to SBP and to eGFP is Interferon gamma (IFNγ).

“TNF-SBP-eGFP” refers to a RUSH system as afore-described, wherein said cytokine fused to SBP and to eGFP is Tumour Necrosis Factor (TNF).

“IL-7-SBP-eGFP” refers to a RUSH system as afore-described, wherein said cytokine fused to SBP and to eGFP is Interleukin-7 (IL-7).

“IL-15-SBP-eGFP” refers to a RUSH system as afore-described, wherein said cytokine fused to SBP and to eGFP is Interleukin-15 (IL-15).

“tPa6-IL-15-SBP-eGFP” refers to a RUSH system as afore-described, wherein said cytokine fused to SBP and to eGFP is Interleukin-15 (IL-15) wherein “tPas6” Tissue Plasminogen Activation signal peptide (with SEQ ID NO: 602) replaces the native IL-15 peptide signal.

“CCL5-SBP-eGFP_{hibit}” refers to a RUSH system as afore-described, wherein said cytokine fused to SBP and to eGFP containing HiBit tag (SEQ ID NO: 604) for its quantitative determination using bioluminescence is C-C Chemokine Ligand 5 (CCL).

Methods

T Cell Isolation

T cells were isolated from leukocyte reduction system chamber (LRSC) from blood of healthy donors by negative selection using the EasySep Direct Human T cell isolation kit (STEM cells) or MACSxpress LRSC Pan T Cell Isolation Kit, human (Miltenyi) according to the manufacturer's instructions.

Test Items

RUSH systems constructions were obtained as previously described in Example 1, methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

Primary CD8⁺ T or CD4/CD8⁺ T cells were plated into culture plates and transduced with said lentiviral particle at a MOI between 1 and 5 for 3 days.

Primary CD8⁺ T or CD4/CD8⁺ T cells were plated into culture plates for Flow cytometry assay.

To prevent cytokines released due to the possible presence of biotin in TexMacs buffer, 1 μg/mL of avidin was added to chelate biotin, starting from the day of transduction and following days of cell culture.

Transduced cells either received no treatment or were treated with 40 μM of biotin for 15 minutes, 60 minutes, more than 4 hours or overnight (O/N).

Flow Cytometry

After incubation with biotin, the cells were immediately transfer on ice, to inhibit or slow down the traffic of the cargo, followed by centrifugation (300 g, 4° C., 5 minutes), washed twice in cold 1×PBS (300 g, 4° C., 5 minutes) and incubated with live/dead fixable staining (20 minutes, on ice; Thermofisher). The cells were then washed twice in cold PBS (300 g, 4° C., 5 minutes) or FACS buffer (1×PBS, 1% BSA, 0.05% sodium azide, 1 mL EDTA 0.5 M, filtered and kept at 4° C.) and, when not analyzed immediately, the cells were fixed in 3% PFA-1×PBS (10 minutes, RT) and washed twice in 1×PBS.

Results

GFP Staining in Primary T Cells

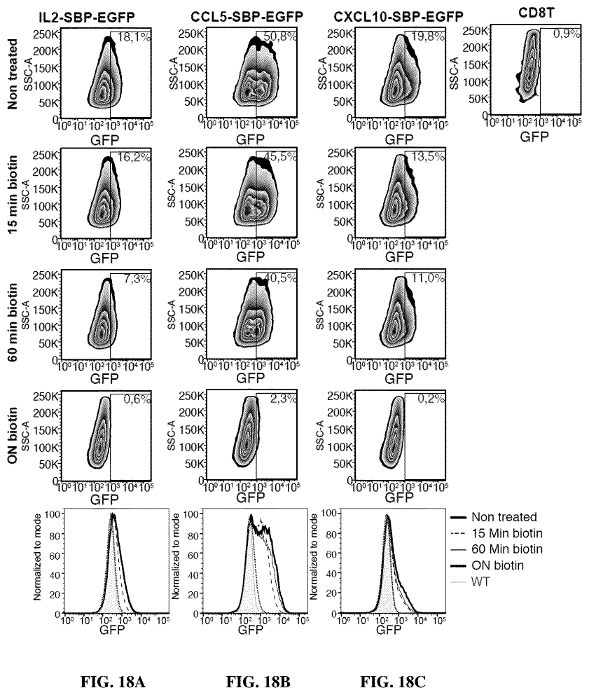

Flow cytometry assays of primary CD8⁺ T cells transduced with IL-2-SBP-eGFP, CCL5-SBP-eGFP or CXCL10-SBP-eGFP are respectively shown in FIGS. 18A, 18B and 18C.

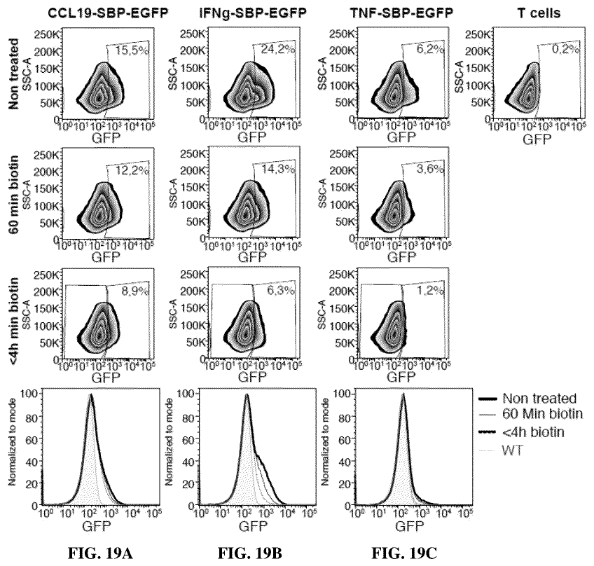

Flow cytometry assays of primary T cells transduced with CCL19-SBP-eGFP, IFNg-SBP-eGFP or TNF-SBP-eGFP are respectively shown in FIGS. 19A, 19B and 19C.

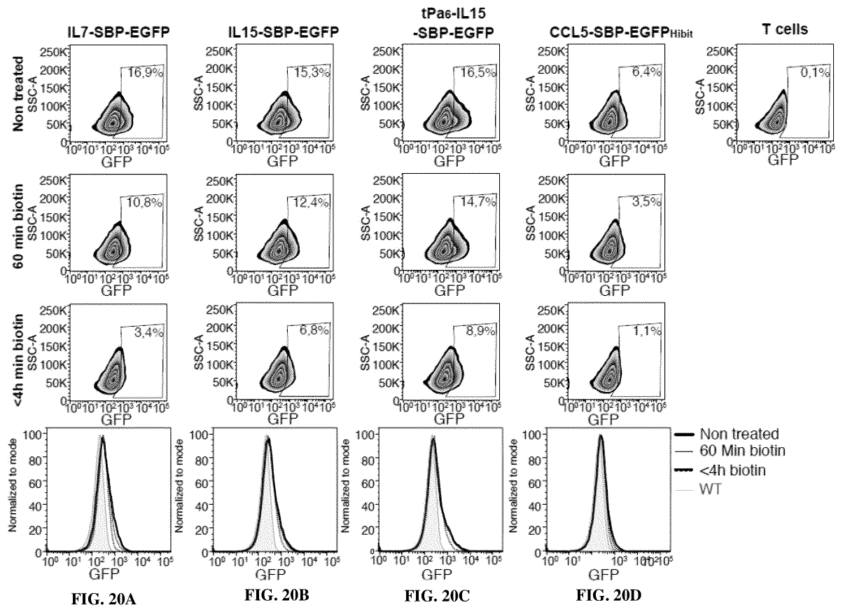

Flow cytometry assays of primary T cells transduced with IL-7-SBP-eGFP, IL-15-SBP-eGFP, tPa6-IL-15-SBP-eGFP or CCL5-SBP-eGFP_{hibit} are respectively shown in FIGS. 20A, 20B, 20C and 20D.

T cells were transduced with the cytokines IL-2, CCL5 and CXCL10 with an efficiency of transduction (absence of biotin) of 18, 51 and 20% respectively (respectively FIGS. 18A, 18B and 18C). When biotin was added, for these three cytokines, a significant decrease in the GFP-expressing cells was observed, reaching almost zero when biotin was added O/N, suggesting that these cytokines are efficiently secreted in the medium (FIGS. 18A, 18B and 18C).

The cytokines CCL19, IFNγ and TNF were expressed in about 16, 24 and 6% of T cells respectively, (respectively FIGS. 19A, 19B and 19C). Once again, addition of biotin led to cytokine secretion, as assessed by the decrease of GFP-expressing cells (FIGS. 19A, 19B and 19C).

IL-7, IL-15, tPa6-IL-15 and CCL5 with GFP tagged with HiBit were also used to transduce T cells with an efficiency of 17, 15, 17 and 6% respectively (respectively FIGS. 20A, 20B, 20C and 20D). The treatment with biotin led to the decrease of GFP-expressing cells, due to cytokine secretion (FIGS. 20A, 20B, 20C and 20D).

### Example 11

The expression of cytokines in a RUSH system in primary macrophages was evaluated.

Material

Cells

Macrophages cultured in RPMI (Gibco) supplemented with 5% of FBS, 100 μM of penicillin and streptomycin (Invitrogen) and 25 ng/mL of macrophage colony-stimulating factor (M-CSF; ImmunoTools).

Test Item

A RUSH system with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“CCL5-SBP-eGFP” refers to a RUSH system as afore-described, wherein said cytokine fused to SBP and to eGFP is C-C Chemokine Ligand 5 (CCL5).

Methods

Monocyte Purification

Monocytes were isolated from PBMCs, previously separated using Ficoll-Paque (GE Healthcare), by CD14⁺ magnetic microbeads (Miltenyi), followed by 7 days of differentiation into macrophages using differentiation medium composed of RPMI (Gibco) supplemented with 5% of FBS, 100 sM of penicillin and streptomycin (Invitrogen) and 25 ng/mL of macrophage colony-stimulating factor (M-CSF; ImmunoTools).

Test Item

The RUSH system construction was obtained as previously described in Example 1, methods section.

Study Design

Production of lentiviral particles containing aforementioned RUSH system using the packaging plasmid psPAX2 (12260; Addgene) and envelop plasmid pVSVG (pMD2.G; 12259, Addgene) was done in HEK293FT cells.

Primary human monocytes were transduced with RUSH CCL5-SBP-eGFP at MOI 5 supplemented with Vpx particles, followed by 7 days differentiation into macrophages in the presence of GM-CSF.

After 7 days of transduction, cells were detached and plated onto coverslips and later live-imaged using a spinning-dish before (non-treated) and after addition of 40 μM of biotin.

Images were taken after 0 minutes, 10 minutes, 15 minutes, 20 minutes, 30 minutes, 40 minutes, 50 minutes, 60 minutes or 80 minutes of biotin treatment.

Live Imaging

RUSH-transduced macrophages seeded onto a 25 mm-diameter glass coverslip were placed into a L-shape tubing Chamlide (Live Cell Instrument) and filled with pre-warmed Leibovitz's medium (Invitrogen). The cells were imaged 1-2 minutes before the addition of biotin and at time zero, biotin was added and the time-lapse acquisition continued at 37° C. in a thermostat-controlled chamber. The images were acquired using an Eclipse 80i microscope (Nikon) equipped with spinning disk confocal head (Perkin) and a CoolSnapHQ2 camera (Roper Scientific). The images were analyzed using Fiji—ImageJ software (Molecular Device).

Results

Imaging CCL5-SBP-eGFP Traffic

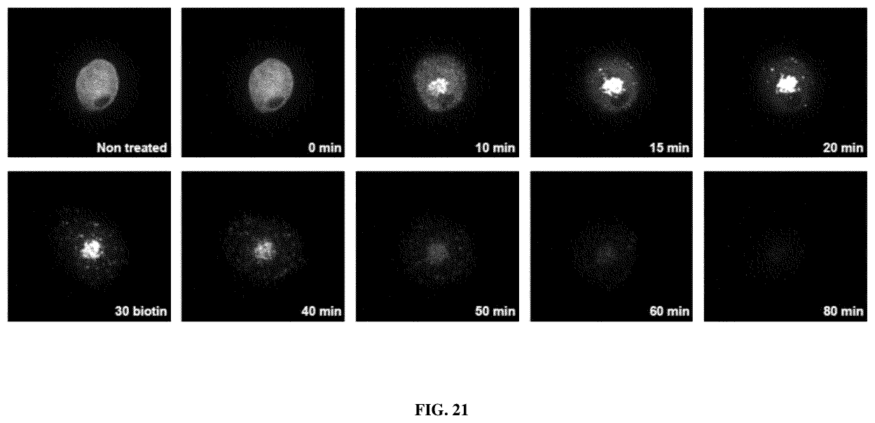

Real-time images of primary monocytes-derived macrophages transduced with CCL5-SBP-eGFP are shown on FIG. 21.

In the absence of biotin, CCL5 was retained in the cells in an ER-like structure and, upon biotin addition, it trafficked to the ER and then to cell surface, where it was secreted (FIG. 21).

### Example 12

Material

Cells

Jurkat cells cultured in Roswell Park Memorial Institute (RPMI)-1640 supplemented with 10% FBS or RPMI medium supplemented with 14 pg/mL of avidin to chelate the existing biotin present in the medium.

HEK293FT reporter cells cultured in DMEM (Dulbecco's modified Eagle medium) supplemented with 10% Fetal Bovine Serum, 1 mM sodium pyruvate and 100 μM of normocin.

Test Items

Two different RUSH systems were tested.

A first RUSH system with a single CMV promoter in a lentiviral vector comprising a streptavidin fused to a KDEL endoplasmic reticulum-retention signal (SEQ ID NO: 10) followed by an IVS-IRES signal and a cytokine fused to streptavidin binding peptide (SBP) with SEQ ID NO: 605 and to eGFP (see WO2010142785, also illustrated in FIG. 24A).

A second/third RUSH system with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide SBP) with SEQ ID NO: 605 and to eGFP under the control of a weaker promoter PGK or a strong promoter sFFv (as described herein and illustrated in FIG. 24B).

These systems were used to express IL-2 and CCL5 as follows: under the control of a weaker promoter PGK (pPGK-IL-2 GFP or pPGK-CCL5 GFP), downstream of an IVS-IRES (ivsIRES-IL-2 GFP or ivsIRES-CCL5 GFP) or under the control of a strong promoter sFFv (prsFFv-IL-2 GFP or prsFFV-CCL5 GFP).

“ivsIRES-IL-2 GFP vector” refers to a RUSH system with a single promoter and an IVS-IRES, wherein said cytokine fused to SBP and to eGFP is Interleukin-2 (IL-2).

“pPGK-IL-2 GFP vector” refers to a RUSH system with a weaker promoter PGK, wherein said cytokine fused to SBP and to eGFP is Interleukin-2 (IL-2).

“prsFFv-IL-2 GFP vector” refers to a RUSH system with a strong promoter sFFv, wherein said cytokine fused to SBP and to eGFP is Interleukin-2 (IL-2).

“ivsIRES-CCL5 GFP vector” refers to a RUSH system with a single promoter and an IVS-IRES, wherein said cytokine fused to SBP and to eGFP is C-C Chemokine Ligand 5 (CCL5).

“pPGK-CCL5 GFP vector” refers to a RUSH system with a weaker promoter PGK, wherein said cytokine fused to SBP and to eGFP is C-C Chemokine Ligand 5 (CCL5).

“prsFFv-CCL5 GFP vector” refers to a RUSH system with a strong promoter sFFV, wherein said cytokine fused to SBP and to eGFP is C-C Chemokine Ligand 5 (CCL5).

Methods

Test Items

RUSH systems constructions were obtained as described in Example 1, methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

Transduction of HEK293FT and Jurkat cells were done using the same volume v/v of particles for each construct increasingly.

The expression of the cytokine was evaluated by measuring GFP using flow cytometry, three days later for HEK293FT and more than six days later for Jurkat cells.

Results

Quantification of the Percentage of Transduced Cells

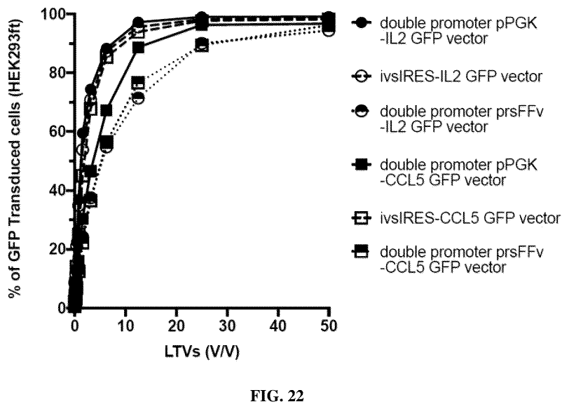

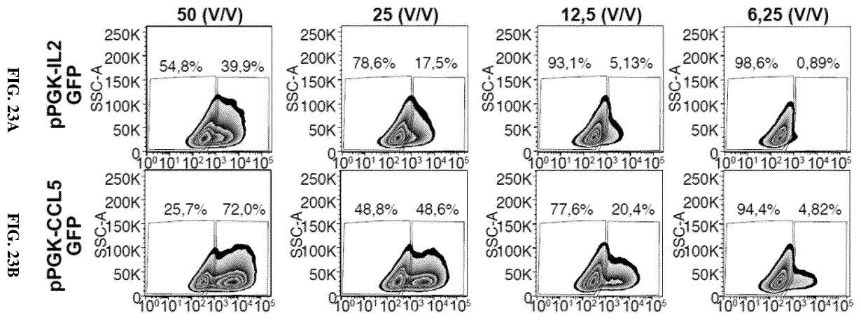

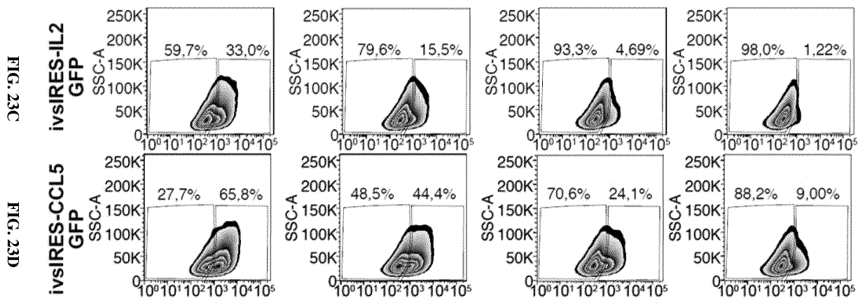

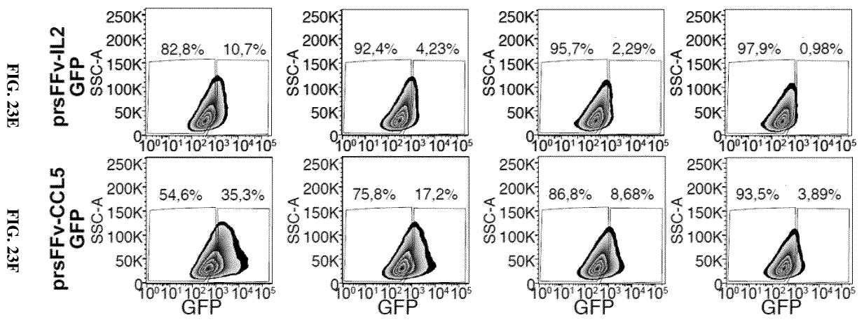

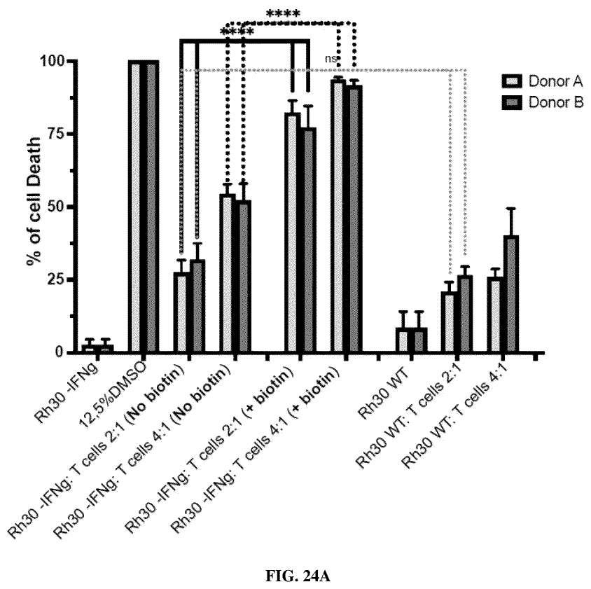

Graph illustrating the percentage of transduced Jurkat cells with the various RUSH constructs is shown in FIG. 22.

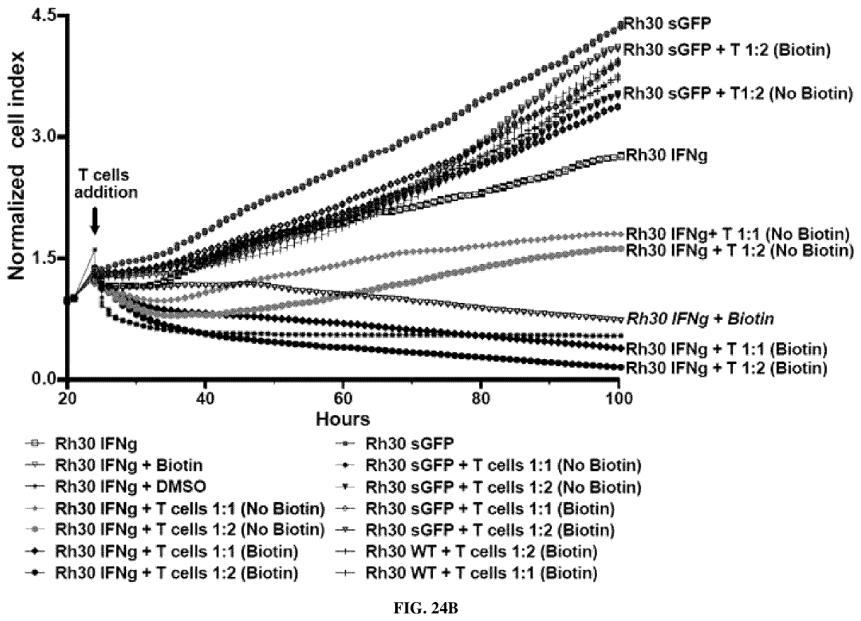

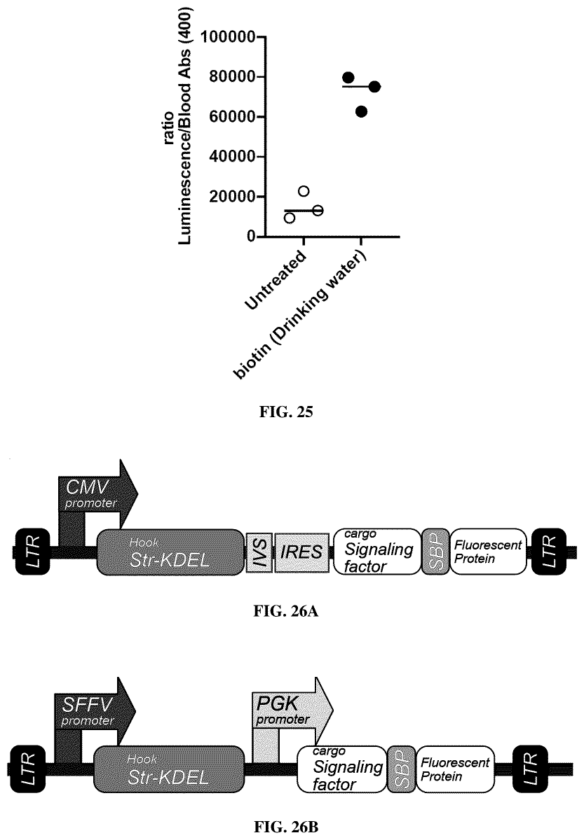

Graphs illustrating the percentage of transduced HeLa cells with the double-promoter pPGK-IL-2-eGFP vector, the double-promoter pPGK-CCL5-eGFP vector, the IVS-IRES-IL-2-eGFP vector, IVS-RES-CCL5-eGFP vector, prsFFv-IL-2-eGFP vector or prsFFv-CCL5-eGFP vectors are respectively shown in FIGS. 23A, 23B, 23C, 23D, 23D 23E and 23F.

The transduction efficiency in HEK293FT was similar for double promoter with the PGK and IVS-IRES vectors while for the double promoter with the sFFv was slightly lower (FIG. 22).

In Jurkat cells, the expression of the cytokines was lower when using lentiviruses produced with an IVS-IRES (FIGS. 23C and 23D) in comparison to the weaker promoter PGK (FIGS. 23A and 23B), with a major difference in the intensity of GFP expressing cells.

The expression of the cytokine using double promoter with the strong promoter sFFv was significantly impaired (FIGS. 23E and 23F). The weaker expression of the cytokine using the stronger promoter sFFv could be due higher cytokine leakage. This suggests that lentiviruses generated with the double promoter using a weaker promoter are more efficient for the transduction of cytokine-RUSH systems in T cells.

### Example 13

Material

Cells

CD4/CD8 from PBMCs cells were generated by activation using T Cell TransAct™ human in TexMacs buffer containing recombinant IL-7 (10 ng/mL, Miltenyi) and IL-15 (10 ng/mL, Miltenyi). After 3 days, T Cell TransAct™ was removed and fresh TexMacs buffer containing recombinant IL-7(10 ng/mL, Miltenyi) and IL-15 (10 ng/mL, Miltenyi) was added. The cells were maintained in culture for at least additional 4-6 days followed by flow cytometry evaluation of the percentage of CD3⁺ T or CD4⁺/CD8⁺ T cells before incubation with target cells.

Rh30 luciferase cells cultured in DMEM (Dulbecco's modified Eagle medium) supplemented with 10% Fetal Bovine Serum, 1 mM sodium pyruvate and 100 μM of penicillin and streptomycin.

Test Items

A RUSH system with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to eGFP under the control of a weaker promoter PGK.

“IFNg-SBP-eGFP” refers to a RUSH system with a double promoter as afore-described, wherein said cytokine fused to SBP and to eGFP is Interferon gamma (IFNγ).

“ss-SBP-eGFP” refers to a RUSH system with a double promoter as afore-described, wherein said single peptide of IL-2 (ss) upstream to SBP fused to eGFP.

Methods

Test Items

The RUSH system construction was obtained as described in Example 1, methods section.

Study Design

Production of lentiviral particles containing aforementioned RUSH system using the packaging plasmid psPAX2 (12260; Addgene) and envelop plasmid pVSVG (pMD2.G; 12259, Addgene) was done in HEK293FT cells.

Rh30 cells were transduced with said RUSH system (-IFN), (-GFP) or not (WT), then co-cultured with T cells from different donors at indicated ratios (Rh30 cells:T cells) varying from 1:1 to 4:1 in presence or in absence of biotin in X-ViVO medium.

Bioluminescence Assay

Percentage of Rh30 death was assessed by Bioluminescence assay upon luciferin substrate addition assay after 72 hours with FLUOstar OPTIMA (BMG LabTech).

The percentage of cell survival was calculated by taking the percentage of survival (luminescence) for each point and dividing it by the highest value of survival (luminescence) obtained. Cell death value was then obtained by subtracting 100% of death to the survival obtained.

Real-Time Cell Death of Rh30 Co-Cultured with T Cells

Real-time cell death measurements were performed using xCELLigence RTCA eSight—Imaging & Impedance. Briefly, 5 000 target cells were plated in a 96-well plate (ACEA Biosciences, San Diego, CA, USA) in complete DMEM medium and the next day the effector cells were added at indicated E:T ratios in X-VIVO medium (2-fold volume compared to DMEM). Cell index (relative impedance) was monitored in real-time every 15 minutes for about four days at 37° C. and 5% CO₂.

Results

Cell Death Induced by IFN Activated T Cells

A significant target cell killing by the T cells was observed in the presence of biotin in comparison to no biotin in the different Rh30:T cells ratios, for both donors (FIGS. 24A and 24B).

The increase in target cell killing was induced by the presence of biotin that promoted the secretion of IFNγ by the target cells and, consequently, T cell activation and killing. Upon biotin addition, IFNγ was secreted by the target cells leading to a significant increase of their killing by T cells at both ratios and in both donors. Of note, Rh30 target cells non-transduced with IFNγ (WT) were also killed by the T cells in a similar manner as the Rh30-IFNγ (FIG. 24A). These results were confirmed by real-time cell death measurements using xCELLigence RTCA eSight (FIG. 24B). A higher cell death by T cells induced by IFN 7 secretion was observed upon biotin addition. Interestingly, the secretion of IFNγ by the presence of biotin and in the absence of T cells also induced cell death, although to a lesser extent (FIG. 24B). This suggests that IFNγ can induce cancer cell death with or even without the presence of T cells.

### Example 14

Cytokine Secretion from Tumour Transduced Cells Implanted in NSG Mice Upon Addition of Biotin in Drinking Water.

Material

Cells

MCA205 mouse cells cultured in DMEM (Dulbecco's modified Eagle medium) supplemented with 10% Fetal Bovine Serum, 1 mM sodium pyruvate and 100 μM of penicillin and streptomycin.

Test Items

RUSH systems with a double promoter in a lentiviral vector comprising a streptavidin fused to a KDEL endoplasmic reticulum-retention signal (SEQ ID NO: 10) under sFFv strong promoter followed by a cytokine fused to streptavidin binding peptide (SBP) with SEQ ID NO: 605 and to NLuc (NanoKAZ) with SEQ ID NO: 638 under the control of a weaker promoter PGK.

“CCL5-SBP-NLUC” refers to a RUSH system as afore-described, wherein said cytokine fused to SBP and to NLuc is C-C Chemokine Ligand 5 (CCL5).

Methods

Test Items

RUSH systems constructions were obtained as described in Example 1, methods section.

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

MCA205 cells were plated into culture plates and transduced with said lentiviral particle at a MOI between 1 and 5 for 2 days.

Transduced MCA205 mouse cells in PBS were subcutaneously injected in immunodeficient NSG mouse (NOD scid gamma mouse).

Cytokine Activity in Blood

When the tumor reached a volume higher than 350 mm³, biotin dissolved in mice drinking water (about 0.5 mg/mL) and supplemented with 1% of sucrose was given to the animals. After more than 3 days, a drop of blood (about 20 μL) was taken from the caudal vein and mixed with heparin-PBS. The blood was kept at 4° C. for less than one hour and 5-10 μL of blood in heparin-PBS was diluted with 5 μM of luciferin (50 μL total volume) in a 96-well ViewPlate Black (Perkin-Elmer) and the luminescence measurement with FLUOstar OPTIMA (BMG LabTech) as well as the absorbance of the blood at 400 nm. The activity/luminescence value of the nLUC fused to the cytokine was divided by the absorbance of the blood at 400 nm.

Animal Experiments

NGS mice were housed in SPF conditions in the animal facilities in Institute Curie. Live animal experiments were performed in accordance to the national guidelines.

One million of transduced MCA205 mouse fibrosarcoma cells in PBS were subcutaneously injected in immunodeficient NSG mouse (NOD scid gamma mouse).

Results

Cytokine secretion from tumor transduced cells implanted in NSG mice was induced upon addition of biotin in drinking water during more than three days (FIG. 25). This experiment shows that in vivo release of cytokines induced by biotin treatment of the mouse is made possible using RUSH constructs.

### Example 15

Material

Cells

HEK293 ft and HeLa cells cultured in DMEM (Dulbecco's modified Eagle medium) supplemented with 10% Fetal Bovine Serum (FBS), 1 mM sodium pyruvate and 100 μM of penicillin and streptomycin, at 37° C. and 5% of CO₂.

Jurkat cells cultured in Roswell Park Memorial Institute (RPMI)-1640 without biotin supplemented with 10% FBS at 37° C. and 5% of CO₂.

Test Items

RUSH systems with a double promoter in a lentiviral vector comprising a streptavidin (hook protein) fused to a KDEL endoplasmic reticulum-retention signal (SEQ ID NO: 10) under the control of (i) a sFFv strong promoter or (ii) a PGK weaker promoter, followed by a cytokine fused to streptavidin binding peptide (SBP) with SEQ ID NO: 605 and to eGFP (i) under the control of a weaker promoter PGK, UbC or SV40, or (ii) under the control of a strong promoter prsFFv, or (iii) downstream of an IVS-IRES signal.

“prsFFv-pPGK-CCL5 GFP” refers to a RUSH system with the hook protein under the control of a sFFv strong promoter, followed by a cytokine under the control of a weaker promoter PGK, wherein said cytokine fused to SBP and to eGFP is C-C Chemokine Ligand 5 (CCL5).

“prsFFv-prsFFV CCL5 GFP” refers to a RUSH system with the hook protein under the control of a sFFv strong promoter, followed by a cytokine under the control of a strong promoter sFFv, wherein said cytokine fused to SBP and to eGFP is C-C Chemokine Ligand 5 (CCL5).

“prsFFv-pUBC-CCL5 GFP” refers to a RUSH system with the hook protein under the control of a sFFv strong promoter, followed by a cytokine under the control of a weaker promoter UbC, wherein said cytokine fused to SBP and to eGFP is C-C Chemokine Ligand 5 (CCL5).

“prsFFv-pSV40-CCL5 GFP” refers to a RUSH system with the hook protein under the control of a sFFv strong promoter, followed by a cytokine under the control of a weaker promoter SV40, wherein said cytokine fused to SBP and to eGFP is C-C Chemokine Ligand 5 (CCL5).

“pPGK-pPGK-CCL5 GFP” refers to a RUSH system with the hook protein under the control of weaker PGK promoter, followed by a cytokine under the control of the same promoter PGK, wherein said cytokine fused to SBP and to eGFP is C-C Chemokine Ligand 5 (CCL5).

“pPGK-prsFFv-CCL5 GFP” refers to a RUSH system with the hook protein under the control of a PGK weaker promoter, followed by a cytokine under the control of a stronger promoter sFFv, wherein said cytokine fused to SBP and to eGFP is C-C Chemokine Ligand 5 (CCL5).

“prsFFv-ivsIRES-CCL5 GFP” refers to a RUSH system with the hook protein under the control of a sFFv strong promoter, followed by a cytokine downstream of an IVS-IRES, wherein said cytokine fused to SBP and to eGFP is C-C Chemokine Ligand 5 (CCL5).

Methods

Study Design

Production of lentiviral particles containing aforementioned RUSH systems using the packaging plasmid psPAX2 (12260; Addgene) and envelop plasmid pVSVG (pMD2.G; 12259, Addgene) was done in HEK293FT cells.

Cells

HeLa cells were plated on a cell culture plate and transduced with said lentiviral particle at a MOI between 1 and 5 for 2 days. Transduced HeLa cells were plated on coverslip for immunofluorescence assay. Transduced cells either received no treatment or were treated with 40 μM of biotin.

Jurkat cells were plated into culture plates and transduced with said lentiviral particle at a MOI between 1 and 5 for 3 days. Jurkat cells were plated into culture plates for western blotting or flow cytometry or plated onto coverslips for immunofluorescence. Transduced cells either received no treatment or were treated with 40 μM of biotin for 15 minutes, 60-75 minutes or overnight (0/N).

For both types of cells, the expression of the cytokine was evaluated by measuring GFP using flow cytometry, 3 days later for HEK293FT and more than 6 days later for Jurkat cells.

Cells for western blotting were treated either treated or not with 40 μM biotin or higher for 60 minutes or overnight (O/N).

Western Blotting

After biotin treatment, the supernatant was recovered and centrifuged for removal of the detached or dead cells at 300 g, 4° C., for 5-10 minutes and kept on ice. While the cells (defined hereafter as pellet) were incubated with protein loading buffer 1×(5 mM Tris-HCl, pH 7.0, 30 mM ethylenediaminetetraacid (EDTA), pH 8.0; 0.01% bromophenol blue, 5% glycerol) for 5 minutes at room temperature, scratched and transfer to a new tube followed by denaturation at 95-100° C. for 10-20 minutes. The supernatant (from adherent or suspension cells) were incubated with StrataClean Resin (Agilent) to collect and concentrate protein present in the supernatant, for at least 2 hours at 4° C. with orbital agitation. Then, the resin was separated from the supernatant by centrifugation (10000-12000 g, 4° C., 5-10 minutes), wash twice in cold 1×PBS with centrifugation between after each wash (10000-12000 g, 4° C., 5-10 minutes), re-suspended in protein loading buffer 1× and denaturated at 95-100° C. for 10-20 minutes. The supernatant of the protein loading buffer was then recovered after centrifugation at 10000-12000 g, for 5 minutes at room temperature. Western blots were done under reducing conditions. Proteins were subjected to criterion TGX Stain Free, 4-20% gel electrophoresis (15 V, 60 minutes; Biorad), transferred using Protein Blotting Using the Trans-Blot® Turbo™ Transfer System (Biorad) according to the manufacturer's instructions. The membrane was washed two to three times in H₂O and once in PBS, 0.05% Tween-20 and blocked using 5% skim milk in 0.05% Tween-20 PBS for 1 hour at room temperature. Cytokines were detected using monoclonal anti-GFP (1/1000; Roche) in 5% skim milk in 0.1% Tween-20 PBS for 1 hour at room temperature or overnight (0/N) at 4° C., followed by the respective horseradish peroxidase (HRP) conjugated secondary polyclonal antibody (1/15000) in 5% skim milk in 0.1% Tween-20 PBS for 1 hour at room temperature. Peroxidase activity was revealed using SuperSignal™ West Pico PLUS Chemiluminescent Substrate (Pierce) in a photoradiograph (ChemiDoc MP Imaging System, Biorad).

Alternatively, the membranes were stained with a loading control, after HRP activity from the first stain was quenched using 15% of hydrogen peroxidase in 0.1% Tween-20 PBS for 30 minutes to 1 hour at room temperature. The loading control used was anti-vinculin (1/2000; Sigma) in 5% skim milk in 0.1% Tween-20 PBS for 1 hour at room temperature or overnight (O/N) at 4° C., followed by the respective horseradish peroxidase (HRP) conjugated secondary polyclonal antibody (1/15000) in 5% skim milk in 0.1% Tween-20 PBS for 1 hour at room temperature. The molecular weight (M) ladder used was PageRuler Plus Prestained Protein Ladder (Thermofisher).

Flow Cytometry

After incubation with biotin, the cells were immediately transferred to ice, to inhibit or slow down the traffic of the cargo, followed by centrifugation (300 g, 4° C., 5 minutes), washed twice with 1×PBS with centrifugation between after each wash (300 g, 4° C., 5 minutes), and incubated with live/dead fixable staining (20 minutes, 4° C.; Thermofisher). The cells were then washed in FACS buffer (1×PBS, 1% BSA, 0.05% sodium azide, 1 mL EDTA 0.5 M, filtered and kept at 4° C.) and, when not analyzed immediately, the cells were fixed in 3% PFA-1×PBS (10 minutes, room temperature) and washed twice in FACS buffer.

Results

Quantification of the Percentage of Transduced Cells

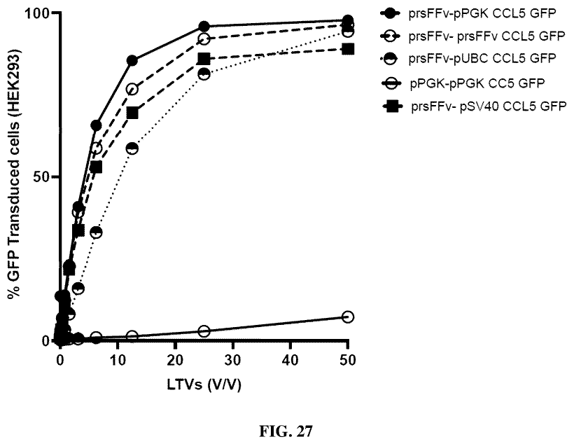

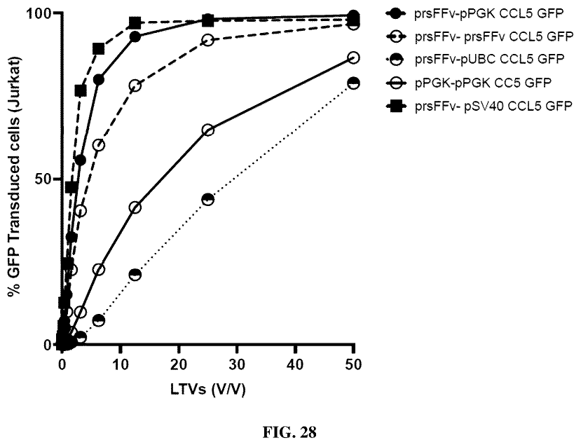

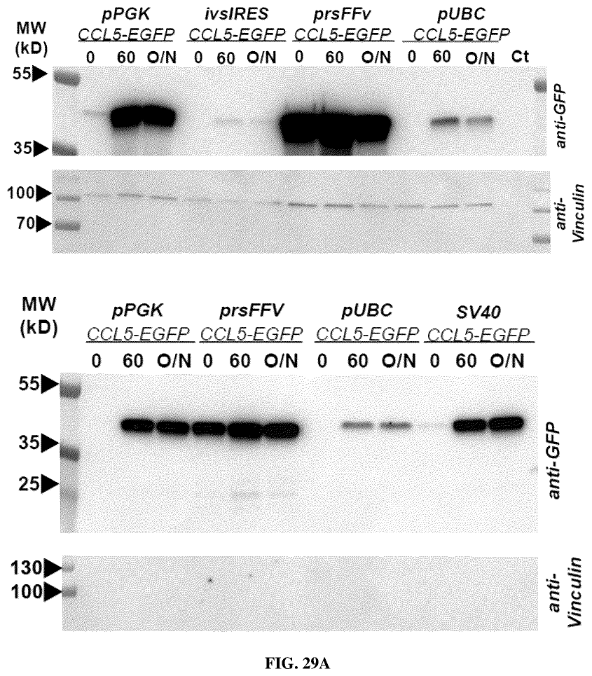

Graph illustrating the percentage of transduced HEK293 ft and Jurkat cells with the various RUSH constructs is shown in FIGS. 27 and 28, respectively.

The transduction efficiency in HEK293FT was similar for the constructions with the hook protein under the control of a sFFv promoter followed by a cytokine under the control of the weaker promoters PGK, UbC or SV40, or of the strong promoter sFFv (FIG. 27), but very weak when the combination of two weaker promoters (PGK-PGK) was used.

In Jurkat cells, the transduction efficiency was similar for the constructions with the hook protein under the control of a sFFv promoter followed by a cytokine under the control of the weaker promoters PGK or SV40 and feebler under control of the strong promoter sFFv (FIG. 28). The combination of stronger promoter sFFv and weaker promoter UbC was the weakest, as well as the combination of two weaker promoters (PGK-PGK).

Evaluation of the Leakage in RUSH System

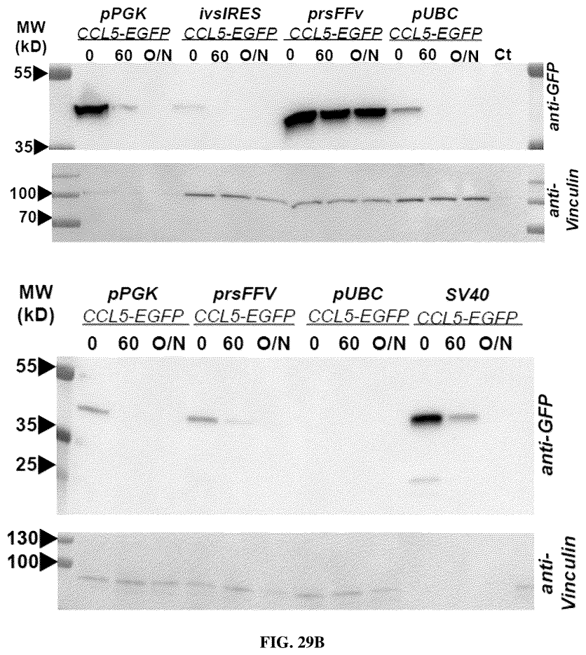

To evaluate the efficiency of retention/release in RUSH system using the different combination aforementioned, Western blot photographs of culture medium and cell extract of transduced HeLa cells was performed (FIGS. 29A and 29B, respectively).

Western Blotting—HeLa Cells

In HeLa cells transduced with the hook protein under the control of a strong promoter sFFv and the cytokine under control of the weaker promoters PGK, UbC or SV40, in the absence of biotin (t=0), no cytokine or very low levels were detected in the cell medium (FIG. 29A) as it remained intracellularly retained (FIG. 29B).

When the cytokine was under the control of the strong sFFv promoter, in the absence of biotin (t=0), the cytokine was secreted in the medium (FIG. 29A).

The expression of the cytokine using an IVS-IRES (as described in WO2010142785 and illustrated in FIG. 24A) was very weak in comparison to the double promoter system, almost indetectable by Western Blot.

Upon addition of biotin (t=60 or O/N), the cytokine was secreted in the cell medium for all constructs (FIG. 29A).

These results demonstrate that using a double promoter system with a combination of strong-strong promoters (sFFv-sFFv) leads to high leakage of the cytokine in the cell medium in absence of biotin, while a combination of a strong-weaker promoter is associated with a high retention in absence of biotin, followed by cytokine release when biotin is added.

Western Blotting—Jurkat Cells

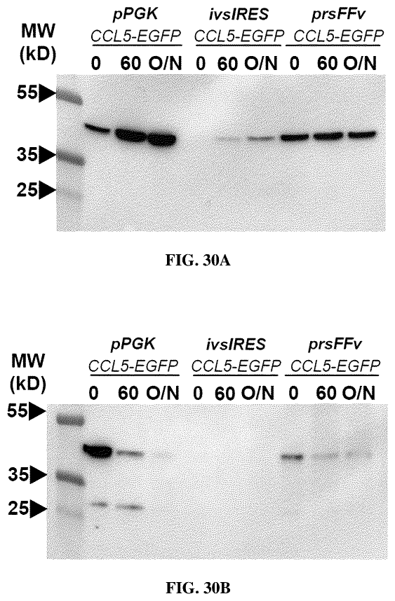

Western blot photographs of the culture medium and cell extract of Jurkat cells transduced with the hook protein under the control of a strong promoter sFFv and the cytokine under control of the weaker promoter PGK, the strong promoter sFFv or downstream of an IVS-IRES are shown in FIGS. 30 A and B.

When using the strong-weaker promoter combination sFFv-PGK, in the absence of biotin (t=0), a small amount of cytokine was released in the cell medium (FIG. 30 A), but, upon biotin addition (t=60 or O/N), its secretion increased significantly (FIG. 30 A).

When using a combination of strong-strong promoters (sFFv-sFFv), the leakage was much higher in the absence of biotin (t=0); the addition of biotin (t=60 or O/N) did not increase cytokine secretion, suggesting that RUSH dynamics was impaired (FIG. 30 A).

The expression of the cytokine using an IVS-IRES (as described in WO2010142785 and illustrated in FIG. 24A) strongly impaired cytokine expression in Jurkat cells (FIG. 30 B), with only a very low amount of cytokine released in the cell medium (FIG. 30 A).

Flow Cytometry

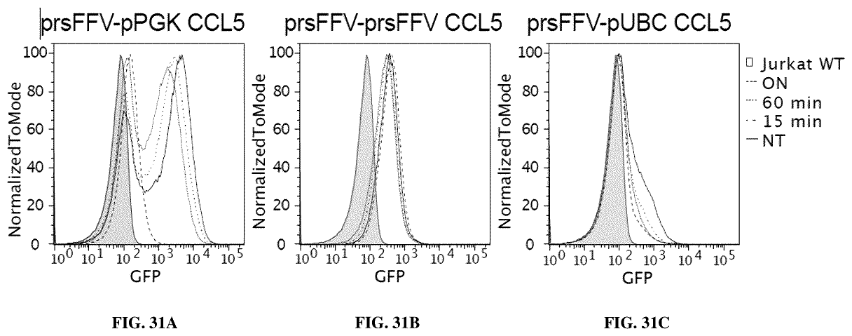

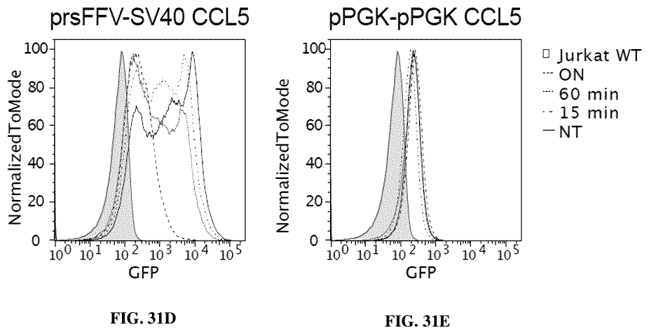

Flow cytometry staining graphs of Jurkat cells transduced with different promoter combinations are shown in FIGS. 31 A to E.

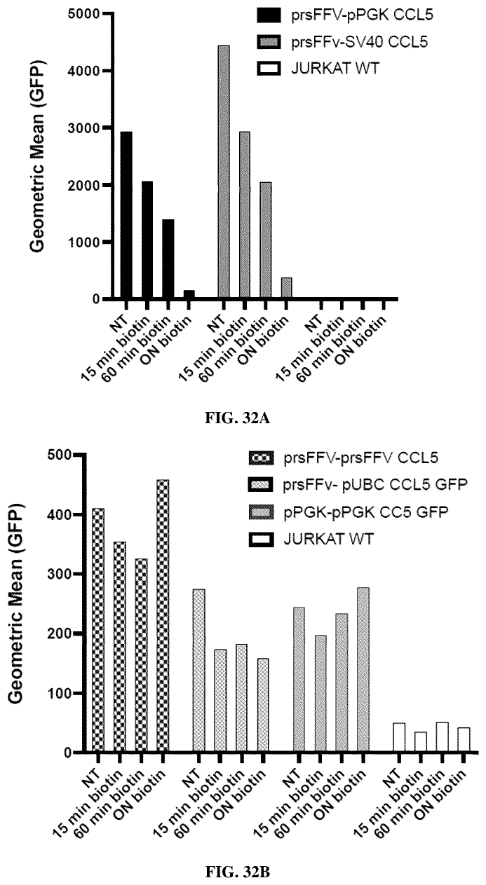

Flow cytometry staining graphs of Jurkat cells transduced with different promoter combinations were also represented in histograms with GFP expression as geometric mean in FIGS. 32 A and B.

Flow cytometry was used to evaluate GFP expression that should be proportional to the amount of intracellular cytokine. Without biotin treatment (NT), the strong-weaker promoter combinations sFFv-PGK (FIG. 31 A) and sFFv-SV40 (FIG. 31 D) shown the highest intracellular GFP of the cytokine, and, upon addition of biotin, it decreases significantly as the cytokine is secreted (FIG. 32 A).

When using a strong-weaker promoter combination sFFv-UbC (FIG. 31 C), the total expression of cytokine was impaired; however, we could still observe a decrease in the intensity of intracellular GFP upon addition of biotin (FIG. 31 C and FIG. 32 B).

For the strong-strong promoter combination sFFv-sFFv (FIG. 31 B), the absence or presence of biotin does not impact the amount of secreted biotin, which is constantly secreted in the cell medium (FIG. 32 B). Similar results were obtained for the weaker-weaker promoter PGK-PGK (FIG. 31 E and FIG. 32 B).

These results demonstrate that the strong-strong promoter combination sFFv-sFFv is not efficient to retain the cytokine in the absence of biotin, leading to high leakage. The weaker-weaker promoter combination PGK-PGK is also not efficient in the retention/release using the RUSH system.

Altogether, these results demonstrate that the RUSH system comprising a double promoter according to the invention outperforms the IVS-IRES RUSH system previously described in WO2010142785 and allows expression and ultimately, use of this RUSH system in primary cells.

These results also show that the double promoter combination to be used in the RUSH system according to the invention is not trivial, but requires the hook protein (e.g., a streptavidin fused to a cellular compartment-retention peptide) be under the control of a strong promoter (e.g., but without limitation, sFFv) and that the protein of interest fused to a hook protein-binding domain (e.g., a cytokine or else fused to a streptavidin binding peptide) be under the control of a weaker promoter (e.g., but without limitation, PGK, UbC or SV40).

1-18. (canceled)

19. A polynucleotide comprising a gene encoding a hook protein and a gene encoding a protein of interest, said protein of interest being either a secretory protein or a cell membrane-anchored protein, wherein:
said gene encoding the hook protein is under the control of a first transcription-activating signal,
said gene encoding the protein of interest is under the control of a second transcription- activating signal, said second transcription-activating signal allowing a lower rate or frequency of transcription initiation than the first transcription-activating signal,
said hook protein is fused to a cellular compartment-retention peptide, and
said protein of interest is fused to a hook protein-binding domain;
wherein the hook protein is one of a pair of proteins comprising the hook protein and the hook protein-binding domain, wherein the hook protein has specific binding affinity for the hook protein-binding domain;
preferably wherein the hook protein is a biotin-binding protein.

20. The polynucleotide according to claim 19, wherein the first transcription-activating signal is a selected from the group comprising SFFV, CMV, CAG, EF1, EF1A, GAL1, GAL10, GPD, ADH and GAP promoter.

21. The polynucleotide according to claim 19, wherein the second transcription-activating signal is selected from the group comprising PGK, SV40, UbC, vav, thymidine kinase promoter (TK), and MSCV promoter.

22. The polynucleotide according to claim 19, wherein the first transcription-activating signal is a SFFV promoter, and the second transcription-activating signal is selected from the group comprising PGK, SV40, and UbC promoter.

23. The polynucleotide according to claim 19, wherein the cellular compartment-retention peptide is a peptide or peptidic domain derived from a transmembrane domain of a protein anchored in the membrane of the cellular compartment or cell membrane, or of a cellular compartment-resident protein.

24. The polynucleotide according to claim 19, wherein the cellular compartment-retention peptide is selected from the group comprising or consisting of endoplasmic reticulum-retention peptides, Golgi-retention peptides, mitochondrion-retention peptides, nucleus-retention peptides, vesicle-retention peptides and plasma membrane-retention peptides.

25. The polynucleotide according to claim 19, wherein the cellular compartment-retention peptide is an endoplasmic reticulum-retention peptide;
preferably, the endoplasmic reticulum-retention peptide comprises:
an amino acid sequence selected from SEQ ID NOs: 10 to 38,
a RR, RXR, DXE, DIE, or SKK peptidic motif, wherein X is any amino acid residue, or
the endoplasmic reticulum-retention peptide of the isoform p33 of the invariant chain, of ribophorin I, of ribophorin II, of a SEC61 subunit, or of cytochrome b5;
more preferably the endoplasmic reticulum-retention peptide comprises a KDEL (SEQ ID NO: 10), K(X)KXX (SEQ ID NO: 17), RR, RXR, or RXXR (SEQ ID NO: 19) peptidic motif, wherein X is any amino acid residue.

26. The polynucleotide according to claim 19, wherein said hook protein is a natural or synthetic biotin-binding protein belonging to the avidin-like superfamily; preferably selected from the group comprising avidin, streptavidin, tamavidin, bradavidin, rhizavidin, neutravidin, extravidin, captavidin, and traptavidin; more preferably said hook protein is streptavidin.

27. The polynucleotide according to claim 19, wherein said hook protein-binding domain is a biotin-binding protein-binding protein or peptide;
preferably, said hook protein-binding domain comprises an amino acid sequence selected from the group consisting of SEQ ID NOs: 605 to 634, DVE, VEA and EAW.

28. The polynucleotide according to claim 19, wherein said protein of interest is a cytokine; preferably said protein of interest is a cytokine selected from the group comprising or consisting of interleukin-12 (IL-12) and interleukin-2 (IL-2).

29. A vector comprising the polynucleotide according to claim 19.

30. A system of at least two polynucleotides, comprising:
a) a first polynucleotide comprising a gene encoding a hook protein, and
b) a second polynucleotide comprising a gene encoding a protein of interest, said protein of interest being either a secretory protein or a cell membrane-anchored protein,
wherein:
said gene encoding the hook protein is under the control of a first transcription-activating signal,
said gene encoding the protein of interest is under the control of a second transcription-activating signal, said second transcription-activating signal allowing a lower rate or frequency of transcription initiation than the first transcription-activating signal,
said hook protein is fused to a cellular compartment-retention peptide, and
said protein of interest is fused to a hook protein-binding domain;
wherein the hook protein is one of a pair of proteins comprising the hook protein and the hook protein-binding domain, wherein the hook protein has specific binding affinity for the hook protein-binding domain;
preferably wherein the hook protein is a biotin-binding protein.

31. A cell comprising the polynucleotide according to claim 19.

32. A composition comprising the polynucleotide according to claim 19.

33. A method of modulating the secretion or cell membrane-anchorage of a protein of interest, comprising the steps of:
(a) transducing a cell with the polynucleotide according to claim 19.
(b) having the transduced cell of step (a) express a hook protein fused to a cellular compartment-retention peptide and the protein of interest fused to a hook protein- binding domain,
wherein the hook protein is one of a pair of proteins comprising the hook protein and the hook protein-binding domain, wherein the hook protein has specific binding affinity for the hook protein-binding domain,
thereby trapping said protein of interest, upon its expression, in said cell to a cellular compartment of the cell, and
(c) contacting said cell with a competing molecule, wherein said competing molecule binds to the hook protein,
thereby releasing said protein of interest from the cellular compartment of the cell and allowing its secretion or cell membrane-anchorage.

34. A method of treatment or prevention of a disease in a subject in need thereof comprising administering to said subject an effective amount of the polynucleotide according to claim 19.

35. A method of treatment or prevention of a disease in a subject in need thereof comprising administering to said subject an effective amount of the polynucleotide according to claim 19, wherein:
(a) in a first step, the polynucleotide according to claim 19 is to be administered to the subject, thereby having a cell of the subject expressing a hook protein fused to a cellular compartment-retention peptide and a protein of interest fused to a hook protein-binding domain; and
(b) in a second step, a competing molecule is to be administered to the subject.

36. The method of modulating the secretion or cell membrane-anchorage of a protein of interest, comprising the steps of:
(a) transducing a cell with the polynucleotide according to claim 19,
(b) having the transduced cell of step (a) express a hook protein fused to a cellular compartment-retention peptide and the protein of interest fused to a hook protein-binding domain, wherein the hook protein is one of a pair of proteins comprising the hook protein and the hook protein-binding domain, wherein the hook protein has specific binding affinity for the hook protein-binding domain,
thereby trapping said protein of interest, upon its expression, in said cell to a cellular compartment of the cell, and
(c) contacting said cell with a competing molecule, wherein said competing molecule binds to the hook protein,
thereby releasing said protein of interest from the cellular compartment of the cell and allowing its secretion or cell membrane-anchorage;
wherein said competing molecule is biotin or a derivative thereof, wherein said biotin derivative preferably has a structure of Formula (I): 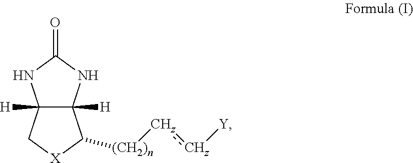
wherein:
X is selected from H₂, O, S, Se, SO, and SO₂,
Y is selected from CONH(CH₂)₄CH(NH₂)COOH, COOH, and OH,
n is 1, 2 or 3, and
z is 1 or 2;
wherein said biotin derivative is more preferably selected from the group consisting of biocytin, dethiobiotin, selenobiotin, biotin sulfoxide, oxybiotin, biotinol, norbiotin, homobiotin, α-dehydrobiotin, and biotin sulfone.

37. A method of treatment or prevention of a disease in a subject in need thereof comprising administering to said subject an effective amount of the polynucleotide according to claim 19, wherein:
(a) in a first step, the polynucleotide is to be administered to the subject, thereby having a cell of the subject expressing a hook protein fused to a cellular compartment-retention peptide and a protein of interest fused to a hook protein- binding domain; and
(b) in a second step, a competing molecule is to be administered to the subject;
wherein said competing molecule is biotin or a derivative thereof, wherein said biotin derivative preferably has a structure of Formula (I): 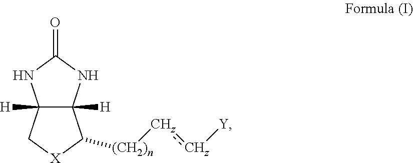
wherein:
X is selected from H₂, O, S, Se, SO, and SO₂,
Y is selected from CONH(CH₂)₄CH(NH₂)COOH, COOH, and OH,
n is 1, 2 or 3, and
z is 1 or 2;
wherein said biotin derivative is more preferably selected from the group consisting of biocytin, dethiobiotin, selenobiotin, biotin sulfoxide, oxybiotin, biotinol, norbiotin, homobiotin, α-dehydrobiotin, and biotin sulfone.


SEQUENCE LISTING (642 sequences)

SEQ ID NO: 1 - protein, 152 aa, Gallus gallus
  Avidin
MVHATSPLLLLLLLSLALVAPGLSARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 2 - protein, 183 aa, Streptomyces avidinii
  Streptavidin
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 3 - protein, 143 aa, Pleurotus cornucopiae
  Tamavidin
MKDVQSLLTGTWYNELGSTMNLTANKDGSLTGTYHSNVGEVPPTYHLSGRYNLQPPSGQGVTLGWAVSFENTSANVHSVSTWSGQYFSEPAEVILTQWLLSRSSEREDLWQSTHVGHDEFSKTKPTKEKIAQAQLLRRGLKFE

SEQ ID NO: 4 - protein, 141 aa, Pleurotus cornucopiae
  Tamavidin
MSDVQSSLTGTWYNELNSKMELTANKDGTLTGKYLSKVGDVYVPYPLSGRYNLQPPAGQGVALGWAVSWENSKIHSATTWSGQFFSESSPVILTQWLLSSSTARGDVWESTLVGNDSFTKTAPTEQQIAHAQLHCRAPRLK

SEQ ID NO: 5 - protein, 163 aa, Bradyrhizobium diazoefficiens
  Bradavidin
MRHFNGMLLAMIASTSLIGPLPAYAQSVNWTWTNQYGSTLAITSFNSNTGAITGTYTNNAANSCDEGKPQGVTGWLAYGNTGTAISFSVNFLGCGSTTVWTGQLNNATGFQGLWYLSLAEAVAWNGISAGADTFTFSSGDKALLTKSGVDLKAGSEKLSNTKK

SEQ ID NO: 6 - protein, 155 aa, Rhizobium etli
  Rhizavidin
MIRTNAVAALVFAVATSALAFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 7 - protein, 2549 aa, Homo sapiens
  mTOR
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 8 - protein, 754 aa, Homo sapiens
  Non-specific serine/threonine protein kinase
MNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 9 - protein, 133 aa, Artificial Sequence
  FKBP-binding domain
VSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTY

SEQ ID NO: 10 - protein, 4 aa, Artificial Sequence
  Endoplasmic reticulum-retention peptide
KDEL

SEQ ID NO: 11 - protein, 4 aa, Artificial Sequence
  Endoplasmic reticulum-retention peptide
KTEL

SEQ ID NO: 12 - protein, 4 aa, Artificial Sequence
  Endoplasmic reticulum-retention peptide
HNEL

SEQ ID NO: 13 - protein, 4 aa, Artificial Sequence
  Endoplasmic reticulum-retention peptide
KEDL

SEQ ID NO: 14 - protein, 4 aa, Artificial Sequence
  Endoplasmic reticulum-retention peptide
HDEL

SEQ ID NO: 15 - protein, 4 aa, Artificial Sequence
  Endoplasmic reticulum-retention peptide
KEEL

SEQ ID NO: 16 - protein, 4 aa, Artificial Sequence
  Endoplasmic reticulum-retention peptide
QEDL

SEQ ID NO: 17 - protein, 5 aa, Artificial Sequence
  Endoplasmic reticulum-retention peptide
  SITE 2: Xaa represents any amino acid residue or is absent
  SITE 4..5: Xaa represents any amino acid residue
KXKXX

SEQ ID NO: 18 - protein, 4 aa, Artificial Sequence
  Endoplasmic reticulum-retention peptide
  SITE 3: Xaa represents any amino acid residue
HKXK

SEQ ID NO: 19 - protein, 4 aa, Artificial Sequence
  Endoplasmic reticulum-retention peptide
  SITE 2..3: Xaa represents any amino acid residue
RXXR

SEQ ID NO: 20 - protein, 7 aa, Artificial Sequence
  Endoplasmic reticulum-retention peptide
  SITE 6: Xaa represents any amino acid residue
EFFDAXE

SEQ ID NO: 21 - protein, 46 aa, Artificial Sequence
  Endoplasmic reticulum-retention peptide
MAGARRGWRLAPVRRGVCGPRARPLMRPLWLLFAVSFFGWTGALDG

SEQ ID NO: 22 - protein, 15 aa, Artificial Sequence
  Endoplasmic reticulum-retention peptide
MGSLWSKISQLFVDA

SEQ ID NO: 23 - protein, 28 aa, Artificial Sequence
  Endoplasmic reticulum-retention peptide
MRVKEKYQHLWRWGWRWGTMLLGMLMIC

SEQ ID NO: 24 - protein, 21 aa, Artificial Sequence
  Endoplasmic reticulum-retention peptide
MRLSWFRVLTVLSICLSAVAT

SEQ ID NO: 25 - protein, 29 aa, Artificial Sequence
  Endoplasmic reticulum-retention peptide
MDPVVVLGLCLSCLLLLSLWKQSYGGGKL

SEQ ID NO: 26 - protein, 35 aa, Artificial Sequence
  Endoplasmic reticulum-retention peptide
HALSYWKPFLVNMCVATVLTAGAYLCYRFLFNSNT

SEQ ID NO: 27 - protein, 44 aa, Artificial Sequence
  Endoplasmic reticulum-retention peptide
SPRGSRPSWGPTDPRRRSRNLGKVIDTLTCGFADLMGYIPLVGA

SEQ ID NO: 28 - protein, 24 aa, Artificial Sequence
  Endoplasmic reticulum-retention peptide
MALWMRFLPLLALLVLWEPKPAQA

SEQ ID NO: 29 - protein, 17 aa, Artificial Sequence
  Endoplasmic reticulum-retention peptide
MEAMWLLCVALAVLAWG

SEQ ID NO: 30 - protein, 27 aa, Artificial Sequence
  Endoplasmic reticulum-retention peptide
IPHDLCHNGEKSKKPSKIKSLFKKKSK

SEQ ID NO: 31 - protein, 69 aa, Artificial Sequence
  Endoplasmic reticulum-retention peptide
RPSNNNTTTTTTTDATQPHHHHHHHRHRDAGVKNVTNNSKTTESSSSSSAAKEKPKHKKGLLHKLKKKL

SEQ ID NO: 32 - protein, 40 aa, Artificial Sequence
  Endoplasmic reticulum-retention peptide
VVQAITFIFKSLGLKCVQFLPQVMPTFLNVIRVCDGAIRE

SEQ ID NO: 33 - protein, 21 aa, Artificial Sequence
  Endoplasmic reticulum-retention peptide
TFCSTALLITALALVCTLLYL

SEQ ID NO: 34 - protein, 18 aa, Artificial Sequence
  Endoplasmic reticulum-retention peptide
GVMLGSIFCALITMLGHI

SEQ ID NO: 35 - protein, 23 aa, Artificial Sequence
  Endoplasmic reticulum-retention peptide
FLAAFVVMLCITIVTIVVYIYLV

SEQ ID NO: 36 - protein, 29 aa, Artificial Sequence
  Endoplasmic reticulum-retention peptide
WGVLAGIAYFSMVGNWAKVLVVLLLFAGV

SEQ ID NO: 37 - protein, 10 aa, Artificial Sequence
  Endoplasmic reticulum-retention peptide
TAPASINTPN

SEQ ID NO: 38 - protein, 22 aa, Artificial Sequence
  Endoplasmic reticulum-retention peptide
WWQLTLGAICALPLAGLLACCA

SEQ ID NO: 39 - protein, 296 aa, Homo sapiens
  p33 isoform of the invariant chain
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 40 - protein, 607 aa, Homo sapiens
  Ribophorin I
MEAPAAGLFLLLLLGTWAPAPGSASSEAPPLINEDVKRTVDLSSHLAKVTAEVVLAHLGGGSTSRATSFLLALEPELEARLAHLGVQVKGEDEEENNLEVRETKIKGKSGRFFTVKLPVALDPGAKISVIVETVYTHVLHPYPTQITQSEKQFVVFEGNHYFYSPYPTKTQTMRVKLASRNVESYTKLGNPTRSEDLLDYGPFRDVPAYSQDTFKVHYENNSPFLTITSMTRVIEVSHWGNIAVEENVDLKHTGAVLKGPFSRYDYQRQPDSGISSIRSFKTILPAAAQDVYYRDEIGNVSTSHLLILDDSVEMEIRPRFPLFGGWKTHYIVGYNLPSYEYLYNLGDQYALKMRFVDHVFDEQVIDSLTVKIILPEGAKNIEIDSPYEISRAPDELHYTYLDTFGRPVIVAYKKNLVEQHIQDIVVHYTFNKVLMLQEPLLVVAAFYILFFTVIIYVRLDFSITKDPAAEARMKVACITEQVLTLVNKRIGLYRHFDETVNRYKQSRDISTLNSGKKSLETEHKALTSEIALLQSRLKTEGSDLCDRVSEMQKLDAQVKELVLKSAVEAERLVAGKLKKDTYIENEKLISGKRQELVTKIDHILDAL

SEQ ID NO: 41 - protein, 23 aa, Artificial Sequence
  Endoplasmic reticulum signal peptide of ribophorin I
MEAPAAGLFLLLLLGTWAPAPGS

SEQ ID NO: 42 - protein, 631 aa, Homo sapiens
  Ribophorin II
MAPPGSSTVFLLALTIIASTWALTPTHYLTKHDVERLKASLDRPFTNLESAFYSIVGLSSLGAQVPDAKKACTYIRSNLDPSNVDSLFYAAQASQALSGCEISISNETKDLLLAAVSEDSSVTQIYHAVAALSGFGLPLASQEALSALTARLSKEETVLATVQALQTASHLSQQADLRSIVEEIEDLVARLDELGGVYLQFEEGLETTALFVAATYKLMDHVGTEPSIKEDQVIQLMNAIFSKKNFESLSEAFSVASAAAVLSHNRYHVPVVVVPEGSASDTHEQAILRLQVTNVLSQPLTQATVKLEHAKSVASRATVLQKTSFTPVGDVFELNFMNVKFSSGYYDFLVEVEGDNRYIANTVELRVKISTEVGITNVDLSTVDKDQSIAPKTTRVTYPAKAKGTFIADSHQNFALFFQLVDVNTGAELTPHQTFVRLHNQKTGQEVVFVAEPDNKNVYKFELDTSERKIEFDSASGTYTLYLIIGDATLKNPILWNVADVVIKFPEEEAPSTVLSQNLFTPKQEIQHLFREPEKRPPTVVSNTFTALILSPLLLLFALWIRIGANVSNFTFAPSTIIFHLGHAAMLGLMYVYWTQLNMFQTLKYLAILGSVTFLAGNRMLAQQAVKRTAH

SEQ ID NO: 43 - protein, 22 aa, Artificial Sequence
  Endoplasmic reticulum signal peptide of ribophorin II
MAPPGSSTVFLLALTIIASTWA

SEQ ID NO: 44 - protein, 476 aa, Homo sapiens
  Sec61 alpha
MAIKFLEVIKPFCVILPEIQKPERKIQFKEKVLWTAITLFIFLVCCQIPLFGIMSSDSADPFYWMRVILASNRGTLMELGISPIVTSGLIMQLLAGAKIIEVGDTPKDRALFNGAQKLFGMIITIGQSIVYVMTGMYGDPSEMGAGICLLITIQLFVAGLIVLLLDELLQKGYGLGSGISLFIATNICETIVWKAFSPTTVNTGRGMEFEGAIIALFHLLATRTDKVRALREAFYRQNLPNLMNLIATIFVFAVVIYFQGFRVDLPIKSARYRGQYNTYPIKLFYTSNIPIILQSALVSNLYVISQMLSARFSGNLLVSLLGTWSDTSSGGPARAYPVGGLCYYLSPPESFGSVLEDPVHAVVYIVFMLGSCAFFSKTWIEVSGSSAKDVAKQLKEQQMVMRGHRETSMVHELNRYIPTAAAFGGLCIGALSVLADFLGAIGSGTGILLAVTIIYQYFEIFVKEQSEVGSMGALLF

SEQ ID NO: 45 - protein, 96 aa, Homo sapiens
  Sec61 beta
MPGPTPSGTNVGSSGRSPSKAVAARAAGSTVRQRKNASCGTRSAGRTTSAGTGGMWRFYTEDSPGLKVGPVPVLVMSLLFIASVFMLHIWGKYTRS

SEQ ID NO: 46 - protein, 68 aa, Homo sapiens
  Sec61 gamma
MDQVMQFVEPSRQFVKDSIRLVKRCTKPDRKEFQKIAMATAIGFAIMGFIGFFVKLIHIPINNIIVGG

SEQ ID NO: 47 - protein, 134 aa, Homo sapiens
  Cytochrome b5
MAEQSDEAVKYYTLEEIQKHNHSKSTWLILHHKVYDLTKFLEEHPGGEEVLREQAGGDATENFEDVGHSTDAREMSKTFIIGELHPDDRPKLNKPPETLITTIDSSSSWWTNWVIPAISAVAVALMYRLYMAED

SEQ ID NO: 48 - protein, 23 aa, Artificial Sequence
  Endoplasmic reticulum transmembrane domain of cytochrome b5
WWTNWVIPAISAVAVALMYRLYM

SEQ ID NO: 49 - protein, 7 aa, Artificial Sequence
  Golgi-retention peptide
  SITE 2..5: Xaa represents any amino acid residue
YXXXXLL

SEQ ID NO: 50 - protein, 5 aa, Artificial Sequence
  Golgi-retention peptide
  SITE 2..3: Xaa represents any amino acid residue
LXXKN

SEQ ID NO: 51 - protein, 4 aa, Artificial Sequence
  Golgi-retention peptide
YKGL

SEQ ID NO: 52 - protein, 4 aa, Artificial Sequence
  Golgi-retention peptide
YAGL

SEQ ID NO: 53 - protein, 4 aa, Artificial Sequence
  Golgi-retention peptide
YKNL

SEQ ID NO: 54 - protein, 4 aa, Artificial Sequence
  Golgi-retention peptide
YEGL

SEQ ID NO: 55 - protein, 5 aa, Artificial Sequence
  Golgi-retention peptide
LLWKN

SEQ ID NO: 56 - protein, 6 aa, Artificial Sequence
  Golgi-retention peptide
QVSAPA

SEQ ID NO: 57 - protein, 10 aa, Artificial Sequence
  Golgi-retention peptide
CPSDSEEDEG

SEQ ID NO: 58 - protein, 14 aa, Artificial Sequence
  Golgi-retention peptide
DDFEDSESTDTEEE

SEQ ID NO: 59 - protein, 34 aa, Artificial Sequence
  Golgi-retention peptide
MAKTAMAYKEKMKELSMLSLICSCFYPEPRNINI

SEQ ID NO: 60 - protein, 27 aa, Artificial Sequence
  Golgi-retention peptide
MASPGSGFWSFGSEDGSGDSENPGTAR

SEQ ID NO: 61 - protein, 30 aa, Artificial Sequence
  Golgi-retention peptide
RLLDSSNATWQRDQPDTHRLSRLDAHVMSM

SEQ ID NO: 62 - protein, 9 aa, Artificial Sequence
  Golgi-retention peptide
  SITE 6..7: Xaa represents any amino acid residue
WEKIMXXFF

SEQ ID NO: 63 - protein, 43 aa, Artificial Sequence
  Golgi-retention peptide
MKFREPLLGGSAAMPGASLQRACRLLVAVCALHLGVTLVYYLA

SEQ ID NO: 64 - protein, 731 aa, Homo sapiens
  Golgin-84
MSWFVDLAGKAEDLLNRVDQGAATALSRKDNASNIYSKNTDYTELHQQNTDLIYQTGPKSTYISSAADNIRNQKATILAGTANVKVGSRTPVEASHPVENASVPRPSSHFVRRKKSEPDDELLFDFLNSSQKEPTGRVEIRKEKGKTPVFQSSQTSSVSSVNPSVTTIKTIEENSFGSQTHEAASNSDSSHEGQEESSKENVSSNAACPDHTPTPNDDGKSHELSNLRLENQLLRNEVQSLNQEMASLLQRSKETQEELNKARARVEKWNADHSKSDRMTRGLRAQVDDLTEAVAAKDSQLAVLKVRLQEADQLLSTRTEALEALQSEKSRIMQDQSEGNSLQNQALQTFQERLHEADATLKREQESYKQMQSEFAARLNKVEMERQNLAEAITLAERKYSDEKKRVDELQQQVKLYKLNLESSKQELIDYKQKATRILQSKEKLINSLKEGSGFEGLDSSTASSMELEELRHEKEMQREEIQKLMGQIHQLRSELQDMEAQQVNEAESAREQLQDLHDQIAGQKASKQELETELERLKQEFHYIEEDLYRTKNTLQSRIKDRDEEIQKLRNQLTNKTLSNSSQSELENRLHQLTETLIQKQTMLESLSTEKNSLVFQLERLEQQMNSASGSSSNGSSINMSGIDNGEGTRLRNVPVLFNDTETNLAGMYGKVRKAASSIDQFSIRLGIFLRRYPIARVFVIIYMALLHLWVMIVLLTYTPEMHHDQPYGK

SEQ ID NO: 65 - protein, 21 aa, Artificial Sequence
  Golgi transmembrane domain of golgin-84
VFVIIYMALLHLWVMIVLLTY

SEQ ID NO: 66 - protein, 3259 aa, Homo sapiens
  Giantin
MLSRLSGLANVVLHELSGDDDTDQNMRAPLDPELHQESDMEFNNTTQEDVQERLAYAEQLVVELKDIIRQKDVQLQQKDEALQEERKAADNKIKKLKLHAKAKLTSLNKYIEEMKAQGGTVLPTEPQSEEQLSKHDKSSTEEEMEIEKIKHKLQEKEELISTLQAQLTQAQAEQPAQSSTEMEEFVMMKQQLQEKEEFISTLQAQLSQTQAEQAAQQVVREKDARFETQVRLHEDELLQLVTQADVETEMQQKLRVLQRKLEEHEESLVGRAQVVDLLQQELTAAEQRNQILSQQLQQMEAEHNTLRNTVETEREESKILLEKMELEVAERKLSFHNLQEEMHHLLEQFEQAGQAQAELESRYSALEQKHKAEMEEKTSHILSLQKTGQELQSACDALKDQNSKLLQDKNEQAVQSAQTIQQLEDQLQQKSKEISQFLNRLPLQQHETASQTSFPDVYNEGTQAVTEENIASLQKRVVELENEKGALLLSSIELEELKAENEKLSSQITLLEAQNRTGEADREVSEISIVDIANKRSSSAEESGQDVLENTFSQKHKELSVLLLEMKEAQEEIAFLKLQLQGKRAEEADHEVLDQKEMKQMEGEGIAPIKMKVFLEDTGQDFPLMPNEESSLPAVEKEQASTEHQSRTSEEISLNDAGVELKSTKQDGDKSLSAVPDIGQCHQDELERLKSQILELELNFHKAQEIYEKNLDEKAKEISNLNQLIEEFKKNADNNSSAFTALSEERDQLLSQVKELSMVTELRAQVKQLEMNLAEAERQRRLDYESQTAHDNLLTEQIHSLSIEAKSKDVKIEVLQNELDDVQLQFSEQSTLIRSLQSQLQNKESEVLEGAERVRHISSKVEELSQALSQKELEITKMDQLLLEKKRDVETLQQTIEEKDQQVTEISFSMTEKMVQLNEEKFSLGVEIKTLKEQLNLLSRAEEAKKEQVEEDNEVSSGLKQNYDEMSPAGQISKEELQHEFDLLKKENEQRKRKLQAALINRKELLQRVSRLEEELANLKDESKKEIPLSETERGEVEEDKENKEYSEKCVTSKCQEIEIYLKQTISEKEVELQHIRKDLEEKLAAEEQFQALVKQMNQTLQDKTNQIDLLQAEISENQAIIQKLITSNTDASDGDSVALVKETVVISPPCTGSSEHWKPELEEKILALEKEKEQLQKKLQEALTSRKAILKKAQEKERHLREELKQQKDDYNRLQEQFDEQSKENENIGDQLRQLQIQVRESIDGKLPSTDQQESCSSTPGLEEPLFKATEQHHTQPVLESNLCPDWPSHSEDASALQGGTSVAQIKAQLKEIEAEKVELELKVSSTTSELTKKSEEVFQLQEQINKQGLEIESLKTVSHEAEVHAESLQQKLESSQLQIAGLEHLRELQPKLDELQKLISKKEEDVSYLSGQLSEKEAALTKIQTEIIEQEDLIKALHTQLEMQAKEHDERIKQLQVELCEMKQKPEEIGEESRAKQQIQRKLQAALISRKEALKENKSLQEELSLARGTIERLTKSLADVESQVSAQNKEKDTVLGRLALLQEERDKLITEMDRSLLENQSLSSSCESLKLALEGLTEDKEKLVKEIESLKSSKIAESTEWQEKHKELQKEYEILLQSYENVSNEAERIQHVVEAVRQEKQELYGKLRSTEANKKETEKQLQEAEQEMEEMKEKMRKFAKSKQQKILELEEENDRLRAEVHPAGDTAKECMETLLSSNASMKEELERVKMEYETLSKKFQSLMSEKDSLSEEVQDLKHQIEGNVSKQANLEATEKHDNQTNVTEEGTQSIPGETEEQDSLSMSTRPTCSESVPSAKSANPAVSKDFSSHDEINNYLQQIDQLKERIAGLEEEKQKNKEFSQTLENEKNTLLSQISTKDGELKMLQEEVTKMNLLNQQIQEELSRVTKLKETAEEEKDDLEERLMNQLAELNGSIGNYCQDVTDAQIKNELLESEMKNLKKCVSELEEEKQQLVKEKTKVESEIRKEYLEKIQGAQKEPGNKSHAKELQELLKEKQQEVKQLQKDCIRYQEKISALERTVKALEFVQTESQKDLEITKENLAQAVEHRKKAQAELASFKVLLDDTQSEAARVLADNLKLKKELQSNKESVKSQMKQKDEDLERRLEQAEEKHLKEKKNMQEKLDALRREKVHLEETIGEIQVTLNKKDKEVQQLQENLDSTVTQLAAFTKSMSSLQDDRDRVIDEAKKWERKFSDAIQSKEEEIRLKEDNCSVLKDQLRQMSIHMEELKINISRLEHDKQIWESKAQTEVQLQQKVCDTLQGENKELLSQLEETRHLYHSSQNELAKLESELKSLKDQLTDLSNSLEKCKEQKGNLEGIIRQQEADIQNSKFSYEQLETDLQASRELTSRLHEEINMKEQKIISLLSGKEEAIQVAIAELRQQHDKEIKELENLLSQEEEENIVLEEENKKAVDKTNQLMETLKTIKKENIQQKAQLDSFVKSMSSLQNDRDRIVGDYQQLEERHLSIILEKDQLIQEAAAENNKLKEEIRGLRSHMDDLNSENAKLDAELIQYREDLNQVITIKDSQQKQLLEVQLQQNKELENKYAKLEEKLKESEEANEDLRRSFNALQEEKQDLSKEIESLKVSISQLTRQVTALQEEGTLGLYHAQLKVKEEEVHRLSALFSSSQKRIAELEEELVCVQKEAAKKVGEIEDKLKKELKHLHHDAGIMRNETETAEERVAELARDLVEMEQKLLMVTKENKGLTAQIQSFGRSMSSLQNSRDHANEELDELKRKYDASLKELAQLKEQGLLNRERDALLSETAFSMNSTEENSLSHLEKLNQQLLSKDEQLLHLSSQLEDSYNQVQSFSKAMASLQNERDHLWNELEKFRKSEEGKQRSAAQPSTSPAEVQSLKKAMSSLQNDRDRLLKELKNLQQQYLQINQEITELHPLKAQLQEYQDKTKAFQIMQEELRQENLSWQHELHQLRMEKSSWEIHERRMKEQYLMAISDKDQQLSHLQNLIRELRSSSSQTQPLKVQYQRQASPETSASPDGSQNLVYETELLRTQLNDSLKEIHQKELRIQQLNSNFSQLLEEKNTLSIQLCDTSQSLRENQQHYGDLLNHCAVLEKQVQELQAGPLNIDVAPGAPQEKNGVHRKSDPEELREPQQSFSEAQQQLCNTRQEVNELRKLLEEERDQRVAAENALSVAEEQIRRLEHSEWDSSRTPIIGSCGTQEQALLIDLTSNSCRRTRSGVGWKRVLRSLCHSRTRVPLLAAIYFLMIHVLLILCFTGHL

SEQ ID NO: 67 - protein, 21 aa, Artificial Sequence
  Golgi transmembrane domain of giantin
VPLLAAIYFLMIHVLLILCFT

SEQ ID NO: 68 - protein, 437 aa, Homo sapiens
  TGN46
MRFVVALVLLNVAAAGAVPLLATESVKQEEAGVRPSAGNVSTHPSLSQRPGGSTKSHPEPQTPKDSPSKSSAEAQTPEDTPNKSGAEAKTQKDSSNKSGAEAKTQKGSTSKSGSEAQTTKDSTSKSHPELQTPKDSTGKSGAEAQTPEDSPNRSGAEAKTQKDSPSKSGSEAQTTKDVPNKSGADGQTPKDGSSKSGAEDQTPKDVPNKSGAEKQTPKDGSNKSGAEEQGPIDGPSKSGAEEQTSKDSPNKVVPEQPSRKDHSKPISNPSDNKELPKADTNQLADKGKLSPHAFKTESGEETDLISPPQEEVKSSEPTEDVEPKEAEDDDTGPEEGSPPKEEKEKMSGSASSENREGTLSDSTGSEKDDLYPNGSGNGSAESSHFFAYLVTAAILVAVLYIAHHNKRKIIAFVLEGKRSKVTRRPKASDYQRLDQKS

SEQ ID NO: 69 - protein, 21 aa, Artificial Sequence
  Golgi transmembrane domain of TGN46
SSHFFAYLVTAAILVAVLYIA

SEQ ID NO: 70 - protein, 32 aa, Artificial Sequence
  Mitochondrion-retention peptide
MDLLPPKPKYNPLRNESLSSLEEGASGSTPPE

SEQ ID NO: 71 - protein, 23 aa, Artificial Sequence
  Mitochondrion-retention peptide
MSFFNISRRFYSQIVKKSVKIKR

SEQ ID NO: 72 - protein, 47 aa, Artificial Sequence
  Mitochondrion-retention peptide
MFAAVARRTATGTLPGAVTLFGRKSFEGYYRDYPLHPRNFGVMASLF

SEQ ID NO: 73 - protein, 22 aa, Artificial Sequence
  Mitochondrion-retention peptide
LVPRYRGLFHHISKLDGGVRFL

SEQ ID NO: 74 - protein, 34 aa, Artificial Sequence
  Mitochondrion-retention peptide
MIFPVARYALRWLRRPEDRAFSRAAMEMALRGVR

SEQ ID NO: 75 - protein, 46 aa, Artificial Sequence
  Mitochondrion-retention peptide
MAGRLLGKALAAVSLSLALASVTIRSSRCRGIQAFRNSFSSSWFHL

SEQ ID NO: 76 - protein, 23 aa, Artificial Sequence
  Mitochondrion-retention peptide
PNNSGFIYQQHGGQQKRARFDHQ

SEQ ID NO: 77 - protein, 12 aa, Artificial Sequence
  Mitochondrion-retention peptide
MKSFITRNKTAI

SEQ ID NO: 78 - protein, 23 aa, Artificial Sequence
  Mitochondrion-retention peptide
MFRMLAKASVTLGSRAASWVRNM

SEQ ID NO: 79 - protein, 61 aa, Artificial Sequence
  Mitochondrion-retention peptide
MFSNLSKRWAQRTLSKSFYSTATGAASKSGKLTQKLVTAGVAAAGITASTLLYADSLTAEA

SEQ ID NO: 80 - protein, 9 aa, Artificial Sequence
  Mitochondrion-retention peptide
MLSSSFERN

SEQ ID NO: 81 - protein, 63 aa, Artificial Sequence
  Mitochondrion-retention peptide
MAFWGWRAAAALRLWGRVVERVEAGGGVGPFQACGCRLVLGGRDDVSAGLRGSHGARGEPLDP

SEQ ID NO: 82 - protein, 17 aa, Artificial Sequence
  Mitochondrion-retention peptide
MRRMPRCGIRLPLLRPS

SEQ ID NO: 83 - protein, 61 aa, Artificial Sequence
  Mitochondrion-retention peptide
MVDRGLGRHLWRLTRRGPPAAADAVAPRPLMGFYEAATQNQADCQLWALLRRGLTTASTLR

SEQ ID NO: 84 - protein, 42 aa, Artificial Sequence
  Mitochondrion-retention peptide
MAFLRSMWGVLSALGRSGAELCTGCGSRLRSPFSFVYLPRWF

SEQ ID NO: 85 - protein, 30 aa, Artificial Sequence
  Mitochondrion-retention peptide
HSLLPALCDSKIRSKALGSDHCPITLYLAL

SEQ ID NO: 86 - protein, 47 aa, Artificial Sequence
  Mitochondrion-retention peptide
AIRILVCEGSGLLAAANDILAARAQRPAARGSTSGGESRLRGERARP

SEQ ID NO: 87 - protein, 40 aa, Artificial Sequence
  Mitochondrion-retention peptide
RGFWANLMQILRDITGVTAICSFLYTKLGIAPFGDPVTMF

SEQ ID NO: 88 - protein, 20 aa, Artificial Sequence
  Mitochondrion-retention peptide
MRTVFSQIPRFKQVNQYIRM

SEQ ID NO: 89 - protein, 11 aa, Artificial Sequence
  Mitochondrion-retention peptide
MAAACRSVKGL

SEQ ID NO: 90 - protein, 46 aa, Artificial Sequence
  Mitochondrion-retention peptide
MISASRAAAARLVGTAASRSPAAARPQDGWNGLSHEAFRFVSRRDY

SEQ ID NO: 91 - protein, 10 aa, Artificial Sequence
  Mitochondrion-retention peptide
RHFWADSKSS

SEQ ID NO: 92 - protein, 20 aa, Artificial Sequence
  Mitochondrion-retention peptide
MTVPYLNSNRNVASYLQSNS

SEQ ID NO: 93 - protein, 53 aa, Artificial Sequence
  Mitochondrion-retention peptide
MYRYLAKALLPSRAGPAALGSAANHSAALLGRGRGQPAAASQPGLALAARRHY

SEQ ID NO: 94 - protein, 37 aa, Artificial Sequence
  Mitochondrion-retention peptide
MFIRNFVNIIGSQKSITKTIARNYFSDNSKLIIPRHG

SEQ ID NO: 95 - protein, 92 aa, Artificial Sequence
  Mitochondrion-retention peptide
MLRFTNCSCKTFVKSSYKLNIRRMNSSFRTETDAFGEIHVPADKYWGAQTQRSFQNFKIGGARERMPLPLVHAFGVLKKSAAIVNESLGGLD

SEQ ID NO: 96 - protein, 15 aa, Artificial Sequence
  Mitochondrion-retention peptide
RHSTRTQHLSVETSY

SEQ ID NO: 97 - protein, 13 aa, Artificial Sequence
  Mitochondrion-retention peptide
VVRVTRTWVPKGL

SEQ ID NO: 98 - protein, 30 aa, Artificial Sequence
  Mitochondrion-retention peptide
MLATRALSLIGKRAISTSVCLRAHGSVVKS

SEQ ID NO: 99 - protein, 13 aa, Artificial Sequence
  Mitochondrion-retention peptide
VRGHPKSRGNFPP

SEQ ID NO: 100 - protein, 39 aa, Artificial Sequence
  Mitochondrion-retention peptide
MLARGLPLRSALVKACPPILSTVGEGWGHHRVGTGEGAG

SEQ ID NO: 101 - protein, 30 aa, Artificial Sequence
  Mitochondrion-retention peptide
FSRSFSTPLPLSTKTLISLSPPHRTFAVRA

SEQ ID NO: 102 - protein, 13 aa, Artificial Sequence
  Mitochondrion-retention peptide
TRTWVPKGLKSPP

SEQ ID NO: 103 - protein, 36 aa, Artificial Sequence
  Mitochondrion-retention peptide
VTHGYNVQRYNQLRKNFGFYRAYSSLRGCKSVFRRI

SEQ ID NO: 104 - protein, 30 aa, Artificial Sequence
  Mitochondrion-retention peptide
MEFIYIYMNFFFFFSVGRKFKKFNKVRVVR

SEQ ID NO: 105 - protein, 35 aa, Artificial Sequence
  Mitochondrion-retention peptide
MVGRNSAIAAGVCGALFIGYCIYFDRKRRSDPNFK

SEQ ID NO: 106 - protein, 29 aa, Artificial Sequence
  Mitochondrion-retention peptide
LLRLIGLTTIFSATALGFLAHKRGLLVRV

SEQ ID NO: 107 - protein, 16 aa, Artificial Sequence
  Mitochondrion-retention peptide
MALLRGVFIVAAKRTP

SEQ ID NO: 108 - protein, 14 aa, Artificial Sequence
  Mitochondrion-retention peptide
MSTPSIVIASARTA

SEQ ID NO: 109 - protein, 22 aa, Artificial Sequence
  Mitochondrion-retention peptide
KTLLSLALVGACITLGAYLGHK

SEQ ID NO: 110 - protein, 29 aa, Artificial Sequence
  Mitochondrion-retention peptide
MKSFITRNKTAILATVAATGTAIGAYYYY

SEQ ID NO: 111 - protein, 12 aa, Artificial Sequence
  Mitochondrion-retention peptide
MLSLRQSIRFFK

SEQ ID NO: 112 - protein, 28 aa, Artificial Sequence
  Mitochondrion-retention peptide
MGRTVVVLGGGISGLAASYHLSRAPCPP

SEQ ID NO: 113 - protein, 80 aa, Artificial Sequence
  Mitochondrion-retention peptide
MLKYKPLLKISKNCEAAILRASKTRLNTIRAYGSTVPKSKSFEQDSRKRTQSWTALRVGAILAATSSVAYLNWHNGQIDN

SEQ ID NO: 114 - protein, 20 aa, Artificial Sequence
  Mitochondrion-retention peptide
CLRRSWNSVTYFQNIGRISL

SEQ ID NO: 115 - protein, 27 aa, Artificial Sequence
  Mitochondrion-retention peptide
MVLPRLYTATSRAAFKAAKQSAPLLST

SEQ ID NO: 116 - protein, 9 aa, Artificial Sequence
  Mitochondrion-retention peptide
MQRSIFARF

SEQ ID NO: 117 - protein, 32 aa, Artificial Sequence
  Mitochondrion-retention peptide
MLSNLRILLNKAALRKAHTSMVRNFRYGKPVQ

SEQ ID NO: 118 - protein, 23 aa, Artificial Sequence
  Mitochondrion-retention peptide
MATSASSHLSKAIKHMYMKLPQG

SEQ ID NO: 119 - protein, 27 aa, Artificial Sequence
  Mitochondrion-retention peptide
VAVHNFLNEGAWNEIVEWERRFGKGLM

SEQ ID NO: 120 - protein, 34 aa, Artificial Sequence
  Mitochondrion-retention peptide
PKDMTPKAALLQVLGRINSKYATEPPFDRHDWYV

SEQ ID NO: 121 - protein, 32 aa, Artificial Sequence
  Mitochondrion-retention peptide
ESRKGQERFNRWFLTGMTVAGVVLLGSLFSRK

SEQ ID NO: 122 - protein, 22 aa, Artificial Sequence
  Mitochondrion-retention peptide
MANLMMRLPISLRSFSVSASSS

SEQ ID NO: 123 - protein, 15 aa, Artificial Sequence
  Mitochondrion-retention peptide
MWRCGGRQGLGVLRR

SEQ ID NO: 124 - protein, 30 aa, Artificial Sequence
  Mitochondrion-retention peptide
MAVFRSGLLVLTTPLASLAPRLASILTSAA

SEQ ID NO: 125 - protein, 10 aa, Artificial Sequence
  Mitochondrion-retention peptide
RALAHGVRVL

SEQ ID NO: 126 - protein, 42 aa, Artificial Sequence
  Mitochondrion-retention peptide
MRSYEFHLARMSGATLCVVTGYRLLTSKWLADRIEDYRQRVI

SEQ ID NO: 127 - protein, 36 aa, Artificial Sequence
  Mitochondrion-retention peptide
RPTGFWENLKRILRDITGITALCGFLYNKLGMAPFG

SEQ ID NO: 128 - protein, 22 aa, Artificial Sequence
  Mitochondrion-retention peptide
TELLVGIATVSGCGAVVYCISK

SEQ ID NO: 129 - protein, 25 aa, Artificial Sequence
  Mitochondrion-retention peptide
RYSLKVLLSYHGWMFAEHGKMSRST

SEQ ID NO: 130 - protein, 29 aa, Artificial Sequence
  Mitochondrion-retention peptide
MSVLTPLLLRGLTGSARRLPVPRAKIHSL

SEQ ID NO: 131 - protein, 5 aa, Artificial Sequence
  Nucleus-retention peptide
TAKRS

SEQ ID NO: 132 - protein, 8 aa, Artificial Sequence
  Nucleus-retention peptide
KKKGSKTS

SEQ ID NO: 133 - protein, 10 aa, Artificial Sequence
  Nucleus-retention peptide
GKISKHWTGI

SEQ ID NO: 134 - protein, 49 aa, Artificial Sequence
  Nucleus-retention peptide
SYNDFGNYNNQSSNFGPMKGGNFGGRSSGPYGGGGQYFAKPRNQGGYGG

SEQ ID NO: 135 - protein, 19 aa, Artificial Sequence
  Nucleus-retention peptide
SNFGPMKGGNFGGRSSGPY

SEQ ID NO: 136 - protein, 7 aa, Artificial Sequence
  Nucleus-retention peptide
PPTKKIR

SEQ ID NO: 137 - protein, 16 aa, Artificial Sequence
  Nucleus-retention peptide
KNQKQGKGKTCKKGKK

SEQ ID NO: 138 - protein, 19 aa, Artificial Sequence
  Nucleus-retention peptide
KRKRGRPGRPPSTNKKPRK

SEQ ID NO: 139 - protein, 4 aa, Artificial Sequence
  Nucleus-retention peptide
RKRR

SEQ ID NO: 140 - protein, 5 aa, Artificial Sequence
  Nucleus-retention peptide
KRGKK

SEQ ID NO: 141 - protein, 5 aa, Artificial Sequence
  Nucleus-retention peptide
RKKRR

SEQ ID NO: 142 - protein, 8 aa, Artificial Sequence
  Nucleus-retention peptide
KKSRKEKK

SEQ ID NO: 143 - protein, 27 aa, Artificial Sequence
  Nucleus-retention peptide
RSGGNHRRNGRGGRGGYNRRNNGYHPY

SEQ ID NO: 144 - protein, 8 aa, Artificial Sequence
  Nucleus-retention peptide
SKKRSLED

SEQ ID NO: 145 - protein, 8 aa, Artificial Sequence
  Nucleus-retention peptide
PPRSKKRI

SEQ ID NO: 146 - protein, 10 aa, Artificial Sequence
  Nucleus-retention peptide
KSAKISKPLH

SEQ ID NO: 147 - protein, 8 aa, Artificial Sequence
  Nucleus-retention peptide
KNKEISMP

SEQ ID NO: 148 - protein, 16 aa, Artificial Sequence
  Nucleus-retention peptide
KKMIEEKKAVLKRKMK

SEQ ID NO: 149 - protein, 19 aa, Artificial Sequence
  Nucleus-retention peptide
NFNHIHKRIRRVADKYLSG

SEQ ID NO: 150 - protein, 9 aa, Artificial Sequence
  Nucleus-retention peptide
CNKRKYSLN

SEQ ID NO: 151 - protein, 5 aa, Artificial Sequence
  Nucleus-retention peptide
GKRKK

SEQ ID NO: 152 - protein, 6 aa, Artificial Sequence
  Nucleus-retention peptide
KKKRER

SEQ ID NO: 153 - protein, 6 aa, Artificial Sequence
  Nucleus-retention peptide
EEKRKR

SEQ ID NO: 154 - protein, 10 aa, Artificial Sequence
  Nucleus-retention peptide
GDRRAAPARP

SEQ ID NO: 155 - protein, 13 aa, Artificial Sequence
  Nucleus-retention peptide
MISEALRKAIGKR

SEQ ID NO: 156 - protein, 10 aa, Artificial Sequence
  Nucleus-retention peptide
MSERKRREKL

SEQ ID NO: 157 - protein, 21 aa, Artificial Sequence
  Nucleus-retention peptide
  SITE 4..6: Xaa represents any amino acid residue
  SITE 11..16: Xaa represents any amino acid residue
GGGXXXKNRRXXXXXXRGGRN

SEQ ID NO: 158 - protein, 25 aa, Artificial Sequence
  Nucleus-retention peptide
GNLDIQRPKRKRKNSRVTFSEDDEI

SEQ ID NO: 159 - protein, 31 aa, Artificial Sequence
  Nucleus-retention peptide
INPEDVDPSVGRFRNMVQTAVVPVKKKRVEG

SEQ ID NO: 160 - protein, 84 aa, Artificial Sequence
  Nucleus-retention peptide
GSGQIQLWQFLLELLSDSANASCITWEGTNGEFKMTDPDEVARRWGERKSKPNMNYDKLSRALRYYYDKNIMTKVHGKRYAYKF

SEQ ID NO: 161 - protein, 19 aa, Artificial Sequence
  Nucleus-retention peptide
  SITE 10..15: Xaa represents any amino acid residue
HRKYEAPRHXXXXXXPRKR

SEQ ID NO: 162 - protein, 20 aa, Artificial Sequence
  Nucleus-retention peptide
HSAELALNVITKKRKGYEDD

SEQ ID NO: 163 - protein, 23 aa, Artificial Sequence
  Nucleus-retention peptide
DYVSKKSKHEEEEWTDDDLVESL

SEQ ID NO: 164 - protein, 5 aa, Artificial Sequence
  Nucleus-retention peptide
KGRKR

SEQ ID NO: 165 - protein, 12 aa, Artificial Sequence
  Nucleus-retention peptide
KGVKRKADTTTP

SEQ ID NO: 166 - protein, 8 aa, Artificial Sequence
  Nucleus-retention peptide
KILKKRHI

SEQ ID NO: 167 - protein, 4 aa, Artificial Sequence
  Nucleus-retention peptide
KKIR

SEQ ID NO: 168 - protein, 4 aa, Artificial Sequence
  Nucleus-retention peptide
KKYR

SEQ ID NO: 169 - protein, 6 aa, Artificial Sequence
  Nucleus-retention peptide
KKKKRK

SEQ ID NO: 170 - protein, 18 aa, Artificial Sequence
  Nucleus-retention peptide
KKQALKEKELGNDAYKKK

SEQ ID NO: 171 - protein, 17 aa, Artificial Sequence
  Nucleus-retention peptide
KKSTALSRELGKIMRRR

SEQ ID NO: 172 - protein, 16 aa, Artificial Sequence
  Nucleus-retention peptide
KKKTVINDLLHYKKEK

SEQ ID NO: 173 - protein, 21 aa, Artificial Sequence
  Nucleus-retention peptide
  SITE 3..17: Xaa represents any amino acid residue
KKXXXXXXXXXXXXXXXKKRK

SEQ ID NO: 174 - protein, 17 aa, Artificial Sequence
  Nucleus-retention peptide
KNLEAIVSVIAERNKKK

SEQ ID NO: 175 - protein, 20 aa, Artificial Sequence
  Nucleus-retention peptide
  SITE 4..14: Xaa represents any amino acid residue
KRKXXXXXXXXXXXKKKSKK

SEQ ID NO: 176 - protein, 34 aa, Artificial Sequence
  Nucleus-retention peptide
  SITE 4..25: Xaa represents any amino acid residue
KRKXXXXXXXXXXXXXXXXXXXXXXKELQKQITK

SEQ ID NO: 177 - protein, 13 aa, Artificial Sequence
  Nucleus-retention peptide
KRQQLKENNKLRR

SEQ ID NO: 178 - protein, 18 aa, Artificial Sequence
  Nucleus-retention peptide
KRVAKRKLIEQNRERRRK

SEQ ID NO: 179 - protein, 15 aa, Artificial Sequence
  Nucleus-retention peptide
  SITE 3..11: Xaa represents any amino acid residue
KRXXXXXXXXXKTKK

SEQ ID NO: 180 - protein, 6 aa, Artificial Sequence
  Nucleus-retention peptide
KSKKQK

SEQ ID NO: 181 - protein, 7 aa, Artificial Sequence
  Nucleus-retention peptide
KSRKRKL

SEQ ID NO: 182 - protein, 15 aa, Artificial Sequence
  Nucleus-retention peptide
KTLETVPLERKKREK

SEQ ID NO: 183 - protein, 8 aa, Artificial Sequence
  Nucleus-retention peptide
KVLKKRRL

SEQ ID NO: 184 - protein, 24 aa, Artificial Sequence
  Nucleus-retention peptide
  SITE 2..3: Xaa represents any amino acid residue
  SITE 5..20: Xaa represents any amino acid residue
  SITE 22..23: Xaa represents any amino acid residue
KXXKXXXXXXXXXXXXXXXXKXXK

SEQ ID NO: 185 - protein, 88 aa, Artificial Sequence
  Nucleus-retention peptide
  SITE 2..31: Xaa represents any amino acid residue
  SITE 33..87: Xaa represents any amino acid residue
LXXXXXXXXXXXXXXXXXXXXXXXXXXXXXXRXXXXXXXXXXXXXXXXXXXXXXXXXXXXXXXXXXXXXXXXXXXXXXXXXXXXXXXR

SEQ ID NO: 186 - protein, 12 aa, Artificial Sequence
  Nucleus-retention peptide
NKIPIKDLLNPQ

SEQ ID NO: 187 - protein, 54 aa, Artificial Sequence
  Nucleus-retention peptide
MEQRGQNAPAASGARKRHGPGPREARGARPGPRVPKTLVLVVAAVLLLVSAESA

SEQ ID NO: 188 - protein, 63 aa, Artificial Sequence
  Nucleus-retention peptide
MGTLYARHVNAGSTGPIKSTIRIYFKPKHVKAWIPRPPRLCQYEKAKNVNFQPSGVTTTRQSI

SEQ ID NO: 189 - protein, 22 aa, Artificial Sequence
  Nucleus-retention peptide
CNGCRNMLLYPRGATNVCCALC

SEQ ID NO: 190 - protein, 22 aa, Artificial Sequence
  Nucleus-retention peptide
CANCRTTLMYPYGAPSVKCAVC

SEQ ID NO: 191 - protein, 22 aa, Artificial Sequence
  Nucleus-retention peptide
CGGCRTLLMYTRGATSVRCSCC

SEQ ID NO: 192 - protein, 10 aa, Artificial Sequence
  Nucleus-retention peptide
MVQLRPRASR

SEQ ID NO: 193 - protein, 16 aa, Artificial Sequence
  Nucleus-retention peptide
KRFKRRWMVRKMKTKK

SEQ ID NO: 194 - protein, 25 aa, Artificial Sequence
  Nucleus-retention peptide
RRDKTLLKEKRKRREELFIEQKKRK

SEQ ID NO: 195 - protein, 7 aa, Artificial Sequence
  Nucleus-retention peptide
NYKKPKL

SEQ ID NO: 196 - protein, 8 aa, Artificial Sequence
  Nucleus-retention peptide
PDGKRKRK

SEQ ID NO: 197 - protein, 11 aa, Artificial Sequence
  Nucleus-retention peptide
PEEVKKRKKAV

SEQ ID NO: 198 - protein, 9 aa, Artificial Sequence
  Nucleus-retention peptide
PKKKSRKSS

SEQ ID NO: 199 - protein, 13 aa, Artificial Sequence
  Nucleus-retention peptide
PKRRTARKQLPKR

SEQ ID NO: 200 - protein, 7 aa, Artificial Sequence
  Nucleus-retention peptide
PLKRKKL

SEQ ID NO: 201 - protein, 5 aa, Artificial Sequence
  Nucleus-retention peptide
  SITE 3..4: Xaa represents any amino acid residue
PPXXR

SEQ ID NO: 202 - protein, 4 aa, Artificial Sequence
  Nucleus-retention peptide
PRPR

SEQ ID NO: 203 - protein, 10 aa, Artificial Sequence
  Nucleus-retention peptide
REKKKFLKRR

SEQ ID NO: 204 - protein, 14 aa, Artificial Sequence
  Nucleus-retention peptide
REKKRQFEMKRKLH

SEQ ID NO: 205 - protein, 15 aa, Artificial Sequence
  Nucleus-retention peptide
RESGKKRKRKRLKPT

SEQ ID NO: 206 - protein, 14 aa, Artificial Sequence
  Nucleus-retention peptide
RKCSQTQCPRKVIK

SEQ ID NO: 207 - protein, 23 aa, Artificial Sequence
  Nucleus-retention peptide
RKRKLPCDTPGQGLTCSGEKRRR

SEQ ID NO: 208 - protein, 8 aa, Artificial Sequence
  Nucleus-retention peptide
RKRPVRRR

SEQ ID NO: 209 - protein, 26 aa, Artificial Sequence
  Nucleus-retention peptide
  SITE 3..12: Xaa represents any amino acid residue
  SITE 15..24: Xaa represents any amino acid residue
RKXXXXXXXXXXRKXXXXXXXXXXKK

SEQ ID NO: 210 - protein, 32 aa, Artificial Sequence
  Nucleus-retention peptide
  SITE 10..28: Xaa represents any amino acid residue
RLKKLKCSKXXXXXXXXXXXXXXXXXXXKTKR

SEQ ID NO: 211 - protein, 24 aa, Artificial Sequence
  Nucleus-retention peptide
RLRRDAGGRGGVYEHLGGAPRRRK

SEQ ID NO: 212 - protein, 18 aa, Artificial Sequence
  Nucleus-retention peptide
GKGRPRRGFKTRRTQKSS

SEQ ID NO: 213 - protein, 15 aa, Artificial Sequence
  Nucleus-retention peptide
  SITE 5..8: Xaa represents any amino acid residue
RRERXXXXRPRKIPR

SEQ ID NO: 214 - protein, 6 aa, Artificial Sequence
  Nucleus-retention peptide
RRGLKR

SEQ ID NO: 215 - protein, 6 aa, Artificial Sequence
  Nucleus-retention peptide
RRKRQR

SEQ ID NO: 216 - protein, 5 aa, Artificial Sequence
  Nucleus-retention peptide
RRKRR

SEQ ID NO: 217 - protein, 5 aa, Artificial Sequence
  Nucleus-retention peptide
RRRGL

SEQ ID NO: 218 - protein, 4 aa, Artificial Sequence
  Nucleus-retention peptide
RRRR

SEQ ID NO: 219 - protein, 30 aa, Artificial Sequence
  Nucleus-retention peptide
RSRARSASLGTTTQGWDPPPLRRPSRARRR

SEQ ID NO: 220 - protein, 24 aa, Artificial Sequence
  Nucleus-retention peptide
RGGRKRPLRPPLVSLARTPLCRRR

SEQ ID NO: 221 - protein, 19 aa, Artificial Sequence
  Nucleus-retention peptide
RVTIRTVRVRRPPKGKHRK

SEQ ID NO: 222 - protein, 8 aa, Artificial Sequence
  Nucleus-retention peptide
  SITE 4: Xaa represents any amino acid residue
STPXKRLI

SEQ ID NO: 223 - protein, 57 aa, Artificial Sequence
  Nucleus-retention peptide
SGVVLPSTGVVKSSGGGGGGDSDHSDLEASVVKEAESSRVVDPEKRPRKRGRKPANG

SEQ ID NO: 224 - protein, 36 aa, Artificial Sequence
  Nucleus-retention peptide
DVKIIGREAMIRVQSSKNNHPAARVMGALKDLDLEL

SEQ ID NO: 225 - protein, 55 aa, Artificial Sequence
  Nucleus-retention peptide
GREEPLNHVEAERQRREKLNQRFYALRAVVPNVSKMDKASLLGDAISYINELKAK

SEQ ID NO: 226 - protein, 39 aa, Artificial Sequence
  Nucleus-retention peptide
SHAAGSKQHESIPGKAKKPKVKKKEKGKKEKGKKKEAPH

SEQ ID NO: 227 - protein, 11 aa, Artificial Sequence
  Nucleus-retention peptide
SKLCLGWLWGM

SEQ ID NO: 228 - protein, 6 aa, Artificial Sequence
  Nucleus-retention peptide
TKKQKT

SEQ ID NO: 229 - protein, 19 aa, Artificial Sequence
  Nucleus-retention peptide
VRILESWFAKNIENPYLDT

SEQ ID NO: 230 - protein, 65 aa, Artificial Sequence
  Nucleus-retention peptide
VRVNVKREYDHMNGSRESPVDCSVSKCNKLVGGGEANPMNYNSYMDEKNGPPPPNMTTNERRVIV

SEQ ID NO: 231 - protein, 52 aa, Artificial Sequence
  Nucleus-retention peptide
YHYGLLTCESCKGFFKRTVQNQKRYTCIENQNCQIDKTQRKRCPYCRFKKCI

SEQ ID NO: 232 - protein, 36 aa, Artificial Sequence
  Nucleus-retention peptide
DVGMKLEAVRADRMRGGRNKFGPMYKRDRALKQQKK

SEQ ID NO: 233 - protein, 30 aa, Artificial Sequence
  Nucleus-retention peptide
YNDFGNYNNQSSNFGPMKGGNFGGRSSGPY

SEQ ID NO: 234 - protein, 16 aa, Artificial Sequence
  Nucleus-retention peptide
LRLTLLELVRRLNGNG

SEQ ID NO: 235 - protein, 35 aa, Artificial Sequence
  Nucleus-retention peptide
RRRKVEAGRTKLAHFRQRKTKGDSSHSEKKTAKRK

SEQ ID NO: 236 - protein, 13 aa, Artificial Sequence
  Nucleus-retention peptide
RRRHIVRKRTLRR

SEQ ID NO: 237 - protein, 14 aa, Artificial Sequence
  Nucleus-retention peptide
KRRQQKIRKYTMRR

SEQ ID NO: 238 - protein, 15 aa, Artificial Sequence
  Nucleus-retention peptide
RRWKEELDEELQRKR

SEQ ID NO: 239 - protein, 7 aa, Artificial Sequence
  Nucleus-retention peptide
KKLKKKK

SEQ ID NO: 240 - protein, 18 aa, Artificial Sequence
  Nucleus-retention peptide
KKYIDGQKKKCGEERRRV

SEQ ID NO: 241 - protein, 5 aa, Artificial Sequence
  Nucleus-retention peptide
RKRKL

SEQ ID NO: 242 - protein, 4 aa, Artificial Sequence
  Nucleus-retention peptide
KRRR

SEQ ID NO: 243 - protein, 17 aa, Artificial Sequence
  Nucleus-retention peptide
KRSAEGGNPPKPLKKLR

SEQ ID NO: 244 - protein, 7 aa, Artificial Sequence
  Nucleus-retention peptide
PKKLRPP

SEQ ID NO: 245 - protein, 47 aa, Artificial Sequence
  Nucleus-retention peptide
  SITE 3..28: Xaa represents any amino acid residue
RRXXXXXXXXXXXXXXXXXXXXXXXXXXRKGFYKRKQCKPSRGRKRG

SEQ ID NO: 246 - protein, 21 aa, Artificial Sequence
  Nucleus-retention peptide
EGKVTKRKHDNEGSGSKRPKV

SEQ ID NO: 247 - protein, 31 aa, Artificial Sequence
  Nucleus-retention peptide
  SITE 2..12: Xaa represents any amino acid residue
RXXXXXXXXXXXHRGFYRKRQCRSSQGQRRG

SEQ ID NO: 248 - protein, 11 aa, Artificial Sequence
  Nucleus-retention peptide
PEKKKEKQEKK

SEQ ID NO: 249 - protein, 11 aa, Artificial Sequence
  Nucleus-retention peptide
GRKKRRQRRRA

SEQ ID NO: 250 - protein, 9 aa, Artificial Sequence
  Nucleus-retention peptide
MRKTKLAPT

SEQ ID NO: 251 - protein, 17 aa, Artificial Sequence
  Nucleus-retention peptide
SHSSMAKRAKKESQDFI

SEQ ID NO: 252 - protein, 43 aa, Artificial Sequence
  Nucleus-retention peptide
AVEGKPLSRAAQLRERRKDLHVSGKPSPRYALKKRALEAKKNK

SEQ ID NO: 253 - protein, 19 aa, Artificial Sequence
  Nucleus-retention peptide
SGYGKVSRRGGHQNSYKPY

SEQ ID NO: 254 - protein, 27 aa, Artificial Sequence
  Nucleus-retention peptide
RDRGYDKADREEGKERRHHRREELAPY

SEQ ID NO: 255 - protein, 33 aa, Artificial Sequence
  Nucleus-retention peptide
GEGERPAQNEKRKEKNIKRGGNRFEPYANPTKR

SEQ ID NO: 256 - protein, 27 aa, Artificial Sequence
  Nucleus-retention peptide
FKSSQEEVRSYSDPPLKFMSVQRPGPY

SEQ ID NO: 257 - protein, 20 aa, Artificial Sequence
  Nucleus-retention peptide
  SITE 3..4: Xaa represents any amino acid residue
  SITE 6: Xaa represents any amino acid residue
  SITE 9..10: Xaa represents any amino acid residue
  SITE 13: Xaa represents any amino acid residue
  SITE 16..18: Xaa represents any amino acid residue
RKXXLXVYXXVIXKRXXXKR

SEQ ID NO: 258 - protein, 6 aa, Artificial Sequence
  Nucleus-retention peptide
KKKRKR

SEQ ID NO: 259 - protein, 12 aa, Artificial Sequence
  Nucleus-retention peptide
KRGSRGGKKGRK

SEQ ID NO: 260 - protein, 4 aa, Artificial Sequence
  Nucleus-retention peptide
KKGR

SEQ ID NO: 261 - protein, 7 aa, Artificial Sequence
  Nucleus-retention peptide
KHSQSRR

SEQ ID NO: 262 - protein, 11 aa, Artificial Sequence
  Nucleus-retention peptide
KNRRAKCRQQR

SEQ ID NO: 263 - protein, 6 aa, Artificial Sequence
  Nucleus-retention peptide
KPNKRK

SEQ ID NO: 264 - protein, 16 aa, Artificial Sequence
  Nucleus-retention peptide
KRRAGDLLEDSPKRPK

SEQ ID NO: 265 - protein, 4 aa, Artificial Sequence
  Nucleus-retention peptide
KRRK

SEQ ID NO: 266 - protein, 7 aa, Artificial Sequence
  Nucleus-retention peptide
RARKRLK

SEQ ID NO: 267 - protein, 6 aa, Artificial Sequence
  Nucleus-retention peptide
KRRRER

SEQ ID NO: 268 - protein, 7 aa, Artificial Sequence
  Nucleus-retention peptide
KARKRLK

SEQ ID NO: 269 - protein, 36 aa, Artificial Sequence
  Nucleus-retention peptide
MEEAVTMAPAAVSSAVVGDPMEYNAILRRKLEEDLE

SEQ ID NO: 270 - protein, 31 aa, Artificial Sequence
  Nucleus-retention peptide
MPTEERVRKKESNRESARRSRYRKAAHLKEL

SEQ ID NO: 271 - protein, 20 aa, Artificial Sequence
  Nucleus-retention peptide
KRKGDEVDGVDEVAKKKSKK

SEQ ID NO: 272 - protein, 11 aa, Artificial Sequence
  Nucleus-retention peptide
GKKNQKHKLKM

SEQ ID NO: 273 - protein, 30 aa, Artificial Sequence
  Nucleus-retention peptide
KRKGTTRGMGAKSRKFINMYGFDPTDFSYI

SEQ ID NO: 274 - protein, 14 aa, Artificial Sequence
  Nucleus-retention peptide
KKQRYMEIQETLKK

SEQ ID NO: 275 - protein, 18 aa, Artificial Sequence
  Nucleus-retention peptide
FKRKHKKDISQNKRAVRR

SEQ ID NO: 276 - protein, 15 aa, Artificial Sequence
  Nucleus-retention peptide
  SITE 4..11: Xaa represents any amino acid residue
  SITE 14: Xaa represents any amino acid residue
KRKXXXXXXXXKKXK

SEQ ID NO: 277 - protein, 77 aa, Artificial Sequence
  Nucleus-retention peptide
NVRQLLSGEQGADILMVQEAGSLPSSAVRTSRVIQHGGTPIEEYTWNLGTRSRPNMVYIYYSRLDVGANRVNLAIVS

SEQ ID NO: 278 - protein, 4 aa, Artificial Sequence
  Nucleus-retention peptide
KKRR

SEQ ID NO: 279 - protein, 5 aa, Artificial Sequence
  Nucleus-retention peptide
RKRKR

SEQ ID NO: 280 - protein, 4 aa, Artificial Sequence
  Nucleus-retention peptide
KRPR

SEQ ID NO: 281 - protein, 17 aa, Artificial Sequence
  Nucleus-retention peptide
RKQVSSHIQVLARRKLR

SEQ ID NO: 282 - protein, 9 aa, Artificial Sequence
  Nucleus-retention peptide
KRCFKKGAR

SEQ ID NO: 283 - protein, 14 aa, Artificial Sequence
  Nucleus-retention peptide
KCYRFRVKKNHRHR

SEQ ID NO: 284 - protein, 4 aa, Artificial Sequence
  Nucleus-retention peptide
RPRK

SEQ ID NO: 285 - protein, 10 aa, Artificial Sequence
  Nucleus-retention peptide
KQRHLDKKQR

SEQ ID NO: 286 - protein, 8 aa, Artificial Sequence
  Nucleus-retention peptide
KCLKFYSK

SEQ ID NO: 287 - protein, 6 aa, Artificial Sequence
  Nucleus-retention peptide
KRKKRK

SEQ ID NO: 288 - protein, 17 aa, Artificial Sequence
  Nucleus-retention peptide
  SITE 4..15: Xaa represents any amino acid residue
KRKXXXXXXXXXXXXKR

SEQ ID NO: 289 - protein, 11 aa, Artificial Sequence
  Nucleus-retention peptide
LKCLRYRFKKH

SEQ ID NO: 290 - protein, 8 aa, Artificial Sequence
  Nucleus-retention peptide
KRPRSPSS

SEQ ID NO: 291 - protein, 25 aa, Artificial Sequence
  Nucleus-retention peptide
KRKVLGSSQNSSGSEASETPVKRRK

SEQ ID NO: 292 - protein, 8 aa, Artificial Sequence
  Nucleus-retention peptide
PDRKRLRK

SEQ ID NO: 293 - protein, 12 aa, Artificial Sequence
  Nucleus-retention peptide
PRGSGPRRAAST

SEQ ID NO: 294 - protein, 17 aa, Artificial Sequence
  Nucleus-retention peptide
RRKTTPSYSGQYRTARR

SEQ ID NO: 295 - protein, 28 aa, Artificial Sequence
  Nucleus-retention peptide
ARRPSCSPEQHGGKVARLQPPPTKAQPA

SEQ ID NO: 296 - protein, 15 aa, Artificial Sequence
  Nucleus-retention peptide
RGGRRGRRRGRGRGG

SEQ ID NO: 297 - protein, 9 aa, Artificial Sequence
  Nucleus-retention peptide
PAAKRVKLD

SEQ ID NO: 298 - protein, 10 aa, Artificial Sequence
  Nucleus-retention peptide
RQRRNELKRS

SEQ ID NO: 299 - protein, 37 aa, Artificial Sequence
  Nucleus-retention peptide
MHGDTPTLHEYMLDLQPETTDLYCYEQLSDSSEEEDE

SEQ ID NO: 300 - protein, 12 aa, Artificial Sequence
  Nucleus-retention peptide
MRHKRSAKRTKR

SEQ ID NO: 301 - protein, 6 aa, Artificial Sequence
  Nucleus-retention peptide
RKRRKR

SEQ ID NO: 302 - protein, 20 aa, Artificial Sequence
  Nucleus-retention peptide
IASRRGLVRYSRIGQRGSMH

SEQ ID NO: 303 - protein, 13 aa, Artificial Sequence
  Nucleus-retention peptide
MNKIPIKDLLNPQ

SEQ ID NO: 304 - protein, 16 aa, Artificial Sequence
  Nucleus-retention peptide
VRILESWFAKNIENPY

SEQ ID NO: 305 - protein, 13 aa, Artificial Sequence
  Nucleus-retention peptide
GRRKAAKRKWAAQ

SEQ ID NO: 306 - protein, 6 aa, Artificial Sequence
  Nucleus-retention peptide
KKRKRR

SEQ ID NO: 307 - protein, 8 aa, Artificial Sequence
  Nucleus-retention peptide
RYKMKRAR

SEQ ID NO: 308 - protein, 15 aa, Artificial Sequence
  Nucleus-retention peptide
RGRKSSGFPKSVKSR

SEQ ID NO: 309 - protein, 11 aa, Artificial Sequence
  Nucleus-retention peptide
LRDGRAKLARD

SEQ ID NO: 310 - protein, 8 aa, Artificial Sequence
  Nucleus-retention peptide
KCHKDLPR

SEQ ID NO: 311 - protein, 8 aa, Artificial Sequence
  Nucleus-retention peptide
ARKRKPSP

SEQ ID NO: 312 - protein, 16 aa, Artificial Sequence
  Nucleus-retention peptide
REPADLEMNLTVPVRR

SEQ ID NO: 313 - protein, 17 aa, Artificial Sequence
  Nucleus-retention peptide
RRTQISSDHFPVGVSRR

SEQ ID NO: 314 - protein, 4 aa, Artificial Sequence
  Nucleus-retention peptide
RSKR

SEQ ID NO: 315 - protein, 16 aa, Artificial Sequence
  Nucleus-retention peptide
RRPALRSPPLGTRKRK

SEQ ID NO: 316 - protein, 14 aa, Artificial Sequence
  Nucleus-retention peptide
DIPPRPKRARVNLR

SEQ ID NO: 317 - protein, 5 aa, Artificial Sequence
  Nucleus-retention peptide
PIKKK

SEQ ID NO: 318 - protein, 8 aa, Artificial Sequence
  Nucleus-retention peptide
GSIIRKWN

SEQ ID NO: 319 - protein, 31 aa, Artificial Sequence
  Nucleus-retention peptide
  SITE 4..21: Xaa represents any amino acid residue
  SITE 26..28: Xaa represents any amino acid residue
KRKXXXXXXXXXXXXXXXXXXKRRKXXXKRR

SEQ ID NO: 320 - protein, 14 aa, Artificial Sequence
  Nucleus-retention peptide
FDYTPNSKRQRCGM

SEQ ID NO: 321 - protein, 14 aa, Artificial Sequence
  Nucleus-retention peptide
KKKKKEEEGEGKKK

SEQ ID NO: 322 - protein, 5 aa, Artificial Sequence
  Nucleus-retention peptide
KRPRP

SEQ ID NO: 323 - protein, 8 aa, Artificial Sequence
  Nucleus-retention peptide
RGRRRRQR

SEQ ID NO: 324 - protein, 5 aa, Artificial Sequence
  Nucleus-retention peptide
RKRRR

SEQ ID NO: 325 - protein, 13 aa, Artificial Sequence
  Nucleus-retention peptide
PPRIYPQLPSAPT

SEQ ID NO: 326 - protein, 8 aa, Artificial Sequence
  Nucleus-retention peptide
PRPRKIPR

SEQ ID NO: 327 - protein, 42 aa, Artificial Sequence
  Nucleus-retention peptide
HSHKKKKIRTSPTFRRPKTLRLRRQPKYPRKSAPRRNKLDHY

SEQ ID NO: 328 - protein, 8 aa, Artificial Sequence
  Nucleus-retention peptide
PKKNRLRR

SEQ ID NO: 329 - protein, 6 aa, Artificial Sequence
  Nucleus-retention peptide
KRKRRP

SEQ ID NO: 330 - protein, 18 aa, Artificial Sequence
  Nucleus-retention peptide
  SITE 4..14: Xaa represents any amino acid residue
RRRXXXXXXXXXXXKRRK

SEQ ID NO: 331 - protein, 9 aa, Artificial Sequence
  Nucleus-retention peptide
SKRVAKRKL

SEQ ID NO: 332 - protein, 17 aa, Artificial Sequence
  Nucleus-retention peptide
RRERNKMAAAKCRNRRR

SEQ ID NO: 333 - protein, 17 aa, Artificial Sequence
  Nucleus-retention peptide
KRMRNRIAASKCRKRKL

SEQ ID NO: 334 - protein, 8 aa, Artificial Sequence
  Nucleus-retention peptide
PLLKKIKQ

SEQ ID NO: 335 - protein, 8 aa, Artificial Sequence
  Nucleus-retention peptide
PAKRARRG

SEQ ID NO: 336 - protein, 21 aa, Artificial Sequence
  Nucleus-retention peptide
KVNSRKRRKEVPGPNGATEED

SEQ ID NO: 337 - protein, 13 aa, Artificial Sequence
  Nucleus-retention peptide
LKDVRKRKLGPGH

SEQ ID NO: 338 - protein, 13 aa, Artificial Sequence
  Nucleus-retention peptide
YKRPCKRSFIRFI

SEQ ID NO: 339 - protein, 9 aa, Artificial Sequence
  Nucleus-retention peptide
KKEKKKSKK

SEQ ID NO: 340 - protein, 7 aa, Artificial Sequence
  Nucleus-retention peptide
GKKRSKA

SEQ ID NO: 341 - protein, 23 aa, Artificial Sequence
  Nucleus-retention peptide
RAIKRRPGLDFDDDGEGNSKFLR

SEQ ID NO: 342 - protein, 17 aa, Artificial Sequence
  Nucleus-retention peptide
RKRKKMPASQRSKRRKT

SEQ ID NO: 343 - protein, 24 aa, Artificial Sequence
  Nucleus-retention peptide
KRPACTLKPECVQQLLVCSQEAKK

SEQ ID NO: 344 - protein, 14 aa, Artificial Sequence
  Nucleus-retention peptide
PRGRRQPIPKARQP

SEQ ID NO: 345 - protein, 6 aa, Artificial Sequence
  Nucleus-retention peptide
PRRGPR

SEQ ID NO: 346 - protein, 17 aa, Artificial Sequence
  Nucleus-retention peptide
KKSKKGRQEALERLKKA

SEQ ID NO: 347 - protein, 17 aa, Artificial Sequence
  Nucleus-retention peptide
RKEWLTNFMEDRRQRKL

SEQ ID NO: 348 - protein, 17 aa, Artificial Sequence
  Nucleus-retention peptide
KKQTTLAFKPIKKGKKR

SEQ ID NO: 349 - protein, 17 aa, Artificial Sequence
  Nucleus-retention peptide
RKCLQAGMNLEARKTKK

SEQ ID NO: 350 - protein, 9 aa, Artificial Sequence
  Nucleus-retention peptide
GKKKYKLKH

SEQ ID NO: 351 - protein, 7 aa, Artificial Sequence
  Nucleus-retention peptide
KKKYKLK

SEQ ID NO: 352 - protein, 7 aa, Artificial Sequence
  Nucleus-retention peptide
KSKKKAQ

SEQ ID NO: 353 - protein, 12 aa, Artificial Sequence
  Nucleus-retention peptide
RQARRNRRRRWR

SEQ ID NO: 354 - protein, 18 aa, Artificial Sequence
  Nucleus-retention peptide
KRAAEDDEDDDVDTKKQK

SEQ ID NO: 355 - protein, 7 aa, Artificial Sequence
  Nucleus-retention peptide
RRKGKEK

SEQ ID NO: 356 - protein, 7 aa, Artificial Sequence
  Nucleus-retention peptide
RRSMKRK

SEQ ID NO: 357 - protein, 19 aa, Artificial Sequence
  Nucleus-retention peptide
RKRAFHGDDPFGEGPPDKK

SEQ ID NO: 358 - protein, 42 aa, Artificial Sequence
  Nucleus-retention peptide
MAPSAKATAAKKAVVKGTNGKKALKVRTSATFRLPKTLKLAR

SEQ ID NO: 359 - protein, 7 aa, Artificial Sequence
  Nucleus-retention peptide
KTRKHRG

SEQ ID NO: 360 - protein, 7 aa, Artificial Sequence
  Nucleus-retention peptide
KHRKHPG

SEQ ID NO: 361 - protein, 9 aa, Artificial Sequence
  Nucleus-retention peptide
KKKKRKREK

SEQ ID NO: 362 - protein, 38 aa, Artificial Sequence
  Nucleus-retention peptide
NQSSNFGPMKGGNFGGRSSGPYGGGGQYFAKPRNQGGY

SEQ ID NO: 363 - protein, 15 aa, Artificial Sequence
  Nucleus-retention peptide
YNNQSSNFGPMKGGN

SEQ ID NO: 364 - protein, 13 aa, Artificial Sequence
  Nucleus-retention peptide
MNKIPIKDLLNPG

SEQ ID NO: 365 - protein, 8 aa, Artificial Sequence
  Nucleus-retention peptide
PQSRKKLR

SEQ ID NO: 366 - protein, 29 aa, Artificial Sequence
  Nucleus-retention peptide
  SITE 5..24: Xaa represents any amino acid residue
KRQRXXXXXXXXXXXXXXXXXXXXKKSKK

SEQ ID NO: 367 - protein, 11 aa, Artificial Sequence
  Nucleus-retention peptide
VNEAFETLKRC

SEQ ID NO: 368 - protein, 13 aa, Artificial Sequence
  Nucleus-retention peptide
CKRKTTNADRRKA

SEQ ID NO: 369 - protein, 6 aa, Artificial Sequence
  Nucleus-retention peptide
QRKRQK

SEQ ID NO: 370 - protein, 17 aa, Artificial Sequence
  Nucleus-retention peptide
HRIEEKRKRTYETFKSI

SEQ ID NO: 371 - protein, 8 aa, Artificial Sequence
  Nucleus-retention peptide
PPQKKIKS

SEQ ID NO: 372 - protein, 11 aa, Artificial Sequence
  Nucleus-retention peptide
  SITE 2: Xaa represents any amino acid residue
  SITE 9..10: Xaa represents any amino acid residue
SXGTKRSZXXM

SEQ ID NO: 373 - protein, 8 aa, Artificial Sequence
  Nucleus-retention peptide
  SITE 5..7: Xaa represents any amino acid residue
TKRSXXXM

SEQ ID NO: 374 - protein, 9 aa, Artificial Sequence
  Nucleus-retention peptide
PPRKKRTVV

SEQ ID NO: 375 - protein, 18 aa, Artificial Sequence
  Nucleus-retention peptide
KRKKEMANKSAPEAKKKK

SEQ ID NO: 376 - protein, 16 aa, Artificial Sequence
  Nucleus-retention peptide
  SITE 3..12: Xaa represents any amino acid residue
KRXXXXXXXXXXKKKL

SEQ ID NO: 377 - protein, 16 aa, Artificial Sequence
  Nucleus-retention peptide
KRPAATKKAGQAKKKK

SEQ ID NO: 378 - protein, 7 aa, Artificial Sequence
  Nucleus-retention peptide
PQPKKKP

SEQ ID NO: 379 - protein, 10 aa, Artificial Sequence
  Nucleus-retention peptide
  SITE 3: Xaa represents any amino acid residue
KRXPQPKKKP

SEQ ID NO: 380 - protein, 7 aa, Artificial Sequence
  Nucleus-retention peptide
RIRKKLR

SEQ ID NO: 381 - protein, 7 aa, Artificial Sequence
  Nucleus-retention peptide
LKRKLQR

SEQ ID NO: 382 - protein, 7 aa, Artificial Sequence
  Nucleus-retention peptide
RRMKWKK

SEQ ID NO: 383 - protein, 7 aa, Artificial Sequence
  Nucleus-retention peptide
KVTKNKS

SEQ ID NO: 384 - protein, 8 aa, Artificial Sequence
  Nucleus-retention peptide
KRRGKPGP

SEQ ID NO: 385 - protein, 10 aa, Artificial Sequence
  Nucleus-retention peptide
PYLNKRKGKP

SEQ ID NO: 386 - protein, 7 aa, Artificial Sequence
  Nucleus-retention peptide
PKKARED

SEQ ID NO: 387 - protein, 7 aa, Artificial Sequence
  Nucleus-retention peptide
VSRKRPR

SEQ ID NO: 388 - protein, 11 aa, Artificial Sequence
  Nucleus-retention peptide
APKRKSGVSKC

SEQ ID NO: 389 - protein, 12 aa, Artificial Sequence
  Nucleus-retention peptide
EEDGPQKKKRRL

SEQ ID NO: 390 - protein, 12 aa, Artificial Sequence
  Nucleus-retention peptide
TEKKQGKSILYD

SEQ ID NO: 391 - protein, 11 aa, Artificial Sequence
  Nucleus-retention peptide
REKKEKEQKEK

SEQ ID NO: 392 - protein, 11 aa, Artificial Sequence
  Nucleus-retention peptide
LEKKVKKKFDW

SEQ ID NO: 393 - protein, 28 aa, Artificial Sequence
  Nucleus-retention peptide
YLTQETNKVETYKEQPLKTPGKKKKGKP

SEQ ID NO: 394 - protein, 7 aa, Artificial Sequence
  Nucleus-retention peptide
RVHPYQR

SEQ ID NO: 395 - protein, 18 aa, Artificial Sequence
  Nucleus-retention peptide
SDREDGNYCPPVKRERTS

SEQ ID NO: 396 - protein, 8 aa, Artificial Sequence
  Nucleus-retention peptide
KKKRERLD

SEQ ID NO: 397 - protein, 18 aa, Artificial Sequence
  Nucleus-retention peptide
MPKTRRRPRRSQRKRPPT

SEQ ID NO: 398 - protein, 5 aa, Artificial Sequence
  Nucleus-retention peptide
PRRRK

SEQ ID NO: 399 - protein, 17 aa, Artificial Sequence
  Nucleus-retention peptide
KRPMNAFMVWAQAARRK

SEQ ID NO: 400 - protein, 5 aa, Artificial Sequence
  Nucleus-retention peptide
RPRRK

SEQ ID NO: 401 - protein, 17 aa, Artificial Sequence
  Nucleus-retention peptide
KRPMNAFIVWSRDQRRK

SEQ ID NO: 402 - protein, 8 aa, Artificial Sequence
  Nucleus-retention peptide
APTKRKGS

SEQ ID NO: 403 - protein, 7 aa, Artificial Sequence
  Nucleus-retention peptide
PNKKKRK

SEQ ID NO: 404 - protein, 7 aa, Artificial Sequence
  Nucleus-retention peptide
PKKKRKV

SEQ ID NO: 405 - protein, 11 aa, Artificial Sequence
  Nucleus-retention peptide
SDKKVRSRLIE

SEQ ID NO: 406 - protein, 9 aa, Artificial Sequence
  Nucleus-retention peptide
KKKRRSREK

SEQ ID NO: 407 - protein, 32 aa, Artificial Sequence
  Nucleus-retention peptide
RKRIREDRKATTAQKVQQMKQRLNENERKRKR

SEQ ID NO: 408 - protein, 20 aa, Artificial Sequence
  Nucleus-retention peptide
KDCVINKHHRNRCQYCRLQR

SEQ ID NO: 409 - protein, 8 aa, Artificial Sequence
  Nucleus-retention peptide
GRKRKKRT

SEQ ID NO: 410 - protein, 20 aa, Artificial Sequence
  Nucleus-retention peptide
PKRPRDRHDGELGGRKRARG

SEQ ID NO: 411 - protein, 11 aa, Artificial Sequence
  Nucleus-retention peptide
EYLSRKGKLEL

SEQ ID NO: 412 - protein, 6 aa, Artificial Sequence
  Nucleus-retention peptide
KAKRQR

SEQ ID NO: 413 - protein, 10 aa, Artificial Sequence
  Nucleus-retention peptide
IKYFKKFPKD

SEQ ID NO: 414 - protein, 5 aa, Artificial Sequence
  Nucleus-retention peptide
RKRKK

SEQ ID NO: 415 - protein, 13 aa, Artificial Sequence
  Nucleus-retention peptide
MASQGTKRSYEQM

SEQ ID NO: 416 - protein, 19 aa, Artificial Sequence
  Nucleus-retention peptide
KRGINDRNFWRGENGRKTR

SEQ ID NO: 417 - protein, 14 aa, Artificial Sequence
  Nucleus-retention peptide
RRMLGAPLRQRRVR

SEQ ID NO: 418 - protein, 13 aa, Artificial Sequence
  Nucleus-retention peptide
VRTTKGKRKRIDV

SEQ ID NO: 419 - protein, 11 aa, Artificial Sequence
  Nucleus-retention peptide
RSSRGKRRRIE

SEQ ID NO: 420 - protein, 15 aa, Artificial Sequence
  Nucleus-retention peptide
  SITE 4..12: Xaa represents any amino acid residue
KRKXXXXXXXXXKIR

SEQ ID NO: 421 - protein, 15 aa, Artificial Sequence
  Nucleus-retention peptide
  SITE 5..13: Xaa represents any amino acid residue
KRKRXXXXXXXXXKK

SEQ ID NO: 422 - protein, 19 aa, Artificial Sequence
  Nucleus-retention peptide
  SITE 5..14: Xaa represents any amino acid residue
RLRKXXXXXXXXXXRKFKK

SEQ ID NO: 423 - protein, 48 aa, Artificial Sequence
  Nucleus-retention peptide
RKDRRGGRMLKHKRQRDDGEGRGEVGSAGDMRAANLWPSPLMIKRSKK

SEQ ID NO: 424 - protein, 17 aa, Artificial Sequence
  Nucleus-retention peptide
  SITE 3..13: Xaa represents any amino acid residue
KRXXXXXXXXXXXKRSR

SEQ ID NO: 425 - protein, 28 aa, Artificial Sequence
  Nucleus-retention peptide
  SITE 5..17: Xaa represents any amino acid residue
RQKRXXXXXXXXXXXXXKRRSSPSKDGR

SEQ ID NO: 426 - protein, 17 aa, Artificial Sequence
  Nucleus-retention peptide
  SITE 3..13: Xaa represents any amino acid residue
KRXXXXXXXXXXXKKTR

SEQ ID NO: 427 - protein, 16 aa, Artificial Sequence
  Nucleus-retention peptide
  SITE 4..11: Xaa represents any amino acid residue
KRKXXXXXXXXKKAKV

SEQ ID NO: 428 - protein, 21 aa, Artificial Sequence
  Nucleus-retention peptide
  SITE 6..17: Xaa represents any amino acid residue
RKKRKXXXXXXXXXXXXKKSK

SEQ ID NO: 429 - protein, 20 aa, Artificial Sequence
  Nucleus-retention peptide
  SITE 3..12: Xaa represents any amino acid residue
KKXXXXXXXXXXRKRGRPRK

SEQ ID NO: 430 - protein, 18 aa, Artificial Sequence
  Nucleus-retention peptide
  SITE 3..13: Xaa represents any amino acid residue
KKXXXXXXXXXXXRLRKK

SEQ ID NO: 431 - protein, 21 aa, Artificial Sequence
  Nucleus-retention peptide
  SITE 6..16: Xaa represents any amino acid residue
KKRARXXXXXXXXXXXRQRKK

SEQ ID NO: 432 - protein, 22 aa, Artificial Sequence
  Nucleus-retention peptide
  SITE 4..15: Xaa represents any amino acid residue
KLRXXXXXXXXXXXXRREIQKR

SEQ ID NO: 433 - protein, 18 aa, Artificial Sequence
  Nucleus-retention peptide
  SITE 7..13: Xaa represents any amino acid residue
RAIKTKXXXXXXXKLKSK

SEQ ID NO: 434 - protein, 17 aa, Artificial Sequence
  Nucleus-retention peptide
  SITE 3..12: Xaa represents any amino acid residue
RKXXXXXXXXXXRKLKK

SEQ ID NO: 435 - protein, 9 aa, Artificial Sequence
  Nucleus-retention peptide
GKRQVLIRP

SEQ ID NO: 436 - protein, 9 aa, Artificial Sequence
  Nucleus-retention peptide
GIIKPISKH

SEQ ID NO: 437 - protein, 4 aa, Artificial Sequence
  Nucleus-retention peptide
KKRK

SEQ ID NO: 438 - protein, 5 aa, Artificial Sequence
  Nucleus-retention peptide
KKLKK

SEQ ID NO: 439 - protein, 24 aa, Artificial Sequence
  Nucleus-retention peptide
DNPGKKRKSTDDNEGPSPKRRVIT

SEQ ID NO: 440 - protein, 10 aa, Artificial Sequence
  Nucleus-retention peptide
CSVKRKRDDD

SEQ ID NO: 441 - protein, 31 aa, Artificial Sequence
  Nucleus-retention peptide
SKPFSCGRSGKGHKRKTPFGNTERKTPVKKL

SEQ ID NO: 442 - protein, 17 aa, Artificial Sequence
  Nucleus-retention peptide
KRELELLKHNPKRRKIT

SEQ ID NO: 443 - protein, 4 aa, Artificial Sequence
  Nucleus-retention peptide
RRRK

SEQ ID NO: 444 - protein, 28 aa, Artificial Sequence
  Nucleus-retention peptide
YIFEVKKPEDEVLICIQQRPKRSTRREG

SEQ ID NO: 445 - protein, 40 aa, Artificial Sequence
  Nucleus-retention peptide
MADAFEVHYIADWADISSEYPKLHILTTSGRVLSVLAELS

SEQ ID NO: 446 - protein, 24 aa, Artificial Sequence
  Nucleus-retention peptide
MMPSSVSWGILLAGLCCLVPVSLA

SEQ ID NO: 447 - protein, 30 aa, Artificial Sequence
  Nucleus-retention peptide
MAGPATQSPMKLMALQLLLWHSALWTVQEA

SEQ ID NO: 448 - protein, 46 aa, Artificial Sequence
  Nucleus-retention peptide
MQRVNMIMAESPSLITICLLGYLLSAECTVFLDHENANKILNRPKR

SEQ ID NO: 449 - protein, 21 aa, Artificial Sequence
  Nucleus-retention peptide
MKGSLLLLLVSNLLLCQSVAP

SEQ ID NO: 450 - protein, 24 aa, Artificial Sequence
  Nucleus-retention peptide
MKWVTFISLLFLFSSAYSRGVFRR

SEQ ID NO: 451 - protein, 19 aa, Artificial Sequence
  Nucleus-retention peptide
MWWRLWWLLLLLLLLPMWA

SEQ ID NO: 452 - protein, 32 aa, Artificial Sequence
  Nucleus-retention peptide
MLFNLRILLNNAAFRNGHNFMVRNFRCGQPLQ

SEQ ID NO: 453 - protein, 29 aa, Artificial Sequence
  Nucleus-retention peptide
MSVLTPLLLRGLTGSARRLPVPRAKIHSL

SEQ ID NO: 454 - protein, 29 aa, Artificial Sequence
  Nucleus-retention peptide
MSVLTPLLLRGLTGSARRLPVPRAKIHSL

SEQ ID NO: 455 - protein, 83 aa, Artificial Sequence
  Nucleus-retention peptide
MVTKITLSPQNFRIQKQETTLLKEKSTEKNSLAKSILAVKNHFIELRSKLSERFISHKNTMLSFVDTRTLLLLAVTSCLATCQ

SEQ ID NO: 456 - protein, 32 aa, Artificial Sequence
  Nucleus-retention peptide
MGSSQAPRMGSVGGHGLMALLMAGLILPGILA

SEQ ID NO: 457 - protein, 17 aa, Artificial Sequence
  Nucleus-retention peptide
MAGIFYFLFSFLFGICD

SEQ ID NO: 458 - protein, 22 aa, Artificial Sequence
  Nucleus-retention peptide
MENRLLRVFLVWAALTMDGASA

SEQ ID NO: 459 - protein, 27 aa, Artificial Sequence
  Nucleus-retention peptide
MARQGCFGSYQVISLFTFAIGVNLCLG

SEQ ID NO: 460 - protein, 19 aa, Artificial Sequence
  Nucleus-retention peptide
MSRLPVLLLLQLLVRPGLQ

SEQ ID NO: 461 - protein, 29 aa, Artificial Sequence
  Nucleus-retention peptide
MKQQKRLYARLLTLLFALIFLLPHSSASA

SEQ ID NO: 462 - protein, 22 aa, Artificial Sequence
  Nucleus-retention peptide
MATPLPPPSPRHLRLLRLLLSG

SEQ ID NO: 463 - protein, 18 aa, Artificial Sequence
  Nucleus-retention peptide
MKAPGRLVLIILCSVVFS

SEQ ID NO: 464 - protein, 13 aa, Artificial Sequence
  Nucleus-retention peptide
MLQLWKLLCGVLT

SEQ ID NO: 465 - protein, 13 aa, Artificial Sequence
  Nucleus-retention peptide
MLYLQGWSMPAVA

SEQ ID NO: 466 - protein, 48 aa, Artificial Sequence
  Nucleus-retention peptide
MDNVQPKIKHRPFCFSVKGHVKMLRLDIINSLVTTVFMLIVSVLALIP

SEQ ID NO: 467 - protein, 32 aa, Artificial Sequence
  Nucleus-retention peptide
MPCLDQQLTVHALPCPAQPSSLAFCQVGFLTA

SEQ ID NO: 468 - protein, 46 aa, Artificial Sequence
  Nucleus-retention peptide
MKTLFNPAPAIADLDPQFYTLSDVFCCNESEAEILTGLTVGSAADA

SEQ ID NO: 469 - protein, 19 aa, Artificial Sequence
  Nucleus-retention peptide
MKPLLVVFVFLFLWDPVLA

SEQ ID NO: 470 - protein, 21 aa, Artificial Sequence [sequence omitted]

SEQ ID NO: 471 - protein, 70 aa, Artificial Sequence
  Nucleus-retention peptide
MVLTKPLQRNGSMMSFENVKEKSREGGPHAHTPEEELCFVVTHTPQVQTTLNLFFHIFKVLTQPLSLLWG

SEQ ID NO: 472 - protein, 32 aa, Artificial Sequence
  Nucleus-retention peptide
MATPPFRLIRKMFSFKVSRWMGLACFRSLAAS

SEQ ID NO: 473 - protein, 29 aa, Artificial Sequence
  Nucleus-retention peptide
MSFFQLLMKRKELIPLVVFMTVAAGGASS

SEQ ID NO: 474 - protein, 41 aa, Artificial Sequence
  Nucleus-retention peptide
MVSALRGAPLIRVHSSPVSSPSVSGPAALVSCLSSQSSALS

SEQ ID NO: 475 - protein, 20 aa, Artificial Sequence
  Nucleus-retention peptide
MMGSPVSHLLAGFCVWVVLG

SEQ ID NO: 476 - protein, 23 aa, Artificial Sequence
  Nucleus-retention peptide
MASMAAVLTWALALLSAFSATQA

SEQ ID NO: 477 - protein, 45 aa, Artificial Sequence
  Nucleus-retention peptide
MVLMWTSGDAFKTAYFLLKGAPLQFSVCGLLQVLVDLAILGQATA

SEQ ID NO: 478 - protein, 68 aa, Artificial Sequence
  Nucleus-retention peptide
MDFVAGAIGGVCGVAVGYPLDTVKVRIQTEPLYTGIWHCVRDTYHRERVWGFYRGLSLPVCTVSLVSS

SEQ ID NO: 479 - protein, 49 aa, Artificial Sequence
  Nucleus-retention peptide
MEKPLFPLVPLHWFGFGYTALVVSGGIVGYVKTGSVPSLAAGLLFGSLA

SEQ ID NO: 480 - protein, 15 aa, Artificial Sequence
  Nucleus-retention peptide
MGLLLPLALCILVLC

SEQ ID NO: 481 - protein, 26 aa, Artificial Sequence
  Nucleus-retention peptide
MGIQTSPVLLASLGVGLVTLLGLAVG

SEQ ID NO: 482 - protein, 25 aa, Artificial Sequence
  Nucleus-retention peptide
MSDLLLLGLIGGLTLLLLLTLLAFA

SEQ ID NO: 483 - protein, 29 aa, Artificial Sequence
  Nucleus-retention peptide
METVVIVAIGVLATIFLASFAALVLVCRQ

SEQ ID NO: 484 - protein, 35 aa, Artificial Sequence
  Nucleus-retention peptide
MAGSVECTWGWGHCAPSPLLLWTLLLFAAPFGLLG

SEQ ID NO: 485 - protein, 57 aa, Artificial Sequence
  Nucleus-retention peptide
MMPSRTNLATGIPSSKVKYSRLSSTDDGYIDLQFKKTPPKIPYKAIALATVLFLIGA

SEQ ID NO: 486 - protein, 36 aa, Artificial Sequence
  Nucleus-retention peptide
MALPQMCDGSHLASTLRYCMTVSGTVVLVAGTLCFA

SEQ ID NO: 487 - protein, 21 aa, Artificial Sequence
  Nucleus-retention peptide
MKKWFVAAGIGAGLLMLSSAA

SEQ ID NO: 488 - protein, 21 aa, Artificial Sequence
  Nucleus-retention peptide
MKQSTIALALLPLLFTPVTKA

SEQ ID NO: 489 - protein, 21 aa, Artificial Sequence
  Nucleus-retention peptide
MKKTAIAIAVALAGFATVAQA

SEQ ID NO: 490 - protein, 21 aa, Artificial Sequence
  Nucleus-retention peptide
MKKLMLAIFFSVLSFPSFSQS

SEQ ID NO: 491 - protein, 23 aa, Artificial Sequence
  Nucleus-retention peptide
MKKNIAFLLASMFVFSIATNAYA

SEQ ID NO: 492 - protein, 32 aa, Artificial Sequence
  Nucleus-retention peptide
MFAKRFKTSLLPLFAGFLLLFHLVLAGPAAAS

SEQ ID NO: 493 - protein, 19 aa, Artificial Sequence
  Nucleus-retention peptide
MRFPSIFTAVLFAASSALA

SEQ ID NO: 494 - protein, 19 aa, Artificial Sequence
  Nucleus-retention peptide
MRFPSIFTTVLFAASSALA

SEQ ID NO: 495 - protein, 19 aa, Artificial Sequence
  Nucleus-retention peptide
MRFPSIFTSVLFAASSALA

SEQ ID NO: 496 - protein, 19 aa, Artificial Sequence
  Nucleus-retention peptide
MRFPSIFTHVLFAASSALA

SEQ ID NO: 497 - protein, 19 aa, Artificial Sequence
  Nucleus-retention peptide
MRFPSIFTIVLFAASSALA

SEQ ID NO: 498 - protein, 19 aa, Artificial Sequence
  Nucleus-retention peptide
MRFPSIFTFVLFAASSALA

SEQ ID NO: 499 - protein, 19 aa, Artificial Sequence
  Nucleus-retention peptide
MRFPSIFTEVLFAASSALA

SEQ ID NO: 500 - protein, 19 aa, Artificial Sequence
  Nucleus-retention peptide
MRFPSIFTGVLFAASSALA

SEQ ID NO: 501 - protein, 21 aa, Artificial Sequence
  Nucleus-retention peptide
MRSSPLLRSAVVAALPVLALA

SEQ ID NO: 502 - protein, 22 aa, Artificial Sequence
  Nucleus-retention peptide
MGAAAVRWHLCVLLALGTRGRL

SEQ ID NO: 503 - protein, 17 aa, Artificial Sequence
  Nucleus-retention peptide
MRSSLVLFFVSAWTALA

SEQ ID NO: 504 - protein, 32 aa, Artificial Sequence
  Nucleus-retention peptide
MLRGPGPGRLLLLAVLCLGTSVRCTETGKSKR

SEQ ID NO: 505 - protein, 26 aa, Artificial Sequence
  Nucleus-retention peptide
MLRGPGPGLLLLAVQCLGTAVPSTGA

SEQ ID NO: 506 - protein, 31 aa, Artificial Sequence
  Nucleus-retention peptide
MRRGALTGLLLVLCLSVVLRAAPSATSKKRR

SEQ ID NO: 507 - protein, 10 aa, Artificial Sequence
  Lysosome-retention peptide
DSWGILFSHP

SEQ ID NO: 508 - protein, 8 aa, Artificial Sequence
  Lysosome-retention peptide
  SITE 3..5: Xaa represents any amino acid residue
DEXXXLLI

SEQ ID NO: 509 - protein, 6 aa, Artificial Sequence
  Lysosome-retention peptide
  SITE 2..4: Xaa represents any amino acid residue
EXXXLL

SEQ ID NO: 510 - protein, 13 aa, Artificial Sequence
  Lysosome-retention peptide
  SITE 4..11: Xaa represents any amino acid residue
EEEXXXXXXXXLI

SEQ ID NO: 511 - protein, 4 aa, Artificial Sequence
  Lysosome-retention peptide
YTPL

SEQ ID NO: 512 - protein, 6 aa, Artificial Sequence
  Lysosome-retention peptide
NQPLLT

SEQ ID NO: 513 - protein, 34 aa, Artificial Sequence
  Lysosome-retention peptide
RRPGQRRPPPPPPPPPLPPPPPPPPLPPLPLPPL

SEQ ID NO: 514 - protein, 30 aa, Artificial Sequence
  Lysosome-retention peptide
  SITE 22..23: Xaa represents any amino acid residue
RGQGSMDEGTADERAPLIRTYXXVIFLWCM

SEQ ID NO: 515 - protein, 6 aa, Artificial Sequence
  Lysosome-retention peptide
EKITLL

SEQ ID NO: 516 - protein, 9 aa, Artificial Sequence
  Lysosome-retention peptide
SSLKILSKG

SEQ ID NO: 517 - protein, 23 aa, Artificial Sequence
  Lysosome-retention peptide
PNQALLRILKETEFKKIKVLGSG

SEQ ID NO: 518 - protein, 5 aa, Artificial Sequence
  Lysosome-retention peptide
YLLNT

SEQ ID NO: 519 - protein, 4 aa, Artificial Sequence
  Lysosome-retention peptide
YGSI

SEQ ID NO: 520 - protein, 5 aa, Artificial Sequence
  Lysosome-retention peptide
GYDQL

SEQ ID NO: 521 - protein, 5 aa, Artificial Sequence
  Lysosome-retention peptide
YFPQA

SEQ ID NO: 522 - protein, 7 aa, Artificial Sequence
  Lysosome-retention peptide
LLDLLDE

SEQ ID NO: 523 - protein, 22 aa, Artificial Sequence
  Lysosome-retention peptide
LKQETEVELYNEFPEPMKLDKN

SEQ ID NO: 524 - protein, 20 aa, Artificial Sequence
  Lysosome-retention peptide
RRRAVVKEPLLPPEDDTRDN

SEQ ID NO: 525 - protein, 6 aa, Artificial Sequence
  Lysosome-retention peptide
PGYRHV

SEQ ID NO: 526 - protein, 5 aa, Artificial Sequence
  Lysosome-retention peptide
  SITE 3..4: Xaa represents any amino acid residue
GYXXI

SEQ ID NO: 527 - protein, 17 aa, Artificial Sequence
  Lysosome-retention peptide
GTEKCVWGPSYWCQNME

SEQ ID NO: 528 - protein, 5 aa, Artificial Sequence
  Lysosome-retention peptide
  SITE 2..4: Xaa represents any amino acid residue
KXXXQ

SEQ ID NO: 529 - protein, 6 aa, Artificial Sequence
  Lysosome-retention peptide
EQEPLL

SEQ ID NO: 530 - protein, 4 aa, Artificial Sequence
  Lysosome-retention peptide
YPAF

SEQ ID NO: 531 - protein, 4 aa, Artificial Sequence
  Lysosome-retention peptide
YEMA

SEQ ID NO: 532 - protein, 11 aa, Artificial Sequence
  Lysosome-retention peptide
  SITE 3..4: Xaa represents any amino acid residue
GYXXVILFWCM

SEQ ID NO: 533 - protein, 10 aa, Artificial Sequence
  Lysosome-retention peptide
SLLKGRQGIY

SEQ ID NO: 534 - protein, 6 aa, Artificial Sequence
  Lysosome-retention peptide
ETERLL

SEQ ID NO: 535 - protein, 6 aa, Artificial Sequence
  Lysosome-retention peptide
EEHSLL

SEQ ID NO: 536 - protein, 8 aa, Artificial Sequence
  Lysosome-retention peptide
DDSDEDLL

SEQ ID NO: 537 - protein, 6 aa, Artificial Sequence
  Lysosome-retention peptide
EQERLL

SEQ ID NO: 538 - protein, 8 aa, Artificial Sequence
  Lysosome-retention peptide
CINCCKVL

SEQ ID NO: 539 - protein, 18 aa, Artificial Sequence
  Peroxisome-retention peptide
  SITE 2..7: Xaa represents any amino acid residue
  SITE 10: Xaa represents any amino acid residue
KXXXXXXILXLFILKLFI

SEQ ID NO: 540 - protein, 20 aa, Artificial Sequence
  Peroxisome-retention peptide
KVFFSRLIQILKIMVPRTFC

SEQ ID NO: 541 - protein, 20 aa, Artificial Sequence
  Peroxisome-retention peptide
MTVPYLNSNRNVASYLQSNS

SEQ ID NO: 542 - protein, 9 aa, Artificial Sequence
  Peroxisome-retention peptide
  SITE 3..7: Xaa represents any amino acid residue
SVXXXXXQL

SEQ ID NO: 543 - protein, 20 aa, Artificial Sequence
  Peroxisome-retention peptide
MNGDQNSDVYAQEKQDFVQH

SEQ ID NO: 544 - protein, 26 aa, Artificial Sequence
  Peroxisome-retention peptide
MHRLQVVLGHLAGRPESSSALQAAPC

SEQ ID NO: 545 - protein, 15 aa, Artificial Sequence
  Peroxisome-retention peptide
EALREALQHCPKNKL

SEQ ID NO: 546 - protein, 7 aa, Artificial Sequence
  Peroxisome-retention peptide
LSSNSKF

SEQ ID NO: 547 - protein, 25 aa, Artificial Sequence
  Peroxisome-retention peptide
GFATKFYTEEGNLDWVYNNTPVFFI

SEQ ID NO: 548 - protein, 16 aa, Artificial Sequence
  Peroxisome-retention peptide
MERLRQIASQATAASA

SEQ ID NO: 549 - protein, 4 aa, Artificial Sequence
  Peroxisome-retention peptide
KANL

SEQ ID NO: 550 - protein, 4 aa, Artificial Sequence
  Peroxisome-retention peptide
RPSI

SEQ ID NO: 551 - protein, 4 aa, Artificial Sequence
  Peroxisome-retention peptide
APKI

SEQ ID NO: 552 - protein, 29 aa, Artificial Sequence
  Secretory vesicle-retention peptide
MKELTMLSLSFFFVSALLAAAANPLLSAA

SEQ ID NO: 553 - protein, 48 aa, Artificial Sequence
  Secretory vesicle-retention peptide
MRISKPHLRSISIQCYLCLLLNSHFLTEAGIHVFILGCFSAGLPKTEA

SEQ ID NO: 554 - protein, 55 aa, Artificial Sequence
  Secretory vesicle-retention peptide
MNKIYSLKYSHITGGLIAVSELSGRVSSRATGKKKHKRILALCFLGLLQSSYSFA

SEQ ID NO: 555 - protein, 27 aa, Artificial Sequence
  Secretory vesicle-retention peptide
MARQGCLGSFQVISLFTFAISVNICLG

SEQ ID NO: 556 - protein, 27 aa, Artificial Sequence
  Secretory vesicle-retention peptide
MQRARPTLWAAALTLLVLLRGPPVARA

SEQ ID NO: 557 - protein, 20 aa, Artificial Sequence
  Secretory vesicle-retention peptide
MKSVYFVAGLFIMLAQGSWQ

SEQ ID NO: 558 - protein, 29 aa, Artificial Sequence
  Secretory vesicle-retention peptide
MNIKKFAKQATVLTFTTALLAGGATQAFA

SEQ ID NO: 559 - protein, 49 aa, Artificial Sequence
  Secretory vesicle-retention peptide
MQQDGTQQDRIKQSPAPLNGMSRRGFLGGAGTLALATASGLLLPGTAHA

SEQ ID NO: 560 - protein, 41 aa, Artificial Sequence
  Secretory vesicle-retention peptide
MGSYALPRSGVRRSIRVLLLALVVGVLGTATALIAPPGAHA

SEQ ID NO: 561 - protein, 56 aa, Artificial Sequence
  Secretory vesicle-retention peptide
MNKVFKVIWCKTSQTWIAVSELSKAFSLSTTTDIPKKTKIFIAAAPLLFLSFNTNA

SEQ ID NO: 562 - protein, 13 aa, Artificial Sequence
  Secretory vesicle-retention peptide
SDPRLRQFLQKSK

SEQ ID NO: 563 - protein, 13 aa, Artificial Sequence
  Secretory vesicle-retention peptide
CQDLTTESNLLAC

SEQ ID NO: 564 - protein, 16 aa, Artificial Sequence
  Secretory vesicle-retention peptide
LPTDTTTFKRIFLKRM

SEQ ID NO: 565 - protein, 32 aa, Artificial Sequence
  Plasma membrane-retention peptide
MRVKEKYQHLWRWGWKWGTMLLGILMICSATE

SEQ ID NO: 566 - protein, 4 aa, Artificial Sequence
  Plasma membrane-retention peptide
CAAL

SEQ ID NO: 567 - protein, 6 aa, Artificial Sequence
  Plasma membrane-retention peptide
KKKKKK

SEQ ID NO: 568 - protein, 5 aa, Artificial Sequence
  Plasma membrane-retention peptide
RRRRR

SEQ ID NO: 569 - protein, 21 aa, Artificial Sequence
  Plasma membrane-retention peptide
MRLLLALLGVLLSVPGPPVLS

SEQ ID NO: 570 - protein, 12 aa, Artificial Sequence
  Plasma membrane-retention peptide
CSIMNLMCGSTC

SEQ ID NO: 571 - protein, 45 aa, Artificial Sequence
  Plasma membrane-retention peptide
GHKSEEKREKMKRTLLKDWKTRLSYFLQNSSTPGKPKTGKKSKQQ

SEQ ID NO: 572 - protein, 18 aa, Artificial Sequence
  Plasma membrane-retention peptide
RSTLKLTTLQCQYSTVMD

SEQ ID NO: 573 - protein, 18 aa, Artificial Sequence
  Plasma membrane-retention peptide
RQGLHNMEDVYELIENSH

SEQ ID NO: 574 - protein, 30 aa, Artificial Sequence
  Plasma membrane-retention peptide
MDCRKMARFSYSVIWIMAISKVFELGLVAG

SEQ ID NO: 575 - protein, 16 aa, Artificial Sequence
  Plasma membrane-retention peptide
MPAWGALFLLWATAEA

SEQ ID NO: 576 - protein, 4 aa, Artificial Sequence
  Plasma membrane-retention peptide
RDYR

SEQ ID NO: 577 - protein, 55 aa, Artificial Sequence
  Plasma membrane-retention peptide
KMALRVALNNKQSGQITVKTSSSDHLSLAIAGLVPIALSIYQKFKPGVSPSYSIY

SEQ ID NO: 578 - protein, 21 aa, Artificial Sequence
  Plasma membrane-retention peptide
MGSKIVQVFLMLALFATSALA

SEQ ID NO: 579 - protein, 21 aa, Artificial Sequence
  Plasma membrane-retention peptide
MNSKAMQALIFLGFLATSCLA

SEQ ID NO: 580 - protein, 12 aa, Artificial Sequence
  Plasma membrane-retention peptide
MGAAASIQTTVN

SEQ ID NO: 581 - protein, 11 aa, Artificial Sequence
  Plasma membrane-retention peptide
FALLGTHGASG

SEQ ID NO: 582 - protein, 7 aa, Artificial Sequence
  Plasma membrane-retention peptide
RRRTFLK

SEQ ID NO: 583 - protein, 10 aa, Artificial Sequence
  Plasma membrane-retention peptide
MGGKWSKSSV

SEQ ID NO: 584 - protein, 6 aa, Artificial Sequence
  Plasma membrane-retention peptide
DDPERE

SEQ ID NO: 585 - protein, 16 aa, Artificial Sequence
  Plasma membrane-retention peptide
EEANTGENNSLLHPMS

SEQ ID NO: 586 - protein, 6 aa, Artificial Sequence
  Plasma membrane-retention peptide
SRRGLV

SEQ ID NO: 587 - protein, 6 aa, Artificial Sequence
  Plasma membrane-retention peptide
SRRRFL

SEQ ID NO: 588 - protein, 6 aa, Artificial Sequence
  Plasma membrane-retention peptide
SRRQFI

SEQ ID NO: 589 - protein, 6 aa, Artificial Sequence
  Plasma membrane-retention peptide
QRRDFL

SEQ ID NO: 590 - protein, 52 aa, Artificial Sequence
  Plasma membrane-retention peptide
MNKIYSIKYSAATGGLIAVSELAKKVICKTNRKISAALLSLAVISYTNIIYA

SEQ ID NO: 591 - protein, 40 aa, Artificial Sequence
  Plasma membrane-retention peptide
MNPNQKIITIGSICMVIGIVSLMLQIGNIISIWVSHSIQT

SEQ ID NO: 592 - protein, 16 aa, Artificial Sequence
  Plasma membrane-retention peptide
LRCLACSCFRTPVWPR

SEQ ID NO: 593 - protein, 10 aa, Artificial Sequence
  Plasma membrane-retention peptide
MGCGCSSHPE

SEQ ID NO: 594 - protein, 9 aa, Artificial Sequence
  Plasma membrane-retention peptide
MPFVNKQFN

SEQ ID NO: 595 - protein, 28 aa, Artificial Sequence
  Plasma membrane-retention peptide
DEQNAKNAAQDRNSNKSSKGFFSKLGCC

SEQ ID NO: 596 - protein, 10 aa, Artificial Sequence
  Plasma membrane-retention peptide
MLCCMRRTKQ

SEQ ID NO: 597 - protein, 14 aa, Artificial Sequence
  Plasma membrane-retention peptide
VTNGSTYILVPLSH

SEQ ID NO: 598 - protein, 7 aa, Artificial Sequence
  Plasma membrane-retention peptide
AETENFV

SEQ ID NO: 599 - protein, 18 aa, Artificial Sequence
  Plasma membrane-retention peptide
RARHRRNVDRVSIGSYRT

SEQ ID NO: 600 - protein, 4 aa, Artificial Sequence
  Plasma membrane-retention peptide
YEDQ

SEQ ID NO: 601 - protein, 19 aa, Artificial Sequence
  Plasma membrane-retention peptide
LLVTSLLLCELPHPAFLIP

SEQ ID NO: 602 - protein, 22 aa, Artificial Sequence
  Signal peptide of tissue plasminogen activator
MDAMKRGLCCVLLLCGAVFVSP

SEQ ID NO: 603 - protein, 23 aa, Artificial Sequence
  Signal peptide of CCL5
MKVSAAALAVILIATALCAPASA

SEQ ID NO: 604 - protein, 11 aa, Artificial Sequence
  HiBit tag
VSGWRLFKKIS

SEQ ID NO: 605 - protein, 37 aa, Artificial Sequence
  Biotin-binding protein-binding peptide
DEKTTGWRGGHVVEGLAGELEQLRARLEHHPQGQREP

SEQ ID NO: 606 - protein, 9 aa, Artificial Sequence
  Biotin-binding protein-binding peptide
AWRHPQFGG

SEQ ID NO: 607 - protein, 18 aa, Artificial Sequence
  Biotin-binding protein-binding peptide
AWRHPQFGGAWRHPQFGG

SEQ ID NO: 608 - protein, 27 aa, Artificial Sequence
  Biotin-binding protein-binding peptide
AWRHPQFGGAWRHPQFGGAWRHPQFGG

SEQ ID NO: 609 - protein, 8 aa, Artificial Sequence
  Biotin-binding protein-binding peptide
WSHPQFEK

SEQ ID NO: 610 - protein, 16 aa, Artificial Sequence
  Biotin-binding protein-binding peptide
WSHPQFEKWSHPQFEK

SEQ ID NO: 611 - protein, 24 aa, Artificial Sequence
  Biotin-binding protein-binding peptide
WSHPQFEKWSHPQFEKWSHPQFEK

SEQ ID NO: 612 - protein, 8 aa, Artificial Sequence
  Biotin-binding protein-binding peptide
WSGPQFEK

SEQ ID NO: 613 - protein, 16 aa, Artificial Sequence
  Biotin-binding protein-binding peptide
WSGPQFEKWSGPQFEK

SEQ ID NO: 614 - protein, 24 aa, Artificial Sequence
  Biotin-binding protein-binding peptide
WSGPQFEKWSGPQFEKWSGPQFEK

SEQ ID NO: 615 - protein, 10 aa, Artificial Sequence
  Biotin-binding protein-binding peptide
WHRWPWLVSG

SEQ ID NO: 616 - protein, 20 aa, Artificial Sequence
  Biotin-binding protein-binding peptide
WHRWPWLVSGWHRWPWLVSG

SEQ ID NO: 617 - protein, 30 aa, Artificial Sequence
  Biotin-binding protein-binding peptide
WHRWPWLVSGWHRWPWLVSGWHRWPWLVSG

SEQ ID NO: 618 - protein, 10 aa, Artificial Sequence
  Biotin-binding protein-binding peptide
WHWWYWALDR

SEQ ID NO: 619 - protein, 20 aa, Artificial Sequence
  Biotin-binding protein-binding peptide
WHWWYWALDRWHWWYWALDR

SEQ ID NO: 620 - protein, 30 aa, Artificial Sequence
  Biotin-binding protein-binding peptide
WHWWYWALDRWHWWYWALDRWHWWYWALDR

SEQ ID NO: 621 - protein, 8 aa, Artificial Sequence
  Biotin-binding protein-binding peptide
  SITE 2: Xaa represents any amino acid residue
  SITE 7: Xaa represents G when Xaa at position 8 represents G; or Xaa represents E when Xaa at position 8 represents R or K
  SITE 8: Xaa represents G when Xaa at position 7 represents G; or Xaa represents R or K when Xaa at position 7 represents E
WXHPQFXX

SEQ ID NO: 622 - protein, 4 aa, Artificial Sequence
  Biotin-binding protein-binding peptide
VEAW

SEQ ID NO: 623 - protein, 4 aa, Artificial Sequence
  Biotin-binding protein-binding peptide
DVEA

SEQ ID NO: 624 - protein, 5 aa, Artificial Sequence
  Biotin-binding protein-binding peptide
DVEAW

SEQ ID NO: 625 - protein, 5 aa, Artificial Sequence
  Biotin-binding protein-binding peptide
  SITE 3: Xaa represents E, D or G
DVXAW

SEQ ID NO: 626 - protein, 6 aa, Artificial Sequence
  Biotin-binding protein-binding peptide
  SITE 3: Xaa represents E, D or G
DVXAWL

SEQ ID NO: 627 - protein, 6 aa, Artificial Sequence
  Biotin-binding protein-binding peptide
DVEAWL

SEQ ID NO: 628 - protein, 7 aa, Artificial Sequence
  Biotin-binding protein-binding peptide
  SITE 3: Xaa represents E, D or G
  SITE 7: Xaa represents G or D
DVXAWLX

SEQ ID NO: 629 - protein, 8 aa, Artificial Sequence
  Biotin-binding protein-binding peptide
  SITE 3: Xaa represents E, D or G
  SITE 7: Xaa represents G or D
  SITE 8: Xaa represents A or E
DVXAWLXX

SEQ ID NO: 630 - protein, 9 aa, Artificial Sequence
  Biotin-binding protein-binding peptide
  SITE 3: Xaa represents E, D or G
  SITE 7: Xaa represents G or D
  SITE 8: Xaa represents A or E
DVXAWLXXR

SEQ ID NO: 631 - protein, 9 aa, Artificial Sequence
  Biotin-binding protein-binding peptide
DVEAWLGAR

SEQ ID NO: 632 - protein, 15 aa, Artificial Sequence
  Biotin-binding protein-binding peptide
  SITE 3: Xaa represents E, D or G
  SITE 7: Xaa represents G or D
  SITE 8: Xaa represents A or E
DVXAWLXXRVPLVET

SEQ ID NO: 633 - protein, 6 aa, Artificial Sequence
  Biotin-binding protein-binding peptide
DRATPY

SEQ ID NO: 634 - protein, 8 aa, Artificial Sequence
  Biotin-binding protein-binding peptide
SEKLSNTK

SEQ ID NO: 635 - protein, 108 aa, Homo sapiens
  FKBP1A
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 636 - DNA, 462 bp, Encephalomyocarditis virus
  Internal ribosome entry site
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataa

SEQ ID NO: 637 - DNA, 296 bp, Artificial Sequence
  Intervening sequence
gaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcg

SEQ ID NO: 638 - protein, 171 aa, Artificial Sequence
  NanoKAZ
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 639 - DNA, 408 bp, Artificial Sequence
  sFFv promoter
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 640 - DNA, 501 bp, Artificial Sequence
  PGK promoter
ttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 641 - DNA, 1178 bp, Artificial Sequence
  UbC promoter
ggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 642 - DNA, 344 bp, Artificial Sequence
  SV40 promoter
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct